SEQUENCE LISTING (294 sequences)

SEQ ID NO: 1 - protein, 5 aa, Artificial Sequence
  Description of Artificial Sequence: Synthetic peptide
GGGGS

SEQ ID NO: 2 - protein, 4 aa, Artificial Sequence
  Description of Artificial Sequence: Synthetic peptide
GGSG

SEQ ID NO: 3 - protein, 4 aa, Artificial Sequence
  Description of Artificial Sequence: Synthetic peptide
SGGG

SEQ ID NO: 4 - protein, 4 aa, Artificial Sequence
  Description of Artificial Sequence: Synthetic peptide
GSGS

SEQ ID NO: 5 - protein, 6 aa, Artificial Sequence
  Description of Artificial Sequence: Synthetic peptide
GSGSGS

SEQ ID NO: 6 - protein, 8 aa, Artificial Sequence
  Description of Artificial Sequence: Synthetic peptide
GSGSGSGS

SEQ ID NO: 7 - protein, 10 aa, Artificial Sequence
  Description of Artificial Sequence: Synthetic peptide
GSGSGSGSGS

SEQ ID NO: 8 - protein, 12 aa, Artificial Sequence
  Description of Artificial Sequence: Synthetic peptide
GSGSGSGSGSGS

SEQ ID NO: 9 - protein, 6 aa, Artificial Sequence
  Description of Artificial Sequence: Synthetic peptide
GGSGGS

SEQ ID NO: 10 - protein, 9 aa, Artificial Sequence
  Description of Artificial Sequence: Synthetic peptide
GGSGGSGGS

SEQ ID NO: 11 - protein, 12 aa, Artificial Sequence
  Description of Artificial Sequence: Synthetic peptide
GGSGGSGGSGGS

SEQ ID NO: 12 - protein, 8 aa, Artificial Sequence
  Description of Artificial Sequence: Synthetic peptide
GGSGGGSG

SEQ ID NO: 13 - protein, 12 aa, Artificial Sequence
  Description of Artificial Sequence: Synthetic peptide
GGSGGGSGGGSG

SEQ ID NO: 14 - protein, 16 aa, Artificial Sequence
  Description of Artificial Sequence: Synthetic peptide
GGSGGGSGGGSGGGSG

SEQ ID NO: 15 - protein, 20 aa, Artificial Sequence
  Description of Artificial Sequence: Synthetic peptide
GGSGGGSGGGSGGGSGGGSG

SEQ ID NO: 16 - protein, 10 aa, Artificial Sequence
  Description of Artificial Sequence: Synthetic peptide
GGGGSGGGGS

SEQ ID NO: 17 - protein, 15 aa, Artificial Sequence
  Description of Artificial Sequence: Synthetic peptide
GGGGSGGGGSGGGGS

SEQ ID NO: 18 - protein, 20 aa, Artificial Sequence
  Description of Artificial Sequence: Synthetic peptide
SGGGSGGGSGGGSGGGSGGG

SEQ ID NO: 19 - protein, 4 aa, Artificial Sequence
  Description of Artificial Sequence: Synthetic peptide
GGGG

SEQ ID NO: 20 - protein, 8 aa, Artificial Sequence
  Description of Artificial Sequence: Synthetic peptide
GGGGGGGG

SEQ ID NO: 21 - protein, 12 aa, Artificial Sequence
  Description of Artificial Sequence: Synthetic peptide
GGGGGGGGGGGG

SEQ ID NO: 22 - protein, 16 aa, Artificial Sequence
  Description of Artificial Sequence: Synthetic peptide
GGGGGGGGGGGGGGGG

SEQ ID NO: 23 - protein, 20 aa, Artificial Sequence
  Description of Artificial Sequence: Synthetic peptide
GGGGGGGGGGGGGGGGGGGG

SEQ ID NO: 24 - protein, 5 aa, Artificial Sequence
  Description of Artificial Sequence: Synthetic peptide
GGGGG

SEQ ID NO: 25 - protein, 10 aa, Artificial Sequence
  Description of Artificial Sequence: Synthetic peptide
GGGGGGGGGG

SEQ ID NO: 26 - protein, 15 aa, Artificial Sequence
  Description of Artificial Sequence: Synthetic peptide
GGGGGGGGGGGGGGG

SEQ ID NO: 27 - protein, 20 aa, Artificial Sequence
  Description of Artificial Sequence: Synthetic peptide
GGGGGGGGGGGGGGGGGGGG

SEQ ID NO: 28 - protein, 10 aa, Artificial Sequence
  Description of Artificial Sequence: Synthetic peptide
GENLYFQSGG

SEQ ID NO: 29 - protein, 8 aa, Artificial Sequence
  Description of Artificial Sequence: Synthetic peptide
SACYCELS

SEQ ID NO: 30 - protein, 5 aa, Artificial Sequence
  Description of Artificial Sequence: Synthetic peptide
RSIAT

SEQ ID NO: 31 - protein, 17 aa, Artificial Sequence
  Description of Artificial Sequence: Synthetic peptide
RPACKIPNDLKQKVMNH

SEQ ID NO: 32 - protein, 36 aa, Artificial Sequence
  Description of Artificial Sequence: Synthetic polypeptide
GGSAGGSGSGSSGGSSGASGTGTAGGTGSGSGTGSG

SEQ ID NO: 33 - protein, 17 aa, Artificial Sequence
  Description of Artificial Sequence: Synthetic peptide
AAANSSIDLISVPVDSR

SEQ ID NO: 34 - protein, 36 aa, Artificial Sequence
  Description of Artificial Sequence: Synthetic polypeptide
GGSGGGSEGGGSEGGGSEGGGSEGGGSEGGGSGGGS

SEQ ID NO: 35 - protein, 12 aa, Artificial Sequence
  Description of Artificial Sequence: Synthetic peptide
GGGSGGGSGGGS

SEQ ID NO: 36 - protein, 18 aa, Artificial Sequence
  Description of Artificial Sequence: Synthetic peptide
GGSGGGSGGGSGGGSGGS

SEQ ID NO: 37 - protein, 11 aa, Unknown
  Description of Unknown: albumin binding peptide
DICLPRWGCLW

SEQ ID NO: 38 - protein, 6 aa, Artificial Sequence
  Description of Artificial Sequence: Synthetic 6xHis tag
HHHHHH

SEQ ID NO: 39 - protein, 8 aa, Artificial Sequence
  Description of Artificial Sequence: Synthetic peptide
DYKDDDDK

SEQ ID NO: 40 - protein, 10 aa, Artificial Sequence
  Description of Artificial Sequence: Synthetic peptide
EQKLISEEDL

SEQ ID NO: 41 - protein, 9 aa, Artificial Sequence
  Description of Artificial Sequence: Synthetic peptide
YPYDVPDYA

SEQ ID NO: 42 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 232 aa, Artificial Sequence
  Description of Artificial Sequence: Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 45 - protein, 226 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 226 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 47 - protein, 231 aa, Artificial Sequence
  Description of Artificial Sequence: Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 226 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 49 - skipped

SEQ ID NO: 50 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGK

SEQ ID NO: 52 - protein, 226 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 53 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54-62 - skipped (9 entries)

SEQ ID NO: 63 - protein, 226 aa, Artificial Sequence; identical to SEQ ID NO: 48
  Description of Artificial Sequence: Synthetic polypeptide

SEQ ID NO: 64-70 - skipped (7 entries)

SEQ ID NO: 71 - protein, 945 aa, Artificial Sequence
  Description of Artificial Sequence: Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - skipped

SEQ ID NO: 73 - protein, 945 aa, Artificial Sequence
  Description of Artificial Sequence: Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 74-84 - skipped (11 entries)

SEQ ID NO: 85 - protein, 8 aa, Artificial Sequence
  Description of Artificial Sequence: Synthetic peptide
GFTFNSFA

SEQ ID NO: 86 - protein, 8 aa, Artificial Sequence
  Description of Artificial Sequence: Synthetic peptide
GYTFTDYW

SEQ ID NO: 87-108 - skipped (22 entries)

SEQ ID NO: 109 - protein, 8 aa, Artificial Sequence
  Description of Artificial Sequence: Synthetic peptide
IYPGDGDT

SEQ ID NO: 110-114 - skipped (5 entries)

SEQ ID NO: 115 - protein, 8 aa, Artificial Sequence
  Description of Artificial Sequence: Synthetic peptide
ISGSGGGT

SEQ ID NO: 116-147 - skipped (32 entries)

SEQ ID NO: 148 - protein, 15 aa, Artificial Sequence
  Description of Artificial Sequence: Synthetic peptide
AKDKILWFGEPVFDY

SEQ ID NO: 149 - protein, 13 aa, Artificial Sequence
  Description of Artificial Sequence: Synthetic peptide
ARGDYYGSNSLDY

SEQ ID NO: 150-179 - skipped (30 entries)

SEQ ID NO: 180 - protein, 6 aa, Artificial Sequence
  Description of Artificial Sequence: Synthetic peptide
QSVSSY

SEQ ID NO: 181 - protein, 6 aa, Artificial Sequence
  Description of Artificial Sequence: Synthetic peptide
QDVSTV

SEQ ID NO: 182-210 - skipped (29 entries)

SEQ ID NO: 211 - protein, 9 aa, Artificial Sequence
  Description of Artificial Sequence: Synthetic peptide
QQRSNWPPT

SEQ ID NO: 212 - protein, 9 aa, Artificial Sequence
  Description of Artificial Sequence: Synthetic peptide
QQHYSPPYT

SEQ ID NO: 213 - protein, 32 aa, Artificial Sequence
  Description of Artificial Sequence: Synthetic polypeptide
GGSGGGSGGGSGGGGGSGGGGSGGGGSGGGGS

SEQ ID NO: 214 - protein, 30 aa, Artificial Sequence
  Description of Artificial Sequence: Synthetic polypeptide
  MISC_FEATURE (1)..(30): This sequence may encompass 4-30 residues
GGGGGGGGGGGGGGGGGGGGGGGGGGGGGG

SEQ ID NO: 215 - protein, 30 aa, Artificial Sequence; identical to SEQ ID NO: 214
  Description of Artificial Sequence: Synthetic polypeptide
  MISC_FEATURE (1)..(30): This sequence may encompass 8-30 residues

SEQ ID NO: 216 - protein, 30 aa, Artificial Sequence; identical to SEQ ID NO: 214
  Description of Artificial Sequence: Synthetic polypeptide
  MISC_FEATURE (1)..(30): This sequence may encompass 12-30 residues

SEQ ID NO: 217 - protein, 20 aa, Artificial Sequence
  Description of Artificial Sequence: Synthetic peptide
  MISC_FEATURE (1)..(20): This sequence may encompass 4-20 residues
GGGGGGGGGGGGGGGGGGGG

SEQ ID NO: 218 - protein, 20 aa, Artificial Sequence
  Description of Artificial Sequence: Synthetic peptide
  MISC_FEATURE (1)..(20): This sequence may encompass 8-20 residues
GGGGGGGGGGGGGGGGGGGG

SEQ ID NO: 219 - protein, 20 aa, Artificial Sequence
  Description of Artificial Sequence: Synthetic peptide
  MISC_FEATURE (1)..(20): This sequence may encompass 12-20 residues
GGGGGGGGGGGGGGGGGGGG

SEQ ID NO: 220 - protein, 20 aa, Artificial Sequence
  Description of Artificial Sequence: Synthetic peptide
EPKSCDKTHTCPPCPAPELL

SEQ ID NO: 221 - protein, 15 aa, Artificial Sequence
  Description of Artificial Sequence: Synthetic peptide
DKTHTCPPCPAPELL

SEQ ID NO: 222 - protein, 19 aa, Artificial Sequence
  Description of Artificial Sequence: Synthetic peptide
EPKSCDKTHTCPPCPAPEL

SEQ ID NO: 223 - protein, 105 aa, Artificial Sequence
  Description of Artificial Sequence: Synthetic polypeptide
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 224 - protein, 106 aa, Artificial Sequence [sequence omitted]

SEQ ID NO: 225 - protein, 5 aa, Artificial Sequence
  Description of Artificial Sequence: Synthetic peptide
SYYMN

SEQ ID NO: 226 - protein, 17 aa, Artificial Sequence
  Description of Artificial Sequence: Synthetic peptide
GISGDPSNTYYADSVKG

SEQ ID NO: 227 - protein, 11 aa, Artificial Sequence
  Description of Artificial Sequence: Synthetic peptide
DLPLVYTGFAY

SEQ ID NO: 228 - protein, 11 aa, Artificial Sequence
  Description of Artificial Sequence: Synthetic peptide
SGDNLRHYYVY

SEQ ID NO: 229 - protein, 7 aa, Artificial Sequence
  Description of Artificial Sequence: Synthetic peptide
GDSKRPS

SEQ ID NO: 230 - protein, 8 aa, Artificial Sequence
  Description of Artificial Sequence: Synthetic peptide
QTYTGGAS

SEQ ID NO: 231 - protein, 450 aa, Artificial Sequence
  Description of Artificial Sequence: Synthetic polypeptide
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - protein, 118 aa, Artificial Sequence
  Description of Artificial Sequence: Synthetic polypeptide
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTV

SEQ ID NO: 233 - protein, 214 aa, Artificial Sequence
  Description of Artificial Sequence: Synthetic polypeptide
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 234 - protein, 109 aa, Artificial Sequence
  Description of Artificial Sequence: Synthetic polypeptide
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 235 - protein, 200 aa, Artificial Sequence
  Description of Artificial Sequence: Synthetic polypeptide
  MISC_FEATURE (1)..(200): This sequence may encompass 4-200 residues
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 236 - protein, 180 aa, Artificial Sequence
  Description of Artificial Sequence: Synthetic polypeptide
  MISC_FEATURE (1)..(180): This sequence may encompass 4-180 residues
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 237 - protein, 160 aa, Artificial Sequence
  Description of Artificial Sequence: Synthetic polypeptide
  MISC_FEATURE (1)..(160): This sequence may encompass 4-160 residues
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 238 - protein, 140 aa, Artificial Sequence
  Description of Artificial Sequence: Synthetic polypeptide
  MISC_FEATURE (1)..(140): This sequence may encompass 4-140 residues
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 239 - protein, 40 aa, Artificial Sequence
  Description of Artificial Sequence: Synthetic polypeptide
  MISC_FEATURE (1)..(40): This sequence may encompass 4-40 residues
GGGGGGGGGGGGGGGGGGGGGGGGGGGGGGGGGGGGGGGG

SEQ ID NO: 240 - protein, 100 aa, Artificial Sequence
  Description of Artificial Sequence: Synthetic polypeptide
  MISC_FEATURE (1)..(100): This sequence may encompass 4-100 residues
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 241 - protein, 90 aa, Artificial Sequence
  Description of Artificial Sequence: Synthetic polypeptide
  MISC_FEATURE (1)..(90): This sequence may encompass 4-90 residues
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 242 - protein, 80 aa, Artificial Sequence
  Description of Artificial Sequence: Synthetic polypeptide
  MISC_FEATURE (1)..(80): This sequence may encompass 4-80 residues
GGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 243 - protein, 70 aa, Artificial Sequence
  Description of Artificial Sequence: Synthetic polypeptide
  MISC_FEATURE (1)..(70): This sequence may encompass 4-70 residues
GGGGGGGGGGGGGGGGGGGGGGGGGGGGGGGGGGGGGGGGGGGGGGGGGGGGGGGGGGGGGGGGGGGGGG

SEQ ID NO: 244 - protein, 60 aa, Artificial Sequence
  Description of Artificial Sequence: Synthetic polypeptide
  MISC_FEATURE (1)..(60): This sequence may encompass 4-60 residues
GGGGGGGGGGGGGGGGGGGGGGGGGGGGGGGGGGGGGGGGGGGGGGGGGGGGGGGGGGGG

SEQ ID NO: 245 - protein, 50 aa, Artificial Sequence
  Description of Artificial Sequence: Synthetic polypeptide
  MISC_FEATURE (1)..(50): This sequence may encompass 4-50 residues
GGGGGGGGGGGGGGGGGGGGGGGGGGGGGGGGGGGGGGGGGGGGGGGGGG

SEQ ID NO: 246 - protein, 19 aa, Artificial Sequence
  Description of Artificial Sequence: Synthetic peptide
  MISC_FEATURE (1)..(19): This sequence may encompass 4-19 residues
GGGGGGGGGGGGGGGGGGG

SEQ ID NO: 247 - protein, 18 aa, Artificial Sequence
  Description of Artificial Sequence: Synthetic peptide
  MISC_FEATURE (1)..(18): This sequence may encompass 4-18 residues
GGGGGGGGGGGGGGGGGG

SEQ ID NO: 248 - protein, 17 aa, Artificial Sequence
  Description of Artificial Sequence: Synthetic peptide
  MISC_FEATURE (1)..(17): This sequence may encompass 4-17 residues
GGGGGGGGGGGGGGGGG

SEQ ID NO: 249 - protein, 16 aa, Artificial Sequence
  Description of Artificial Sequence: Synthetic peptide
  MISC_FEATURE (1)..(16): This sequence may encompass 4-16 residues
GGGGGGGGGGGGGGGG

SEQ ID NO: 250 - protein, 15 aa, Artificial Sequence
  Description of Artificial Sequence: Synthetic peptide
  MISC_FEATURE (1)..(15): This sequence may encompass 4-15 residues
GGGGGGGGGGGGGGG

SEQ ID NO: 251 - protein, 14 aa, Artificial Sequence
  Description of Artificial Sequence: Synthetic peptide
  MISC_FEATURE (1)..(14): This sequence may encompass 4-14 residues
GGGGGGGGGGGGGG

SEQ ID NO: 252 - protein, 13 aa, Artificial Sequence
  Description of Artificial Sequence: Synthetic peptide
  MISC_FEATURE (1)..(13): This sequence may encompass 4-13 residues
GGGGGGGGGGGGG

SEQ ID NO: 253 - protein, 12 aa, Artificial Sequence
  Description of Artificial Sequence: Synthetic peptide
  MISC_FEATURE (1)..(12): This sequence may encompass 4-12 residues
GGGGGGGGGGGG

SEQ ID NO: 254 - protein, 11 aa, Artificial Sequence
  Description of Artificial Sequence: Synthetic peptide
  MISC_FEATURE (1)..(11): This sequence may encompass 4-11 residues
GGGGGGGGGGG

SEQ ID NO: 255 - protein, 10 aa, Artificial Sequence
  Description of Artificial Sequence: Synthetic peptide
  MISC_FEATURE (1)..(10): This sequence may encompass 4-10 residues
GGGGGGGGGG

SEQ ID NO: 256 - protein, 9 aa, Artificial Sequence
  Description of Artificial Sequence: Synthetic peptide
  MISC_FEATURE (1)..(9): This sequence may encompass 4-9 residues
GGGGGGGGG

SEQ ID NO: 257 - protein, 8 aa, Artificial Sequence
  Description of Artificial Sequence: Synthetic peptide
  MISC_FEATURE (1)..(8): This sequence may encompass 4-8 residues
GGGGGGGG

SEQ ID NO: 258 - protein, 7 aa, Artificial Sequence
  Description of Artificial Sequence: Synthetic peptide
  MISC_FEATURE (1)..(7): This sequence may encompass 4-7 residues
GGGGGGG

SEQ ID NO: 259 - protein, 6 aa, Artificial Sequence
  Description of Artificial Sequence: Synthetic peptide
  MISC_FEATURE (1)..(6): This sequence may encompass 4-6 residues
GGGGGG

SEQ ID NO: 260 - protein, 5 aa, Artificial Sequence
  Description of Artificial Sequence: Synthetic peptide
  MISC_FEATURE (1)..(5): This sequence may encompass 4-5 residues
GGGGG

SEQ ID NO: 261 - protein, 200 aa, Artificial Sequence; identical to SEQ ID NO: 235
  Description of Artificial Sequence: Synthetic polypeptide
  MISC_FEATURE (1)..(200): This sequence may encompass 6-200 residues

SEQ ID NO: 262 - protein, 200 aa, Artificial Sequence; identical to SEQ ID NO: 235
  Description of Artificial Sequence: Synthetic polypeptide
  MISC_FEATURE (1)..(200): This sequence may encompass 8-200 residues

SEQ ID NO: 263 - protein, 200 aa, Artificial Sequence; identical to SEQ ID NO: 235
  Description of Artificial Sequence: Synthetic polypeptide
  MISC_FEATURE (1)..(200): This sequence may encompass 10-200 residues

SEQ ID NO: 264 - protein, 200 aa, Artificial Sequence; identical to SEQ ID NO: 235
  Description of Artificial Sequence: Synthetic polypeptide
  MISC_FEATURE (1)..(200): This sequence may encompass 12-200 residues

SEQ ID NO: 265 - protein, 200 aa, Artificial Sequence; identical to SEQ ID NO: 235
  Description of Artificial Sequence: Synthetic polypeptide
  MISC_FEATURE (1)..(200): This sequence may encompass 14-200 residues

SEQ ID NO: 266 - protein, 200 aa, Artificial Sequence; identical to SEQ ID NO: 235
  Description of Artificial Sequence: Synthetic polypeptide
  MISC_FEATURE (1)..(200): This sequence may encompass 16-200 residues

SEQ ID NO: 267 - protein, 200 aa, Artificial Sequence; identical to SEQ ID NO: 235
  Description of Artificial Sequence: Synthetic polypeptide
  MISC_FEATURE (1)..(200): This sequence may encompass 18-200 residues

SEQ ID NO: 268 - protein, 200 aa, Artificial Sequence; identical to SEQ ID NO: 235
  Description of Artificial Sequence: Synthetic polypeptide
  MISC_FEATURE (1)..(200): This sequence may encompass 20-200 residues

SEQ ID NO: 269 - protein, 200 aa, Artificial Sequence; identical to SEQ ID NO: 235
  Description of Artificial Sequence: Synthetic polypeptide
  MISC_FEATURE (1)..(200): This sequence may encompass 30-200 residues

SEQ ID NO: 270 - protein, 200 aa, Artificial Sequence; identical to SEQ ID NO: 235
  Description of Artificial Sequence: Synthetic polypeptide
  MISC_FEATURE (1)..(200): This sequence may encompass 40-200 residues

SEQ ID NO: 271 - protein, 200 aa, Artificial Sequence; identical to SEQ ID NO: 235
  Description of Artificial Sequence: Synthetic polypeptide
  MISC_FEATURE (1)..(200): This sequence may encompass 50-200 residues

SEQ ID NO: 272 - protein, 200 aa, Artificial Sequence; identical to SEQ ID NO: 235
  Description of Artificial Sequence: Synthetic polypeptide
  MISC_FEATURE (1)..(200): This sequence may encompass 60-200 residues

SEQ ID NO: 273 - protein, 200 aa, Artificial Sequence; identical to SEQ ID NO: 235
  Description of Artificial Sequence: Synthetic polypeptide
  MISC_FEATURE (1)..(200): This sequence may encompass 70-200 residues

SEQ ID NO: 274 - protein, 200 aa, Artificial Sequence; identical to SEQ ID NO: 235
  Description of Artificial Sequence: Synthetic polypeptide
  MISC_FEATURE (1)..(200): This sequence may encompass 80-200 residues

SEQ ID NO: 275 - protein, 200 aa, Artificial Sequence; identical to SEQ ID NO: 235
  Description of Artificial Sequence: Synthetic polypeptide
  MISC_FEATURE (1)..(200): This sequence may encompass 90-200 residues

SEQ ID NO: 276 - protein, 200 aa, Artificial Sequence; identical to SEQ ID NO: 235
  Description of Artificial Sequence: Synthetic polypeptide
  MISC_FEATURE (1)..(200): This sequence may encompass 100-200 residues

SEQ ID NO: 277 - protein, 200 aa, Artificial Sequence; identical to SEQ ID NO: 235
  Description of Artificial Sequence: Synthetic polypeptide
  MISC_FEATURE (1)..(200): This sequence may encompass 120-200 residues

SEQ ID NO: 278 - protein, 200 aa, Artificial Sequence; identical to SEQ ID NO: 235
  Description of Artificial Sequence: Synthetic polypeptide
  MISC_FEATURE (1)..(200): This sequence may encompass 140-200 residues

SEQ ID NO: 279 - protein, 200 aa, Artificial Sequence; identical to SEQ ID NO: 235
  Description of Artificial Sequence: Synthetic polypeptide
  MISC_FEATURE (1)..(200): This sequence may encompass 160-200 residues

SEQ ID NO: 280 - protein, 200 aa, Artificial Sequence; identical to SEQ ID NO: 235
  Description of Artificial Sequence: Synthetic polypeptide
  MISC_FEATURE (1)..(200): This sequence may encompass 180-200 residues

SEQ ID NO: 281 - protein, 200 aa, Artificial Sequence; identical to SEQ ID NO: 235
  Description of Artificial Sequence: Synthetic polypeptide
  MISC_FEATURE (1)..(200): This sequence may encompass 190-200 residues

SEQ ID NO: 282 - protein, 24 aa, Artificial Sequence
  Description of Artificial Sequence: Synthetic peptide
DYKDDDDKDYKDDDDKDYKDDDDK

SEQ ID NO: 283 - protein, 30 aa, Artificial Sequence
  Description of Artificial Sequence: Synthetic polypeptide
EQKLISEEDLEQKLISEEDLEQKLISEEDL

SEQ ID NO: 284 - protein, 27 aa, Artificial Sequence
  Description of Artificial Sequence: Synthetic peptide
YPYDVPDYAYPYDVPDYAYPYDVPDYA

SEQ ID NO: 285 - protein, 214 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 286 - protein, 697 aa, Artificial Sequence
  Description of Artificial Sequence: Synthetic polypeptide
QLLESGGGLVQPGGSLRLSCAASGFTFDDYGMSWVRQAPGKGLEWVSDISWNGGKTHYVDSVKGQFTISRDNSKNTLYLQMNSLRAEDTAVYYCARGSLFHDSSGFYFGHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 287 - protein, 216 aa, Artificial Sequence
  Description of Artificial Sequence: Synthetic polypeptide
QSVLTQPPSASGTPGQRVTISCSGSSSNIGDNYVSWYQQLPGTAPKLLIYRDSQRPSGVPDRFSGSKSGTSASLAISGLRSEDEADYYCQSYDSSLSGSVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 288 - protein, 698 aa, Artificial Sequence
  Description of Artificial Sequence: Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 289 - protein, 697 aa, Artificial Sequence
  Description of Artificial Sequence: Synthetic polypeptide
QLLESGGGLVQPGGSLRLSCAASGFTFDDYGMSWVRQAPGKGLEWVSDISWNGGKTHYVDSVKGQFTISRDNSKNTLYLQMNSLRAEDTAVYYCARGSLFHDSSGFYFGHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 290 - protein, 720 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 291 - protein, 451 aa, Artificial Sequence
  Description of Artificial Sequence: Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 292 - protein, 473 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 293 - protein, 698 aa, Artificial Sequence
  Description of Artificial Sequence: Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 294 - protein, 945 aa, Artificial Sequence
  Description of Artificial Sequence: Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2021/0269546 A1
(43) Pub. Date: Sep. 2, 2021

(54) COMPOSITIONS AND METHODS RELATED TO ENGINEERED Fc-ANTIGEN BINDING DOMAIN CONSTRUCTS TARGETED TO CD38

(71) Applicant: Momenta Pharmaceuticals, Inc., Cambridge, MA (US)

(72) Inventors: Anthony Manning, Cambridge, MA (US); Amit Choudhury, Acton, MA (US); Daniel Ortiz, Stoneham, MA (US); Jonathan C. Lansing, Reading, MA (US)

(21) Appl. No.: 17/259,491

(22) PCT Filed: Jul. 11, 2019
(86) PCT No.: PCT/US19/41468

§ 371 Date: Jan. 11, 2021

Related U.S. Application Data
(60) Provisional application No. 62/696,759, filed on Jul. 11, 2018.
(60) Provisional application No. 62/733,036, filed on Sep. 18, 2018.
(60) Provisional application No. 62/744,067, filed on Oct. 10, 2018.

(51) Int. Cl. C07K 16/28; A61P 35/00

(52) U.S. Cl. CPC C07K 16/2896; A61P 35/00; A61K 2039/505; C07K 2317/526; C07K 2317/55; C07K 2317/524

(57) ABSTRACT

Fc-antigen binding constructs having a CD38 binding domain and two or more Fc domains are described as are methods for using such constructs. Also described are polypeptides making up such constructs. Fc domain monomers that are included in the constructs can include amino acid substitutions that promote homodimerization or heterodimerization.

### CROSS-REFERENCE TO RELATED APPLICATIONS

This application is a National Stage application under 35 U.S.C. § 371 of International Application No. PCT/US2019/041468, having an International Filing Date of Jul. 11, 2019, which claims priority to U.S. Application Ser. No. 62/744,067, filed on Oct. 10, 2018, U.S. Application Ser. No. 62/733,036, filed on Sep. 18, 2018, and to U.S. Application Ser. No. 62/696,759, filed on Jul. 11, 2018. The disclosure of the prior applications are considered part of the disclosure of this application, and are incorporated in their entirety into this application.

### SEQUENCE LISTING

The instant application contains a Sequence Listing which has been submitted electronically in ASCII format and is hereby incorporated by reference in its entirety. Said ASCII copy, created on Aug. 23, 2019, is named 14131-0189W01_SL.txt and is 186,505 bytes in size.

### SUMMARY

CD38 is a type II transmembrane glycoprotein expressed at high density on normal and malignant plasmablasts and plasma cells and at low levels on certain lymphoid and myeloid cells. Darzalex (daratumumab) is an anti-CD38 cytolytic monoclonal antibody approved for relapsed, refractory multiple myeloma and for newly-diagnosed multiple myeloma.

### SUMMARY OF THE DISCLOSURE

The present disclosure features compositions and methods for combining a CD38 binding domain with at least two Fc domains to generate new therapeutics with unique biological activity.

In some instances, the present disclosure contemplates combining a CD38 binding domain of a known CD38 targeted single Fc-domain containing therapeutic, e.g., a known therapeutic CD38 antibody, with at least two Fc domains to generate a novel therapeutic with a biological activity greater than that of a known CD38 antibody. To generate such constructs, the disclosure provides various methods for the assembly of constructs having at least two, e.g., multiple, Fc domains, and to control homodimerization and heterodimerization of such, to assemble molecules of discrete size from a limited number of polypeptides. The properties of these constructs allow for the efficient generation of substantially homogenous pharmaceutical compositions. Such homogeneity in a pharmaceutical composition is desirable in order to ensure the safety, efficacy, uniformity, and reliability of the pharmaceutical composition.

In a first aspect, the disclosure features an Fc-antigen binding domain construct including enhanced effector function, where the Fc-antigen binding domain construct includes a CD38 binding domain and a first Fc domain joined to a second Fc domain by a linker, where the Fc-antigen binding domain construct has enhanced effector function in an antibody-dependent cytotoxicity (ADCC) assay, an antibody-dependent cellular phagocytosis (ADCP), and/or complement-dependent cytotoxicity (CDC) assay relative to a construct having a single Fc domain and the CD38 binding domain.

In a second aspect, the disclosure features a composition including a substantially homogenous population of an Fc-antigen binding domain construct including a CD38 binding domain and a first Fc domain joined to a second Fc domain by a linker.

In a third aspect, the disclosure features an Fc-antigen binding domain construct including a CD38 binding domain and a first Fc domain joined to a second Fc domain by a linker, where the Fc-antigen binding domain construct includes a biological activity that is not exhibited by a construct having a single Fc domain and the CD38 binding domain.

In a fourth aspect, the disclosure features a composition including a substantially homogenous population of an Fc-antigen binding domain construct including a) a first polypeptide including i) a first Fc domain monomer, ii) a second Fc domain monomer, and iii) a linker joining the first Fc domain monomer and the second Fc domain monomer; b) a second polypeptide including a third Fc domain monomer; c) a third polypeptide including a fourth Fc domain monomer; and d) a CD38 binding domain joined to the first polypeptide, second polypeptide, or third polypeptide; where the first Fc domain monomer and the third Fc domain monomer combine to form a first Fc domain and the second Fc domain monomer and the fourth Fc domain monomer combine to form a second Fc domain.

In some embodiments of the fourth aspect, the CD38 binding domain is joined to the first polypeptide and the second polypeptide or the third polypeptide, or to the second polypeptide and the third polypeptide, or the CD38binding domain is joined to the first polypeptide, the second polypeptide, and the third polypeptide.

In a fifth aspect, the disclosure features an Fc-antigen binding domain construct including enhanced effector function, where the Fc-antigen binding domain construct includes: a) a first polypeptide including i) a first Fc domain monomer, ii) a second Fc domain monomer, and iii) a linker joining the first Fc domain monomer and the second Fc domain monomer; b) a second polypeptide including a third Fc domain monomer; c) a third polypeptide including a fourth Fc domain monomer; and d) a CD38 binding domain joined to the first polypeptide, second polypeptide, or third polypeptide; where the first Fc domain monomer and the third Fc domain monomer combine to form a first Fc domain and the second Fc domain monomer and the fourth Fc domain monomer combine to form a second Fc domain, and where the Fc-antigen binding domain construct has enhanced effector function in an antibody-dependent cytotoxicity (ADCC) assay, an antibody-dependent cellular phagocytosis (ADCP), and/or complement-dependent cytotoxicity (CDC) assay relative to a construct having a single Fc domain and the CD38 binding domain.

In some embodiments of the fifth aspect, the single Fc domain construct is an antibody. In a sixth aspect, the disclosure features an Fc-antigen binding domain construct including: a) a first polypeptide including i) a first Fc domain monomer, ii) a second Fc domain monomer, and iii) a linker joining the first Fc domain monomer and the second Fc domain monomer; b) a second polypeptide including a third Fc domain monomer; c) a third polypeptide including a fourth Fc domain monomer; and d) a CD38 binding domain joined to the first polypeptide, second polypeptide, or third polypeptide; where the first Fc domain monomer and the third Fc domain monomer combine to form a first Fc domain and the second Fc domain monomer and the fourth Fc domain monomer combine to form a second Fc domain, and where the Fc-antigen binding domain construct includes a biological activity that is not exhibited by a construct having a single Fc domain and the CD38 binding domain.

In some embodiments of the sixth aspect, the biological activity is an Fc receptor mediated effector function, such as ADCC, ADCP and/or CDC activity (e.g., ADCC and ADCP activity, ADCC and CDC activity, ADCP and CDC activity, or ADCC, ADCP, and CDC activity).

In a seventh aspect, the disclosure features an Fc-antigen binding domain construct including: a) a first polypeptide including: i) a first Fc domain monomer, ii) a second Fc domain monomer, and iii) a spacer joining the first Fc domain monomer and the second Fc domain monomer; b) a second polypeptide including a third Fc domain monomer; c) a third polypeptide including a fourth Fc domain monomer; and d) a CD38 binding domain joined to the first polypeptide, second polypeptide, or third polypeptide; where the first Fc domain monomer and the third Fc domain monomer combine to form a first Fc domain and the second Fc domain monomer and the fourth Fc domain monomer combine to form a second Fc domain.

In some embodiments of the fifth, sixth, and seventh aspects of the disclosure, the CD38 binding domain is joined to the first polypeptide and the second polypeptide or the third polypeptide, or to the second polypeptide and the third polypeptide, or the CD38 binding domain is joined to the first polypeptide, the second polypeptide, and the third polypeptide.

In some embodiments of the first, second, third and fourth aspects of the disclosure, the CD38 binding domain is a Fab or the V_{H} of a Fab.

In some embodiments of the fourth, fifth, sixth, and seventh aspects of the disclosure, the binding domain is part of the amino acid sequence of the first, second, or third polypeptide, and, in some embodiments, CD38 binding domain is a scFv.

In some embodiments of the fourth, fifth, sixth, and seventh aspects of the disclosure, the CD38 binding domain includes a V_{H} domain and a C_{H}1 domain, and where the V_{H} and C_{H}1 domains are part of the amino acid sequence of the first, second, or third polypeptide. In some embodiments, the CD38 binding domain further includes a V_{L} domain, where, in some embodiments the Fc-antigen binding domain construct includes a fourth polypeptide including the V_{L} domain. In some embodiments, the V_{H} domain includes a set of CDR-H1, CDR-H2 and CDR-H3 sequences set forth in Table 1, the V_{H} domain includes CDR-H1, CDR-H2, and CDR-H3 of a VH domain including a sequence of an antibody set forth in Table 2, the V_{H} domain includes CDR-H1, CDR-H2, and CDR-H3 of a V_{H} sequence of an antibody set forth in Table 2, and the V_{H} sequence, excluding the CDR-H1, CDR-H2, and CDR-H3 sequence, is at least 95% identical, at least 97% identical, at least 99% identical, or at least 99.5% identical to the V_{H} sequence of an antibody set forth in Table 2, or the V_{H} domain includes a V_{H} sequence of an antibody set forth in Table 2.

In some embodiments of the fourth, fifth, sixth, and seventh aspects of the disclosure, the CD38 binding domain includes a set of CDR-H1, CDR-H2, CDR-H3, CDR-L1, CDR-L2, and CDR-L3 sequences set forth in Table 1, CD38 binding domain includes CDR-H1, CDR-H2, CDR-H3, CDR-L1, CDR-L2, and CDR-L3 sequences from a set of a V_{H} and a V_{L} sequence of an antibody set forth in Table 2, the CD38 binding domain includes a V_{H} domain including CDR-H1, CDR-H2, and CDR-H3 of a V_{H} sequence of an antibody set forth in Table 2, and a V_{L} domain including CDR-L1, CDR-L2, and CDR-L3 of a V_{L} sequence of an antibody set forth in Table 2, where the V_{H} and the V_{L} domain sequences, excluding the CDR-H1, CDR-H2, CDR-H3, CDR-L1, CDR-L2, and CDR-L3 sequences, are at least 95% identical, at least 97% identical, at least 99% identical, or at least 99.5% identical to the V_{H} and V_{L} sequences of an antibody set forth in Table 2, or CD38 binding domain includes a set of a V_{H} and a V_{L} sequences of an antibody set forth in Table 2.

In some embodiments of the fourth, fifth, sixth, and seventh aspects of the disclosure, the Fc-antigen binding domain construct, further includes an IgG C_{L} antibody constant domain and an IgG C_{H}1 antibody constant domain, where the IgG C_{H}1 antibody constant domain is attached to the N-terminus of the first polypeptide or the second polypeptide by way of a linker.

In some embodiments of the fourth, fifth, sixth, and seventh aspects of the disclosure, the first Fc domain monomer and the third Fc domain monomer include complementary dimerization selectivity modules that promote dimerization between the first Fc domain monomer and the third Fc domain monomer.

In some embodiments of the fourth, fifth, sixth, and seventh aspects of the disclosure, the second Fc domain monomer and the fourth Fc domain monomer include complementary dimerization selectivity modules that promote dimerization between the second Fc domain monomer and the fourth Fc domain monomer.

In some embodiments of the fourth, fifth, sixth, and seventh aspects of the disclosure, the dimerization selectivity modules include an engineered cavity into the C_{H}3 domain of one of the Fc domain monomers and an engineered protuberance into the C_{H}3 domain of the other of the Fc domain monomers, where the engineered cavity and the engineered protuberance are positioned to form a protuberance-into-cavity pair of Fc domain monomers. In some embodiments, the engineered protuberance includes at least one modification selected from S354C, T366W, T366Y, T394W, T394F, and F405W, and the engineered cavity includes at least one modification selected from Y349C, T366S, L368A, Y407V, Y407T, Y407A, F405A, and T394S. In some embodiments, one of the Fc domain monomers includes Y407V and Y349C and the other of the Fc domain monomers includes T366W and S354C.

In some embodiments of the fourth, fifth, sixth, and seventh aspects of the disclosure, the dimerization selectivity modules include a negatively-charged amino acid into the C_{H}3 domain of one of the domain monomers and a positively-charged amino acid into the C_{H}3 domain of the other of the Fc domain monomers, where the negatively-charged amino acid and the positively-charged amino acid are positioned to promote formation of an Fc domain. In some embodiments, each of the first Fc domain monomer and third Fc domain monomer includes D399K and either K409D or K409E, each of the first Fc domain monomer and third Fc domain monomer includes K392D and D399K, each of the first Fc domain monomer and third Fc domain monomer includes E357K and K370E, each of the first Fc domain monomer and third Fc domain monomer includes D356K and K439D, each of the first Fc domain monomer and third Fc domain monomer includes K392E and D399K, each of the first Fc domain monomer and third Fc domain monomer includes E357K and K370D, each of the first Fc domain monomer and third Fc domain monomer includes D356K and K439E, each of the second Fc domain monomer and fourth Fc domain monomer includes S354C and T366W and the third and fourth polypeptides each include Y349C, T366S, L368A, and Y407V, each of the third and fourth polypeptides includes S354C and T366W and the second Fc domain monomer and fourth Fc domain monomer each include Y349C, T366S, L368A, and Y407V, each of the second Fc domain monomer and fourth Fc domain monomer includes E357K or E357R and the third and fourth polypeptides each include K370D or K370E, each of the second Fc domain monomer and fourth Fc domain monomer include K370D or K370E and the third and fourth polypeptides each include E357K or 357R, each of the second Fc domain monomer and fourth Fc domain monomer include K409D or K409E and the third and fourth polypeptides each include D399K or D399R, or each of the second Fc domain monomer and fourth Fc domain monomer include D399K or D399R and the third and fourth polypeptides each include K409D or K409E.

In some embodiments of the fourth, fifth, sixth, and seventh aspects of the disclosure, the second polypeptide and the third polypeptide have the same amino acid sequence.

In some embodiments of the fourth, fifth, sixth, and seventh aspects of the disclosure, one or more linker in the Fc-antigen binding domain construct is a bond.

In some embodiments of the fourth, fifth, sixth, and seventh aspects of the disclosure, one or more linker in the Fc-antigen binding domain construct is a spacer. In some embodiments, the spacer includes a polypeptide having the sequence GGGGGGGGGGGGGGGGGGGG (SEQ ID NO: 23), GGGGS (SEQ ID NO: 1), GGSG (SEQ ID NO: 2), SGGG (SEQ ID NO: 3), GSGS (SEQ ID NO: 4), GSGSGS (SEQ ID NO: 5), GSGSGSGS (SEQ ID NO: 6), GSGSGSGSGS (SEQ ID NO: 7), GSGSGSGSGSGS (SEQ ID NO: 8), GGSGGS (SEQ ID NO: 9), GGSGGSGGS (SEQ ID NO: 10), GGSGGSGGSGGS (SEQ ID NO: 11), GGSG (SEQ ID NO: 2), GGSG (SEQ ID NO: 2), GGSGGGSG (SEQ ID NO: 12), GGSGGGSGGGSGGGGGSGGGGSGGGGSGGGGS (SEQ ID NO: 213), GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), GGSGGGSEGGGSEGGGSEGGGSEGGGSEGGGSGGGS (SEQ ID NO: 34), GGGSGGGSGGGS (SEQ ID NO: 35), SGGGSGGGSGGGSGGGSGGG (SEQ ID NO: 18), GGSGGGSGGGSGGGSGGS (SEQ ID NO: 36), GGGG (SEQ ID NO: 19), GGGGGGGG (SEQ ID NO: 20), GGGGGGGGGGGG (SEQ ID NO: 21), or GGGGGGGGGGGGGGGG (SEQ ID NO: 22). In some embodiments, the spacer is a glycine spacer, for example, one consisting of 4 to 30 (SEQ ID NO: 214), 8 to 30 (SEQ ID NO: 215), or 12 to 30 (SEQ ID NO: 216) glycine residues, such as a spacer consisting of 20 glycine residues (SEQ ID NO: 23).

In some embodiments of the fourth, fifth, sixth, and seventh aspects of the disclosure, the CD38 binding domain is joined to the Fc domain monomer by a linker. In some embodiments, the linker is a spacer.

In some embodiments of the fourth, fifth, sixth, and seventh aspects of the disclosure, at least one of the Fc domains includes at least one amino acid modification at EU position I253. In some embodiments, the each amino acid modification at position I253 is independently selected from I253A, I253C, I253D, I253E, I253F, I253G, I253H, I253I, I253K, I253L, I253M, I253N, I253P, I253Q, I253R, I253S, I253T, I253V, I253W, and I253Y. In some embodiments, each amino acid modification at position I253 is I253A.

In some embodiments of the fourth, fifth, sixth, and seventh aspects of the disclosure, at least one of the Fc domains includes at least one amino acid modification at EU position R292. In some embodiments, each amino acid modification at position R292 is independently selected from R292D, R292E, R292L, R292P, R292Q, R292R, R292T, and R292Y. In some embodiments, each amino acid modification at position R292 is R292P.

In some embodiments of the fourth, fifth, sixth, and seventh aspects of the disclosure, one or more of the Fc domain monomers includes an IgG hinge domain, an IgG C_{H}2 antibody constant domain, and an IgG C_{H}3 antibody constant domain. In some embodiments, each of the Fc domain monomers includes an IgG hinge domain, an IgG C_{H}2 antibody constant domain, and an IgG C_{H}3 antibody constant domain. In some embodiments, the IgG is of a subtype selected from the group consisting of IgG1, IgG2a, IgG2b, IgG3, and IgG4.

In some embodiments of the fourth, fifth, sixth, and seventh aspects of the disclosure, the N-terminal Asp in each of the fourth, fifth, sixth, and seventh polypeptides is mutated to Gln.

In some embodiments of the fourth, fifth, sixth, and seventh aspects of the disclosure, one or more of the fourth, fifth, sixth, and seventh polypeptides lack a C-terminal lysine. In some embodiments, each of the fourth, fifth, sixth, and seventh polypeptides lacks a C-terminal lysine.

In some embodiments of the fourth, fifth, sixth, and seventh aspects of the disclosure, the Fc-antigen binding domain construct further includes an albumin-binding peptide joined to the N-terminus or C-terminus of one or more of the polypeptides by a linker.

In an eighth aspect, the disclosure features a cell culture medium including a population of Fc-antigen binding domain constructs, where at least 50% of the Fc-antigen binding domain constructs, on a molar basis, are structurally identical, and where the Fc-antigen binding domain constructs are present in the culture medium at a concentration of at least 0.1 mg/L, 10 mg/L, 25 mg/L, 50 mg/L, 75 mg/L, or 100 mg/L.

In some embodiments of the eighth aspect of the disclosure, at least 75%%, at least 85%, or at least 95% of the Fc-antigen binding domain constructs, on a molar basis, are structurally identical.

In a ninth aspect, the disclosure features a cell culture medium including a population of Fc-antigen binding domain constructs, where at least 50% of the Fc-antigen binding domain constructs, on a molar basis, include: a) a first polypeptide including i) a first Fc domain monomer, ii) a second Fc domain monomer, and iii) a linker joining the first Fc domain monomer and the second Fc domain monomer; b) a second polypeptide including a third Fc domain monomer; c) a third polypeptide including a fourth Fc domain monomer; and d) a CD38 binding domain joined to the first polypeptide, second polypeptide, or third polypeptide; where the first Fc domain monomer and the third Fc domain monomer combine to form a first Fc domain and the second Fc domain monomer and the fourth Fc domain monomer combine to form a second Fc domain.

In some embodiments of the ninth aspect of the disclosure at least 75%, at least 85%, or at least 95% of the Fc-antigen binding domain constructs, on a molar basis, include the first Fc domain, the second Fc domain, and the CD38 binding domain.

In a tenth aspect, the disclosure features a method of manufacturing an Fc-antigen binding domain construct, the method including: a) culturing a host cell expressing: (1) a first polypeptide including i) a first Fc domain monomer, ii) a second Fc domain monomer, and iii) a linker joining the first Fc domain monomer and the second Fc domain monomer; (2) a second polypeptide including a third Fc domain monomer; (3) a third polypeptide including a fourth Fc domain monomer; and (4) a CD38 binding domain; where the first Fc domain monomer and the third Fc domain monomer combine to form a first Fc domain and the second Fc domain monomer and the fourth Fc domain monomer combine to form a second Fc domain; where the CD38 binding domain is joined to the first polypeptide, second polypeptide, or third polypeptide, thereby forming an Fc-antigen binding domain construct; and where at least 50% of the Fc-antigen binding domain constructs in a cell culture supernatant, on a molar basis, are structurally identical, and b) purifying the Fc-antigen binding domain construct from the cell culture supernatant.

In some embodiments of the ninth and tenth aspects of the disclosure, the CD38 binding domain is joined to the first polypeptide and the second polypeptide or the third polypeptide, or to the second polypeptide and the third polypeptide, or the CD38 binding domain is joined to the first polypeptide, the second polypeptide, and the third polypeptide.

In some embodiments of the ninth and tenth aspects of the disclosure, the CD38 binding domain is a Fab or a V_{H}.

In some embodiments of the ninth and tenth aspects of the disclosure, the CD38 binding domain is part of the amino acid sequence of the first, second, or third polypeptide, and, in some embodiments, the CD38 binding domain is a scFv.

In some embodiments of the ninth and tenth aspects of the disclosure, CD38 binding domain includes a V_{H} domain and a C_{H}1 domain, and where the V_{H} and C_{H}1 domains are part of the amino acid sequence of the first, second, or third polypeptide. In some embodiments, the CD38 binding domain further includes a V_{L} domain, where, in some embodiments the Fc-antigen binding domain construct includes a fourth polypeptide including the V_{L} domain. In some embodiments, the V_{H} domain includes a set of CDR-H1, CDR-H2 and CDR-H3 sequences set forth in Table 1, the V_{H} domain includes CDR-H1, CDR-H2, and CDR-H3 of a VH domain including a sequence of an antibody set forth in Table 2, the V_{H} domain includes CDR-H1, CDR-H2, and CDR-H3 of a V_{H} sequence of an antibody set forth in Table 2, and the V_{H} sequence, excluding the CDR-H1, CDR-H2, and CDR-H3 sequence, is at least 95% identical, at least 97% identical, at least 99% identical, or at least 99.5% identical to the V_{H} sequence of an antibody set forth in Table 2, or the V_{H} domain includes a V_{H} sequence of an antibody set forth in Table 2.

In some embodiments of the ninth and tenth aspects of the disclosure, the CD38 binding domain includes a set of CDR-H1, CDR-H2, CDR-H3, CDR-L1, CDR-L2, and CDR-L3 sequences set forth in Table 1, CD38 binding domain includes CDR-H1, CDR-H2, CDR-H3, CDR-L1, CDR-L2, and CDR-L3 sequences from a set of a V_{H} and a V_{L} sequences of an antibody set forth in Table 2, CD38 binding domain includes a V_{H} domain including CDR-H1, CDR-H2, and CDR-H3 of a V_{H} sequence of an antibody set forth in Table 2, and a V_{L} domain including CDR-L1, CDR-L2, and CDR-L3 of a V_{L} sequence of an antibody set forth in Table 2, where the V_{H} and the V_{L} domain sequences, excluding the CDR-H1, CDR-H2, CDR-H3, CDR-L1, CDR-L2, and CDR-L3 sequences, are at least 95% identical, at least 97% identical, at least 99% identical, or at least 99.5% identical to the V_{H} and V_{L} sequences of an antibody set forth in Table 2, or the CD38 binding domain includes a set of a V_{H} and a V_{L} sequence of an antibody set forth in Table 2.

In some embodiments of the ninth and tenth aspects of the disclosure, the Fc-antigen binding domain construct, further includes an IgG C_{L} antibody constant domain and an IgG C_{H}1 antibody constant domain, where the IgG C_{H}1 antibody constant domain is attached to the N-terminus of the first polypeptide or the second polypeptide by way of a linker.

In some embodiments of the ninth and tenth aspects of the disclosure, the first Fc domain monomer and the third Fc domain monomer include complementary dimerization selectivity modules that promote dimerization between the first Fc domain monomer and the third Fc domain monomer.

In some embodiments of the ninth and tenth aspects of the disclosure, the second Fc domain monomer and the fourth Fc domain monomer include complementary dimerization selectivity modules that promote dimerization between the second Fc domain monomer and the fourth Fc domain monomer.

In some embodiments of the ninth and tenth aspects of the disclosure, the dimerization selectivity modules include an engineered cavity into the C_{H}3 domain of one of the Fc domain monomers and an engineered protuberance into the C_{H}3 domain of the other of the Fc domain monomers, where the engineered cavity and the engineered protuberance are positioned to form a protuberance-into-cavity pair of Fc domain monomers. In some embodiments, the engineered protuberance includes at least one modification selected from S354C, T366W, T366Y, T394W, T394F, and F405W, and the engineered cavity includes at least one modification selected from Y349C, T366S, L368A, Y407V, Y407T, Y407A, F405A, and T394S. In some embodiments, one of the Fc domain monomers includes Y407V and Y349C and the other of the Fc domain monomers includes T366W and S354C.

In some embodiments of the ninth and tenth aspects of the disclosure, the dimerization selectivity modules include a negatively-charged amino acid into the C_{H}3 domain of one of the domain monomers and a positively-charged amino acid into the C_{H}3 domain of the other of the Fc domain monomers, where the negatively-charged amino acid and the positively-charged amino acid are positioned to promote formation of an Fc domain. In some embodiments, each of the first Fc domain monomer and third Fc domain monomer includes D399K and either K409D or K409E, each of the first Fc domain monomer and third Fc domain monomer includes K392D and D399K, each of the first Fc domain monomer and third Fc domain monomer includes E357K and K370E, each of the first Fc domain monomer and third Fc domain monomer includes D356K and K439D, each of the first Fc domain monomer and third Fc domain monomer includes K392E and D399K, each of the first Fc domain monomer and third Fc domain monomer includes E357K and K370D, each of the first Fc domain monomer and third Fc domain monomer includes D356K and K439E, each of the second Fc domain monomer and fourth Fc domain monomer includes S354C and T366W and the third and fourth polypeptides each include Y349C, T366S, L368A, and Y407V, each of the third and fourth polypeptides includes S354C and T366W and the second Fc domain monomer and fourth Fc domain monomer each include Y349C, T366S, L368A, and Y407V, each of the second Fc domain monomer and fourth Fc domain monomer includes E357K or E357R and the third and fourth polypeptides each include K370D or K370E, each of the second Fc domain monomer and fourth Fc domain monomer include K370D or K370E and the third and fourth polypeptides each include E357K or 357R, each of the second Fc domain monomer and fourth Fc domain monomer include K409D or K409E and the third and fourth polypeptides each include D399K or D399R, or each of the second Fc domain monomer and fourth Fc domain monomer include D399K or D399R and the third and fourth polypeptides each include K409D or K409E.

In some embodiments of the ninth and tenth aspects of the disclosure, the second polypeptide and the third polypeptide have the same amino acid sequence.

In some embodiments of the ninth and tenth aspects of the disclosure, one or more linker in the Fc-antigen binding domain construct is a bond.

In some embodiments of the ninth and tenth aspects of the disclosure, one or more linker in the Fc-antigen binding domain construct is a spacer. In some embodiments, the spacer includes a polypeptide having the sequence GGGGGGGGGGGGGGGGGGGG (SEQ ID NO: 23), GGGGS (SEQ ID NO: 1), GGSG (SEQ ID NO: 2), SGGG (SEQ ID NO: 3), GSGS (SEQ ID NO: 4), GSGSGS (SEQ ID NO: 5), GSGSGSGS (SEQ ID NO: 6), GSGSGSGSGS (SEQ ID NO: 7), GSGSGSGSGSGS (SEQ ID NO: 8), GGSGGS (SEQ ID NO: 9), GGSGGSGGS (SEQ ID NO: 10), GGSGGSGGSGGS (SEQ ID NO: 11), GGSG (SEQ ID NO: 2), GGSG (SEQ ID NO: 2), GGSGGGSG (SEQ ID NO: 12), GGSGGGSGGGSGGGGGSGGGGSGGGGSGGGGS (SEQ ID NO: 213), GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), GGSGGGSEGGGSEGGGSEGGGSEGGGSEGGGSGGGS (SEQ ID NO: 34), GGGSGGGSGGGS (SEQ ID NO: 35), SGGGSGGGSGGGSGGGSGGG (SEQ ID NO: 18), GGSGGGSGGGSGGGSGGS (SEQ ID NO: 36), GGGG (SEQ ID NO: 19), GGGGGGGG (SEQ ID NO: 20), GGGGGGGGGGGG (SEQ ID NO: 21), or GGGGGGGGGGGGGGGG (SEQ ID NO: 22). In some embodiments, the spacer is a glycine spacer, for example, one consisting of 4 to 30 (SEQ ID NO: 214), 8 to 30 (SEQ ID NO: 215), or 12 to 30 (SEQ ID NO: 216) glycine residues, such as a spacer consisting of 20 glycine residues (SEQ ID NO: 23).

In some embodiments of the ninth and tenth aspects of the disclosure, the CD38 binding domain is joined to the Fc domain monomer by a linker. In some embodiments, the linker is a spacer.

In some embodiments of the ninth and tenth aspects of the disclosure, at least one of the Fc domains includes at least one amino acid modification at position I253. In some embodiments, the each amino acid modification at position I253 is independently selected from I253A, I253C, I253D, I253E, I253F, I253G, I253H, I253I, I253K, I253L, I253M, I253N, I253P, I253Q, I253R, I253S, I253T, I253V, I253W, and I253Y. In some embodiments, each amino acid modification at position I253 is I253A.

In some embodiments of the ninth and tenth aspects of the disclosure, at least one of the Fc domains includes at least one amino acid modification at position R292. In some embodiments, each amino acid modification at position R292 is independently selected from R292D, R292E, R292L, R292P, R292Q, R292R, R292T, and R292Y. In some embodiments, each amino acid modification at position R292 is R292P.

In some embodiments of the ninth and tenth aspects of the disclosure, one or more of the Fc domain monomers includes an IgG hinge domain, an IgG C_{H}2 antibody constant domain, and an IgG C_{H}3 antibody constant domain. In some embodiments, each of the Fc domain monomers includes an IgG hinge domain, an IgG C_{H}2 antibody constant domain, and an IgG C_{H}3 antibody constant domain. In some embodiments, the IgG is of a subtype selected from the group consisting of IgG1, IgG2a, IgG2b, IgG3, and IgG4.

In some embodiments of the ninth and tenth aspects of the disclosure, the N-terminal Asp in each of the first, second, third, and fourth polypeptides is mutated to Gln.

In some embodiments of the ninth and tenth aspects of the disclosure, one or more of the first, second, third, and fourth polypeptides lack a C-terminal lysine. In some embodiments, each of the first, second, third, and fourth polypeptides lacks a C-terminal lysine.

In some embodiments of the ninth and tenth aspects of the disclosure, the Fc-antigen binding domain construct further includes an albumin-binding peptide joined to the N-terminus or C-terminus of one or more of the polypeptides by a linker.

In some embodiments of the eleventh aspect of the disclosure, the first Fc domain monomer and the third Fc domain monomer include complementary dimerization selectivity modules that promote dimerization between the first Fc domain monomer and the third Fc domain monomer, where the second Fc domain monomer and the fourth Fc domain monomer include complementary dimerization selectivity modules that promote dimerization between the second Fc domain monomer and the fourth Fc domain monomer, and where the second polypeptide and the third polypeptide have different amino acid sequences.

In some embodiments of the eleventh aspect of the disclosure, the first CD38 binding domain is joined to the first polypeptide and the second CD38 binding domain is joined to the second polypeptide and the third polypeptide.

In some embodiments of the eleventh aspect of the disclosure each of the second Fc domain monomer and the fourth Fc domain monomer includes E357K and K370D, and each of the first Fc domain monomer and the third Fc domain monomer includes K370D and E357K.

In some embodiments of the twelfth aspect of the disclosure, the first Fc domain monomer and the third Fc domain monomer include complementary dimerization selectivity modules that promote dimerization between the first Fc domain monomer and the third Fc domain monomer, where the second Fc domain monomer and the fourth Fc domain monomer include complementary dimerization selectivity modules that promote dimerization between the second Fc domain monomer and the fourth Fc domain monomer, and where the second polypeptide and the third polypeptide have different amino acid sequences.

In some embodiments of the twelfth aspect of the disclosure, each of the second Fc domain monomer and the fourth Fc domain monomer includes D399K and K409D, and each of the first Fc domain monomer and the third Fc domain monomer includes E357K and K370D.

In some embodiments of the eleventh and twelfth aspects of the disclosure, the first or CD38 binding domain is a Fab or a V_{H} domain. In some embodiments of the eleventh and twelfth aspects of the disclosure, the first and second CD38 binding domain is a Fab. In some embodiments of the ninth aspect of the disclosure, the first, second, and third CD38 binding domain is a Fab or a V_{H} domain.

In some embodiments of the eleventh and twelfth aspects of the disclosure, the first or second CD38 binding domain is a scFv. In some embodiments of the eleventh and twelfth aspects of the disclosure, the first and second CD38 binding domain is a scFv. In some embodiments of the ninth aspect of the disclosure, the first, second, and third CD38 binding domain is a scFv.

In some embodiments of the eleventh aspect of the disclosure, the first or second CD38 domain includes a V_{H} domain and a C_{H}1 domain, and where the V_{H} and C_{H}1 domains are part of the amino acid sequence of the first, second, or third polypeptide. In some embodiments, the CD38 binding domain further includes a V_{L} domain, where, in some embodiments the Fc-antigen binding domain construct includes a fourth polypeptide including the V_{L} domain. In some embodiments, the V_{H} domain includes a set of CDR-H1, CDR-H2 and CDR-H3 sequences set forth in Table 1, the V_{H} domain includes CDR-H1, CDR-H2, and CDR-H3 of a VH domain including a sequence of an antibody set forth in Table 2, the V_{H} domain includes CDR-H1, CDR-H2, and CDR-H3 of a V_{H} sequence of an antibody set forth in Table 2, and the V_{H} sequence, excluding the CDR-H1, CDR-H2, and CDR-H3 sequence, is at least 95% identical, at least 97% identical, at least 99% identical, or at least 99.5% identical to the V_{H} sequence of an antibody set forth in Table 2, or the V_{H} domain includes a V_{H} sequence of an antibody set forth in Table 2.

In some embodiments of the twelfth aspect of the disclosure, the first, second, or third CD38 binding domain includes a V_{H} domain and a C_{H}1 domain, and where the V_{H} and C_{H}1 domains are part of the amino acid sequence of the first, second, or third polypeptide. In some embodiments, the CD38 binding domain further includes a V_{L} domain, where, in some embodiments the Fc-antigen binding domain construct includes a fourth polypeptide including the V_{L} domain. In some embodiments, the V_{H} domain includes a set of CDR-H1, CDR-H2 and CDR-H3 sequences set forth in Table 1, the V_{H} domain includes CDR-H1, CDR-H2, and CDR-H3 of a V_{H} domain including a sequence of an antibody set forth in Table 2, the V_{H} domain includes CDR-H1, CDR-H2, and CDR-H3 of a V_{H} sequence of an antibody set forth in Table 2, and the V_{H} sequence, excluding the CDR-H1, CDR-H2, and CDR-H3 sequence, is at least 95% identical, at least 97% identical, at least 99% identical, or at least 99.5% identical to the V_{H} sequence of an antibody set forth in Table 2, or the V_{H} domain includes a V_{H} sequence of an antibody set forth in Table 2.

In some embodiments of the eleventh aspect of the disclosure, the first or second CD38 binding domain includes a set of CDR-H1, CDR-H2, CDR-H3, CDR-L1, CDR-L2, and CDR-L3 sequences set forth in Table 1, the CD38 binding domain includes CDR-H1, CDR-H2, CDR-H3, CDR-L1, CDR-L2, and CDR-L3 sequences from a set of a V_{H} and a V_{L} sequence of an antibody set forth in Table 2, the CD38 binding domain includes a V_{H} domain including CDR-H1, CDR-H2, and CDR-H3 of a V_{H} sequence of an antibody set forth in Table 2, and a V_{L} domain including CDR-L1, CDR-L2, and CDR-L3 of a V_{L} sequences of an antibody set forth in Table 2, where the V_{H} and the V_{L} domain sequences, excluding the CDR-H1, CDR-H2, CDR-H3, CDR-L1, CDR-L2, and CDR-L3 sequences, are at least 95% identical, at least 97% identical, at least 99% identical, or at least 99.5% identical to the V_{H} and V_{L} sequences of an antibody set forth in Table 2, or the CD38 binding domain includes a set of a V_{H} and a V_{L} sequence of an antibody set forth in Table 2.

In some embodiments of the twelfth aspect of the disclosure, the first, second, or third CD38 binding domain includes a set of CDR-H1, CDR-H2, CDR-H3, CDR-L1, CDR-L2, and CDR-L3 sequences set forth in Table 1, the CD38 binding domain includes CDR-H1, CDR-H2, CDR-H3, CDR-L1, CDR-L2, and CDR-L3 sequences from a set of a V_{H} and a V_{L} sequence of an antibody set forth in Table 2, the CD38 binding domain includes a V_{H} domain including CDR-H1, CDR-H2, and CDR-H3 of a V_{H} sequence of an antibody set forth in Table 2, and a V_{L} domain including CDR-L1, CDR-L2, and CDR-L3 of a V_{L} sequence of an antibody set forth in Table 2, where the V_{H} and the V_{L} domain sequences, excluding the CDR-H1, CDR-H2, CDR-H3, CDR-L1, CDR-L2, and CDR-L3 sequences, are at least 95% identical, at least 97% identical, at least 99% identical, or at least 99.5% identical to the V_{H} and V_{L} sequences of an antibody set forth in Table 2, or the CD38 binding domain includes a set of a V_{H} and a V_{L} sequences of an antibody set forth in Table 2.

In some embodiments of the eleventh and twelfth aspects of the disclosure, the Fc-antigen binding domain construct, further includes an IgG C_{L} antibody constant domain and an IgG C_{H}1 antibody constant domain, where the IgG C_{H}1 antibody constant domain is attached to the N-terminus of the first polypeptide or the second polypeptide by way of a linker.

In some embodiments of the eleventh and twelfth aspects of the disclosure, the first Fc domain monomer and the third Fc domain monomer include complementary dimerization selectivity modules that promote dimerization between the first Fc domain monomer and the third Fc domain monomer.

In some embodiments of the eleventh and twelfth aspects of the disclosure, the second Fc domain monomer and the fourth Fc domain monomer include complementary dimerization selectivity modules that promote dimerization between the second Fc domain monomer and the fourth Fc domain monomer.

In some embodiments of the eleventh and twelfth aspects of the disclosure, the dimerization selectivity modules include an engineered cavity into the C_{H}3 domain of one of the Fc domain monomers and an engineered protuberance into the C_{H}3 domain of the other of the Fc domain monomers, where the engineered cavity and the engineered protuberance are positioned to form a protuberance-into-cavity pair of Fc domain monomers. In some embodiments, the engineered protuberance includes at least one modification selected from S354C, T366W, T366Y, T394W, T394F, and F405W, and the engineered cavity includes at least one modification selected from Y349C, T366S, L368A, Y407V, Y407T, Y407A, F405A, and T394S. In some embodiments, one of the Fc domain monomers includes Y407V and Y349C and the other of the Fc domain monomers includes T366W and S354C.

In some embodiments of the eleventh and twelfth aspects of the disclosure, the dimerization selectivity modules include a negatively-charged amino acid into the C_{H}3 domain of one of the domain monomers and a positively-charged amino acid into the C_{H}3 domain of the other of the Fc domain monomers, where the negatively-charged amino acid and the positively-charged amino acid are positioned to promote formation of an Fc domain. In some embodiments, each of the first Fc domain monomer and third Fc domain monomer includes D399K and either K409D or K409E, each of the first Fc domain monomer and third Fc domain monomer includes K392D and D399K, each of the first Fc domain monomer and third Fc domain monomer includes E357K and K370E, each of the first Fc domain monomer and third Fc domain monomer includes D356K and K439D, each of the first Fc domain monomer and third Fc domain monomer includes K392E and D399K, each of the first Fc domain monomer and third Fc domain monomer includes E357K and K370D, each of the first Fc domain monomer and third Fc domain monomer includes D356K and K439E, each of the second Fc domain monomer and fourth Fc domain monomer includes S354C and T366W and the third and fourth polypeptides each include Y349C, T366S, L368A, and Y407V, each of the third and fourth polypeptides includes S354C and T366W and the second Fc domain monomer and fourth Fc domain monomer each include Y349C, T366S, L368A, and Y407V, each of the second Fc domain monomer and fourth Fc domain monomer includes E357K or E357R and the third and fourth polypeptides each include K370D or K370E, each of the second Fc domain monomer and fourth Fc domain monomer include K370D or K370E and the third and fourth polypeptides each include E357K or 357R, each of the second Fc domain monomer and fourth Fc domain monomer include K409D or K409E and the third and fourth polypeptides each include D399K or D399R, or each of the second Fc domain monomer and fourth Fc domain monomer include D399K or D399R and the third and fourth polypeptides each include K409D or K409E.

In some embodiments of the eleventh and twelfth aspects of the disclosure, one or more linker in the Fc-antigen binding domain construct is a bond.

In some embodiments of the eleventh and twelfth aspects of the disclosure, one or more linker in the Fc-antigen binding domain construct is a spacer. In some embodiments, the spacer includes a polypeptide having the sequence GGGGGGGGGGGGGGGGGGGG (SEQ ID NO: 23), GGGGS (SEQ ID NO: 1), GGSG (SEQ ID NO: 2), SGGG (SEQ ID NO: 3), GSGS (SEQ ID NO: 4), GSGSGS (SEQ ID NO: 5), GSGSGSGS (SEQ ID NO: 6), GSGSGSGSGS (SEQ ID NO: 7), GSGSGSGSGSGS (SEQ ID NO: 8), GGSGGS (SEQ ID NO: 9), GGSGGSGGS (SEQ ID NO: 10), GGSGGSGGSGGS (SEQ ID NO: 11), GGSG (SEQ ID NO: 2), GGSG (SEQ ID NO: 2), GGSGGGSG (SEQ ID NO: 12), GGSGGGSGGGSGGGGGSGGGGSGGGGSGGGGS (SEQ ID NO: 213), GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), GGSGGGSEGGGSEGGGSEGGGSEGGGSEGGGSGGGS (SEQ ID NO: 34), GGGSGGGSGGGS (SEQ ID NO: 35), SGGGSGGGSGGGSGGGSGGG (SEQ ID NO: 18), GGSGGGSGGGSGGGSGGS (SEQ ID NO: 36), GGGG (SEQ ID NO: 19), GGGGGGGG (SEQ ID NO: 20), GGGGGGGGGGGG (SEQ ID NO: 21), or GGGGGGGGGGGGGGGG (SEQ ID NO: 22). In some embodiments, the spacer is a glycine spacer, for example, one consisting of 4 to 30 (SEQ ID NO: 214), 8 to 30 (SEQ ID NO: 215), or 12 to 30 (SEQ ID NO: 216) glycine residues, such as a spacer consisting of 20 glycine residues (SEQ ID NO: 23).

In some embodiments of the eleventh and twelfth aspects of the disclosure, one or more of the CD38 binding domains is joined to the Fc domain monomer by a linker. In some embodiments, the linker is a spacer.

In some embodiments of the eleventh and twelfth aspects of the disclosure, at least one of the Fc domains includes at least one amino acid modification at position I253. In some embodiments, the each amino acid modification at position I253 is independently selected from I253A, I253C, I253D, I253E, I253F, I253G, I253H, I253I, I253K, I253L, I253M, I253N, I253P, I253Q, I253R, I253S, I253T, I253V, I253W, and I253Y. In some embodiments, each amino acid modification at position I253 is I253A.

In some embodiments of the eleventh and twelfth aspects of the disclosure, at least one of the Fc domains includes at least one amino acid modification at position R292. In some embodiments, each amino acid modification at position R292 is independently selected from R292D, R292E, R292L, R292P, R292Q, R292R, R292T, and R292Y. In some embodiments, each amino acid modification at position R292 is R292P.

In some embodiments of the eleventh and twelfth aspects of the disclosure, one or more of the Fc domain monomers includes an IgG hinge domain, an IgG C_{H}2 antibody constant domain, and an IgG C_{H}3 antibody constant domain. In some embodiments, each of the Fc domain monomers includes an IgG hinge domain, an IgG C_{H}2 antibody constant domain, and an IgG C_{H}3 antibody constant domain. In some embodiments, the IgG is of a subtype selected from the group consisting of IgG1, IgG2a, IgG2b, IgG3, and IgG4.

In some embodiments of the eleventh and twelfth aspects of the disclosure, the N-terminal Asp in each of the first, second, third, and fourth polypeptides is mutated to Gln.

In some embodiments of the eleventh and twelfth aspects of the disclosure, one or more of the first, second, third, and fourth polypeptides lack a C-terminal lysine. In some embodiments, each of the first, second, third, and fourth polypeptides lacks a C-terminal lysine.

In some embodiments of the eleventh and twelfth aspects of the disclosure, the Fc-antigen binding domain construct further includes an albumin-binding peptide joined to the N-terminus or C-terminus of one or more of the polypeptides by a linker.

In a thirteenth aspect, the disclosure features a composition including a substantially homogenous population of an Fc-antigen binding domain construct including: a) a first polypeptide including i) a first Fc domain monomer, ii) a second Fc domain monomer, and iii) a first linker joining the first Fc domain monomer and the second Fc domain monomer; and b) a second polypeptide including i) a third Fc domain monomer, ii) a fourth Fc domain monomer, and iv) a second linker joining the third Fc domain monomer and the fourth Fc domain monomer; and c) a third polypeptide including a fifth Fc domain monomer; d) a fourth polypeptide including an sixth Fc domain monomer; and d) a CD38 binding domain joined to the first polypeptide, second polypeptide, third polypeptide, or fourth polypeptide; where the first Fc domain monomer and the third Fc domain monomer combine to form a first Fc domain and the second Fc domain monomer and the fifth Fc domain monomer combine to form a second Fc domain, the fourth Fc domain monomer and the sixth Fc domain monomer combine to form a third Fc domain.

In some embodiments of the thirteenth aspect of the disclosure, each of the first and third Fc domain monomers includes a complementary dimerization selectivity module that promote dimerization between the first Fc domain monomer and the third Fc domain monomer, each of the second and fifth Fc domain monomers includes a complementary dimerization selectivity module that promote dimerization between the second Fc domain monomer and the fifth Fc domain monomer, and each of the fourth and sixth Fc domain monomers includes a complementary dimerization selectivity module that promote dimerization between the fourth Fc domain monomer and the sixth Fc domain monomer.

In an fourteenth aspect, the disclosure features a composition including a substantially homogenous population of an Fc-antigen binding domain construct including: a) a first polypeptide including i) a first Fc domain monomer, ii) a second Fc domain monomer, and iii) a first linker joining the first Fc domain monomer and the second Fc domain monomer; and b) a second polypeptide including i) a third Fc domain monomer, ii) a fourth Fc domain monomer, and iv) a second linker joining the third Fc domain monomer and the fourth Fc domain monomer; and c) a third polypeptide including a fifth Fc domain monomer; d) a fourth polypeptide including an sixth Fc domain monomer; and e) a CD38 binding domain joined to the first polypeptide, second polypeptide, third polypeptide, or fourth polypeptide; wherein the second Fc domain monomer and the fourth Fc domain monomer combine to form a first Fc domain and the first Fc domain monomer and the fifth Fc domain monomer combine to form a second Fc domain, the third Fc domain monomer and the sixth Fc domain monomer combine to form a third Fc domain.

In some embodiments of the fourteenth aspect of the disclosure, each of the second and fourth Fc domain monomers includes a complementary dimerization selectivity module that promote dimerization between the second Fc domain monomer and the fourth Fc domain monomer, each of the first and fifth Fc domain monomers includes a complementary dimerization selectivity module that promote dimerization between the first Fc domain monomer and the fifth Fc domain monomer, and each of the third and sixth Fc domain monomers includes a complementary dimerization selectivity module that promote dimerization between the third Fc domain monomer and the sixth Fc domain monomer.

In a fifteenth aspect, the disclosure features a composition including a substantially homogenous population of an Fc-antigen binding domain construct including: a) a first polypeptide including i) a first Fc domain monomer, ii) a second Fc domain monomer, iii) a third Fc domain monomer, iv) a first linker joining the first Fc domain monomer and the second Fc domain monomer; and v) a second linker joining the second Fc domain monomer and the third Fc domain monomer; b) a second polypeptide including i) a fourth Fc domain monomer, ii) a fifth Fc domain monomer, iii) a sixth Fc domain monomer, iv) a third linker joining the fourth Fc domain monomer and the fifth Fc domain monomer; and v) a fourth linker joining the fifth Fc domain monomer and the sixth Fc domain monomer; c) a third polypeptide including a seventh Fc domain monomer; d) a fourth polypeptide including an eighth Fc domain monomer; e) a fifth polypeptide including a ninth Fc domain monomer; f) a sixth polypeptide including a tenth Fc domain monomer; and g) a CD38 binding domain joined to the first polypeptide, second polypeptide, third polypeptide, fourth polypeptide, fifth polypeptide, or sixth polypeptide; where the second Fc domain monomer and the fifth Fc domain monomer combine to form a first Fc domain and the first Fc domain monomer and the seventh Fc domain monomer combine to form a second Fc domain, the fourth Fc domain monomer and the eighth Fc domain monomer combine to form a third Fc domain, the third Fc domain monomer and the ninth Fc domain monomer combine to form a fourth Fc domain, and the sixth Fc domain monomer and the tenth Fc domain monomer combine to form a fifth Fc domain.

In some embodiments of the fifteenth aspect of the disclosure, each of the second and fifth Fc domain monomers includes a complementary dimerization selectivity module that promote dimerization between the second Fc domain monomer and the fifth Fc domain monomer, each of the first and seventh Fc domain monomers includes a complementary dimerization selectivity module that promote dimerization between the first Fc domain monomer and the seventh Fc domain monomer, each of the fourth and eighth Fc domain monomers includes a complementary dimerization selectivity module that promote dimerization between the fourth Fc domain monomer and the eighth Fc domain monomer, each of the third and ninth Fc domain monomers includes a complementary dimerization selectivity module that promote dimerization between the third Fc domain monomer and the ninth Fc domain monomer, and each of the sixth and tenth Fc domain monomers includes a complementary dimerization selectivity module that promote dimerization between the sixth Fc domain monomer and the tenth Fc domain monomer.

In some embodiments of the thirteenth, fourteenth, and fifteenth aspects of the disclosure, the CD38 binding domain is a Fab or a V_{H} domain

In some embodiments of the thirteenth, fourteenth, and fifteenth aspects of the disclosure, the CD38 binding domain is part of the amino acid sequence of one or more of the polypeptides, and, in some embodiments, the CD38 binding domain is a scFv.

In some embodiments of the thirteenth, fourteenth, and fifteenth aspects of the disclosure, the CD38 binding domain includes a V_{H} domain and a C_{H}1 domain, and where the V_{H} and C_{H}1 domains are part of the amino acid sequence of the first, second, or third polypeptide. In some embodiments, the CD38 binding domain further includes a V_{L} domain, where, in some embodiments the Fc-antigen binding domain construct includes a fourth polypeptide including the V_{L} domain. In some embodiments, the V_{H} domain includes a set of CDR-H1, CDR-H2 and CDR-H3 sequences set forth in Table 1, the V_{H} domain includes CDR-H1, CDR-H2, and CDR-H3 of a VH domain including a sequence of an antibody set forth in Table 2, the V_{H} domain includes CDR-H1, CDR-H2, and CDR-H3 of a V_{H} sequence of an antibody set forth in Table 2, and the V_{H} sequence, excluding the CDR-H1, CDR-H2, and CDR-H3 sequence, is at least 95% identical, at least 97% identical, at least 99% identical, or at least 99.5% identical to the V_{H} sequence of an antibody set forth in Table 2, or the V_{H} domain includes a V_{H} sequence of an antibody set forth in Table 2.

In some embodiments of the thirteenth, fourteenth, and fifteenth aspects of the disclosure, the CD38 binding domain includes a set of CDR-H1, CDR-H2, CDR-H3, CDR-L1, CDR-L2, and CDR-L3 sequences set forth in Table 1, the CD38 binding domain includes CDR-H1, CDR-H2, CDR-H3, CDR-L1, CDR-L2, and CDR-L3 sequences from a set of a V_{H} and a V_{L} sequences of an antibody set forth in Table 2, the CD38 binding domain includes a V_{H} domain including CDR-H1, CDR-H2, and CDR-H3 of a V_{H} sequence of an antibody set forth in Table 2, and a V_{L} domain including CDR-L1, CDR-L2, and CDR-L3 of a V_{L} sequence of an antibody set forth in Table 2, where the V_{H} and the V_{L} domain sequences, excluding the CDR-H1, CDR-H2, CDR-H3, CDR-L1, CDR-L2, and CDR-L3 sequences, are at least 95% identical, at least 97% identical, at least 99% identical, or at least 99.5% identical to the V_{H} and V_{L} sequences of an antibody set forth in Table 2, or the CD38 binding domain includes a set of a V_{H} and a V_{L} sequences of an antibody set forth in Table 2.

In some embodiments of the thirteenth, fourteenth, and fifteenth aspects of the disclosure, the Fc-antigen binding domain construct, further includes an IgG C_{L} antibody constant domain and an IgG C_{H}1 antibody constant domain, where the IgG C_{H}1 antibody constant domain is attached to the N-terminus of the first polypeptide or the second polypeptide by way of a linker.

In some embodiments of the thirteenth, fourteenth, and fifteenth aspects of the disclosure, the dimerization selectivity modules include an engineered cavity into the C_{H}3 domain of one of the Fc domain monomers and an engineered protuberance into the C_{H}3 domain of the other of the Fc domain monomers, where the engineered cavity and the engineered protuberance are positioned to form a protuberance-into-cavity pair of Fc domain monomers. In some embodiments, the engineered protuberance includes at least one modification selected from S354C, T366W, T366Y, T394W, T394F, and F405W, and the engineered cavity includes at least one modification selected from Y349C, T366S, L368A, Y407V, Y407T, Y407A, F405A, and T394S. In some embodiments, one of the Fc domain monomers includes Y407V and Y349C and the other of the Fc domain monomers includes T366W and S354C.

In some embodiments of the thirteenth, fourteenth, and fifteenth aspects of the disclosure, the dimerization selectivity modules include a negatively-charged amino acid into the C_{H}3 domain of one of the domain monomers and a positively-charged amino acid into the C_{H}3 domain of the other of the Fc domain monomers, where the negatively-charged amino acid and the positively-charged amino acid are positioned to promote formation of an Fc domain. In some embodiments, each of the first Fc domain monomer and third Fc domain monomer includes D399K and either K409D or K409E, each of the first Fc domain monomer and third Fc domain monomer includes K392D and D399K, each of the first Fc domain monomer and third Fc domain monomer includes E357K and K370E, each of the first Fc domain monomer and third Fc domain monomer includes D356K and K439D, each of the first Fc domain monomer and third Fc domain monomer includes K392E and D399K, each of the first Fc domain monomer and third Fc domain monomer includes E357K and K370D, each of the first Fc domain monomer and third Fc domain monomer includes D356K and K439E, each of the second Fc domain monomer and fourth Fc domain monomer includes S354C and T366W and the third and fourth polypeptides each include Y349C, T366S, L368A, and Y407V, each of the third and fourth polypeptides includes S354C and T366W and the second Fc domain monomer and fourth Fc domain monomer each include Y349C, T366S, L368A, and Y407V, each of the second Fc domain monomer and fourth Fc domain monomer includes E357K or E357R and the third and fourth polypeptides each include K370D or K370E, each of the second Fc domain monomer and fourth Fc domain monomer include K370D or K370E and the third and fourth polypeptides each include E357K or 357R, each of the second Fc domain monomer and fourth Fc domain monomer include K409D or K409E and the third and fourth polypeptides each include D399K or D399R, or each of the second Fc domain monomer and fourth Fc domain monomer include D399K or D399R and the third and fourth polypeptides each include K409D or K409E.

In some embodiments of the thirteenth, fourteenth, and fifteenth aspects of the disclosure, one or more linker in the Fc-antigen binding domain construct is a bond.

In some embodiments of the thirteenth, fourteenth, and fifteenth aspects of the disclosure, one or more linker in the Fc-antigen binding domain construct is a spacer. In some embodiments, the spacer includes a polypeptide having the sequence GGGGGGGGGGGGGGGGGGGG (SEQ ID NO: 23), GGGGS (SEQ ID NO: 1), GGSG (SEQ ID NO: 2), SGGG (SEQ ID NO: 3), GSGS (SEQ ID NO: 4), GSGSGS (SEQ ID NO: 5), GSGSGSGS (SEQ ID NO: 6), GSGSGSGSGS (SEQ ID NO: 7), GSGSGSGSGSGS (SEQ ID NO: 8), GGSGGS (SEQ ID NO: 9), GGSGGSGGS (SEQ ID NO: 10), GGSGGSGGSGGS (SEQ ID NO: 11), GGSG (SEQ ID NO: 2), GGSG (SEQ ID NO: 2), GGSGGGSG (SEQ ID NO: 12), GGSGGGSGGGSGGGGGSGGGGSGGGGSGGGGS (SEQ ID NO: 213), GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), GGSGGGSEGGGSEGGGSEGGGSEGGGSEGGGSGGGS (SEQ ID NO: 34), GGGSGGGSGGGS (SEQ ID NO: 35), SGGGSGGGSGGGSGGGSGGG (SEQ ID NO: 18), GGSGGGSGGGSGGGSGGS (SEQ ID NO: 36), GGGG (SEQ ID NO: 19), GGGGGGGG (SEQ ID NO: 20), GGGGGGGGGGGG (SEQ ID NO: 21), or GGGGGGGGGGGGGGGG (SEQ ID NO: 22). In some embodiments, the spacer is a glycine spacer, for example, one consisting of 4 to 30 (SEQ ID NO: 214), 8 to 30 (SEQ ID NO: 215), or 12 to 30 (SEQ ID NO: 216) glycine residues, such as a spacer consisting of 20 glycine residues (SEQ ID NO: 23).

In some embodiments of the thirteenth, fourteenth, and fifteenth aspects of the disclosure, the CD38 binding domain is joined to the Fc domain monomer by a linker. In some embodiments, the linker is a spacer.

In some embodiments of the thirteenth, fourteenth, and fifteenth aspects of the disclosure, at least one of the Fc domains includes at least one amino acid modification at position I253. In some embodiments, the each amino acid modification at position I253 is independently selected from I253A, I253C, I253D, I253E, I253F, I253G, I253H, I253I, I253K, I253L, I253M, I253N, I253P, I253Q, I253R, I253S, I253T, I253V, I253W, and I253Y. In some embodiments, each amino acid modification at position I253 is I253A.

In some embodiments of the thirteenth, fourteenth, and fifteenth aspects of the disclosure, at least one of the Fc domains includes at least one amino acid modification at position R292. In some embodiments, each amino acid modification at position R292 is independently selected from R292D, R292E, R292L, R292P, R292Q, R292R, R292T, and R292Y. In some embodiments, each amino acid modification at position R292 is R292P.

In some embodiments of the thirteenth, fourteenth, and fifteenth aspects of the disclosure, one or more of the Fc domain monomers includes an IgG hinge domain, an IgG C_{H}2 antibody constant domain, and an IgG C_{H}3 antibody constant domain. In some embodiments, each of the Fc domain monomers includes an IgG hinge domain, an IgG C_{H}2 antibody constant domain, and an IgG C_{H}3 antibody constant domain. In some embodiments, the IgG is of a subtype selected from the group consisting of IgG1, IgG2a, IgG2b, IgG3, and IgG4.

In some embodiments of the thirteenth, fourteenth, and fifteenth aspects of the disclosure, the N-terminal Asp in each of the polypeptides is mutated to Gln.

In some embodiments of the thirteenth, fourteenth, and fifteenth aspects of the disclosure, one or more of the polypeptides lack a C-terminal lysine. In some embodiments, each of the polypeptides lacks a C-terminal lysine.

In some embodiments of the thirteenth, fourteenth, and fifteenth aspects of the disclosure, the Fc-antigen binding domain construct further includes an albumin-binding peptide joined to the N-terminus or C-terminus of one or more of the polypeptides by a linker.

In a sixteenth aspect, the disclosure features an Fc-antigen binding domain construct including: a) a first polypeptide including i) a first Fc domain monomer, ii) a second Fc domain monomer, and iii) a linker joining the first Fc domain monomer and the second Fc domain monomer; b) a second polypeptide including a third Fc domain monomer; c) a third polypeptide including a fourth Fc domain monomer; and d) a first CD38 binding domain joined to the first polypeptide; and e) a second CD38 binding domain joined to the second polypeptide and/or third polypeptide; where the first Fc domain monomer and the third Fc domain monomer combine to form a first Fc domain and the second Fc domain monomer and the fourth Fc domain monomer combine to form a second Fc domain, where the first and the second CD38 binding domains bind different antigens, and where the Fc-antigen binding domain construct has enhanced effector function in an antibody-dependent cytotoxicity (ADCC) assay, an antibody-dependent cellular phagocytosis (ADCP) and/or complement-dependent cytotoxicity (CDC) assay relative to a construct having a single Fc domain and the CD38 binding domain.

In a twenty sixth aspect, the disclosure features an Fc-antigen binding domain construct including: a) a first polypeptide including i) a first Fc domain monomer, ii) a second Fc domain monomer, and iii) a first linker joining the first Fc domain monomer and the second Fc domain monomer; and b) a second polypeptide including iv) a third Fc domain monomer, v) a fourth Fc domain monomer, and vi) a second linker joining the third Fc domain monomer and the fourth Fc domain monomer; and c) a third polypeptide including a fifth Fc domain monomer; d) a fourth polypeptide including an sixth Fc domain monomer; and d) a CD38 binding domain joined to the first polypeptide, second polypeptide, third polypeptide, or fourth polypeptide, where the first Fc domain monomer and the third Fc domain monomer combine to form a first Fc domain and the second Fc domain monomer and the fifth Fc domain monomer combine to form a second Fc domain, the fourth Fc domain monomer and the sixth Fc domain monomer combine to form a third Fc domain, and where the Fc-antigen binding domain construct has enhanced effector function in an antibody-dependent cytotoxicity (ADCC) assay, an antibody-dependent cellular phagocytosis (ADCP) and/or complement-dependent cytotoxicity (CDC) assay relative to a construct having a single Fc domain and the CD38 binding domain.

In a twenty seventh aspect, the disclosure features a Fc-antigen binding domain construct including: a) a first polypeptide including i) a first Fc domain monomer, ii) a second Fc domain monomer, and iii) a first linker joining the first Fc domain monomer and the second Fc domain monomer; and b) a second polypeptide including iv) a third Fc domain monomer, v) a fourth Fc domain monomer, and vi) a second linker joining the third Fc domain monomer and the fourth Fc domain monomer; and c) a third polypeptide including a fifth Fc domain monomer; d) a fourth polypeptide including an sixth Fc domain monomer; and e) a CD38 binding domain joined to the first polypeptide, second polypeptide, third polypeptide, or fourth polypeptide; where the first Fc domain monomer and the third Fc domain monomer combine to form a first Fc domain and the second Fc domain monomer and the fifth Fc domain monomer combine to form a second Fc domain, the fourth Fc domain monomer and the sixth Fc domain monomer combine to form a third Fc domain, and where the Fc-antigen binding domain construct includes a biological activity that is not exhibited by a construct having a single Fc domain and the CD38 binding domain.

In a twenty eighth aspect, the disclosure features an Fc-antigen binding domain construct including: a) a first polypeptide including i) a first Fc domain monomer, ii) a second Fc domain monomer, and iii) a first spacer joining the first Fc domain monomer and the second Fc domain monomer; and b) a second polypeptide including iv) a third Fc domain monomer, v) a fourth Fc domain monomer, and vi) a second spacer joining the third Fc domain monomer and the fourth Fc domain monomer; and c) a third polypeptide including a fifth Fc domain monomer; d) a fourth polypeptide including an sixth Fc domain monomer; and e) a CD38 binding domain joined to the first polypeptide, second polypeptide, third polypeptide, or fourth polypeptide; where the first Fc domain monomer and the third Fc domain monomer combine to form a first Fc domain and the second Fc domain monomer and the fifth Fc domain monomer combine to form a second Fc domain, the fourth Fc domain monomer and the sixth Fc domain monomer combine to form a third Fc domain.

In a twenty ninth aspect, the disclosure features a cell culture medium including a population of Fc-antigen binding domain constructs, where at least 50% of the Fc-antigen binding domain constructs, on a molar basis, include: a) a first polypeptide including i) a first Fc domain monomer, ii) a second Fc domain monomer, and iii) a first linker joining the first Fc domain monomer and the second Fc domain monomer; and b) a second polypeptide including iv) a third Fc domain monomer, v) a fourth Fc domain monomer, and vi) a second linker joining the third Fc domain monomer and the fourth Fc domain monomer; and c) a third polypeptide including a fifth Fc domain monomer; d) a fourth polypeptide including an sixth Fc domain monomer; and e) a CD38 binding domain joined to the first polypeptide, second polypeptide, third polypeptide, or fourth polypeptide; where the first Fc domain monomer and the third Fc domain monomer combine to form a first Fc domain and the second Fc domain monomer and the fifth Fc domain monomer combine to form a second Fc domain, the fourth Fc domain monomer and the sixth Fc domain monomer combine to form a third Fc domain.

In a thirtieth aspect, the disclosure features a method of manufacturing an Fc-antigen binding domain construct, the method including: a) culturing a host cell expressing: (1) a first polypeptide including i) a first Fc domain monomer, ii) a second Fc domain monomer, and iii) a first linker joining the first Fc domain monomer and the second Fc domain monomer; and (2) a second polypeptide including iv) a third Fc domain monomer, v) a fourth Fc domain monomer, and vi) a second linker joining the third Fc domain monomer and the fourth Fc domain monomer; and (3) a third polypeptide including a fifth Fc domain monomer; (4) a fourth polypeptide including an sixth Fc domain monomer; and (5) a CD38 binding domain joined to the first polypeptide, second polypeptide, third polypeptide, or fourth polypeptide; where the first Fc domain monomer and the third Fc domain monomer combine to form a first Fc domain and the second Fc domain monomer and the fifth Fc domain monomer combine to form a second Fc domain, the fourth Fc domain monomer and the sixth Fc domain monomer combine to form a third Fc domain, and where at least 50% of the Fc-antigen binding domain constructs in a cell culture supernatant, on a molar basis, are structurally identical, and b) purifying the Fc-antigen binding domain construct from the cell culture supernatant.

In some embodiments of the twenty sixth, twenty seventh, twenty eighth, twenty ninth, and thirtieth aspect of the disclosure, each of the first and third Fc domain monomers includes a complementary dimerization selectivity module that promote dimerization between the first Fc domain monomer and the third Fc domain monomer, each of the second and fifth Fc domain monomers includes a complementary dimerization selectivity module that promote dimerization between the second Fc domain monomer and the fifth Fc domain monomer, and each of the fourth and sixth Fc domain monomers includes a complementary dimerization selectivity module that promote dimerization between the fourth Fc domain monomer and the sixth Fc domain monomer.

In some embodiments of all aspects of the disclosure, the Fc-antigen binding domain construct has reduced fucosylation. Thus, in some embodiments, less than 40%, 30%, 20%, 15%, 10% or 5% of the Fc domain monomers in a composition comprising an Fc-antigen binding domain construct are fucosylated.

In some embodiments of all aspects of the disclosure, the Fc domain monomer comprises the amino acid sequence of FIG. 24A (SEQ ID NO: 43) with up to 10 (9, 8, 7, 6, 5, 4, 3, 2 or 1) single amino acid changes in the CH3 domain.

In some embodiments of all aspects of the disclosure, the Fc domain monomer comprises the amino acid sequence of FIG. 24B (SEQ ID NO: 45) with up to 10 (9, 8, 7, 6, 5, 4, 3, 2 or 1) single amino acid changes in the CH3 domain.

In some embodiments of all aspects of the disclosure, the Fc domain monomer comprises the amino acid sequence of FIG. 24C (SEQ ID NO: 47) with up to 10 (9, 8, 7, 6, 5, 4, 3, 2 or 1) single amino acid changes in the CH3 domain.

In some embodiments of all aspects of the disclosure, the Fc domain monomer comprises the amino acid sequence of FIG. 24D (SEQ ID NO: 42) with up to 10 (9, 8, 7, 6, 5, 4, 3, 2 or 1) single amino acid changes in the CH3 domain.

In some embodiments of all aspects of the disclosure, for example, when the Fc domain monomer is at the carboxy-terminal end of a polypeptide, the Fc domain monomer does not include K447. In other embodiments, for example, when the Fc domain monomer is not at the carboxy-terminal end of a polypeptide, the Fc domain monomer includes K447.

In some embodiments of all aspects of the disclosure, for example, when the Fc domain monomer is amino terminal to a linker, the Fc domain monomer does not include the portion of the hinge from E216 to C220, inclusive, but does include the portion of the hinge from D221 to L235, inclusive. In other embodiments, for example, when the Fc domain monomer is carboxy-terminal to a CH1 domain, the Fc domain monomer includes the portion of the hinge from E216 to L235, inclusive. In some embodiments of all aspects of the disclosure, a hinge domain, for example a hinge domain at the amino terminus of a polypeptide, has an Asp to Gln mutation at EU position 221.

As noted above, the Fc-antigen binding domain constructs of the disclosure are assembled from polypeptides, including polypeptides comprising two or more IgG1 Fc domain monomers, and such polypeptides are an aspect of the present disclosure.

In a forty first aspect, the disclosure features a polypeptide comprising a CD38 binding domain; a linker; a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain, wherein at least one Fc domain monomer comprises mutations forming an engineered protuberance.

In various embodiments of the forty first aspect: the CD38 binding domain comprises an antibody heavy chain variable domain; the CD38 binding domain comprises an antibody light chain variable domain; the first IgG1 Fc domain monomer comprises two or four reverse charge mutations and the second IgG1 Fc domain monomer comprises mutations forming an engineered protuberance; the first IgG1 Fc domain monomer comprises mutations forming an engineered protuberance and the second IgG1 Fc domain monomer comprises two or four reverse charge mutations; both the first IgG1 Fc domain monomer and the second IgG constant domain monomer comprise mutations forming an engineered protuberance; the polypeptide comprises a third linker and a third IgG1 Fc domain monomer wherein the first IgG1 Fc domain monomer, the second IgG1 Fc domain monomer and the third IgG1 Fc domain monomer each comprise mutations forming an engineered protuberance; the polypeptide comprises a third linker and a third IgG1 Fc domain monomer wherein both the first IgG1 Fc domain monomer and the second IgG1 Fc domain monomer each comprise mutations forming an engineered protuberance and the third IgG1 Fc domain monomer comprises two or four reverse charge mutations; the polypeptide comprises a third linker and third IgG1 Fc domain monomer wherein both the first IgG1 Fc domain monomer and the third IgG1 Fc domain monomer each comprise mutations forming an engineered protuberance and the second IgG1 domain monomer comprises two or four reverse charge mutations; the polypeptide comprises a third linker and a third IgG1 Fc domain monomer wherein both the second IgG1 Fc domain monomer and the third IgG1 Fc domain monomer each comprise mutations forming an engineered protuberance and the first IgG1 domain monomer comprises two or four reverse charge mutations.

In various embodiments of the forty first aspect: the IgG1 Fc domain monomers comprising mutations forming an engineered protuberance further comprise one, two or three reverse charge mutations; the mutations forming an engineered protuberance and the reverse charge mutations are in the CH3 domain; the mutations are within the sequence from EU Numbering position G341 to EU Numbering position K447, inclusive; the mutations are single amino acid changes; the second linker and the optional third linker comprise or consist of an amino acid sequence selected from the group consisting of: GGGGGGGGGGGGGGGGGGGG (SEQ ID NO: 23), GGGGS (SEQ ID NO: 1), GGSG (SEQ ID NO: 2), SGGG (SEQ ID NO: 3), GSGS (SEQ ID NO: 4), GSGSGS (SEQ ID NO: 5), GSGSGSGS (SEQ ID NO: 6), GSGSGSGSGS (SEQ ID NO: 7), GSGSGSGSGSGS (SEQ ID NO: 8), GGSGGS (SEQ ID NO: 9), GGSGGSGGS (SEQ ID NO: 10), GGSGGSGGSGGS (SEQ ID NO: 11), GGSG (SEQ ID NO: 2), GGSG (SEQ ID NO: 2), GGSGGGSG (SEQ ID NO: 12), GGSGGGSGGGSGGGGGSGGGGSGGGGSGGGGS (SEQ ID NO: 213), GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), GGSGGGSEGGGSEGGGSEGGGSEGGGSEGGGSGGGS (SEQ ID NO: 34), GGGSGGGSGGGS (SEQ ID NO: 35), SGGGSGGGSGGGSGGGSGGG (SEQ ID NO: 18), GGSGGGSGGGSGGGSGGS (SEQ ID NO: 36), GGGG (SEQ ID NO: 19), GGGGGGGG (SEQ ID NO: 20), GGGGGGGGGGGG (SEQ ID NO: 21) and GGGGGGGGGGGGGGGG (SEQ ID NO: 22); the second linker and the optional third linker is a glycine spacer; the second linker and the optional third linker independently consist of 4 to 30 (SEQ ID NO: 214), 4 to 20 (SEQ ID NO: 217), 8 to 30 (SEQ ID NO: 215), 8 to 20 (SEQ ID NO: 218), 12 to 20 (SEQ ID NO: 219) or 12 to 30 (SEQ ID NO: 216) glycine residues; the second linker and the optional third linker consist of 20 glycine residues (SEQ ID NO: 23); at least one of the Fc domain monomers comprises a single amino acid mutation at EU Numbering position I253 each amino acid mutation at EU Numbering position I253 is independently selected from the group consisting of I253A, I253C, I253D, I253E, I253F, I253G, I253H, I253I, I253K, I253L, I253M, I253N, I253P, I253Q, I253R, I253S, I253T, I253V, I253W, and I253Y; each amino acid mutation at position I253 is I253A; at least one of the Fc domain monomers comprises a single amino acid mutation at EU Numbering position R292; each amino acid mutation at EU Numbering position R292 is independently selected from the group consisting of R292D, R292E, R292L, R292P, R292Q, R292R, R292T, and R292Y; each amino acid mutation at position R292 is R292P; each Fc domain monomer independently comprises or consists of an amino acid sequence selected from the group consisting of EPKSCDKTHTCPPCPAPELL (SEQ ID NO: 220) and DKTHTCPPCPAPELL (SEQ ID NO: 221); the hinge portion of the second Fc domain monomer and the third Fc domain monomer have the amino acid sequence DKTHTCPPCPAPELL (SEQ ID NO: 221); the hinge portion of the first Fc domain monomer has the amino acid sequence EPKSCDKTHTCPPCPAPEL (SEQ ID NO: 222); the hinge portion of the first Fc domain monomer has the amino acid sequence EPKSCDKTHTCPPCPAPEL (SEQ ID NO: 222) and the hinge portion of the second Fc domain monomer and the third Fc domain monomer have the amino acid sequence DKTHTCPPCPAPELL (SEQ ID NO: 221); the CH2 domains of each Fc domain monomer independently comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223) with no more than two single amino acid deletions or substitutions; the CH2 domains of each Fc domain monomer are identical and comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223) with no more than two single amino acid deletions or substitutions; the CH2 domains of each Fc domain monomer are identical and comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223) with no more than two single amino acid substitutions; the CH2 domains of each Fc domain monomer are identical and comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223); the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 10 single amino acid substitutions; the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 8 single amino acid substitutions; the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 6 single amino acid substitutions; wherein the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 5 single amino acid substitutions; the single amino acid substitutions are selected from the group consisting of: T366Y, T366W, T394W, T394Y, F405W, F405A, Y407A, S354C, Y349T, T394F, K409D, K409E, K392D, K392E, K370D, K370E, D399K, D399R, E357K, E357R, D356K, and D356R; each of the Fc domain monomers independently comprises the amino acid sequence of any of SEQ ID NOs:42, 43, 45, and 47 having up to 10 single amino acid substitutions; up to 6 of the single amino acid substitutions are reverse charge mutations in the CH3 domain or are mutations forming an engineered protuberance; the single amino acid substitutions are within the sequence from EU Numbering position G341 to EU Numbering position K447, inclusive; at least one of the mutations forming an engineered protuberance is selected from the group consisting of T366Y, T366W, T394W, T394Y, F405W, S354C, Y349T, and T394F; the two or four reverse charge mutations are selected from: K409D, K409E, K392D. K392E, K370D, K370E, D399K, D399R, E357K, E357R, D356K, and D356R; the CD38 binding domain is a scFv; the CD38 binding domain comprises a VH domain and a CH1 domain; the CD38 binding domain further comprises a VL domain; the VH domain comprises a set of CDR-H1, CDR-H2 and CDR-H3 sequences set forth in Table 1; the VH domain comprises CDR-H1, CDR-H2, and CDR-H3 of a VH domain comprising a sequence of an antibody set forth in Table 2; the VH domain comprises CDR-H1, CDR-H2, and CDR-H3 of a VH sequence of an antibody set forth in Table 2, and the VH sequence, excluding the CDR-H1, CDR-H2, and CDR-H3 sequence, is at least 95% or 98% identical to the VH sequence of an antibody set forth in Table 2; the VH domain comprises a VH sequence of an antibody set forth in Table 2; the CD38 binding domain comprises a set of CDR-H1, CDR-H2, CDR-H3, CDR-L1, CDR-L2, and CDR-L3 sequences set forth in Table 1; the CD38 binding domain comprises CDR-H1, CDR-H2, CDR-H3, CDR-L1, CDR-L2, and CDR-L3 sequences from a set of a VH and a VL sequence of an antibody set forth in Table 2; the CD38 binding domain comprises a VH domain comprising CDR-H1, CDR-H2, and CDR-H3 of a VH sequence of an antibody set forth in Table 2, and a VL domain comprising CDR-L1, CDR-L2, and CDR-L3 of a VL sequence of an antibody set forth in Table 2, wherein the VH and the VL domain sequences, excluding the CDR-H1, CDR-H2, CDR-H3, CDR-L1, CDR-L2, and CDR-L3 sequences, are at least 95% or 98% identical to the VH and VL sequences of an antibody set forth in Table 2; the CD38 binding domain comprises a set of a VH and a VL sequence of an antibody set forth in Table 2; CD38 binding domain comprises an IgG CL antibody constant domain and an IgG CH1 antibody constant domain; the CD38 binding domain comprises a VH domain and CH1 domain and can bind to a polypeptide comprising a VL domain and a CL domain to form a Fab.

Also described is a polypeptide complex comprising two copies of the polypeptide of described above joined by disulfide bonds between cysteine residues within the hinge of first or second IgG1 Fc domain monomers.

Also described is a polypeptide complex comprising a polypeptide described above joined to a second polypeptide comprising and IgG1 Fc domain monomer comprising a hinge domain, a CH2 domain and a CH3 domain, wherein the polypeptide and the second polypeptide are joined by disulfide bonds between cysteine residues within the hinge domain of the first, second or third IgG1 Fc domain monomer of the polypeptide and the hinge domain of the second polypeptide.

In various embodiments of the complexes: the second polypeptide monomer comprises mutations forming an engineered cavity; the mutations forming the engineered cavity are selected from the group consisting of: Y407T, Y407A, F405A, T394S, T394W/Y407A, T366W/T394S, T366S/L368A/Y407V/Y349C, S364H/F405A; the second polypeptide comprises the amino acid sequence of any of SEQ ID NOs: 42, 43, 45, and 47 having up to 10 single amino acid substitutions.

In a forty second aspect, the disclosure features: a polypeptide comprising: aCD38 binding domain; a linker; a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain, wherein at least one Fc domain monomer comprises one, two or three reverse charge amino acid mutations.

In various embodiments of the forty second aspect: the CD38 binding domain comprises an antibody heavy chain variable domain; the CD38 binding domain comprises an antibody light chain variable domain; the first IgG1 Fc domain monomer comprises a set of two reverse charge mutations selected from those in Tables 4A and 4B or a set of four reverse charge mutation selected from those in Tables 4A and 4B and the second IgG1 Fc domain monomer comprises one, two or three reverse charge amino acid mutations selected from Tables 4A and 4B; the first IgG1 Fc domain monomer comprises one, two or three reverse charge amino acid mutations selected from Tables 4A and 4B and the second IgG1 Fc domain monomer comprises a set of two reverse charge mutations selected from those in Tables 4a and 4b or a set of four reverse charge mutation selected from those in Tables 4A and 4B; both the first IgG1 Fc domain monomer and the second IgG constant domain monomer comprise one, two or three reverse charge amino acid mutations selected from Tables 4A and 4B; the polypeptide further comprises a third linker and a third IgG1 Fc domain monomer wherein the first IgG1 Fc domain monomer, the second IgG1 Fc domain monomer and the third IgG1 Fc domain monomer each comprise one, two or three reverse charge amino acid mutations selected from Tables 4A and 4B; the polypeptide further comprises a third linker and a third IgG1 Fc domain monomer wherein both the first IgG1 Fc domain monomer and the second IgG1 Fc domain monomer each comprise one, two or three reverse charge amino acid mutations selected from Tables 4A and 4B and the third IgG1 Fc domain monomer comprises a set of two reverse charge mutations selected from those in Tables 4A and 4B or a set of four reverse charge mutation selected from those in Tables 4A and 4B; the polypeptide further comprises a third linker and third IgG1 Fc domain monomer wherein both the first IgG1 Fc domain monomer and the third IgG1 Fc domain monomer each comprise one, two or three reverse charge amino acid mutations selected from Tables 4A and 4B and the second IgG1 domain monomer comprises a set of two reverse charge mutations selected from those in Tables 4A and 4B or a set of four reverse charge mutation selected from those in Tables 4A and 4B; the polypeptide further comprises a third linker and a third IgG1 Fc domain monomer wherein both the second IgG1 Fc domain monomer and the third IgG1 Fc domain monomer each comprise one, two or three reverse charge amino acid mutations selected from Tables 4A and 4B and the first IgG1 domain monomer comprises a set of two reverse charge mutations selected from those in Tables 4A and 4B or a set of four reverse charge mutation selected from those in Tables 4A and 4B; the IgG1 Fc domain monomers comprising one, two or three reverse charge amino acid mutations selected from Tables 4A and 4B have identical CH3 domains; one, two or three reverse charge amino acid mutations selected from Tables 4A and 4B are in the CH3 domain; the mutations are within the sequence from EU Numbering position G341 to EU Numbering position K447, inclusive; the mutations are each single amino acid changes; the mutations are within the sequence from EU Numbering position G341 to EU Numbering position K446, inclusive; the mutations are single amino acid changes; the second linker and the optional third linker comprise or consist of an amino acid sequence selected from the group consisting of: GGGGGGGGGGGGGGGGGGGG (SEQ ID NO: 23), GGGGS (SEQ ID NO: 1), GGSG (SEQ ID NO: 2), SGGG (SEQ ID NO: 3), GSGS (SEQ ID NO: 4), GSGSGS (SEQ ID NO: 5), GSGSGSGS (SEQ ID NO: 6), GSGSGSGSGS (SEQ ID NO: 7), GSGSGSGSGSGS (SEQ ID NO: 8), GGSGGS (SEQ ID NO: 9), GGSGGSGGS (SEQ ID NO: 10), GGSGGSGGSGGS (SEQ ID NO: 11), GGSG (SEQ ID NO: 2), GGSG (SEQ ID NO: 2), GGSGGGSG (SEQ ID NO: 12), GGSGGGSGGGSGGGGGSGGGGSGGGGSGGGGS (SEQ ID NO: 213), GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), GGSGGGSEGGGSEGGGSEGGGSEGGGSEGGGSGGGS (SEQ ID NO: 34), GGGSGGGSGGGS (SEQ ID NO: 35), SGGGSGGGSGGGSGGGSGGG (SEQ ID NO: 18), GGSGGGSGGGSGGGSGGS (SEQ ID NO: 36), GGGG (SEQ ID NO: 19), GGGGGGGG (SEQ ID NO: 20), GGGGGGGGGGGG (SEQ ID NO: 21) and GGGGGGGGGGGGGGGG (SEQ ID NO: 22); the second linker and the optional third linker is a glycine spacer; the second linker and the optional third linker independently consist of 4 to 30 (SEQ ID NO: 214), 4 to 20 (SEQ ID NO: 217), 8 to 30 (SEQ ID NO: 215), 8 to 20 (SEQ ID NO: 218), 12 to 20 (SEQ ID NO: 219) or 12 to 30 (SEQ ID NO: 216) glycine residues; the second linker and the optional third linker consist of 20 glycine residues (SEQ ID NO: 23); at least one of the Fc domain monomers comprises a single amino acid mutation at EU Numbering position I253 each amino acid mutation at EU Numbering position I253 is independently selected from the group consisting of I253A, I253C, I253D, I253E, I253F, I253G, I253H, I253I, I253K, I253L, I253M, I253N, I253P, I253Q, I253R, I253S, I253T, I253V, I253W, and I253Y; each amino acid mutation at position I253 is I253A; at least one of the Fc domain monomers comprises a single amino acid mutation at EU Numbering position R292; each amino acid mutation at EU Numbering position R292 is independently selected from the group consisting of R292D, R292E, R292L, R292P, R292Q, R292R, R292T, and R292Y; each amino acid mutation at position R292 is R292P; each Fc domain monomer independently comprises or consists of an amino acid sequence selected from the group consisting of EPKSCDKTHTCPPCPAPELL (SEQ ID NO: 220) and DKTHTCPPCPAPELL (SEQ ID NO: 221); the hinge portion of the second Fc domain monomer and the third Fc domain monomer have the amino acid sequence DKTHTCPPCPAPELL (SEQ ID NO: 221); the hinge portion of the first Fc domain monomer has the amino acid sequence EPKSCDKTHTCPPCPAPEL (SEQ ID NO: 222); the hinge portion of the first Fc domain monomer has the amino acid sequence EPKSCDKTHTCPPCPAPEL (SEQ ID NO: 222) and the hinge portion of the second Fc domain monomer and the third Fc domain monomer have the amino acid sequence DKTHTCPPCPAPELL (SEQ ID NO: 221); the CH2 domains of each Fc domain monomer independently comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223) with no more than two single amino acid deletions or substitutions; the CH2 domains of each Fc domain monomer are identical and comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223) with no more than two single amino acid deletions or substitutions; the CH2 domains of each Fc domain monomer are identical and comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223) with no more than two single amino acid substitutions; the CH2 domains of each Fc domain monomer are identical and comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223); the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 10 single amino acid substitutions; the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 8 single amino acid substitutions; the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 6 single amino acid substitutions; wherein the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 5 single amino acid substitutions; the single amino acid substitutions are selected from the group consisting of: T366Y, T366W, T394W, T394Y, F405W, F405A, Y407A, S354C, Y349T, T394F, K409D, K409E, K392D, K392E, K370D, K370E, D399K, D399R, E357K, E357R, D356K, and D356R; each of the Fc domain monomers independently comprises the amino acid sequence of any of SEQ ID NOs:42, 43, 45, and 47 having up to 10 single amino acid substitutions; up to 6 of the single amino acid substitutions are reverse charge mutations in the CH3 domain or are mutations forming an engineered protuberance; the single amino acid substitutions are within the sequence from EU Numbering position G341 to EU Numbering position K447, inclusive; at least one of the mutations forming an engineered protuberance is selected from the group consisting of T366Y, T366W, T394W, T394Y, F405W, S354C, Y349T, and T394F; the two or four reverse charge mutations are selected from: K409D, K409E, K392D. K392E, K370D, K370E, D399K, D399R, E357K, E357R, D356K, and D356R; the CD38 binding domain is a scFv; CD38 binding domain comprises a VH domain and a CH1 domain; the CD38 binding domain further comprises a VL domain; the VH domain comprises a set of CDR-H1, CDR-H2 and CDR-H3 sequences set forth in Table 1; the VH domain comprises CDR-H1, CDR-H2, and CDR-H3 of a VH domain comprising a sequence of an antibody set forth in Table 2; the VH domain comprises CDR-H1, CDR-H2, and CDR-H3 of a VH sequence of an antibody set forth in Table 2, and the VH sequence, excluding the CDR-H1, CDR-H2, and CDR-H3 sequence, is at least 95% or 98% identical to the VH sequence of an antibody set forth in Table 2; the VH domain comprises a VH sequence of an antibody set forth in Table 2; the CD38 binding domain comprises a set of CDR-H1, CDR-H2, CDR-H3, CDR-L1, CDR-L2, and CDR-L3 sequences set forth in Table 1; the CD38 binding domain comprises CDR-H1, CDR-H2, CDR-H3, CDR-L1, CDR-L2, and CDR-L3 sequences from a set of a VH and a VL sequence of an antibody set forth in Table 2; the CD38 binding domain comprises a VH domain comprising CDR-H1, CDR-H2, and CDR-H3 of a VH sequence of an antibody set forth in Table 2, and a VL domain comprising CDR-L1, CDR-L2, and CDR-L3 of a VL sequence of an antibody set forth in Table 2, wherein the VH and the VL domain sequences, excluding the CDR-H1, CDR-H2, CDR-H3, CDR-L1, CDR-L2, and CDR-L3 sequences, are at least 95% or 98% identical to the VH and VL sequences of an antibody set forth in Table 2; the CD38 binding domain comprises a set of a VH and a VL sequence of an antibody set forth in Table 2; the CD38 binding domain comprises an IgG CL antibody constant domain and an IgG CH1 antibody constant domain; the CD38 binding domain comprises a VH domain and CH1 domain and can bind to a polypeptide comprising a VL domain and a CL domain to form a Fab.

Also described is a polypeptide complex comprising two copies of any of the polypeptides described above joined by disulfide bonds between cysteine residues within the hinge of first or second IgG1 Fc domain monomers.

Also described is a polypeptide complex comprising a polypeptide described above joined to a second polypeptide comprising and IgG1 Fc domain monomer comprising a hinge domain, a CH2 domain and a CH3 domain, wherein the polypeptide and the second polypeptide are joined by disulfide bonds between cysteine residues within the hinge domain of the first, second or third IgG1 Fc domain monomer of the polypeptide and the hinge domain of the second polypeptide. In various embodiments: the second polypeptide monomer comprises one, two or three reverse charge mutations; the second polypeptide monomer comprises one, two or three reverse charge mutations selected from Tables 4A and 4B and are complementary to the one, two or three reverse charge mutations selected Tables 4A and 4B in the polypeptide; the second polypeptide comprises the amino acid sequence of any of SEQ ID NOs: 42, 43, 45, and 47 having up to 10 single amino acid substitutions.

In a forty third aspect, the disclosure features a polypeptide comprising: a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain, wherein at least one Fc domain monomer comprises mutations forming an engineered protuberance.

In various embodiments of the forty third aspect: the polypeptide further comprises: an antibody heavy chain variable domain and CH1 domain amino terminal to the first IgG1 monomer or an scFv amino terminal to the first IgG1 monomer; the first IgG1 Fc domain monomer comprises two or four reverse charge mutations and the second IgG1 Fc domain monomer comprises mutations forming an engineered protuberance; the first IgG1 Fc domain monomer comprises mutations forming an engineered protuberance and the second IgG1 Fc domain monomer comprises two or four reverse charge mutations; both the first IgG1 Fc domain monomer and the second IgG constant domain monomer comprise mutations forming an engineered protuberance; the polypeptide comprises a third linker and a third IgG1 Fc domain monomer wherein the first IgG1 Fc domain monomer, the second IgG1 Fc domain monomer and the third IgG1 Fc domain monomer each comprise mutations forming an engineered protuberance; the polypeptide comprises a third linker and a third IgG1 Fc domain monomer wherein both the first IgG1 Fc domain monomer and the second IgG1 Fc domain monomer each comprise mutations forming an engineered protuberance and the third IgG1 Fc domain monomer comprises two or four reverse charge mutations; the polypeptide comprises a third linker and third IgG1 Fc domain monomer wherein both the first IgG1 Fc domain monomer and the third IgG1 Fc domain monomer each comprise mutations forming an engineered protuberance and the second IgG1 domain monomer comprises two or four reverse charge mutations; the polypeptide comprises a third linker and a third IgG1 Fc domain monomer wherein both the second IgG1 Fc domain monomer and the third IgG1 Fc domain monomer each comprise mutations forming an engineered protuberance and the first IgG1 domain monomer comprises two or four reverse charge mutations.

In various embodiments of the forty third aspect: the IgG1 Fc domain monomers comprising mutations forming an engineered protuberance further comprise one, two or three reverse charge mutations;

the mutations forming an engineered protuberance and the reverse charge mutations are in the CH3 domain; the mutations are within the sequence from EU Numbering position G341 to EU Numbering position K447, inclusive; the mutations are single amino acid changes; the second linker and the optional third linker comprise or consist of an amino acid sequence selected from the group consisting of: GGGGGGGGGGGGGGGGGGGG (SEQ ID NO: 23), GGGGS (SEQ ID NO: 1), GGSG (SEQ ID NO: 2), SGGG (SEQ ID NO: 3), GSGS (SEQ ID NO: 4), GSGSGS (SEQ ID NO: 5), GSGSGSGS (SEQ ID NO: 6), GSGSGSGSGS (SEQ ID NO: 7), GSGSGSGSGSGS (SEQ ID NO: 8), GGSGGS (SEQ ID NO: 9), GGSGGSGGS (SEQ ID NO: 10), GGSGGSGGSGGS (SEQ ID NO: 11), GGSG (SEQ ID NO: 2), GGSG (SEQ ID NO: 2), GGSGGGSG (SEQ ID NO: 12), GGSGGGSGGGSGGGGGSGGGGSGGGGSGGGGS (SEQ ID NO: 213), GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), GGSGGGSEGGGSEGGGSEGGGSEGGGSEGGGSGGGS (SEQ ID NO: 34), GGGSGGGSGGGS (SEQ ID NO: 35), SGGGSGGGSGGGSGGGSGGG (SEQ ID NO: 18), GGSGGGSGGGSGGGSGGS (SEQ ID NO: 36), GGGG (SEQ ID NO: 19), GGGGGGGG (SEQ ID NO: 20), GGGGGGGGGGGG (SEQ ID NO: 21) and GGGGGGGGGGGGGGGG (SEQ ID NO: 22); the second linker and the optional third linker is a glycine spacer; the second linker and the optional third linker independently consist of 4 to 30 (SEQ ID NO: 214), 4 to 20 (SEQ ID NO: 217), 8 to 30 (SEQ ID NO: 215), 8 to 20 (SEQ ID NO: 218), 12 to 20 (SEQ ID NO: 219) or 12 to 30 (SEQ ID NO: 216) glycine residues; the second linker and the optional third linker consist of 20 glycine residues (SEQ ID NO: 23); at least one of the Fc domain monomers comprises a single amino acid mutation at EU Numbering position I253 each amino acid mutation at EU Numbering position I253 is independently selected from the group consisting of I253A, I253C, I253D, I253E, I253F, I253G, I253H, I253I, I253K, I253L, I253M, I253N, I253P, I253Q, I253R, I253S, I253T, I253V, I253W, and I253Y; each amino acid mutation at position I253 is I253A; at least one of the Fc domain monomers comprises a single amino acid mutation at EU Numbering position R292; each amino acid mutation at EU Numbering position R292 is independently selected from the group consisting of R292D, R292E, R292L, R292P, R292Q, R292R, R292T, and R292Y; each amino acid mutation at position R292 is R292P; each Fc domain monomer independently comprises or consists of an amino acid sequence selected from the group consisting of EPKSCDKTHTCPPCPAPELL (SEQ ID NO: 220) and DKTHTCPPCPAPELL (SEQ ID NO: 221); the hinge portion of the second Fc domain monomer and the third Fc domain monomer have the amino acid sequence DKTHTCPPCPAPELL (SEQ ID NO: 221); the hinge portion of the first Fc domain monomer has the amino acid sequence EPKSCDKTHTCPPCPAPEL (SEQ ID NO: 222); the hinge portion of the first Fc domain monomer has the amino acid sequence EPKSCDKTHTCPPCPAPEL (SEQ ID NO: 222) and the hinge portion of the second Fc domain monomer and the third Fc domain monomer have the amino acid sequence DKTHTCPPCPAPELL (SEQ ID NO: 221); the CH2 domains of each Fc domain monomer independently comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223) with no more than two single amino acid deletions or substitutions; the CH2 domains of each Fc domain monomer are identical and comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223) with no more than two single amino acid deletions or substitutions; the CH2 domains of each Fc domain monomer are identical and comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223) with no more than two single amino acid substitutions; the CH2 domains of each Fc domain monomer are identical and comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223); the CH3 domains of each Fc domain monomer independently comprise the amino acid sequence:

GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 10 single amino acid substitutions; the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 8 single amino acid substitutions; the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 6 single amino acid substitutions; wherein the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 5 single amino acid substitutions; the single amino acid substitutions are selected from the group consisting of: T366Y, T366W, T394W, T394Y, F405W, F405A, Y407A, S354C, Y349T, T394F, K409D, K409E, K392D, K392E, K370D, K370E, D399K, D399R, E357K, E357R, D356K, and D356R; each of the Fc domain monomers independently comprises the amino acid sequence of any of SEQ ID NOs:42, 43, 45, and 47 having up to 10 single amino acid substitutions; up to 6 of the single amino acid substitutions are reverse charge mutations in the CH3 domain or are mutations forming an engineered protuberance; the single amino acid substitutions are within the sequence from EU Numbering position G341 to EU Numbering position K447, inclusive; at least one of the mutations forming an engineered protuberance is selected from the group consisting of T366Y, T366W, T394W, T394Y, F405W, S354C, Y349T, and T394F; the two or four reverse charge mutations are selected from: K409D, K409E, K392D. K392E, K370D, K370E, D399K, D399R, E357K, E357R, D356K, and D356R.

In a forty fourth aspect the disclosure features a polypeptide comprising: a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain, wherein at least one Fc domain monomer comprises one, two or three reverse charge amino acid mutations.

In various embodiments of the forty fourth aspect: the polypeptide further comprises an antibody heavy chain variable domain and CH1 domain amino terminal to the first IgG1 Fc domain monomer or scFv amino terminal to the first IgG1 Fc domain monomer; the first IgG1 Fc domain monomer comprises a set of two reverse charge mutations selected from those in Tables 4A and 4B or a set of four reverse charge mutation selected from those in Tables 4A and 4B and the second IgG1 Fc domain monomer comprises one, two or three reverse charge amino acid mutations selected from Tables 4A and 4B; the first IgG1 Fc domain monomer comprises one, two or three reverse charge amino acid mutations selected from Tables 4A and 4B and the second IgG1 Fc domain monomer comprises a set of two reverse charge mutations selected from those in Tables 4a and 4b or a set of four reverse charge mutation selected from those in Tables 4A and 4B; both the first IgG1 Fc domain monomer and the second IgG constant domain monomer comprise one, two or three reverse charge amino acid mutations selected from Tables 4A and 4B; the polypeptide further comprises a third linker and a third IgG1 Fc domain monomer wherein the first IgG1 Fc domain monomer, the second IgG1 Fc domain monomer and the third IgG1 Fc domain monomer each comprise one, two or three reverse charge amino acid mutations selected from Tables 4A and 4B; the polypeptide further comprises a third linker and a third IgG1 Fc domain monomer wherein both the first IgG1 Fc domain monomer and the second IgG1 Fc domain monomer each comprise one, two or three reverse charge amino acid mutations selected from Tables 4A and 4B and the third IgG1 Fc domain monomer comprises a set of two reverse charge mutations selected from those in Tables 4A and 4B or a set of four reverse charge mutation selected from those in Tables 4A and 4B; the polypeptide further comprises a third linker and third IgG1 Fc domain monomer wherein both the first IgG1 Fc domain monomer and the third IgG1 Fc domain monomer each comprise one, two or three reverse charge amino acid mutations selected from Tables 4A and 4B and the second IgG1 domain monomer comprises a set of two reverse charge mutations selected from those in Tables 4A and 4B or a set of four reverse charge mutation selected from those in Tables 4A and 4B; the polypeptide further comprises a third linker and a third IgG1 Fc domain monomer wherein both the second IgG1 Fc domain monomer and the third IgG1 Fc domain monomer each comprise one, two or three reverse charge amino acid mutations selected from Tables 4A and 4B and the first IgG1 domain monomer comprises a set of two reverse charge mutations selected from those in Tables 4A and 4B or a set of four reverse charge mutation selected from those in Tables 4A and 4BB; the IgG1 Fc domain monomers comprising one, two or three reverse charge amino acid mutations selected from Tables 4A and 4B have identical CH3 domains; one, two or three reverse charge amino acid mutations selected from Tables 4A and 4B are in the CH3 domain; the mutations are within the sequence from EU Numbering position G341 to EU Numbering position K447, inclusive; the mutations are each single amino acid changes; the mutations are within the sequence from EU Numbering position G341 to EU Numbering position K446, inclusive; the mutations are single amino acid changes; the second linker and the optional third linker comprise or consist of an amino acid sequence selected from the group consisting of: GGGGGGGGGGGGGGGGGGGG (SEQ ID NO: 23), GGGGS (SEQ ID NO: 1), GGSG (SEQ ID NO: 2), SGGG (SEQ ID NO: 3), GSGS (SEQ ID NO: 4), GSGSGS (SEQ ID NO: 5), GSGSGSGS (SEQ ID NO: 6), GSGSGSGSGS (SEQ ID NO: 7), GSGSGSGSGSGS (SEQ ID NO: 8), GGSGGS (SEQ ID NO: 9), GGSGGSGGS (SEQ ID NO: 10), GGSGGSGGSGGS (SEQ ID NO: 11), GGSG (SEQ ID NO: 2), GGSG (SEQ ID NO: 2), GGSGGGSG (SEQ ID NO: 12), GGSGGGSGGGSGGGGGSGGGGSGGGGSGGGGS (SEQ ID NO: 213), GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), GGSGGGSEGGGSEGGGSEGGGSEGGGSEGGGSGGGS (SEQ ID NO: 34), GGGSGGGSGGGS (SEQ ID NO: 35), SGGGSGGGSGGGSGGGSGGG (SEQ ID NO: 18), GGSGGGSGGGSGGGSGGS (SEQ ID NO: 36), GGGG (SEQ ID NO: 19), GGGGGGGG (SEQ ID NO: 20), GGGGGGGGGGGG (SEQ ID NO: 21) and GGGGGGGGGGGGGGGG (SEQ ID NO: 22); the second linker and the optional third linker is a glycine spacer; the second linker and the optional third linker independently consist of 4 to 30 (SEQ ID NO: 214), 4 to 20 (SEQ ID NO: 217), 8 to 30 (SEQ ID NO: 215), 8 to 20 (SEQ ID NO: 218), 12 to 20 (SEQ ID NO: 219) or 12 to 30 (SEQ ID NO: 216) glycine residues; the second linker and the optional third linker consist of 20 glycine residues (SEQ ID NO: 23); at least one of the Fc domain monomers comprises a single amino acid mutation at EU Numbering position I253 each amino acid mutation at EU Numbering position I253 is independently selected from the group consisting of I253A, I253C, I253D, I253E, I253F, I253G, I253H, I253I, I253K, I253L, I253M, I253N, I253P, I253Q, I253R, I253S, I253T, I253V, I253W, and I253Y; each amino acid mutation at position I253 is I253A; at least one of the Fc domain monomers comprises a single amino acid mutation at EU Numbering position R292; each amino acid mutation at EU Numbering position R292 is independently selected from the group consisting of R292D, R292E, R292L, R292P, R292Q, R292R, R292T, and R292Y; each amino acid mutation at position R292 is R292P; each Fc domain monomer independently comprises or consists of an amino acid sequence selected from the group consisting of EPKSCDKTHTCPPCPAPELL (SEQ ID NO: 220) and DKTHTCPPCPAPELL (SEQ ID NO: 221); the hinge portion of the second Fc domain monomer and the third Fc domain monomer have the amino acid sequence DKTHTCPPCPAPELL (SEQ ID NO: 221); the hinge portion of the first Fc domain monomer has the amino acid sequence EPKSCDKTHTCPPCPAPEL (SEQ ID NO: 222); the hinge portion of the first Fc domain monomer has the amino acid sequence EPKSCDKTHTCPPCPAPEL (SEQ ID NO: 222) and the hinge portion of the second Fc domain monomer and the third Fc domain monomer have the amino acid sequence DKTHTCPPCPAPELL (SEQ ID NO: 221); the CH2 domains of each Fc domain monomer independently comprise the amino acid sequence: GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223) with no more than two single amino acid deletions or substitutions; the CH2 domains of each Fc domain monomer are identical and comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223) with no more than two single amino acid deletions or substitutions; the CH2 domains of each Fc domain monomer are identical and comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223) with no more than two single amino acid substitutions; the CH2 domains of each Fc domain monomer are identical and comprise the amino acid sequence:
GGPSVFLFPPKPKDTLMISRTPEVTCVVVDVSHEDPEVKFNVVYVDGVEVHNAKTKPREEQYNSTYRVVS VLTVLHQDWLNGKEYKCKVSNKALPAPIEKTISKAK (SEQ ID NO: 223); the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 10 single amino acid substitutions; the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 8 single amino acid substitutions; the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 6 single amino acid substitutions; wherein the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 224) with no more than 5 single amino acid substitutions; the single amino acid substitutions are selected from the group consisting of: T366Y, T366W, T394W, T394Y, F405W, F405A, Y407A, S354C, Y349T, T394F, K409D, K409E, K392D, K392E, K370D, K370E, D399K, D399R, E357K, E357R, D356K, and D356R; each of the Fc domain monomers independently comprises the amino acid sequence of any of SEQ ID NOs:42, 43, 45, and 47 having up to 10 single amino acid substitutions; up to 6 of the single amino acid substitutions are reverse charge mutations in the CH3 domain or are mutations forming an engineered protuberance; the single amino acid substitutions are within the sequence from EU Numbering position G341 to EU Numbering position K447, inclusive; the VH domain or scFv comprises a set of CDR-H1, CDR-H2 and CDR-H3 sequences set forth in Table 1; the VH domain or scFv comprises CDR-H1, CDR-H2, and CDR-H3 of a VH domain comprising a sequence of an antibody set forth in Table 2; the VH domain or scFv comprises CDR-H1, CDR-H2, and CDR-H3 of a VH sequence of an antibody set forth in Table 2, and the VH sequence, excluding the CDR-H1, CDR-H2, and CDR-H3 sequence, is at least 95% or 98% identical to the VH sequence of an antibody set forth in Table 2; the VH domain or scFv comprises a VH sequence of an antibody set forth in Table 2; the VH domain or scFv comprises a set of CDR-H1, CDR-H2, CDR-H3, CDR-L1, CDR-L2, and CDR-L3 sequences set forth in Table 1; the VH domain or scFv comprises CDR-H1, CDR-H2, CDR-H3, CDR-L1, CDR-L2, and CDR-L3 sequences from a set of a VH and a VL sequence of an antibody set forth in Table 2; the VH domain or scFv main comprises a VH domain comprising CDR-H1, CDR-H2, and CDR-H3 of a VH sequence of an antibody set forth in Table 2, and a VL domain comprising CDR-L1, CDR-L2, and CDR-L3 of a VL sequence of an antibody set forth in Table 2, wherein the VH and the VL domain sequences, excluding the CDR-H1, CDR-H2, CDR-H3, CDR-L1, CDR-L2, and CDR-L3 sequences, are at least 95% or 98% identical to the VH and VL sequences of an antibody set forth in Table 2; the VH domain or scFv comprises a set of a VH and a VL sequence of an antibody set forth in Table 2.

Also describes is a nucleic acid molecule encoding any of the forgoing polypeptides of the forty first, forty second, forty third and forty fourth aspects.

Also described is: an expression vector that includes a nucleic acid encoding any of the forgoing polypeptide; host cells containing the nucleic acids or expression vectors; host cells further containing a nucleic acid molecule encoding a polypeptide comprising an antibody VL domain (e.g., a nucleic acid molecule encoding a polypeptide comprising an antibody VL domain and an antibody CL domain); a host cell further containing a nucleic acid molecule encoding a polypeptide comprising an antibody VL domain and an antibody CL domain; a host cells further containing a nucleic acid molecule encoding a polypeptide comprising an IgG1 Fc domain monomer having no more than 10 single amino acid mutations; a host cell further containing a nucleic acid molecule encoding a polypeptide comprising IgG1 Fc domain monomer having no more than 10 single amino acid mutations. In various embodiments: the IgG1 Fc domain monomer comprises the amino acid sequence of any of SEQ ID Nos; 42, 43, 45 and 47 having no more than 10, 8, 6 or 4 single amino acid mutations in the CH3 domain.

Also described is a pharmaceutical composition comprising any of the polypeptide or polypeptide complexes described herein. In various embodiments less than 40%, 30%, 20%, 10%, 5%, 2% of the polypeptides have at least one fucose.

The polypeptides of the of forty first, forty second, forty third and forty fourth aspects of the disclosure are useful as components of the various Fc-antigen binding domain constructs described herein. Thus, the polypeptides of any of the first through fortieth aspects, e.g., those can comprise a CD38 binding domain, can comprise or consist of the polypeptides of any of forty first, forty second, forty third and forty fourth aspects of the disclosure.

Other useful polypeptides for use in all aspects of the disclosure include polypeptides comprising an Fc domain monomer (e.g., comprising or consisting of the amino acid sequence of any of SEQ ID Nos: 42, 43, 45 and 47 with no more than 8, 6, 5, 4, or 3 single amino acid substitutions) having one, two or three mutations forming a cavity (e.g., selected from: Y407T Y407A, F405A, T394S, T394W:Y407T, T394S:Y407A, T366W:T394S, F405T, T3665:L368A:Y407V:Y349C, 5364H:F405A). These polypeptides can optionally include one, two or three reverse charge mutations from Tables 4A and 4B.

Also described herein is an Fc-antigen binding domain construct comprising:

a) a first polypeptide comprising:
i) a first Fc domain monomer,
ii) a second Fc domain monomer
iii) a first CD38 heavy chain binding domain, and
iv) a linker joining the first and second Fc domain monomers;

b) a second polypeptide comprising:
i) a third Fc domain monomer,
ii) a fourth Fc domain monomer
iii) a second CD38 heavy chain binding domain and
iv) a linker joining the third and fourth Fc domain monomers;

c) a third polypeptide comprising a fifth Fc domain monomer;

d) a fourth polypeptide comprising a sixth Fc domain monomer;

e) a fifth polypeptide comprising a first CD38 light chain binding domain; and

f) a sixth polypeptide comprising a second CD38 light chain binding domain;

wherein the first and third Fc domain monomers together form a first Fc domain, the second and fifth Fc domain monomers together form a second Fc domain, the fourth and sixth Fc monomers together form a third Fc domain, the first CD38 heavy chain binding domain and first CD38 light chain binding domain together form a first Fab; and the second CD38 heavy chain binding domain and second CD38 light chain binding domain together form a second Fab.

In various embodiments: the first and second polypeptides are identical in sequence; the third and fourth polypeptides are identical in sequence; the fifth and sixth polypeptides are identical in sequence; the first and second polypeptides are identical in sequence, the third and fourth polypeptides are identical in sequence, and the fifth and sixth polypeptides are identical in sequence; the CH3 domain of each of the Fc domain monomers includes up to 8, 7, 6, 5, 4, 3, 2 or 1 single amino acid substitutions; the CH3 domain of each of the Fc domain monomers includes up to 8, 7, 6, 5, 4, 3, 2 or 1 single amino acid substitutions compared to the amino acid sequence of human IgG; each of the Fc domain monomers independently comprises the amino acid sequence of any of SEQ ID NOs:42, 43, 45, and 47 having up to 10, 8, 7, 6, 5, 4, 3, 2 or 1 single amino acid substitutions; the single amino acids substitutions are only in the CH3 domain; the first and third Fc domain monomers comprise up to 8, 7, 6, 5, 4, 3, 2 or 1 single amino acid substitutions that promote homodimerization between the first and third Fc domain monomers; the second and fifth Fc domain monomers comprise up to 8, 7, 6, 5, 4, 3, 2 or 1 single amino acid substitutions that promote heterodimerization between the second and fifth Fc domain monomers and the fourth and sixth Fc domain monomers comprise up to 8, 7, 6, 5, 4, 3, 2 or 1 single amino acid substitutions that promote heterodimerization between the fourth and sixth Fc domain monomers; the substitutions that promote homodimerization are selected from substitutions in Table 4A and 4B; and the substitutions that promote heterodimerization are selected from substitutions in Table 3.

Also described is anFc-antigen binding domain construct comprising:

a) a first polypeptide comprising:
i) a first Fc domain monomer,
ii) a second Fc domain monomer
iii) a first CD38 heavy chain binding domain, and
iv) a linker joining the first and second Fc domain monomers;

b) a second polypeptide comprising:
i) a third Fc domain monomer,
ii) a fourth Fc domain monomer
iii) a second CD38 heavy chain binding domain and
iv) a linker joining the third and fourth Fc domain monomers;

c) a third polypeptide comprising a fifth Fc domain monomer and a first CD38 light chain binding domain; and

d) a fourth polypeptide comprising a sixth Fc domain monomer and a second CD38 light chain binding domain;

wherein the first and third Fc domain monomers together form a first Fc domain, the second and fifth Fc domain monomers together form a second Fc domain, the fourth and sixth Fc monomers together form a third Fc domain, the first CD38 heavy chain binding domain and first CD38 light chain binding domain together form a first Fab; and the second CD38 heavy chain binding domain and second CD38 light chain binding domain together form a second Fab.

Also described is an Fc-antigen binding domain construct, comprising:

a) a first polypeptide comprising:
i) a first Fc domain monomer,
ii) a second Fc domain monomer
iii) a first CD38 heavy chain binding domain ,and
iv) a linker joining the first and second Fc domain monomers;

b) a second polypeptide comprising:
i) a third Fc domain monomer,
ii) a fourth Fc domain monomer
iii) a second CD38 heavy chain binding domain and
iv) a linker joining the third and fourth Fc domain monomers;

c) a third polypeptide comprising a fifth Fc domain monomer;

d) a fourth polypeptide comprising a sixth Fc domain monomer;

e) a fifth polypeptide comprising a first CD38 light chain binding domain; and

f) a sixth polypeptide comprising a second CD38 light chain binding domain;

wherein the first and fifth Fc domain monomers together form a first Fc domain, the third and sixth Fc domain monomers together form an second Fc domain, the second and fourth Fc monomers together form a third Fc domain, the first CD38 heavy chain binding domain and first CD38 light chain binding domain together form a first Fab; and the second CD38 heavy chain binding domain and second CD38 light chain binding domain together form a second F

n various embodiments: the first and second polypeptides are identical in sequence; third and fourth polypeptides are identical in sequence; the fifth and sixth polypeptides are identical in sequence; the first and second polypeptides are identical in sequence, the third and fourth polypeptides are identical in sequence, and the fifth and sixth polypeptides are identical in sequence; the CH3 domain of each of the Fc domain monomers includes up to 8, 7, 6, 5, 4, 3, 2 or 1 single amino acid substitutions; the CH3 domain of each of the Fc domain monomers includes up to 8, 7, 6, 5, 4, 3, 2 or 1 single amino acid substitutions compared to the amino acid sequence of human IgG1; each of the Fc domain monomers independently comprises the amino acid sequence of any of SEQ ID NOs:42, 43, 45, and 47 having up to 10, 8, 7, 6, 5, 4, 3, 2 or 1 single amino acid substitutions; the single amino acids substitutions are only in the CH3 domain; the second and fourth Fc domain monomers comprise up to 8, 7, 6, 5, 4, 3, 2 or 1 single amino acid substitutions that promote homodimerization between the second and fourth Fc domain monomers; the first and fifth Fc domain monomers comprise up to 8, 7, 6, 5, 4, 3, 2 or 1 single amino acid substitutions that promote heterodimerization between the first and fifth Fc domain monomers and the third and sixth Fc domain monomers comprise up to 8, 7, 6, 5, 4, 3, 2 or 1 single amino acid substitutions that promote heterodimerization between the fourth and sixth Fc domain monomers; the substitutions that promote homodimerization are selected from substitutions in Table 4A and 4B; and the substitutions that promote heterodimerization are selected from substitutions in Table 3.

Also described in an Fc-antigen binding domain construct, comprising:

a) a first polypeptide comprising:
i) a first Fc domain monomer,
ii) a second Fc domain monomer,
iii) a third Fc domain monomer,
iv) a first CD38 heavy chain binding domain,
v) a linker joining the first and he second Fc domain monomers, and
vi) a linker joining the second and third Fc domain monomers;

b) a second polypeptide comprising:
i) a fourth Fc domain monomer,
ii) a fifth Fc domain monomer,
iii) a sixth Fc domain monomer,
iv) a second CD38 heavy chain binding domain,
v) a linker joining the fourth and fifth Fc domain monomers, and
vi) a linker joining the fifth and sixth Fc domain monomers;

c) a third polypeptide comprising a seventh Fc domain monomer;

d) a fourth polypeptide comprising an eighth Fc domain monomer;

e) a fifth polypeptide comprising ninth Fc domain monomer;

f) a sixth polypeptide comprising a tenth Fc domain monomer;

g) a seventh polypeptide comprising a first CD38 light chain binding domain; and

h) an eighth polypeptide comprising a second CD38 light chain binding domain;

wherein the first and seventh Fc domain monomers together form a first Fc domain, the fourth and eighth Fc domain monomers together form an second Fc domain, the second and fifth Fc monomer together form a third Fc domain, the third and ninth Fc domain monomers together form a fourth Fc domain, the sixth and tenth Fc monomers together form a fifth Fc domain, the first CD38 heavy chain binding domain and first CD38 light chain binding domain together form a first Fab; and the second CD38 heavy chain binding domain and second CD38 light chain binding domain together form a second Fab.

In various embodiments: the first and second polypeptides are identical in sequence; the third and fourth polypeptides are identical in sequence; the fifth and sixth polypeptides are identical in sequence; the seventh and eighth polypeptides are identical in sequence; the first and second polypeptides are identical in sequence, the third and fourth polypeptides are identical in sequence, the fifth and sixth polypeptides are identical in sequence, and the seventh and eighth polypeptides are identical in sequence; the CH3 domain of each of the Fc domain monomers includes up to 8, 7, 6, 5, 4, 3, 2 or 1 single amino acid substitutions; the CH3 domain of each of the Fc domain monomers includes up to 8, 7, 6, 5, 4, 3, 2 or 1 single amino acid substitutions compared to the amino acid sequence of human IgG1; the Fc domain monomers independently comprises the amino acid sequence of any of SEQ ID NOs:42, 43, 45, and 47 having up to 10, 8, 7, 6, 5, 4, 3, 2 or 1 single amino acid substitutions; the single amino acids substitutions are only in the CH3 domain; the second and fifth Fc domain monomers comprise up to 8, 7, 6, 5, 4, 3, 2 or 1 single amino acid substitutions that promote homodimerization between the second and fifth Fc domain monomers; the first and seventh Fc domain monomers comprise up to 8, 7, 6, 5, 4, 3, 2 or 1 single amino acid substitutions that promote heterodimerization between the first and seventh Fc domain monomers, the fourth and eighth Fc domain monomers comprise up to 8, 7, 6, 5, 4, 3, 2 or 1 single amino acid substitutions that promote heterodimerization between the fourth and eighth Fc domain monomers, the third and ninth Fc domain monomers comprise up to 8, 7, 6, 5, 4, 3, 2 or 1 single amino acid substitutions that promote heterodimerization between the third and ninth Fc domain monomers, and the sixth and tenth Fc domain monomers comprise up to 8, 7, 6, 5, 4, 3, 2 or 1 single amino acid substitutions that promote heterodimerization between the sixth and tenth Fc domain monomers; the substitutions that promote homodimerization are selected from substitutions in Table 4A and 4B; the substitutions that promote heterodimerization are selected from substitutions in Table 3.

Also described is an Fc-antigen binding domain construct, comprising:

a) a first polypeptide comprising:
i) a first Fc domain monomer,
ii) a second Fc domain monomer,
iii) a third Fc domain monomer,
iv) a first CD38 heavy chain binding domain,
v) a linker joining the first and he second Fc domain monomers, and
vi) a linker joining the second and third Fc domain monomers;

b) a second polypeptide comprising:
i) a fourth Fc domain monomer,
ii) a fifth Fc domain monomer,
iii) a sixth Fc domain monomer,
iv) a second CD38 heavy chain binding domain,
v) a linker joining the fourth and fifth Fc domain monomers, and
vi) a linker joining the fifth and sixth Fc domain monomers;

c) a third polypeptide comprising a seventh Fc domain monomer;

d) a fourth polypeptide comprising an eighth Fc domain monomer;

e) a fifth polypeptide comprising ninth Fc domain monomer and a first CD38 light chain binding domain; and

f) a sixth polypeptide comprising a tenth Fc domain monomer and ; a second CD38 light chain binding domain

wherein the first and seventh Fc domain monomers together form a first Fc domain, the fourth and eighth Fc domain monomers together form an second Fc domain, the second and fifth Fc monomer together form a third Fc domain, the third and ninth Fc domain monomers together form a fourth Fc domain, the sixth and tenth Fc monomers together form a fifth Fc domain, the first CD38 heavy chain binding domain and first CD38 light chain binding domain together form a first Fab; and the second CD38 heavy chain binding domain and second CD38 light chain binding domain together form a second Fab.

Also described is an Fc-antigen binding domain construct, comprising:

a) a first polypeptide comprising:
i) a first Fc domain monomer,
ii) a second Fc domain monomer,
iii) a third Fc domain monomer,
iv) a first CD38 heavy chain binding domain,
v) a linker joining the first and second Fc domain monomers, and
vi) a linker joining the second and to third Fc domain monomers;

b) a second polypeptide comprising:
i) a fourth Fc domain monomer,
ii) a fifth Fc domain monomer,
iii) a sixth Fc domain monomer,
iv) a second CD38 heavy chain binding domain,
v) a linker joining the fourth and fifth Fc domain monomers, and
vi) a linker joining the fifth and sixth Fc domain monomers;

c) a third polypeptide comprising a seventh Fc domain monomer;

d) a fourth polypeptide comprising an eighth Fc domain monomer;

e) a fifth polypeptide comprising ninth Fc domain monomer;

f) a sixth polypeptide comprising a tenth Fc domain monomer;

g) a seventh polypeptide comprising a first CD38 light chain binding domain; and

h) an eighth polypeptide comprising a second CD38 light chain binding domain;

wherein the first and fourth Fc domain monomers together form a first Fc domain, the second and seventh Fc domain monomers together form an second Fc domain, the fifth and eighth Fc monomers together form a third Fc domain, the third and ninth Fc domain monomers together form a fourth Fc domain, the sixth and tenth Fc monomers together form a fifth Fc domain, the first CD38 heavy chain binding domain and first CD38 light chain binding domain together form a first Fab; and the second CD38 heavy chain binding domain and second CD38 light chain binding domain together form a second Fab.

In various embodiments: the first and second polypeptides are identical in sequence; the third and fourth polypeptides are identical in sequence; the fifth and sixth polypeptides are identical in sequence; the seventh and eighth polypeptides are identical in sequence; the first and second polypeptides are identical in sequence, the third and fourth polypeptides are identical in sequence, the fifth and sixth polypeptides are identical in sequence, and the seventh and eighth polypeptides are identical in sequence; the CH3 domain of each of the Fc domain monomers includes up to 8, 7, 6, 5, 4, 3, 2 or 1 single amino acid substitutions; the CH3 domain of each of the Fc domain monomers includes up to 8, 7, 6, 5, 4, 3, 2 or 1 single amino acid substitutions compared to the amino acid sequence of human IgG1; each of the Fc domain monomers independently comprises the amino acid sequence of any of SEQ ID NOs:42, 43, 45, and 47 having up to 10, 8, 7, 6, 5, 4, 3, 2 or 1 single amino acid substitutions; the single amino acids substitutions are only in the CH3 domain; the first and fourth Fc domain monomers comprise up to 8, 7, 6, 5, 4, 3, 2 or 1 single amino acid substitutions that promote homodimerization between the first and fourth Fc domain monomers; the second and seventh Fc domain monomers comprise up to 8, 7, 6, 5, 4, 3, 2 or 1 single amino acid substitutions that promote heterodimerization between the second and seventh Fc domain monomers, the fifth and eighth Fc domain monomers comprise up to 8, 7, 6, 5, 4, 3, 2 or 1 single amino acid substitutions that promote heterodimerization between the fifth and eighth Fc domain monomers, the third and ninth Fc domain monomers comprise up to 8, 7, 6, 5, 4, 3, 2 or 1 single amino acid substitutions that promote heterodimerization between the third and ninth Fc domain monomers, and the sixth and tenth Fc domain monomers comprise up to 8, 7, 6, 5, 4, 3, 2 or 1 single amino acid substitutions that promote heterodimerization between the sixth and tenth Fc domain monomers; the substitutions that promote homodimerization are selected from substitutions in Table 4A and 4B; and the substitutions that promote heterodimerization are selected from substitutions in Table 3.

Also described is an Fc-antigen binding domain construct, comprising:

a) a first polypeptide comprising:
i) a first Fc domain monomer,
ii) a second Fc domain monomer,
iii) a third Fc domain monomer,
iv) a first CD38 heavy chain binding domain,
v) a linker joining the first and second Fc domain monomers, and
vi) a linker joining the second and to third Fc domain monomers;

b) a second polypeptide comprising:
i) a fourth Fc domain monomer,
ii) a fifth Fc domain monomer,
iii) a sixth Fc domain monomer,
iv) a second CD38 heavy chain binding domain,
v) a linker joining the fourth and fifth Fc domain monomers, and
vi) a linker joining the fifth and sixth Fc domain monomers;

c) a third polypeptide comprising a seventh Fc domain monomer;

d) a fourth polypeptide comprising an eighth Fc domain monomer;

e) a fifth polypeptide comprising ninth Fc domain monomer and a first CD38 light chain binding domain;

f) a sixth polypeptide comprising a tenth Fc domain monomer and a second CD38 light chain binding domain;

wherein the first and fourth Fc domain monomers together form a first Fc domain, the second and seventh Fc domain monomers together form an second Fc domain, the fifth and eighth Fc monomers together form a third Fc domain, the third and ninth Fc domain monomers together form a fourth Fc domain, the sixth and tenth Fc monomers together form a fifth Fc domain, the first CD38 heavy chain binding domain and first CD38 light chain binding domain together form a first Fab; and the second CD38 heavy chain binding domain and second CD38 light chain binding domain together form a second Fab.

Also described is an Fc-antigen binding domain construct, comprising:

a) a first polypeptide comprising:
i) a first Fc domain monomer,
ii) a second Fc domain monomer,
iii) a linker joining the first and second Fc domain monomers, and

b) a second polypeptide comprising:
i) a third Fc domain monomer,
ii) a fourth Fc domain monomer
iii) a linker joining the third and fourth Fc domain monomers;

c) a third polypeptide comprising a fifth Fc domain monomer and a first CD38 heavy chain binding domain and;

d) a fourth polypeptide comprising a sixth Fc domain monomer a second CD38 heavy chain binding domain;

e) a fifth polypeptide comprising a first CD38 light chain binding domain; and

f) a sixth polypeptide comprising a second CD38 light chain binding domain;

wherein the first and fifth Fc domain monomers together form a first Fc domain, the third and sixth Fc domain monomers together form an second Fc domain, the second and fourth Fc domain monomers together form a third Fc domain, the first CD38 heavy chain binding domain and first CD38 light chain binding domain together form a first Fab; and the second CD38 heavy chain binding domain and second CD38 light chain binding domain together form a second Fab.

In various embodiments: the first and second polypeptides are identical in sequence; the third and fourth polypeptides are identical in sequence; the fifth and sixth polypeptides are identical in sequence; the first and second polypeptides are identical in sequence, the third and fourth polypeptides are identical in sequence, and the fifth and sixth polypeptides are identical in sequence; the CH3 domain of each of the Fc domain monomers includes up to 8, 7, 6, 5, 4, 3, 2 or 1 single amino acid substitutions; the CH3 domain of each of the Fc domain monomers includes up to 8, 7, 6, 5, 4, 3, 2 or 1 single amino acid substitutions compared to the amino acid sequence of human IgG1; each of the Fc domain monomers independently comprises the amino acid sequence of any of SEQ ID NOs:42, 43, 45, and 47 having up to 10, 8, 7, 6, 5, 4, 3, 2 or 1 single amino acid substitutions; the single amino acids substitutions are only in the CH3 domain; the second and fourth Fc domain monomers comprise up to 8, 7, 6, 5, 4, 3, 2 or 1 single amino acid substitutions that promote homodimerization between the second and fourth Fc domain monomers; the first and fifth Fc domain monomers comprise up to 8, 7, 6, 5, 4, 3, 2 or 1 single amino acid substitutions that promote heterodimerization between the first and fifth Fc domain monomers and the third and sixth Fc domain monomers comprise up to 8, 7, 6, 5, 4, 3, 2 or 1 single amino acid substitutions that promote heterodimerization between the third and sixth Fc domain monomers; the substitutions that promote homodimerization are selected from substitutions in Table 4A and 4B; the substitutions that promote heterodimerization are selected from substitutions in Table 3.

Also described is an Fc-antigen binding domain construct, comprising:

a) a first polypeptide comprising:
i) a first Fc domain monomer,
ii) a second Fc domain monomer,
iii) a first CD38 heavy chain binding domain, and
iv) a linker joining the first and second Fc domain monomers,

b) a second polypeptide comprising:
i) a third Fc domain monomer,
ii) a fourth Fc domain monomer,
iii) a second CD38 heavy chain binding domain, and
iv) a linker joining the third and fourth Fc domain monomers,

c) a third polypeptide comprising a fifth Fc domain monomer and a third CD38 heavy chain binding domain;

d) a fourth polypeptide comprising a sixth Fc domain monomer and a fourth CD38 light chain binding domain;

e) a fifth polypeptide comprising a first CD38 light chain binding domain;

f) a sixth polypeptide comprising a second CD38 light chain binding domain;

g) a seventh polypeptide comprising a third CD38 light chain binding domain; and

h) an eighth polypeptide comprising a fourth CD38 light chain binding domain;

wherein the first and fifth Fc domain monomers together form a first Fc domain, the third and sixth Fc domain monomers together form an second Fc domain, the second and fourth Fc monomers together form a third Fc domain, the first CD38 light chain binding domain and third CD38 heavy chain binding domain together form a first Fab, the second CD38 light chain binding domain and fourth CD38 heavy chain binding domain together form a second Fab, the third CD38 light chain binding domain and first CD38 heavy chain binding domain together form a third Fab; and the fourth CD38 light chain binding domain and second CD38 heavy chain binding domain together form a second Fab

In various embodiments: the first and second polypeptides are identical in sequence; the third and fourth polypeptides are identical in sequence; the fifth, sixth, seventh and eighth polypeptides are identical in sequence; the first and second polypeptides are identical in sequence, the third and fourth polypeptides are identical in sequence, and the fifth, sixth, seventh and eighth polypeptides are identical in sequence; the CH3 domain of each of the Fc domain monomers includes up to 8, 7, 6, 5, 4, 3, 2 or 1 single amino acid substitutions; the CH3 domain of each of the Fc domain monomers includes up to 8, 7, 6, 5, 4, 3, 2 or 1 single amino acid substitutions compared to the amino acid sequence of human IgG1; each of the Fc domain monomers independently comprises the amino acid sequence of any of SEQ ID NOs:42, 43, 45, and 47 having up to 10, 8, 7, 6, 5, 4, 3, 2 or 1 single amino acid substitutions; the single amino acids substitutions are only in the CH3 domain; the second and fourth Fc domain monomers comprise up to 8, 7, 6, 5, 4, 3, 2 or 1 single amino acid substitutions that promote homodimerization between the second and fourth Fc domain monomers; wherein the first and fifth Fc domain monomers comprise up to 8, 7, 6, 5, 4, 3, 2 or 1 single amino acid substitutions that promote heterodimerization between the first and fifth Fc domain monomers and the third and sixth Fc domain monomers comprise up to 8, 7, 6, 5, 4, 3, 2 or 1 single amino acid substitutions that promote heterodimerization between the third and sixth Fc domain monomers; the substitutions that promote homodimerization are selected from substitutions in Table 4A and 4B; and the substitutions that promote heterodimerization are selected from substitutions in Table 3.

In various embodiments: each linker comprise3 or consist of an amino acid sequence selected from the group consisting of:GGGGGGGGGGGGGGGGGGGG (SEQ ID NO: 23), GGGGS (SEQ ID NO: 1), GGSG (SEQ ID NO: 2), SGGG (SEQ ID NO: 3), GSGS (SEQ ID NO: 4), GSGSGS (SEQ ID NO: 5), GSGSGSGS (SEQ ID NO: 6), GSGSGSGSGS (SEQ ID NO: 7), GSGSGSGSGSGS (SEQ ID NO: 8), GGSGGS (SEQ ID NO: 9), GGSGGSGGS (SEQ ID NO: 10), GGSGGSGGSGGS (SEQ ID NO: 11), GGSG (SEQ ID NO: 2), GGSG (SEQ ID NO: 2), GGSGGGSG (SEQ ID NO: 12), GGSGGGSGGGSGGGGGSGGGGSGGGGSGGGGS (SEQ ID NO: 213), GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), GGSGGGSEGGGSEGGGSEGGGSEGGGSEGGGSGGGS (SEQ ID NO: 34), GGGSGGGSGGGS (SEQ ID NO: 35), SGGGSGGGSGGGSGGGSGGG (SEQ ID NO: 18), GGSGGGSGGGSGGGSGGS (SEQ ID NO: 36), GGGG (SEQ ID NO: 19), GGGGGGGG (SEQ ID NO: 20), GGGGGGGGGGGG (SEQ ID NO: 21) and GGGGGGGGGGGGGGGG (SEQ ID NO: 22); at least one of the Fc domain monomers comprises a substitution at EU position I253; each amino acid substitution at EU position I253 is independently selected from the group consisting of I253A, I253C, I253D, I253E, I253F, I253G, I253H, I253I, I253K, I253L, I253M, I253N, I253P, I253Q, I253R, I253S, I253T, I253V, I253W, and I253Y; at least one of the Fc domain monomers comprises a substitution at EU position R292; each amino acid substitution at EU position R292 is independently selected from the group consisting of R292D, R292E, R292L, R292P, R292Q, R292R, R292T, and R292Y; at least one of the Fc domain monomers comprises a substitution selected from the group consisting of: T366Y, T366W, T394W, T394Y, F405W, F405A, Y407A, S354C, Y349T, T394F, K409D, K409E, K392D, K392E, K370D, K370E, D399K, D399R, E357K, E357R, D356K, and D356R; and the hinge of each Fc domain monomer independently comprises or consists of an amino acid sequence selected from the group consisting of EPKSCDKTHTCPPCPAPELL (SEQ ID NO: 220) and DKTHTCPPCPAPELL (SEQ ID NO: 221).

In all aspects of the disclosure, some or all of the Fc domain monomers (e.g., an Fc domain monomer comprising the amino acid sequence of any of SEQ ID Nos; 42, 43, 45 and 47 having no more than 10, 8, 6 or 4 single amino acid substitutions (e.g., in the CH3 domain only) can have one or both of a E345K and E430G amino acid substitution in addition to other amino acid substitutions or modifications. The E345K and E430G amino acid substitutions can increase Fc domain multimerization.

### Definitions:

As used herein, the term “Fc domain monomer” refers to a polypeptide chain that includes at least a hinge domain and second and third antibody constant domains (C_{H}2 and C_{H}3) or functional fragments thereof (e.g., at least a hinge domain or functional fragment thereof, a CH2 domain or functional fragment thereof, and a CH3 domain or functional fragment thereof) (e.g., fragments that that capable of (i) dimerizing with another Fc domain monomer to form an Fc domain, and (ii) binding to an Fc receptor). A preferred Fc domain monomer comprises, from amino to carboxy terminus, at least a portion of IgG1 hinge, an IgG1 CH2 domain and an IgG1 CH3 domain. Thus, an Fc domain monomer, e.g., aa human IgG1 Fc domain monomer can extend from E316 to G446 or K447, from P317 to G446 or K447, from K318 to G446 or K447, from K318 to G446 or K447, from S319 to G446 or K447, from C320 to G446 or K447, from D321 to G446 or K447, from K322 to G446 or K447, from T323 to G446 or K447, from K323 to G446 or K447, from H324 to G446 or K447, from T325 to G446 or K447, or from C326 to G446 or K447. The Fc domain monomer can be any immunoglobulin antibody isotype, including IgG, IgE, IgM, IgA, or IgD (e.g., IgG). Additionally, the Fc domain monomer can be an IgG subtype (e.g., IgG1, IgG2a, IgG2b, IgG3, or IgG4) (e.g., human IgG1). The human IgG1 Fc domain monomer is used in the examples described herein. The full hinge domain of human IgG1 extends from EU Numbering E316 to P230 or L235, the CH2 domain extends from A231 or G236 to K340 and the CH3 domain extends from G341 to K447. There are differing views of the position of the last amino acid of the hinge domain. It is either P230 or L235. In many examples herein the CH3 domain does not include K347. Thus, a CH3 domain can be from G341 to G446. In many examples herein a hinge domain can include E216 to L235. This is true, for example, when the hinge is carboxy terminal to a CH1 domain or a CD38 binding domain. In some case, for example when the hinge is at the amino terminus of a polypeptide, the Asp at EU Numbering 221 is mutated to Gln. An Fc domain monomer does not include any portion of an immunoglobulin that is capable of acting as an antigen-recognition region, e.g., a variable domain or a complementarity determining region (CDR). Fc domain monomers can contain as many as ten changes from a wild-type (e.g., human) Fc domain monomer sequence (e.g., 1-10, 1-8, 1-6, 1-4 amino acid substitutions, additions, or deletions) that alter the interaction between an Fc domain and an Fc receptor. Fc domain monomers can contain as many as ten changes (e.g., single amino acid changes) from a wild-type Fc domain monomer sequence (e.g., 1-10, 1-8, 1-6, 1-4 amino acid substitutions, additions, or deletions) that alter the interaction between Fc domain monomers. In certain embodiments, there are up to 10, 8, 6 or 5 single amino acid substitution on the CH3 domain compared to the human IgG1 CH3 domain sequence:
GQPREPQVYTLPPSRDELTKNQVSLTCLVKGFYPSDIAVEWESNGQPENNYKTTPPVLDSDGSFFLYSKLTVDKSRWQQGNVFSCSV MHEALHNHYTQKSLSLSPG (SEQ ID NO: 224). Examples of suitable changes are known in the art.

As used herein, the term “Fc domain” refers to a dimer of two Fc domain monomers that is capable of binding an Fc receptor. In the wild-type Fc domain, the two Fc domain monomers dimerize by the interaction between the two C_{H}3 antibody constant domains, as well as one or more disulfide bonds that form between the hinge domains of the two dimerizing Fc domain monomers.

In the present disclosure, the term “Fc-antigen binding domain construct” refers to associated polypeptide chains forming at least two Fc domains as described herein and including at least one “antigen binding domain.” Fc-antigen binding domain constructs described herein can include Fc domain monomers that have the same or different sequences. For example, an Fc-antigen binding domain construct can have three Fc domains, two of which includes IgG1 or IgG1-derived Fc domain monomers, and a third which includes IgG2 or IgG2-derived Fc domain monomers. In another example, an Fc-antigen binding domain construct can have three Fc domains, two of which include a “protuberance-into-cavity pair” and a third which does not include a “protuberance-into-cavity pair.” An Fc domain forms the minimum structure that binds to an Fc receptor, e.g., FcγRI, FcγRIIa, FcγRIIb, FcγRIIIa, FcγRIIIb, or FcγRIV.

As used herein, the term “antigen binding domain” refers to a peptide, a polypeptide, or a set of associated polypeptides that is capable of specifically binding a target molecule. In some embodiments, the “antigen binding domain” is the minimal sequence of an antibody that binds with specificity to the antigen bound by the antibody. Surface plasmon resonance (SPR) or various immunoassays known in the art, e.g., Western Blots or ELISAs, can be used to assess antibody specificity for an antigen. In some embodiments, the “antigen binding domain” includes a variable domain or a complementarity determining region (CDR) of an antibody, e.g., one or more CDRs of an antibody set forth in Table 1, one or more CDRs of an antibody set forth in Table 2, or the VH and/or VL domains of an antibody set forth in Table 2. In some embodiments, the CD38 binding domain can include a VH domain and a CH1 domain, optionally with a VL domain. In other embodiments, the antigen (e.g., CD38) binding domain is a Fab fragment of an antibody or a scFv. Thus, a CD38 binding domain can include a “CD38 heavy chain binding domain” that comprises or consists of a VH domain and a CH1 domain and a“ CD38 light chain binding domain” that comprises or consists of a VL domain and a C_{L} domain. A CD38 binding domain may also be a synthetically engineered peptide that binds a target specifically such as a fibronectin-based binding protein (e.g., a fibronectin type III domain (FN3) monobody).

As used herein, the term “Complementarity Determining Regions” (CDRs) refers to the amino acid residues of an antibody variable domain the presence of which are necessary for CD38 binding. Each variable domain typically has three CDR regions identified as CDR-L1, CDR-L2 and CDR-L3, and CDR-H1, CDR-H2, and CDR-H3). Each complementarity determining region may include amino acid residues from a “complementarity determining region” as defined by Kabat (i.e., about residues 24-34 (CDR-L1), 50-56 (CDR-L2), and 89-97 (CDR-L3) in the light chain variable domain and 31-35 (CDR-H1), 50-65 (CDR-H2), and 95-102 (CDR-H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (CDR-L1), 50-52 (CDR-L2), and 91-96 (CDR-L3) in the light chain variable domain and 26-32 (CDR-H1), 53-55 (CDR-H2), and 96-101 (CDR-H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs include amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR includes amino acids from both a CDR as defined by Kabat and those of a hypervariable loop, the FR residues will be adjusted accordingly.

An “Fv” fragment is an antibody fragment which contains a complete antigen recognition and binding site. This region consists of a dimer of one heavy and one light chain variable domain in tight association, which can be covalent in nature, for example, in a scFv. It is in this configuration that the three CDRs of each variable domain interact to define a CD38 binding site on the surface of the V_{H}-V_{L} dimer.

The “Fab” fragment contains a variable and constant domain of the light chain and a variable domain and the first constant domain (C_{H}1) of the heavy chain. F(ab′)₂ antibody fragments include a pair of Fab fragments which are generally covalently linked near their carboxy termini by hinge cysteines.

“Single-chain Fv” or “scFv” antibody fragments include the V_{H} and V_{L} domains of antibody in a single polypeptide chain. Generally, the scFv polypeptide further includes a polypeptide linker between the V_{H} and V_{L} domains, which enables the scFv to form the desired structure for CD38 binding.

As used herein, the term “antibody constant domain” refers to a polypeptide that corresponds to a constant region domain of an antibody (e.g., a C_{L} antibody constant domain, a C_{H}1 antibody constant domain, a C_{H}2 antibody constant domain, or a C_{H}3 antibody constant domain).

As used herein, the term “promote” means to encourage and to favor, e.g., to favor the formation of an Fc domain from two Fc domain monomers which have higher binding affinity for each other than for other, distinct Fc domain monomers. As is described herein, two Fc domain monomers that combine to form an Fc domain can have compatible amino acid modifications (e.g., engineered protuberances and engineered cavities, and/or electrostatic steering mutations) at the interface of their respective C_{H}3 antibody constant domains. The compatible amino acid modifications promote or favor the selective interaction of such Fc domain monomers with each other relative to with other Fc domain monomers which lack such amino acid modifications or with incompatible amino acid modifications. This occurs because, due to the amino acid modifications at the interface of the two interacting C_{H}3 antibody constant domains, the Fc domain monomers to have a higher affinity toward each other than to other Fc domain monomers lacking amino acid modifications.

As used herein, the term “dimerization selectivity module” refers to a sequence of the Fc domain monomer that facilitates the favored pairing between two Fc domain monomers. “Complementary” dimerization selectivity modules are dimerization selectivity modules that promote or favor the selective interaction of two Fc domain monomers with each other. Complementary dimerization selectivity modules can have the same or different sequences. Exemplary complementary dimerization selectivity modules are described herein.

As used herein, the term “engineered cavity” refers to the substitution of at least one of the original amino acid residues in the C_{H}3 antibody constant domain with a different amino acid residue having a smaller side chain volume than the original amino acid residue, thus creating a three dimensional cavity in the C_{H}3 antibody constant domain. The term “original amino acid residue” refers to a naturally occurring amino acid residue encoded by the genetic code of a wild-type C_{H}3 antibody constant domain.

As used herein, the term “engineered protuberance” refers to the substitution of at least one of the original amino acid residues in the C_{H}3 antibody constant domain with a different amino acid residue having a larger side chain volume than the original amino acid residue, thus creating a three dimensional protuberance in the C_{H}3 antibody constant domain. The term “original amino acid residues” refers to naturally occurring amino acid residues encoded by the genetic code of a wild-type C_{H}3 antibody constant domain.

As used herein, the term “protuberance-into-cavity pair” describes an Fc domain including two Fc domain monomers, wherein the first Fc domain monomer includes an engineered cavity in its C_{H}3 antibody constant domain, while the second Fc domain monomer includes an engineered protuberance in its C_{H}3 antibody constant domain. In a protuberance-into-cavity pair, the engineered protuberance in the C_{H}3 antibody constant domain of the first Fc domain monomer is positioned such that it interacts with the engineered cavity of the C_{H}3 antibody constant domain of the second Fc domain monomer without significantly perturbing the normal association of the dimer at the inter-C_{H}3 antibody constant domain interface.

As used herein, the term “heterodimer Fc domain” refers to an Fc domain that is formed by the heterodimerization of two Fc domain monomers, wherein the two Fc domain monomers contain different reverse charge mutations (see, e.g., mutations in Tables 4A and 4B) that promote the favorable formation of these two Fc domain monomers. In an Fc construct having three Fc domains—one carboxyl terminal “stem” Fc domain and two amino terminal “branch” Fc domains—each of the amino terminal “branch” Fc domains may be a heterodimeric Fc domain (also called a “branch heterodimeric Fc domain”).

As used herein, the term “structurally identical,” in reference to a population of Fc-antigen binding domain constructs, refers to constructs that are assemblies of the same polypeptide sequences in the same ratio and configuration and does not refer to any post-translational modification, such as glycosylation.

As used herein, the term “homodimeric Fc domain” refers to an Fc domain that is formed by the homodimerization of two Fc domain monomers, wherein the two Fc domain monomers contain the same reverse charge mutations (see, e.g., mutations in Tables 5 and 6). In an Fc construct having three Fc domains—one carboxyl terminal “stem” Fc domain and two amino terminal “branch” Fc domains—the carboxy terminal “stem” Fc domain may be a homodimeric Fc domain (also called a “stem homodimeric Fc domain”).

As used herein, the term “heterodimerizing selectivity module” refers to engineered protuberances, engineered cavities, and certain reverse charge amino acid substitutions that can be made in the C_{H}3 antibody constant domains of Fc domain monomers in order to promote favorable heterodimerization of two Fc domain monomers that have compatible heterodimerizing selectivity modules. Fc domain monomers containing heterodimerizing selectivity modules may combine to form a heterodimeric Fc domain. Examples of heterodimerizing selectivity modules are shown in Tables 3 and 4.

As used herein, the term “homodimerizing selectivity module” refers to reverse charge mutations in an Fc domain monomer in at least two positions within the ring of charged residues at the interface between C_{H}3 domains that promote homodimerization of the Fc domain monomer to form a homodimeric Fc domain. Examples of homodimerizing selectivity modules are shown in Tables 4 and 5.

As used herein, the term “joined” is used to describe the combination or attachment of two or more elements, components, or protein domains, e.g., polypeptides, by means including chemical conjugation, recombinant means, and chemical bonds, e.g., peptide bonds, disulfide bonds and amide bonds. For example, two single polypeptides can be joined to form one contiguous protein structure through chemical conjugation, a chemical bond, a peptide linker, or any other means of covalent linkage. In some embodiments, a CD38 binding domain is joined to a Fc domain monomer by being expressed from a contiguous nucleic acid sequence encoding both the CD38 binding domain and the Fc domain monomer. In other embodiments, a CD38 binding domain is joined to a Fc domain monomer by way of a peptide linker, wherein the N-terminus of the peptide linker is joined to the C-terminus of the CD38 binding domain through a chemical bond, e.g., a peptide bond, and the C-terminus of the peptide linker is joined to the N-terminus of the Fc domain monomer through a chemical bond, e.g., a peptide bond.

As used herein, the term “associated” is used to describe the interaction, e.g., hydrogen bonding, hydrophobic interaction, or ionic interaction, between polypeptides (or sequences within one single polypeptide) such that the polypeptides (or sequences within one single polypeptide) are positioned to form an Fc-antigen binding domain construct described herein (e.g., an Fc-antigen binding domain construct having three Fc domains). For example, in some embodiments, four polypeptides, e.g., two polypeptides each including two Fc domain monomers and two polypeptides each including one Fc domain monomer, associate to form an Fc construct that has three Fc domains (e.g., as depicted in FIGS. 50 and 51). The four polypeptides can associate through their respective Fc domain monomers. The association between polypeptides does not include covalent interactions.

As used herein, the term “linker” refers to a linkage between two elements, e.g., protein domains. A linker can be a covalent bond or a spacer. The term “bond” refers to a chemical bond, e.g., an amide bond or a disulfide bond, or any kind of bond created from a chemical reaction, e.g., chemical conjugation. The term “spacer” refers to a moiety (e.g., a polyethylene glycol (PEG) polymer) or an amino acid sequence (e.g., a 3-200 amino acid, 3-150 amino acid, or 3-100 amino acid sequence) occurring between two polypeptides or polypeptide domains to provide space and/or flexibility between the two polypeptides or polypeptide domains. An amino acid spacer is part of the primary sequence of a polypeptide (e.g., joined to the spaced polypeptides or polypeptide domains via the polypeptide backbone). The formation of disulfide bonds, e.g., between two hinge regions or two Fc domain monomers that form an Fc domain, is not considered a linker.

As used herein, the term “glycine spacer” refers to a linker containing only glycines that joins two Fc domain monomers in tandem series. A glycine spacer may contain at least 4 (SEQ ID NO: 19), 8 (SEQ ID NO: 20), or 12 (SEQ ID NO: 21) glycines (e.g., 4-30 (SEQ ID NO: 214), 8-30 (SEQ ID NO: 215), or 12-30 (SEQ ID NO: 216) glycines; e.g., 12-30 (SEQ ID NO: 216), 4, 5, 6, 7, 8, 9, 10, 11, 12, 13, 14, 15, 16, 17, 18, 19, 20, 21, 22, 23, 24, 25, 26, 27, 28, 29, or 30 glycines (SEQ ID NO: 214)). In some embodiments, a glycine spacer has the sequence of GGGGGGGGGGGGGGGGGGGG (SEQ ID NO: 27).

As used herein, the term “albumin-binding peptide” refers to an amino acid sequence of 12 to 16 amino acids that has affinity for and functions to bind serum albumin. An albumin-binding peptide can be of different origins, e.g., human, mouse, or rat. In some embodiments of the present disclosure, an albumin-binding peptide is fused to the C-terminus of an Fc domain monomer to increase the serum half-life of the Fc-antigen binding domain construct. An albumin-binding peptide can be fused, either directly or through a linker, to the N- or C-terminus of an Fc domain monomer.

As used herein, the term “purification peptide” refers to a peptide of any length that can be used for purification, isolation, or identification of a polypeptide. A purification peptide may be joined to a polypeptide to aid in purifying the polypeptide and/or isolating the polypeptide from, e.g., a cell lysate mixture. In some embodiments, the purification peptide binds to another moiety that has a specific affinity for the purification peptide. In some embodiments, such moieties which specifically bind to the purification peptide are attached to a solid support, such as a matrix, a resin, or agarose beads. Examples of purification peptides that may be joined to an Fc-antigen binding domain construct are described in detail further herein.

As used herein, the term “multimer” refers to a molecule including at least two associated Fc constructs or Fc-antigen binding domain constructs described herein.

As used herein, the term “polynucleotide” refers to an oligonucleotide, or nucleotide, and fragments or portions thereof, and to DNA or RNA of genomic or synthetic origin, which may be single- or double-stranded, and represent the sense or anti-sense strand. A single polynucleotide is translated into a single polypeptide.

As used herein, the term “polypeptide” describes a single polymer in which the monomers are amino acid residues which are joined together through amide bonds. A polypeptide is intended to encompass any amino acid sequence, either naturally occurring, recombinant, or synthetically produced.

As used herein, the term “amino acid positions” refers to the position numbers of amino acids in a protein or protein domain. The amino acid positions are numbered using the Kabat numbering system (Kabat et al., Sequences of Proteins of Immunological Interest, National Institutes of Health, Bethesda, Md., ed 5, 1991) where indicated (eg.g., for CDR and FR regions), otherwise the EU numbering is used.

FIGS. 24A-24D depict human IgG1 Fc domains numbered using the EU numbering system.

As used herein, the term “amino acid modification” or refers to an alteration of an Fc domain polypeptide sequence that, compared with a reference sequence (e.g., a wild-type, unmutated, or unmodified Fc sequence) may have an effect on the pharmacokinetics (PK) and/or pharmacodynamics (PD) properties, serum half-life, effector functions (e.g., cell lysis (e.g., antibody-dependent cell-mediated toxicity(ADCC) and/or complement dependent cytotoxicity activity (CDC)), phagocytosis (e.g., antibody dependent cellular phagocytosis (ADCP) and/or complement-dependent cellular cytotoxicity (CDCC)), immune activation, and T-cell activation), affinity for Fc receptors (e.g., Fc-gamma receptors (FcγR) (e.g., FcγRI (CD64), FcγRIIa (CD32), FcγRIIb (CD32), FcγRIIIa (CD16a), and/or FcγRIIIb (CD16b)), Fc-alpha receptors (FcaR), Fc-epsilon receptors (FcER), and/or to the neonatal Fc receptor (FcRn)), affinity for proteins involved in the compliment cascade (e.g., C1q), post-translational modifications (e.g., glycosylation, sialylation), aggregation properties (e.g., the ability to form dimers (e.g., homo- and/or heterodimers) and/or multimers), and the biophysical properties (e.g., alters the interaction between C_{H}1 and C_{L}, alters stability, and/or alters sensitivity to temperature and/or pH) of an Fc construct, and may promote improved efficacy of treatment of immunological and inflammatory diseases. An amino acid modification includes amino acid substitutions, deletions, and/or insertions. In some embodiments, an amino acid modification is the modification of a single amino acid. In other embodiment, the amino acid modification is the modification of multiple (e.g., more than one) amino acids. The amino acid modification may include a combination of amino acid substitutions, deletions, and/or insertions. Included in the description of amino acid modifications, are genetic (i.e., DNA and RNA) alterations such as point mutations (e.g., the exchange of a single nucleotide for another), insertions and deletions (e.g., the addition and/or removal of one or more nucleotides) of the nucleotide sequence that codes for an Fc polypeptide.

In certain embodiments, at least one (e.g., one, two, or three) Fc domain monomers within an Fc construct or Fc-antigen binding domain construct include an amino acid modification (e.g., substitution). In some instances, the at least one Fc domain monomers includes one or more (e.g., no more than two, three, four, five, six, seven, eight, nine, ten, or twenty) amino acid modifications (e.g., substitutions).

As used herein, the term “percent (%) identity” refers to the percentage of amino acid (or nucleic acid) residues of a candidate sequence, e.g., the sequence of an Fc domain monomer in an Fc-antigen binding domain construct described herein, that are identical to the amino acid (or nucleic acid) residues of a reference sequence, e.g., the sequence of a wild-type Fc domain monomer, after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

100×(fraction of A/B)

where A is the number of amino acid (or nucleic acid) residues scored as identical in the alignment of the candidate sequence and the reference sequence, and where B is the total number of amino acid (or nucleic acid) residues in the reference sequence. In some embodiments where the length of the candidate sequence does not equal to the length of the reference sequence, the percent amino acid (or nucleic acid) sequence identity of the candidate sequence to the reference sequence would not equal to the percent amino acid (or nucleic acid) sequence identity of the reference sequence to the candidate sequence.

In particular embodiments, a reference sequence aligned for comparison with a candidate sequence may show that the candidate sequence exhibits from 50% to 100% identity (e.g., 50% to 100%, 60% to 100%, 70% to 100%, 80% to 100%, 90% to 100%, 92% to 100%, 95% to 100%, 97% to 100%, 99% to 100%, or 99.5% to 100% identity), across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, or 100% of the length of the reference sequence. When a position in the candidate sequence is occupied by the same amino acid (or nucleic acid) residue as the corresponding position in the reference sequence, then the molecules are identical at that position.

In some embodiments, an Fc domain monomer in an Fc construct described herein (e.g., an Fc-antigen binding domain construct having three Fc domains) may have a sequence that is at least 95% identical (at least 97%, 99%, or 99.5% identical) to the sequence of a wild-type Fc domain monomer (e.g., SEQ ID NO: 42). In some embodiments, an Fc domain monomer in an Fc construct described herein (e.g., an Fc-antigen binding domain construct having three Fc domains) may have a sequence that is at least 95% identical (at least 97%, 99%, or 99.5% identical) to the sequence of any one of SEQ ID NOs: 44, 46, 48, and 50-53. In certain embodiments, an Fc domain monomer in the Fc construct may have a sequence that is at least 95% identical (at least 97%, 99%, or 99.5% identical) to the sequence of SEQ ID NO: 48, 52, and 53.

In some embodiments, a spacer between two Fc domain monomers may have a sequence that is at least 75% identical (at least 75%, 77%, 79%, 81%, 83%, 85%, 87%, 89%, 91%, 93%, 95%, 97%, 99%, 99.5%, or 100% identical) to the sequence of any one of SEQ ID NOs: 1-36 (e.g., SEQ ID NOs: 17, 18, 26, and 27) described further herein.

As used herein, the term “host cell” refers to a vehicle that includes the necessary cellular components, e.g., organelles, needed to express proteins from their corresponding nucleic acids. The nucleic acids are typically included in nucleic acid vectors that can be introduced into the host cell by conventional techniques known in the art (transformation, transfection, electroporation, calcium phosphate precipitation, direct microinjection, etc.). A host cell may be a prokaryotic cell, e.g., a bacterial cell, or a eukaryotic cell, e.g., a mammalian cell (e.g., a CHO cell). As described herein, a host cell is used to express one or more polypeptides encoding desired domains which can then combine to form a desired Fc-antigen binding domain construct.

As used herein, the term “pharmaceutical composition” refers to a medicinal or pharmaceutical formulation that contains an active ingredient as well as one or more excipients and diluents to enable the active ingredient to be suitable for the method of administration. The pharmaceutical composition of the present disclosure includes pharmaceutically acceptable components that are compatible with the Fc-antigen binding domain construct. The pharmaceutical composition is typically in aqueous form for intravenous or subcutaneous administration.

As used herein, a “substantially homogenous population” of polypeptides or of an Fc construct is one in which at least 50% of the polypeptides or Fc constructs in a composition (e.g., a cell culture medium or a pharmaceutical composition) have the same number of Fc domains, as determined by non-reducing SDS gel electrophoresis or size exclusion chromatography. A substantially homogenous population of polypeptides or of an Fc construct may be obtained prior to purification, or after Protein A or Protein G purification, or after any Fab or Fc-specific affinity chromatography only. In various embodiments, at least 55%, 60%, 65%, 70%, 75%, 80%, or 85% of the polypeptides or Fc constructs in the composition have the same number of Fc domains. In other embodiments, up to 85%, 90%, 92%, or 95% of the polypeptides or Fc constructs in the composition have the same number of Fc domains.

As used herein, the term “pharmaceutically acceptable carrier” refers to an excipient or diluent in a pharmaceutical composition. The pharmaceutically acceptable carrier must be compatible with the other ingredients of the formulation and not deleterious to the recipient. In the present disclosure, the pharmaceutically acceptable carrier must provide adequate pharmaceutical stability to the Fc-antigen binding domain construct. The nature of the carrier differs with the mode of administration. For example, for oral administration, a solid carrier is preferred; for intravenous administration, an aqueous solution carrier (e.g., WFI, and/or a buffered solution) is generally used.

As used herein, “therapeutically effective amount” refers to an amount, e.g., pharmaceutical dose, effective in inducing a desired biological effect in a subject or patient or in treating a patient having a condition or disorder described herein. It is also to be understood herein that a “therapeutically effective amount” may be interpreted as an amount giving a desired therapeutic effect, either taken in one dose or in any dosage or route, taken alone or in combination with other therapeutic agents.

As used herein, the term fragment and the term portion can be used interchangeably.

### BRIEF DESCRIPTION OF THE DRAWINGS

FIG. 1 is an illustration of an Fc-antigen binding domain construct (construct 1) containing two Fc domains and a CD38 binding domain. Each Fc domain is a dimer of two Fc domain monomers. Two of the Fc domain monomers (106 and 108) contain a protuberance in its CH3 antibody constant domain, while the other two Fc domain monomers (112 and 114) contain a cavity in the juxtaposed position in its C_{H}3 antibody constant domain. The construct is formed from three Fc domain monomer containing polypeptides. The first polypeptide (102) contains two protuberance-containing Fc domain monomers (106 and 108) linked by a spacer in a tandem series to a CD38 binding domain containing a V_{H} domain (110) on the N-terminus. A V_{L} containing domain (104) is joined to the V_{H} domain. Each of the second and third polypeptides (112 and 114) contains a cavity-containing Fc domain monomer.

FIG. 2 is an illustration of an Fc-antigen binding domain construct (construct 2) containing three Fc domains and a CD38 binding domain. The construct is formed from four Fc domain monomer containing polypeptides. The first polypeptide (202) contains three protuberance-containing Fc domains (206, 208, and 210) linked by spacers in a tandem series to a CD38 binding domain containing a V_{H} domain (212) on the N-terminus. A V_{L} containing domain (204) is joined to the V_{H} domain. Each of the second, third, and fourth polypeptides (214, 216, and 218) contains a cavity-containing Fc domain monomer.

FIG. 3 is an illustration of an Fc-antigen binding domain construct (construct 3) containing two Fc domains and twoCD38 binding domains. The construct is formed from three Fc domain monomer containing polypeptides. The first polypeptide (302) contains two protuberance-containing Fc domain monomers (304 and 306) linked by a spacer in a tandem series. Each of the second and third polypeptides (320 and 322) contains a cavity-containing Fc domain monomer (310 and 314) joined in tandem to a CD38 binding domain containing a V_{H} domain (316 and 318) on the N-terminus. A V_{L} containing domain (308 and 312) is joined to each V_{H} domain.

FIG. 4 is an illustration of an Fc-antigen binding domain construct (construct 4) containing three Fc domains and threeCD38 binding domains. The construct is formed from four Fc domain monomer containing polypeptides. The first polypeptide (402) contains three protuberance-containing Fc domain monomers (404, 406, and 408) linked by spacers in a tandem series. Each of the second, third, and fourth polypeptides (428, 430, and 432) contains a cavity-containing Fc domain monomer (426, 420, and 414) joined in tandem to a CD38 binding domain containing a V_{H} domain (422, 416, and 410) on the N-terminus. A V_{L} containing domain (424, 418, and 412) is joined to each V_{H} domain.

FIG. 5 is an illustration of an Fc-antigen binding domain construct (construct 5) containing two Fc domains and threeCD38 binding domains. The construct is formed from three Fc domain monomer containing polypeptides. The first polypeptide (502) contains two protuberance-containing Fc domain monomers (508 and 506) linked by a spacer in a tandem series with a CD38 binding domain containing a V_{H} domain (510) at the N-terminus. Each of the second and third polypeptides (524 and 526) contains a cavity-containing Fc domain monomer (516 and 522) joined in tandem to a CD38 binding domain containing a V_{H} domain (512 and 518) on the N-terminus. A V_{L} containing domain (504, 514, and 520) is joined to each V_{H} domain.

FIG. 6 is an illustration of an Fc-antigen binding domain construct (construct 6) containing three Fc domains and four CD38 binding domains. The construct is formed from four Fc monomer containing polypeptides. The first polypeptide (602) contains three protuberance-containing Fc domain monomers (606, 608, and 610) linked by spacers in a tandem series with a CD38 binding domain containing a V_{H} domain (612) at the N-terminus. Each of the second, third, and fourth polypeptides (632, 634, and 636) contains a cavity-containing Fc domain monomer (618, 624, and 630) joined in tandem to a CD38 binding domain containing a V_{H} domain (616, 622, and 628) on the N-terminus. A V_{L} containing domain (604, 616, 622, and 628) is joined to each V_{H} domain.

FIG. 7 is an illustration of an Fc-antigen binding domain construct (construct 7) containing three Fc domains and twoCD38 binding domains. This Fc-antigen binding domain construct contains a dimer of two Fc domain monomers (706 and 718), wherein both Fc domain monomers contain different charged amino acids at their C_{H}3-C_{H}3 interface than the WT sequence to promote favorable electrostatic interactions between the two Fc domain monomers. The construct is formed from four Fc domain monomer containing polypeptides. Two polypeptides (702 and 724) each contain a protuberance-containing Fc domain monomer (710 and 720) linked by a spacer in a tandem series to an Fc domain monomer containing different charged amino acids at the C_{H}3-C_{H}3 interface than the WT sequence (706 and 718) and a CD38 binding domain containing a V_{H} domain (712 and 714) on the N-terminus. The third and fourth polypeptides (708 and 722) each contain a cavity-containing Fc domain monomer. A V_{L} containing domain (704 and 716) is joined to each V_{H} domain.

FIG. 8 is an illustration of an Fc-antigen binding domain construct (construct 8) containing three Fc domains and twoCD38 binding domains. The construct is formed of four Fc domain monomer containing polypeptides. Two polypeptides (802 and 828) each contain a protuberance-containing Fc domain monomer (814 and 820) linked by a spacer in a tandem series to an Fc domain monomer containing different charged amino acids at the C_{H}3-C_{H}3 interface than the WT sequence (810 and 816). The third and fourth polypeptides (804 and 826) each contain a cavity-containing Fc domain monomer (808 and 824) joined in tandem to a CD38 binding domain containing a V_{H} domain (812 and 818) at the N-terminus. A V_{L} containing domain (806 and 822) is joined to each V_{H} domain.

FIG. 9 is an illustration of an Fc-antigen binding domain construct (construct 9) containing three Fc domains and fourCD38 binding domains. The construct is formed of four Fc domain monomer containing polypeptides. Two polypeptides (902 and 936) each contain a protuberance-containing Fc domain monomer (918 and 928) linked by a spacer in a tandem series to an Fc domain monomer containing different charged amino acids at the C_{H}3-C_{H}3 interface than the WT sequence (910 and 924) and a CD38 binding domain containing a V_{H} domain (908 and 920) at the N-terminus. The third and fourth polypeptides (904 and 934) contain a cavity-containing Fc domain monomer (916 and 932) joined in a tandem series to a CD38 binding domain containing a V_{H} domain (912 and 926) at the N-terminus. A V_{L} containing domain (906, 914, 922, and 930) is joined to each V_{H} domain.

FIG. 10 is an illustration of an Fc-antigen binding domain construct (construct 10) containing five Fc domains and twoCD38 binding domains. The construct is formed of six Fc domain monomer containing polypeptides. Two polypeptides (1002 and 1032) each contain a protuberance-containing Fc domain monomer (1016 and 1030) linked by spacers in a tandem series to another protuberance-containing Fc domain monomer (1014 and 1028), an Fc domain monomer containing different charged amino acids at the C_{H}3-C_{H}3 interface than the WT sequence (1008 and 1022) and a CD38 binding domain containing a V_{H} domain (1006 and 1018) at the N-terminus. The third, fourth, fifth, and sixth polypeptides (1012, 1010, 1026, and 1024) each contain a cavity-containing Fc domain monomer. A V_{L} containing domain (1004 and 1020) is joined to each VH domain.

FIG. 11 is an illustration of an Fc-antigen binding domain construct (construct 11) containing five Fc domains and fourCD38 binding domains. The construct is formed of six Fc domain monomer containing polypeptides. Two polypeptides (1102 and 1148) contain a protuberance-containing Fc domain monomer (1118 and 1132) linked by spacers in a tandem series to another protuberance-containing Fc domain monomer (1120 and 1130) and an Fc domain monomer containing different charged amino acids at the CH3-CH3 interface than the WT sequence (1124 and 1126). The third, fourth, fifth, and sixth polypeptides (1106, 1104, 1144, and 1146) each contain a cavity-containing Fc domain monomer (1116, 1110, 1134, and 1140) joined in a tandem series to a CD38 binding domain containing a V_{H} domain (1112, 1122, 1138, and 1128) at the N-terminus. A V_{L} containing domain (1108, 1114, 1135, and 1142) is joined to each V_{H} domain.

FIG. 12 is an illustration of an Fc-antigen binding domain construct (construct 12) containing five Fc domains and sixCD38 binding domains. The construct is formed of six Fc domain monomer containing polypeptides. Two polypeptides (1202 and 1256) contain a protuberance-containing Fc domain monomer (1224 and 1230) linked by spacers in a tandem series to another protuberance-containing Fc domain monomer (1226 and 1228), an Fc domain monomer containing different charged amino acids at the C_{H}3-C_{H}3 interface than the WT sequence (1210 and 1244), and a CD38 binding domain containing a V_{H} domain (1250 and 1248) at the N-terminus. The third, fourth, fifth, and sixth polypeptides (1206, 1204, 1254, and 1252) each contain a cavity-containing Fc domain monomer (1222, 1216, 1232, and 1238) joined in a tandem series to a CD38 binding domain containing a V_{H} domain (1218, 1212, 1236, and 1242) at the N-terminus. A V_{L} containing domain (1208, 1214, 1220, 1234, 1240, and 1246) is joined to each V_{H} domain.

FIG. 13 is an illustration of an Fc-antigen binding domain construct (construct 13) containing three Fc domains and twoCD38 binding domains. The construct is formed of four Fc domain monomer containing polypeptides. Two polypeptides (1302 and 1324) contain an Fc domain monomer containing different charged amino acids at the C_{H}3-C_{H}3 interface than the WT sequence (1308 and 1318) linked by a spacer in a tandem series to a protuberance-containing Fc domain monomer (1312 and 1316) and a CD38 binding domain containing a V_{H} domain (1310 and 1314) at the N-terminus. The third and fourth polypeptides (1306 and 1320) contain a cavity-containing Fc domain monomer. A V_{L} containing domain (1304 and 1322) is joined to each V_{H} domain.

FIG. 14 is an illustration of an Fc-antigen binding domain construct (construct 14) containing three Fc domains and twoCD38 binding domains. The construct is formed of four Fc domain monomer containing polypeptides. Two polypeptides (1404 and 1426) contain an Fc domain monomer containing different charged amino acids at the C_{H}3-C_{H}3 interface than the WT sequence (1308 and 1318) linked by a spacer in a tandem series to a protuberance-containing Fc domain monomer (1414 and 1418). The third and fourth polypeptides (1402 and 1428) each contain a cavity-containing Fc domain monomer (1410 and 1422) joined in a tandem series to a CD38 binding domain containing a V_{H} domain (1408 and 1416) at the N-terminus. A V_{L} containing domain (1406 and 1424) is joined to each V_{H} domain.

FIG. 15 is an illustration of an Fc-antigen binding domain construct (construct 15) containing three Fc domains and fourCD38 binding domains. The construct is formed of four Fc domain monomer containing polypeptides. Two polypeptides (1502 and 1536) contain an Fc domain monomer containing different charged amino acids at the C_{H}3-C_{H}3 interface than the WT sequence (1512 and 1524) linked by a spacer in a tandem series to a protuberance-containing Fc domain monomer (1518 and 1522) and a CD38 binding domain containing a VH domain (1514 and 1532) at the N-terminus. The third and fourth polypeptides (1504 and 1534) contain a cavity-containing Fc domain monomer (1510 and 1526) joined in a tandem series toCD38 binding domain containing a V_{H} domain (1508 and 1530) at the N-terminus. A V_{L} containing domain (1506, 1516, 1520, and 1528) is joined to each V_{H} domain.

FIG. 16 is an illustration of an Fc-antigen binding domain construct (construct 16) containing five Fc domains and twoCD38 binding domains. The construct is formed of six Fc domain monomer containing polypeptides. Two polypeptides (1602 and 1632) contain an Fc domain monomer containing different charged amino acids at the C_{H}3-C_{H}3 interface than the WT sequence (1610 and 1624) linked by spacers in a tandem series to a protuberance-containing Fc domain monomer (1612 and 1622), a second protuberance-containing Fc domain monomer (1614 and 1620) and a CD38 binding domain containing a V_{H} domain (1616 and 1618) at the N-terminus. The third, fourth, fifth, and sixth polypeptides (1608, 1606, 1626, and 1628) each contain a cavity-containing Fc domain. A V_{L} containing domain (1604 and 1630) is joined to each V_{H} domain.

FIG. 17 is an illustration of an Fc-antigen binding domain construct (construct 17) containing five Fc domains and fourCD38 binding domains. The construct is formed of six Fc monomer containing polypeptides. Two polypeptides (1702 and 1748) contain an Fc domain monomer containing different charged amino acids at the C_{H}3-C_{H}3 interface than the WT sequence (1718 and 1732) linked by spacers in a tandem series to a protuberance-containing Fc domain monomer (1720 and 1730) and a second protuberance-containing Fc domain monomer (1722 and 1728) at the N-terminus. The third, fourth, fifth, and sixth polypeptides (1706, 1704, 1746, and 1744) contain a cavity-containing Fc domain monomer (1716, 1710, 1734, and 1740) joined in a tandem series to a CD38 binding domain containing a V_{H} domain (1712, 1724, 1738, and 1726) at the N-terminus. A V_{L} containing domain (1708, 1714, 1736, and 1742) is joined to each V_{H} domain.

FIG. 18 is an illustration of an Fc-antigen binding domain construct (construct 18) containing five Fc domains and sixCD38 binding domains. The construct is formed of six Fc domain monomer containing polypeptides. Two polypeptides (1802 and 1856) contain an Fc domain monomer containing different charged amino acids at the C_{H}3-C_{H}3 interface than the WT sequence (1818 and 1838) linked by spacers in a tandem series to a protuberance-containing Fc domain monomer (1820 and 1836), a second protuberance-containing Fc domain monomer (1822 and 1834) and a CD38 binding domain containing a V_{H} domain (1826 and 1830) at the N-terminus. The third, fourth, fifth, and sixth polypeptides (1806, 1804, 1854, and 1852) each contain a cavity-containing Fc domain monomer (1816, 1810, 1840, and 1846) joined in a tandem series to a CD38 binding domain containing a V_{H} domain (1812, 1828, 1844, and 1850) at the N-terminus. A V_{L} containing domain (1808, 1814, 1824, 1832, 1842, and 1848) is joined to each V_{H} domain.

FIG. 19 is an illustration of an Fc-antigen binding domain construct (construct 19) containing five Fc domains and twoCD38 binding domains. The construct is formed of six Fc domain monomer containing polypeptides. Two polypeptides (1902 and 1932) contain a protuberance-containing Fc domain monomer (1912 and 1930) linked by spacers in a tandem series to an Fc domain monomer containing different charged amino acids at the C_{H}3-C_{H}3 interface than the WT sequence (1908 and 1926), a protuberance-containing Fc domain monomer (1916 and 1918), and a CD38 binding domain containing a V_{H} domain (1914 and 1920) at the N-terminus. The third and fourth polypeptides (1910 and 1928) contain cavity-containing Fc domain monomers and the fifth and sixth polypeptides (1906 and 1924) contain cavity-containing Fc domain monomers. A V_{L} containing domain (1904 and 1922) is joined to each V_{H} domain.

FIG. 20 is an illustration of an Fc-antigen binding domain construct (construct 20) containing five Fc domains and fourCD38 binding domains. The construct is formed of six Fc domain monomer containing polypeptides. Two polypeptides (2002 and 2048) contain a protuberance-containing Fc domain monomer (2020 and 2022) linked by spacers in a tandem series to an Fc domain monomer containing different charged amino acids at the C_{H}3-C_{H}3 interface than the WT sequence (2012 and 2030), and a protuberance-containing Fc domain monomer (2040 and 2038) at the N-terminus. The third, fourth, fifth, and sixth polypeptides (2006, 2004, 2046, and 2044) each contain a cavity-containing Fc domain monomer (2018. 2010, 2024, and 2032) joined in a tandem series to a CD38 binding domain containing a V_{H} domain (2014, 2042, 2028, and 2036) at the N-terminus. A V_{L} containing domain (2008, 2016, 2026, and 2034) is joined to each V_{H} domain.

FIG. 21 is an illustration of an Fc-antigen binding domain construct (construct 21) containing five Fc domains and six CD38 binding domains. The construct is formed of six Fc domain monomer containing polypeptides. Two polypeptides (2102 and 2156) contain a protuberance-containing Fc domain monomer (2120 and 2122) linked by spacers in a tandem series to an Fc domain monomer containing different charged amino acids at the C_{H}3-C_{H}3 interface than the WT sequence (2112 and 2130), another protuberance-containing Fc domain monomer (2144 and 2142), and a CD38 binding domain containing a V_{H} domain (2148 and 2138) at the N-terminus. The third, fourth, fifth, and sixth polypeptides (2106, 2104, 2154, and 2152) each contain a cavity-containing Fc domain monomer (2118, 2110, 2124, and 2132) joined in a tandem series to a CD38 binding domain containing a V_{H} domain (2114, 2150, 2128, and 2136) at the N-terminus. A V_{L} containing domain (2108, 2116, 2126, 2134, 2140, and 2146) is joined to each V_{H} domain.

FIG. 22 is three graphs showing the results of CDC, ADCP, and ADCC assays with various anti-CD20 constructs targeting B cells. The first graph shows that the S3Y Fc-antigen binding domain construct can mediate CDC. The middle graph shows that both the SAI and S3Y Fc-antigen binding domain constructs exhibit >100-fold enhanced potency in an ADCP FcγRIIa reporter assay. The third graph shows that the SAI and S3Y Fc-antigen binding domain constructs exhibit enhanced ADCC activity relative to the fucosylated mAb and similar activity to the afucosylated mAb.

FIG. 23 is a schematic representation of three exemplary ways the CD38 binding domain can be joined to the Fc domain of an Fc construct. Panel A shows a heavy chain component of a CD38 binding domain can be expressed as a fusion protein of an Fc chain and a light chain component can be expressed as a separate polypeptide. Panel B shows an scFv expressed as a fusion protein of the long Fc chain. Panel C shows heavy chain and light chain components expressed separately and exogenously added and joined to the Fc-antigen binding domain construct with a chemical bond.

FIG. 24A depicts the amino acid sequence of a human IgG1 (SEQ ID NO: 43) with EU numbering. The hinge region is indicated by a double underline, the CH2 domain is not underlined and the CH3 region is underlined.

FIG. 24B depicts the amino acid sequence of a human IgG1 (SEQ ID NO: 45) with EU numbering. The hinge region, which lacks E216-C220, inclusive, is indicated by a double underline, the CH2 domain is not underlined and the CH3 region is underlined and lacks K447.

FIG. 24C depicts the amino acid sequence of a human IgG1 (SEQ ID NO: 47) with EU numbering. The hinge region is indicated by a double underline, the CH2 domain is not underlined and the CH3 region is underlined and lacks 447K.

FIG. 24D depicts the amino acid sequence of a human IgG1 (SEQ ID NO: 42) with EU numbering. The hinge region, which lacks E216-C220, inclusive, is indicated by a double underline, the CH2 domain is not underlined and the CH3 region is underlined.

FIG. 25. Depicts the results of an analysis of dose dependent binding of an anti-CD38 antibody showing relatively high, moderate, and low cell surface CD38 expression among multiple hematological tumor cell lines. VivoTag645-labeled anti-CD38 antibody binding to live cell surface CD38. Cell surface binding was assessed by FACS analysis.

FIG. 26. Depicts the results of an analysis showing that anti-CD38 constructs have a similar cell binding profile as IgG1 anti-CD38 antibodies that cross-react with the human and cyno CD38. (A) Human CD38 expressing Raji tumor cells were incubated with VivoTag645-labeled-antibodies, SIA-AA-Cyno (anti-Cyno CD38 mAb), S3Y-AA-Cyno—CD38 (Construct 13 with Cyno CD38 Fab), anti-CD38 mAb, S3Y-AA-CD38 (Construct 13 with anti-CD38 Fab), IgG isotype control and SIF1 Control (Fc trimer without Fab regions) at 4° C. for 1 hour. Extent of cell surface binding was assessed by flow cytometry. (B) CHO cells stably expressing cyno CD38 were harvested and cell suspensions were incubated with VivoTag645-labeled-antibodies, SIA-AA-Cyno (anti-Cyno CD38 mAb), S3Y-AA-Cyno CD38 (Construct 13 with anti-Cyno CD38 Fab), anti-CD38 mAb, S3Y-AA-CD38 (Construct 13 with anti-CD38 Fab), IgG isotype control and SIF1 Control (Fc trimer without Fab regions) at 4° C. for 1 hour. Extent of cell surface binding was assessed by flow cytometry. Note: anti-Cyno CD38 mAb cross reactive antibody (S1A-AA-Cyno) and S3Y-AA-Cyno recognize both human and cyno CD38. In addition, S3Y-AA-Cyno CD38 binds cell surface CD38 better than S1A-AA-Cyno (anti-Cyno CD38 mAb).

FIG. 27. Depicts the results of an assessment of CDC activity by anti-CD38 constructs in Daudi cells and Raji cells.

FIG. 28. Depicts the results of an assessment of tumor cell killing by anti-CD38 constructs in whole human blood. Anti-CD38 Construct 13 (S3Y-AA-CD38) demonstrates highly potent tumor cell killing capacity in human whole blood. (A) Effects of anti-CD38 mAb and S3Y-AA-CD38 in killing of Daudi-luciferase tumor cells in whole human blood. (B) Effects of anti-CD38 mAb and S3Y-AA-CD38 in killing of tumor cells in human blood. In both (A) & (B), live Daudi-luciferase cells were quantified by adding luciferin substrate and measuring light emission on a luminometer. % Cell killing is calculated by normalizing the luminescence values of test samples with Spontaneous Lysis Control (0% Cell Lysis) (No Antibody Addition) and Total Lysis Control (100% Cell Lysis). Table show tumor cell killing EC50 value comparisons from whole blood from 3 separate human donors. Values represent mean±SD.

FIG. 29. Depicts the results of an assessment of endogenous B cell depletion from cynomolgus monkey blood. (A) Dose-dependent binding of SIA-AA-Cyno (anti-Cyno CD38 mAb), S3Y-AA-Cyno-011 (Construct 13 with Cyno CD38 Fab), IgG isotype control and SIF1 Control (Fc trimer without Fab regions) to cyno B cells. (B) Dose-dependent increase in frequency of cyno B cell binding to SIA-AA-Cyno, S3Y-AA-Cyno-011 (Construct 13 with Cyno CD38 Fab), IgG isotype control and SIF1 Control (Fc trimer without Fab regions). (C) Dose-dependent increase in B cell depletion with SIA-AA-Cyno, S3Y-AA-Cyno-001 (Construct 13 with Cyno CD38 Fab), IgG isotype control and SIF1 Control (Fc trimer without Fab regions). Anti-CD38 construct (S3Y-AA-CD38) treatment resulted in much greater cell depletion than anti-CD38 mAb. Values are normalized to B cell frequency in untreated control group. (A, B, C) Values plotted in these figures were generated from same monkey blood donor.

FIG. 30. Depicts the results of an assessment of the impact of an anti-CD38 construct in a lymphoma subcutaneous tumor model. SCID mice were subcutaneously inoculated with human lymphoma (Raji) tumor cells. Six days after tumor cell implantation mice were randomized into treatment groups (n=10 in each) & treated intraperitoneally with 0.5 mL normal human serum complement (HSC). Next day (on day 7) mice were again injected intraperitoneally with HSC followed by anti-CD38 mAb (single iv dose of 5.94 mg/kg), or S3Y-AA-CD38 (single iv dose of 10 mg/kg), or PBS (single IV injection). Mice were given 3^{rd} ip injection of HSC on day 8^{th}. Tumor growth was routinely monitored by tumor volume measurement. Points labeled with ** in S3Y-AA-CD38 group had p values of <0.0022 relative to corresponding treatment groups.

FIG. 31A. Depicts the results of a comparison of S3Y-AA-CD38 (inverted triangles) and an anti-CD38 mAb (circles) with respect to ADCC, ADCP and CDC activity in Daudi cells.

FIG. 31B Depicts the results of a comparison of S3Y-AA-CD38 (inverted triangles) and an anti-CD38 mAb (circles) with respect to ADCC, ADCP (measured using a reporter as a surrogate for phagocytosis by macrophages) and CDC activities against Raji tumor cells, which are resistant to anti-CD38 mAb mediated CDC.

FIG. 32. Depicts the results of a study of tumor cell depletion from whole human blood by S3Y-AA-CD38 (inverted triangles) and an anti-CD38 mAb (circles).

FIG. 33. Depicts the results of a study of the complement mediated cytotoxicity of S3Y-AA-CD38 (inverted triangles) and an anti-CD38 mAb (circles) in Daudi cells (left panel, relatively high CD38 expression and relatively low CD55 and CD59 expression) and in Raji cells (right panel, relatively low CD38 expression and relatively high CD55 and CD59 expression).

FIG. 34A. Depicts the results of a study of the ADCC activity (left panel) and CDC activity (right panel) of S3Y-AA-Cyno CD38 (inverted triangles) and an anti-cyno CD38 mAb (circles).

FIG. 34B. Depicts the results of a study of the ADCC activity (left panel), ADCP activity (center panel), and CDC activity (right panel) of S3Y-AA-Cyno CD38 (inverted triangles) and an anti-cyno CD38 mAb (circles). CDC activity was measured using Raji cells, which are resistant to anti-CD38 mAb mediated CDC.

FIG. 35. Depicts the results of a study of tumor cell depletion by S3Y-AA-Cyno CD38 (inverted triangles) and an anti-Cyno CD38 mAb (circles).

FIG. 36. Depicts the results of a study comparing B cell depletion by S3Y-AA-Cyno CD38 (second bar in each pair) and an anti-cyno CD38 mAb (first bar in each pair) in vitro (left panel) and in vivo (right panel).

FIG. 37. Depicts the results of a study comparing plasma cell depletion by S3Y-AA-CD38 (inverted triangles) and an anti-CD38 mAb (circles) in vitro. Percent plasma cell depletion by either anti-CD38 mAb or S3Y-AA-CD38 within total bone marrow mononuclear cells (BM-MNCs) from multiple myeloma patient MM536. Depletion was calculated as the total number of viable CD138+ cells at each concentration, relative to a baseline value from untreated BM-MNCs.

FIG. 38A. Depicts the results of a study showing that S3Y-AA-CD38 binding to FcgRIIa, FcgRIIIa and complement is at least 100-fold greater than an anti-CD38 mAb.

FIG. 38B. Depicts the results of a study showing that S3Y-AA-CD38 binding to FcγRIIA, FcγRIIIA is enhanced by >500× and S3Y-AA-CD38-opsonized tumor cells to human complement protein C1q is enhanced by 12× than an anti-CD38 mAb.

### DETAILED DESCRIPTION

Many therapeutic antibodies function by recruiting elements of the innate immune system through the effector function of the Fc domains, such as antibody-dependent cytotoxicity (ADCC), antibody-dependent cellular phagocytosis (ADCP), and complement-dependent cytotoxicity (CDC). In some instances, the present disclosure contemplates combining a CD38 binding domain of a known single Fc-domain containing therapeutic, e.g., a known therapeutic antibody, with at least two Fc domains to generate a novel therapeutic with unique biological activity. In some instances, a novel therapeutic disclosed herein has a biological activity greater than that of the known Fc-domain containing therapeutic, e.g., a known therapeutic antibody. The presence of at least two Fc domains can enhance effector functions and to activate multiple effector functions, such as ADCC in combination with ADCP and/or CDC, thereby increasing the efficacy of the therapeutic molecules. In order to generate a product with consistent biological function, control of the number of Fc domains is critical. The disclosure features a set of Fc engineering tools to control homodimerization and heterodimerization of the peptides encoding the Fc domain, to assemble molecules of discrete size from a limited number of polypeptide chains. International Publication Nos. WO/2015/168643, WO2017/151971, WO 2017/205436, and WO 2017/205434 disclose Fc engineering tools and methods for assembling molecules with two or more Fc domains, and are herein incorporated by reference in their entirety. The engineering tools include structural features (for example, glycine linkers) that significantly improve manufacturing outcome. The properties of these constructs allow for the efficient generation of substantially homogenous pharmaceutical compositions. Such homogeneity in a pharmaceutical composition is desirable in order to ensure the safety, efficacy, uniformity, and reliability of the pharmaceutical composition. Having a high degree of homogeneity in a pharmaceutical composition also minimizes potential aggregation or degradation of the pharmaceutical product caused by unwanted materials (e.g., degradation products, and/or aggregated products or multimers), as well as limiting off-target and adverse side effects caused by the unwanted materials.

As described in detail herein, we improved homogeneity of the composition by engineering the Fc domain components of the Fc-antigen binding domain constructs using approaches including the use of spacers including only glycine residues to join two Fc domain monomers in tandem series, the use of polypeptide sequences having the terminal lysine residue removed, and the use of two sets of heterodimerizing selectivity modules: (i) heterodimerizing selectivity modules having different reverse charge mutations and (ii) heterodimerizing selectivity modules having engineered cavities and protuberances.

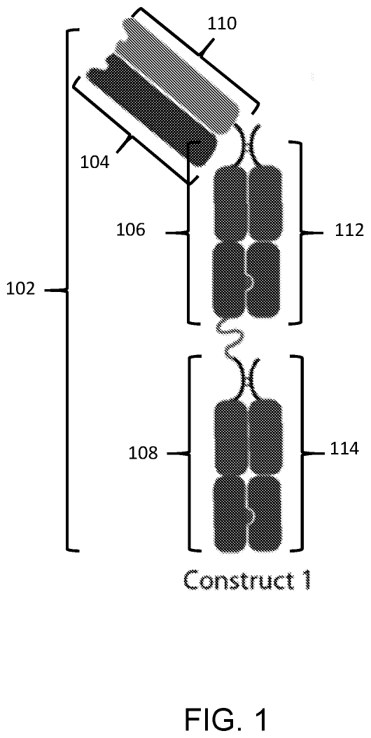

We designed a series of Fc-antigen binding domain constructs in which Fc domains were connected in tandem, using one long peptide chain containing multiple Fc sequences separated by linkers, and multiple copies of a short chain containing a single Fc sequence (Fc-antigen binding domain constructs 1-6; FIG. 1-FIG. 6). Heterodimerizing mutations were introduced into each Fc sequence to ensure assembly into the desired tandem configuration with minimal formation of smaller or larger complexes. Any number of Fc domains can be connected in tandem in this fashion, allowing the creation of constructs with 2, 3, 4, 5, 6, 7, 8, 9, 10, or more Fc domains. For a peptide with N Fc domains, such constructs can be prepared with 1 to N+1 CD38 binding domains, depending whether the CD38 binding domains are introduced into the long peptide chain, the short peptide chain, or both, respectively.

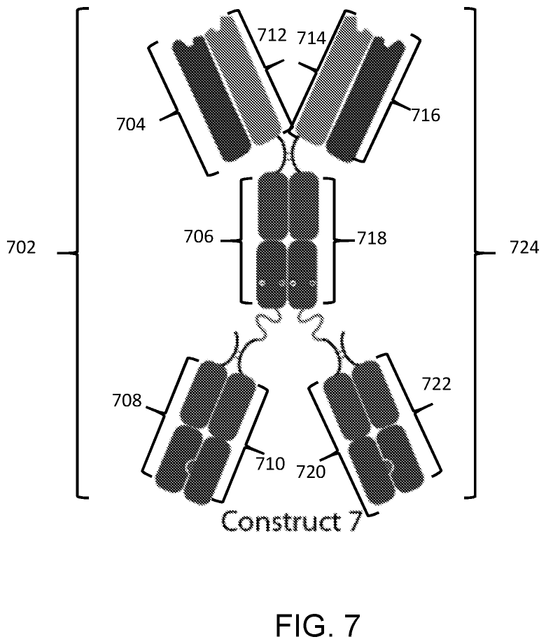

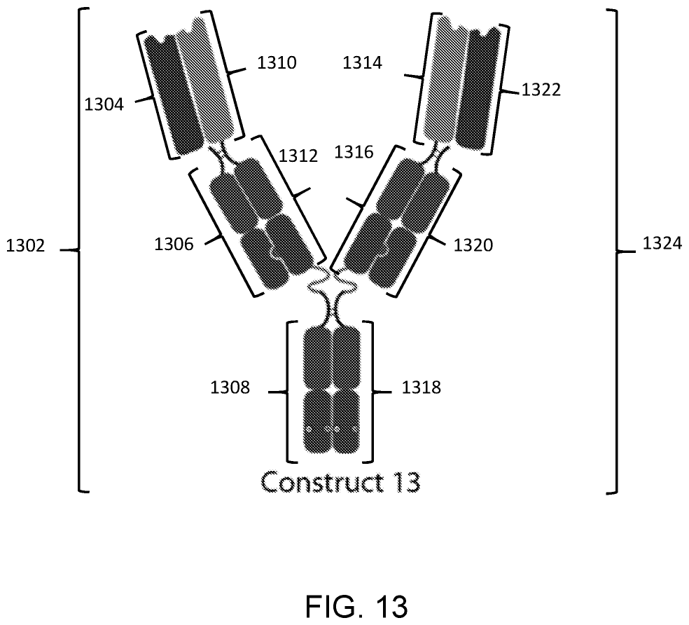

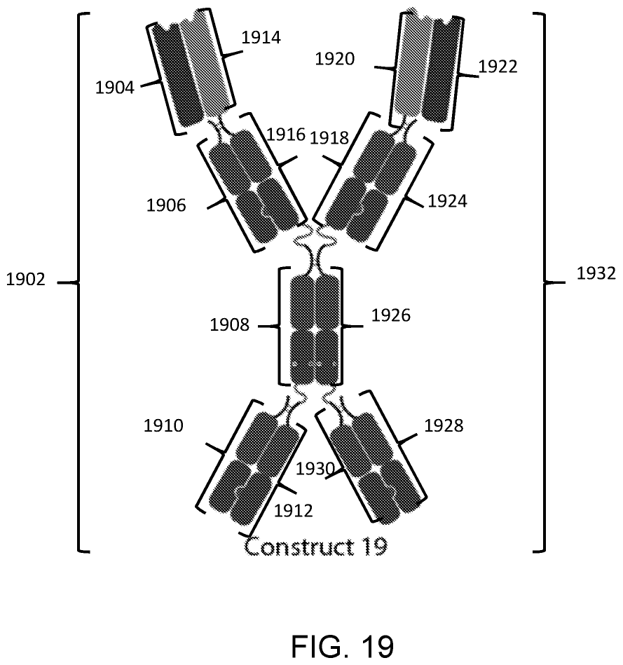

In Fc-antigen binding domain constructs 1-6 (FIG. 1-FIG. 6), Fc domains were connected with a single branch point between the Fc domains. These constructs include two copies of a long peptide chain containing multiple Fc sequences separated by linkers, in which the branching Fc sequence contains homodimerizing mutations and the non-branching Fc domains contain heterodimerizing mutations. Multiple copies of short chains including a single Fc sequence with mutations complementary to the heterodimerizing mutations in the long chains are used to complete the multimeric Fc scaffold. Heterodimerizing Fc domains can be linked to the C-terminal end (e.g., Fc-antigen binding domain constructs 7-12; FIG. 7-FIG. 12), the N-terminal end (e.g., Fc-antigen binding domain constructs 13-18; FIG. 13-FIG. 18), or both ends of the branching Fc domain (e.g., Fc-antigen binding domain constructs 19-21; FIG. 19-FIG. 21). Multiple Fc domains in tandem may be linked to either end of the branching Fc domain. CD38 binding domains may be introduced into the long peptide chains, resulting in twoCD38 binding domains per assembled protein molecule. Alternatively, CD38 binding domains may be introduced into the short peptide chains, resulting in N−1CD38 binding domains per assembled protein molecule, where N is the number of Fc domains in the assembled protein molecule. If CD38 binding domains are introduced into both the short and the long peptide chains, the resulting assembled protein molecule contains N+1CD38 binding domains.

Past engineering efforts for monoclonal antibodies (mAbs) and Fc domains included making mutations in the Fc domain to strengthen binding to FcγRIIIa and thus enhancing the antibody-dependent cell-mediated cytotoxicity (ADCC) response, and a fucosylation of the Fc domain to strengthen binding to FcγRIIIa and thus enhances the ADCC response.

In comparison to antibodies with mutations in the Fc domain to strengthen binding to FcγRIIIa or afucosylation of the Fc domain, the Fc-antigen binding domain constructs disclosed in this disclosure unexpectedly feature stronger binding to multiple classes of Fcγ receptors and enhanced activity of multiple cytotoxicity pathways. The Fc-antigen binding domain constructs of this disclosure can enhance binding to both FcγRIIa and FcγRIIIa compared to their corresponding fucosylated and afucosylated parent monoclonal antibodies (see, Example 46). Further, the Fc-antigen binding domain constructs of this disclosure unexpectedly feature an ability to mediate the complement-dependent cytotoxicity (CDC) pathway and/or the antibody-dependent cellular phagocytosis (ADCP) pathway in addition to enhancing the ADCC pathway response (see, Example 47).

### I. Fc Domain Monomers

An Fc domain monomer includes at least a portion of a hinge domain, a C_{H}2 antibody constant domain, and a C_{H}3 antibody constant domain (e.g., a human IgG1 hinge, a CH2 antibody constant domain, and a C_{H}3 antibody constant domain with optional amino acid substitutions). The Fc domain monomer can be of immunoglobulin antibody isotype IgG, IgE, IgM, IgA, or IgD. The Fc domain monomer may also be of any immunoglobulin antibody isotype (e.g., IgG1, IgG2a, IgG2b, IgG3, or IgG4). The Fc domain monomers may also be hybrids, e.g., with the hinge and C_{H}2 from IgG1 and the C_{H}3 from IgA, or with the hinge and C_{H}2 from IgG1 but the C_{H}3 from IgG3. A dimer of Fc domain monomers is an Fc domain (further defined herein) that can bind to an Fc receptor, e.g., FcγRIIIa, which is a receptor located on the surface of leukocytes. In the present disclosure, the C_{H}3 antibody constant domain of an Fc domain monomer may contain amino acid substitutions at the interface of the C_{H}3-C_{H}3 antibody constant domains to promote their association with each other. In other embodiments, an Fc domain monomer includes an additional moiety, e.g., an albumin-binding peptide or a purification peptide, attached to the N- or C-terminus. In the present disclosure, an Fc domain monomer does not contain any type of antibody variable region, e.g., V_{H}, V_{L}, a complementarity determining region (CDR), or a hypervariable region (HVR).

In some embodiments, an Fc domain monomer in an Fc-antigen binding domain construct described herein (e.g., an Fc-antigen binding domain construct having three Fc domains) may have a sequence that is at least 95% identical (at least 97%, 99%, or 99.5% identical) to the sequence of SEQ ID NO:42. In some embodiments, an Fc domain monomer in an Fc-antigen binding domain construct described herein (e.g., an Fc-antigen binding domain construct having three Fc domains) may have a sequence that is at least 95% identical (at least 97%, 99%, or 99.5% identical) to the sequence of any one of SEQ ID NOs: 44, 46, 48, and 50-53. In certain embodiments, an Fc domain monomer in the Fc-antigen binding domain construct may have a sequence that is at least 95% identical (at least 97%, 99%, or 99.5% identical) to the sequence of any one of SEQ ID NOs: 48, 52, and 53.

SEQ ID NO: 42
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDVVLNGK
EYKCKVSNKALPAPIEKTISKAKGQPREPQVYTLPPSRDELTKNQVSLT
CLVKGFYPSDIAVEVVESNGQPENNYKTTPPVLDSDGSFFLYSKLTVDK
SRWQQGNVFSCSVMHEALHNHYTQKSLSLSPGK
SEQ ID NO: 44
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVCTLPPSRDELTKNQVSLSC
AVKGFYPSDIAVEWESNGQPENNYKTTPPVLDSDGSFFLVSKLTVDKSR
WQQGNVFSCSVMHEALHNHYTQKSLSLSPGK
SEQ ID NO: 46
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVCTLPPSRDELTKNQVSLSC
AVKGFYPSDIAVEWESNGQPENNYKTTPPVLDSDGSFFLVSKLTVDKSR
WQQGNVFSCSVMHEALHNHYTQKSLSLSPG
SEQ ID NO: 48
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVCTLPPSRDELTKNQVSLSC
AVDGFYPSDIAVEWESNGQPENNYKTTPPVLDSDGSFFLVSKLTVDKSR
WQQGNVFSCSVMHEALHNHYTQKSLSLSPG
SEQ ID NO: 50
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPCRDELTKNQVSLWC
LVKGFYPSDIAVEVVESNGQPENNYKTTPPVLDSDGSFFLYSKLTVDKS
RWQQGNVFSCSVMHEALHNHYTQKSLSLSPGK
SEQ ID NO: 51
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEVVESNGQPENNYKTTPPVLKSDGSFFLYSDLTVDKS
RWQQGNVFSCSVMHEALHNHYTQKSLSLSPGK
SEQ ID NO: 52
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEVVESNGQPENNYKTTPPVLKSDGSFFLYSDLTVDKS
RWQQGNVFSCSVMHEALHNHYTQKSLSLSPG
SEQ ID NO: 53
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPCRDKLTKNQVSLWC
LVKGFYPSDIAVEVVESNGQPENNYKTTPPVLDSDGSFFLYSKLTVDKS
RWQQGNVFSCSVMHEALHNHYTQKSLSLSPGK

### II. Fc Domains

As defined herein, an Fc domain includes two Fc domain monomers that are dimerized by the interaction between the C_{H}3 antibody constant domains. An Fc domain forms the minimum structure that binds to an Fc receptor, e.g., Fc-gamma receptors (i.e., Fcγ receptors (FcγR)), Fc-alpha receptors (i.e., Fca receptors (FcαR)), Fc-epsilon receptors (i.e., Fcε receptors (FcεR)), and/or the neonatal Fc receptor (FcRn). In some embodiments, an Fc domain of the present disclosure binds to an Fcγ receptor (e.g., FcγRI (CD64), FcγRIIa (CD32), FcγRIIb (CD32), FcγRIIIa (CD16a), FcγRIIIb (CD16b)), and/or FcγRIV and/or the neonatal Fc receptor (FcRn).

### III CD38 Binding Domains

Antigen binding domains include one or more peptides or polypeptides that specifically bind a target molecule. CD38 binding domains may include the CD38 binding domain of an antibody. In some embodiments, the CD38 binding domain may be a fragment of an antibody or an antibody-construct, e.g., the minimal portion of the antibody that binds to the target antigen. A CD38 binding domain may also be a synthetically engineered peptide that binds a target specifically such as a fibronectin-based binding protein (e.g., a FN3 monobody).

A fragment antigen-binding (Fab) fragment is a region on an antibody that binds to a target antigen. It is composed of one constant and one variable domain of each of the heavy and the light chain. A Fab fragment includes a V_{H}, V_{L}, C_{H}1 and C_{L} domains. The variable domains V_{H} and V_{L} each contain a set of 3 complementarity-determining regions (CDRs) at the amino terminal end of the monomer. The Fab fragment can be of immunoglobulin antibody isotype IgG, IgE, IgM, IgA, or IgD. The Fab fragment monomer may also be of any immunoglobulin antibody isotype (e.g., IgG1, IgG2a, IgG2b, IgG3, or IgG4). In some embodiments, a Fab fragment may be covalently attached to a second identical Fab fragment following protease treatment (e.g., pepsin) of an immunoglobulin, forming an F(ab′)₂ fragment. In some embodiments, the Fab may be expressed as a single polypeptide, which includes both the variable and constant domains fused, e.g. with a linker between the domains.

In some embodiments, only a portion of a Fab fragment may be used as a CD38 binding domain. In some embodiments, only the light chain component (V_{L}+C_{L}) of a Fab may be used, or only the heavy chain component (V_{H}+C_{H}) of a Fab may be used. In some embodiments, a single-chain variable fragment (scFv), which is a fusion protein of the the VH and VL chains of the Fab variable region, may be used. In other embodiments, a linear antibody, which includes a pair of tandem Fd segments (V_{H}-C_{H}1-V_{H}-C_{H}1), which, together with complementary light chain polypeptides form a pair of CD38 binding regions, may be used.

In some embodiments, a CD38 binding domain of the present disclosure includes for a target or antigen listed in Table 1, one, two, three, four, five, or all six of the CDR sequences listed in Table 1 for the listed target or antigen, as provided in further detail below Table 1.

TABLE 1
CDR Sequences

| Antibody | CDR1-IMGT | CDR2-IMGT | CDR3-IMGT | CDR1-IMGT | CDR2-IMGT | CDR3-IMGT |
|---|---|---|---|---|---|---|
| Name | (heavy) | (heavy) | (heavy) | (light) | (light) | (light) |
| anti-CD38 | GFTFNSF | ISGSGGG | AKDKILVVF | QSVSSY | DAS | QQRSNW |
| | A (SEQ ID | T (SEQ ID | GEPVFDY | (SEQ ID | | PPT |
| | NO: 85) | NO: 115) | (SEQ ID | NO: 180) | | (SEQ ID |
| | | | NO: 148) | | | NO: 211) |
| Isatuximab | GYTFTDY | IYPGDGD | ARGDYYG | QDVSTV | SAS | QQHYSPP |
| | W (SEQ ID | T (SEQ ID | SNSLDY | (SEQ ID | | YT |
| | NO: 86) | NO: 109) | (SEQ ID | NO: 181) | | (SEQ ID |
| | | | NO: 149) | | | NO: 212) |
| MOR202 | SYYMN | GISGDPS | DLPLVYT | SGDNLRH | GDSKRPS | QTYTGGA |
| (Kabat | (SEQ ID | NTYYADS | GFAY | YYVY | (SEQ ID | S (SEQ ID |
| Numbering) | NO: 225) | VKG (SEQ | (SEQ ID | (SEQ ID | NO: 229) | NO: 230) |
| | | ID NO: | NO: 227) | NO: 228) | | |
| | | 226) | | | | |

TABLE 2
VH and VL Sequences

| Antibody Name | VH | VL |
|---|---|---|
| Isatuximab | (VH + CH1) | DIVMTQSHLSMSTSLGDPVSITCK |
| | QVQLVQSGAEVAKPGTSVKLSCK | ASQDVSTVVAVVYQQKPGQSPRRL |
| | ASGYTFTDYWMQWVKQRPGQGL | IYSASYRYIGVPDRFTGSGAGTDF |
| | EWIGTIYPGDGDTGYAQKFQGKAT | TFTISSVQAEDLAVYYCQQHYSPP |
| | LTADKSSKTVYMHLSSLASEDSAV | YTFGGGTKLEIKRTVAAPSVFIFPP |
| | YYCARGDYYGSNSLDYWGQGTS | SDEQLKSGTASVVCLLNNFYPREA |
| | VTVSSASTKGPSVFPLAPSSKSTS | KVQWKVDNALQSGNSQESVTEQ |
| | GGTAALGCLVKDYFPEPVTVSWN | DSKDSTYSLSSTLTLSKADYEKHK |
| | SGALTSGVHTFPAVLQSSGLYSLS | VYACEVTHQGLSSPVTKSFNRGE |
| | SVVTVPSSSLGTQTYICNVNHKPS | C (SEQ ID NO: 233) |
| | NTKVDKKVEPKSCDKTHTCPPCP | |
| | APELLGGPSVFLFPPKPKDTLMISR | |
| | TPEVTCVVVDVSHEDPEVKFNWY | |
| | VDGVEVHNAKTKPREEQYN 300 | |
| | STYRVVSVLTVLHQDWLNGKEYK | |
| | CKVSNKALPAPIEKTISKAKGQPRE | |
| | PQVYTLPPSRDELTKNQVSLTCLV | |
| | KGFYPSDIAVEVVESNGQPENNYK | |
| | TTPPVLDSDGSFFLYSKLTVDKSR | |
| | WQQGNVFSCSVMHEALHNHYTQ | |
| | KSLSLSPGK (SEQ ID NO: 231) | |
| MOR202 | QVQLVESGGGLVQPGGSLRLSCAASGF | DIELTQPPSVSVAPGQTARISCSGDNLR |
| | TFSSYYMNWVRQAPGKGLEWVSGISG | HYYVYWYQQKPGQAPVLVIYGDSKRP |
| | DPSNTYYADSVKGRFTISRDNSKNTLYL | SGIPERFSGSNSGNTATLTISGTQAEDE |
| | QMNSLRAEDTAVYYCARDLPLVYTGFA | ADYYCQTYTGGASLVFGGGTKLTVLGQ |
| | YWGQGTLVTV (SEQ ID NO: 232) | (SEQ ID NO: 234) |

The CD38 binding domain of Fc-antigen binding domain construct 1 (110/104 in FIG. 1) can include the three heavy chain and the three light chain CDR sequences of any one of the antibodies listed in Table 1.

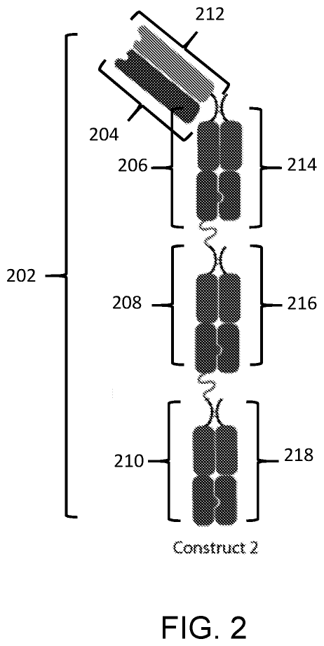

The CD38 binding domain of Fc-antigen binding domain construct 2 (212/204 in FIG. 2) can include the three heavy chain and the three light chain CDR sequences of any one of the antibodies listed in Table 1.

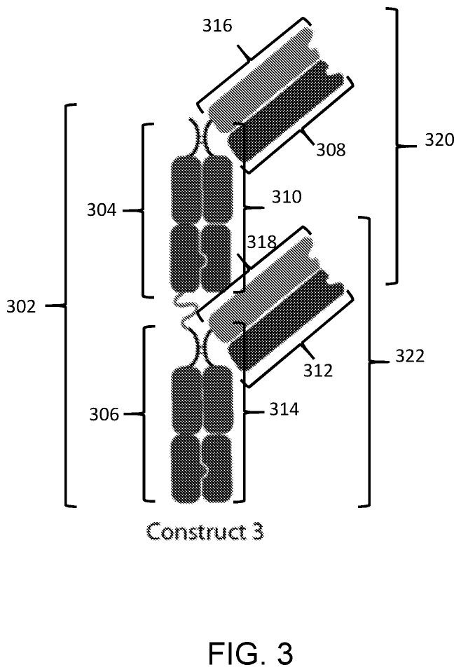

The CD38 binding domains of Fc-antigen binding domain construct 3 (308/316 and 312/318 in FIG. 3) each can include the three heavy chain and the three light chain CDR sequences of any one of the antibodies listed in Table 1.

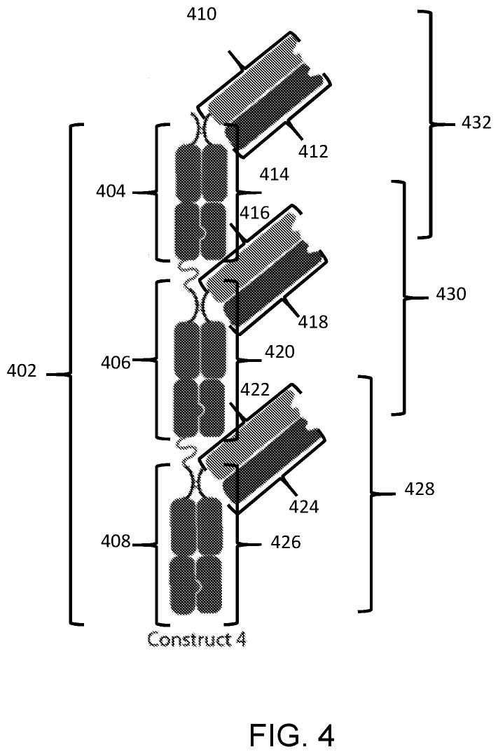

The CD38 binding domains of Fc-antigen binding domain construct 4 (410/412, 416/418 and 422/424 in FIG. 4) each can include the three heavy chain and the three light chain CDR sequences of any one of the antibodies listed in Table 1.

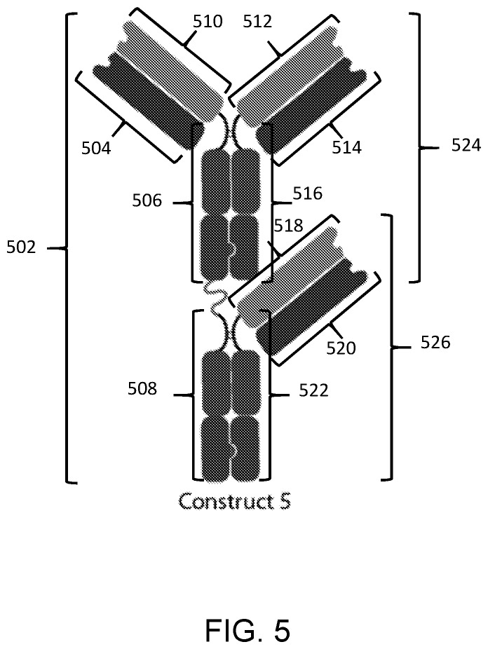

The CD38 binding domains of Fc-antigen binding domain construct 5 (510/504, 512/514 and 518/520 in FIG. 5) each can include the three heavy chain and the three light chain CDR sequences of any one of the antibodies listed in Table 1.

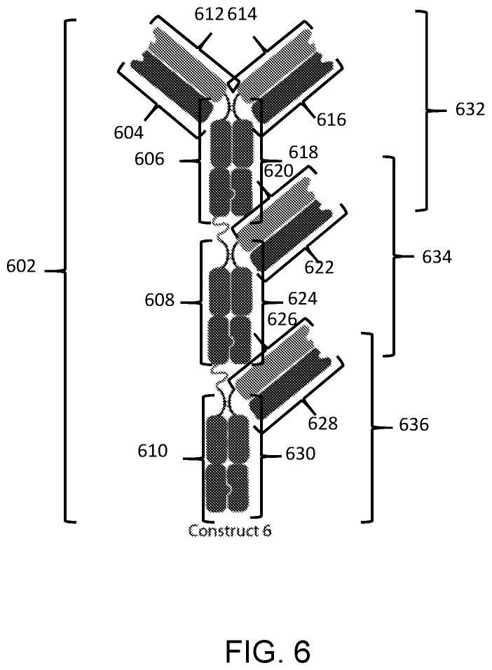

The CD38 binding domains of Fc-antigen binding domain construct 6 (612/604, 614/616, 620/622, and 626/628 in FIG. 6) each can include the three heavy chain and the three light chain CDR sequences of any one of the antibodies listed in Table 1.

The CD38 binding domains of Fc-antigen binding domain construct 7 (712/714 and 714/716 in FIG. 7) each can include the three heavy chain and the three light chain CDR sequences of any one of the antibodies listed in Table 1.

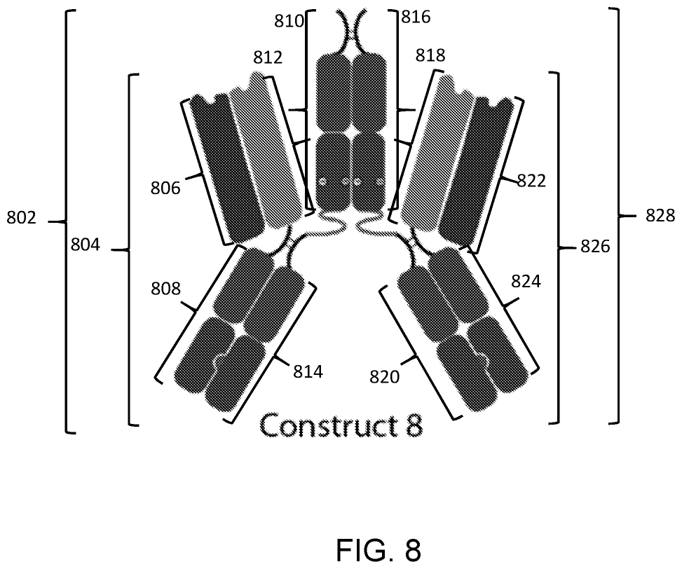

The CD38 binding domains of Fc-antigen binding domain construct 8 (812/806 and 818/822 in FIG. 8) each can include the three heavy chain and the three light chain CDR sequences of any one of the antibodies listed in Table 1.

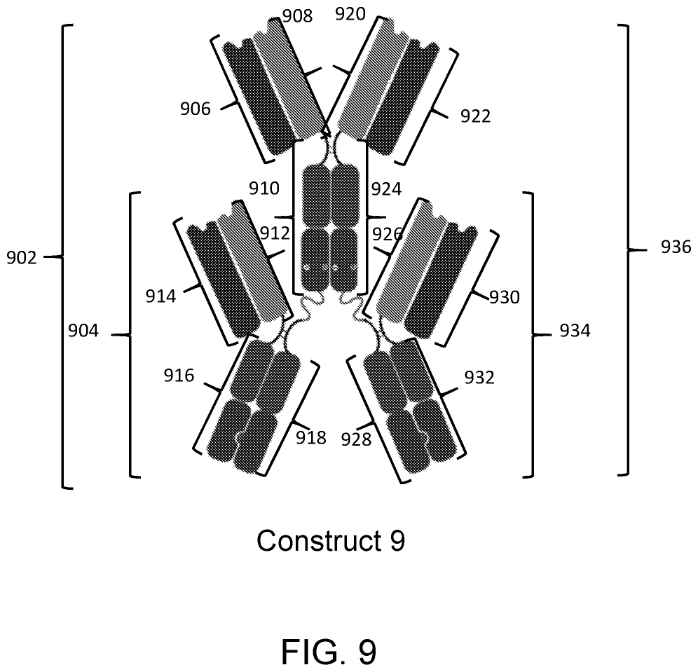

The CD38 binding domains of Fc-antigen binding domain construct 9 (908/906, 920/922, 912/914, and 926/930 in FIG. 9) each can include the three heavy chain and the three light chain CDR sequences of any one of the antibodies listed in Table 1.

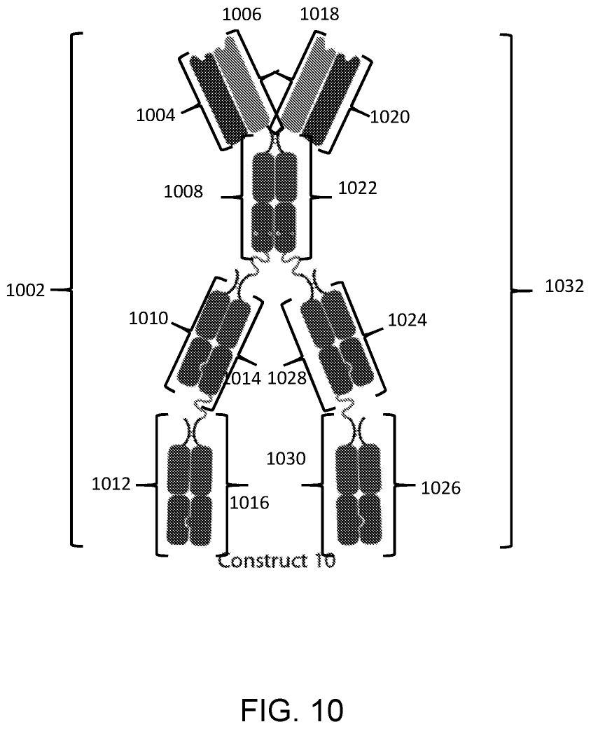

The CD38 binding domains of Fc-antigen binding domain construct 10 (1006/1004 and 1018/1020 in FIG. 10) each can include the three heavy chain and the three light chain CDR sequences of any one of the antibodies listed in Table 1.

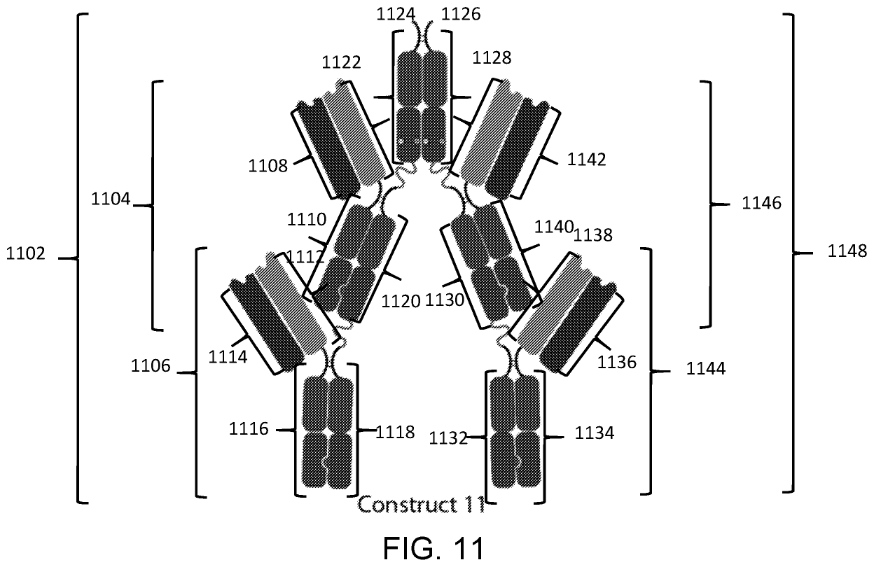

The CD38 binding domains of Fc-antigen binding domain construct 11 (1112/1114, 1122/1108, 1128/1142, and 1138/1136 in FIG. 11) each can include the three heavy chain and the three light chain CDR sequences of any one of the antibodies listed in Table 1.

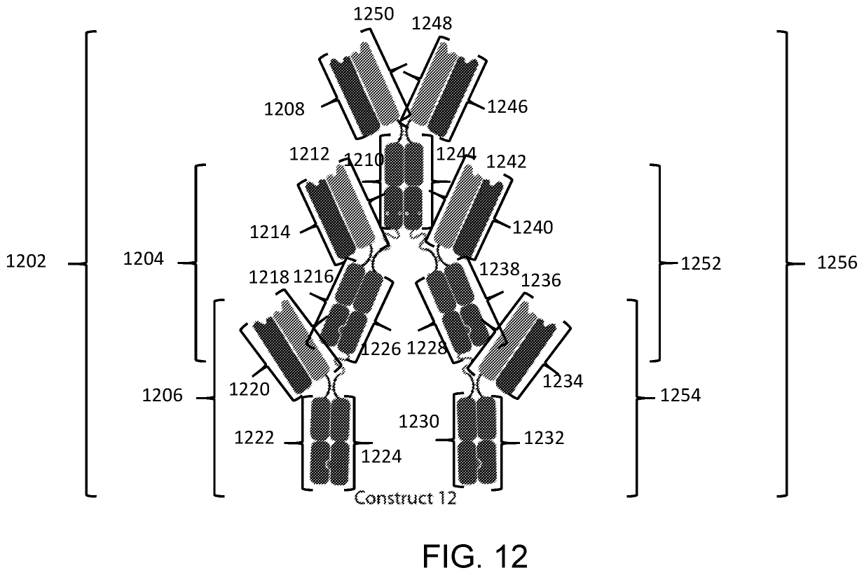

The CD38 binding domains of Fc-antigen binding domain construct 12 (1218/1220, 1212/1214, 1250/1208, 1248/1246, 1242/1240, and 1236/1234 in FIG. 12) each can include the three heavy chain and the three light chain CDR sequences of any one of the antibodies listed in Table 1.

The CD38 binding domains of Fc-antigen binding domain construct 13 (1310/1304 and 1314/1322 in FIG. 13) each can include the three heavy chain and the three light chain CDR sequences of any one of the antibodies listed in Table 1.

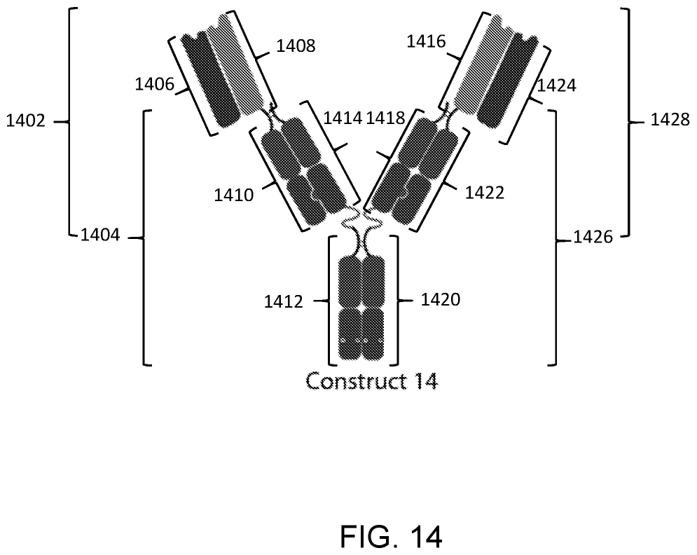

The CD38 binding domains of Fc-antigen binding domain construct 14 (1408/1406 and 1416/1424 in FIG. 14) each can include the three heavy chain and the three light chain CDR sequences of any one of the antibodies listed in Table 1.

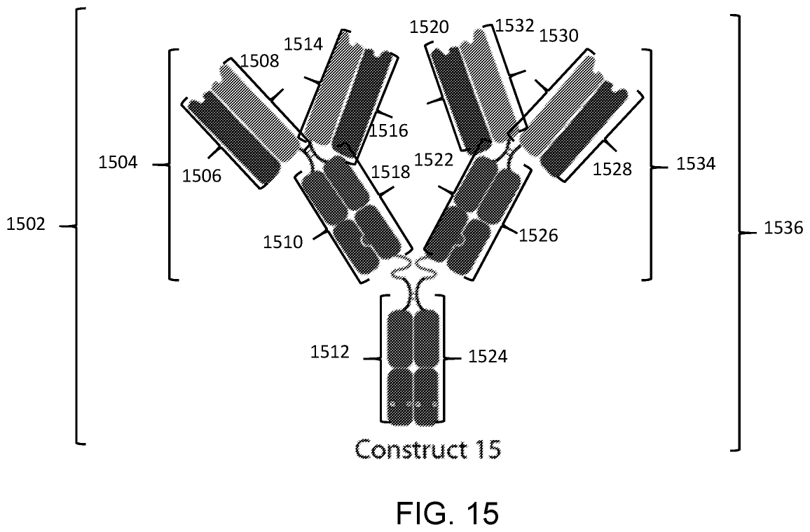

The CD38 binding domains of Fc-antigen binding domain construct 15 (1508/1506, 1514/1516, 1532/1520, and 1530/1528 in FIG. 15) each can include the three heavy chain and the three light chain CDR sequences of any one of the antibodies listed in Table 1.

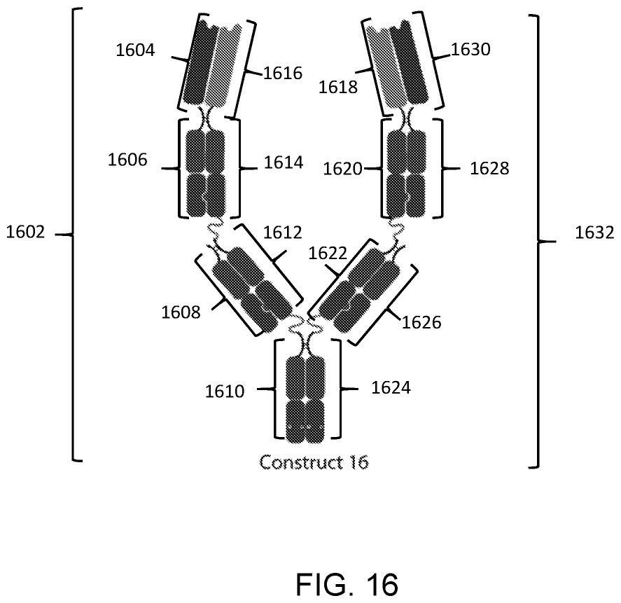

The CD38 binding domains of Fc-antigen binding domain construct 16 (1616/1604 and 1618/1630 in FIG. 16) each can include the three heavy chain and the three light chain CDR sequences of any one of the antibodies listed in Table 1.

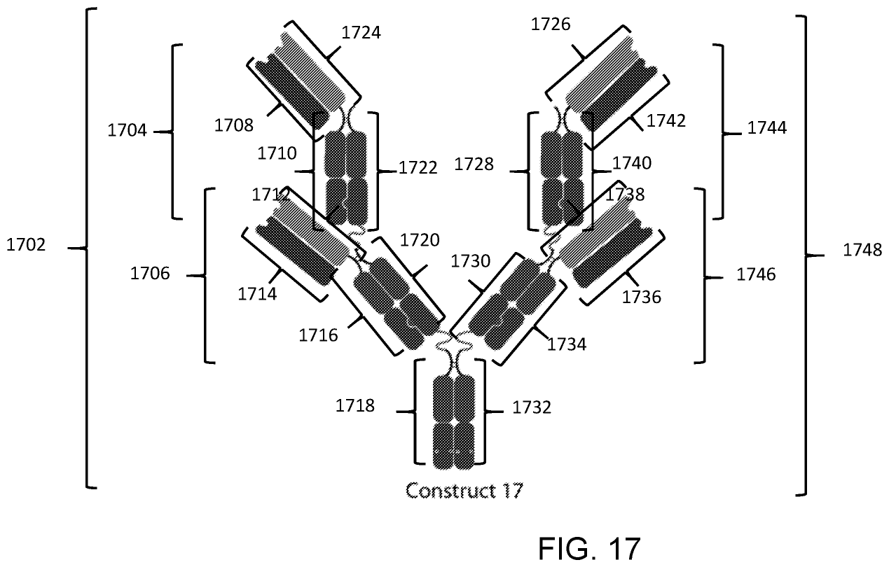

The CD38 binding domains of Fc-antigen binding domain construct 17 (1712/1714, 1724/1708, 1726/1742, and 1738/1736 in FIG. 17) each can include the three heavy chain and the three light chain CDR sequences of any one of the antibodies listed in Table 1.

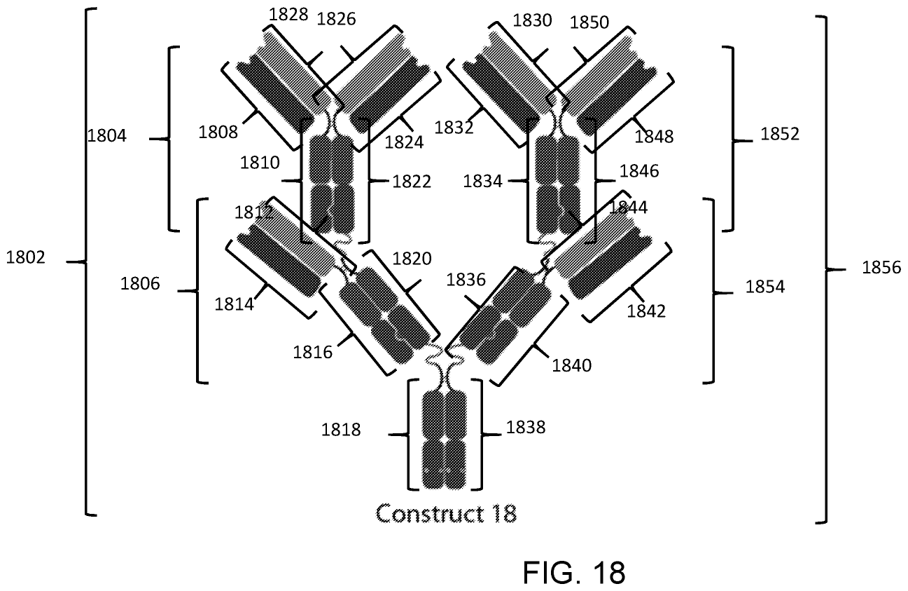

The CD38 binding domains of Fc-antigen binding domain construct 18 (1812/1814, 1828/1808, 1826/1824, 1830/1832, 1850/1848, and 1844/1842 in FIG. 18) each can include the three heavy chain and the three light chain CDR sequences of any one of the antibodies listed in Table 1.

The CD38 binding domains of Fc-antigen binding domain construct 19 (1914/1904 and 1920/1922 in FIG. 19) each can include the three heavy chain and the three light chain CDR sequences of any one of the antibodies listed in Table 1.

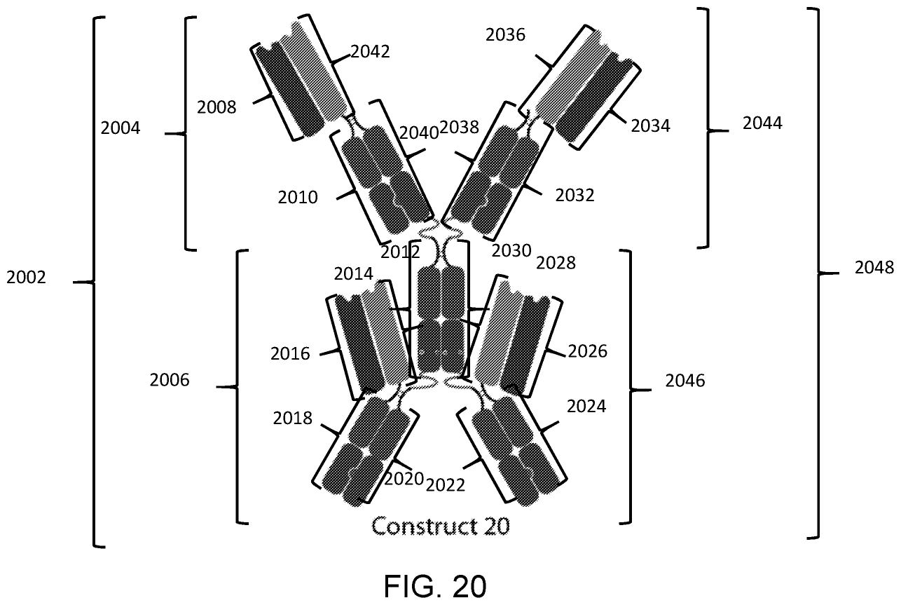

The CD38 binding domains of Fc-antigen binding domain construct 20 (2014/2016, 2042/2008, 2036/2034, and 2028/2026 in FIG. 20) each can include the three heavy chain and the three light chain CDR sequences of any one of the antibodies listed in Table 1.

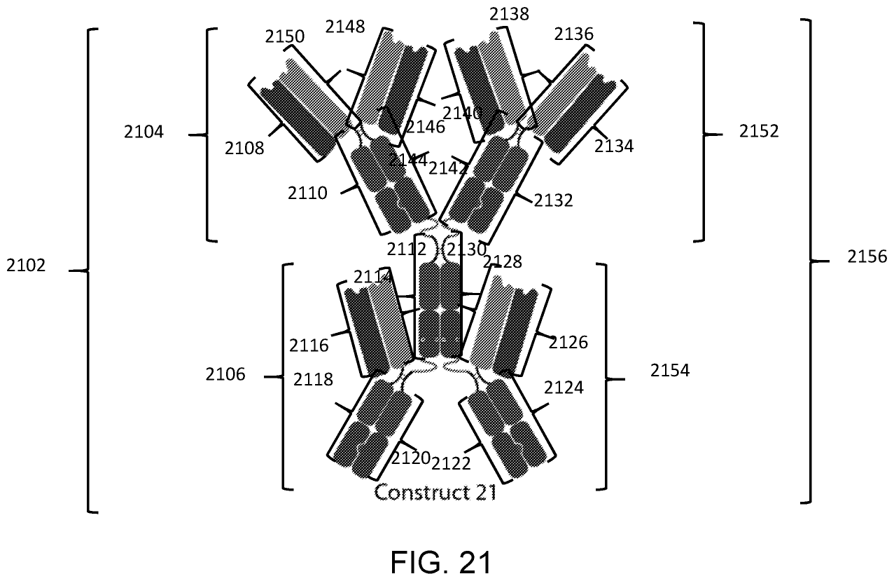

The CD38 binding domains of Fc-antigen binding domain construct 21 (2114/2116, 2150/2108, 2148/2146, 2138/2140, 2136/2134, and 2128/2126 in FIG. 21) each can include the three heavy chain and the three light chain CDR sequences of any one of the antibodies listed in Table 1.

### IV. Dimerization Selectivity Modules

In the present disclosure, a dimerization selectivity module includes components or select amino acids within the Fc domain monomer that facilitate the preferred pairing of two Fc domain monomers to form an Fc domain. Specifically, a dimerization selectivity module is that part of the C_{H}3 antibody constant domain of an Fc domain monomer which includes amino acid substitutions positioned at the interface between interacting C_{H}3 antibody constant domains of two Fc domain monomers. In a dimerization selectivity module, the amino acid substitutions make favorable the dimerization of the two C_{H}3 antibody constant domains as a result of the compatibility of amino acids chosen for those substitutions. The ultimate formation of the favored Fc domain is selective over other Fc domains which form from Fc domain monomers lacking dimerization selectivity modules or with incompatible amino acid substitutions in the dimerization selectivity modules. This type of amino acid substitution can be made using conventional molecular cloning techniques well-known in the art, such as QuikChange® mutagenesis.

In some embodiments, a dimerization selectivity module includes an engineered cavity (of “hole” described further herein) in the C_{H}3 antibody constant domain. In other embodiments, a dimerization selectivity module includes an engineered protuberance (or “knob” described further herein) in the C_{H}3 antibody constant domain. To selectively form an Fc domain, two Fc domain monomers with compatible dimerization selectivity modules, e.g., one C_{H}3 antibody constant domain containing an engineered cavity and the other C_{H}3 antibody constant domain containing an engineered protuberance, combine to form a protuberance-into-cavity (or “knob and hole”) pair of Fc domain monomers. Engineered protuberances and engineered cavities are examples of heterodimerizing selectivity modules, which can be made in the C_{H}3 antibody constant domains of Fc domain monomers in order to promote favorable heterodimerization of two Fc domain monomers that have compatible heterodimerizing selectivity modules. Table 3 lists suitable mutation.

In other embodiments, heterodimerization is achieved by use of an Fc domain monomer with a dimerization selectivity module containing positively-charged amino acid substitutions and an Fc domain monomer with a dimerization selectivity module containing negatively-charged amino acid substitutions may selectively combine to form an Fc domain through the favorable electrostatic steering (described further herein) of the charged amino acids. In some embodiments, an Fc domain monomer may include one of the following positively-charged and negatively-charged amino acid substitutions: K392D, K392E, D399K, K409D, K409E, K439D, and K439E. In one example, an Fc domain monomer containing a positively-charged amino acid substitution, e.g., D356K or E357K, and an Fc domain monomer containing a negatively-charged amino acid substitution, e.g., K370D or K370E, may selectively combine to form an Fc domain through favorable electrostatic steering of the charged amino acids. In another example, an Fc domain monomer containing E357K and an Fc domain monomer containing K370D may selectively combine to form an Fc domain through favorable electrostatic steering of the charged amino acids. In some embodiments, reverse charge amino acid substitutions may be used as heterodimerizing selectivity modules, wherein two Fc domain monomers containing different, but compatible, reverse charge amino acid substitutions combine to form a heterodimeric Fc domain. Table 3 lists various reverse charged dimerization selectivity modules for promoting heterodimerization.

There are additional types of mutations, beyond knob and hole mutations and electrostatic steering mutations, than can be employed to promoting heterodimerization. These mutations are also listed in Table 3.

In other embodiments, two Fc domain monomers include homodimerizing selectivity modules containing identical reverse charge mutations in at least two positions within the ring of charged residues at the interface between C_{H}3 domains. Homodimerizing selectivity modules are reverse charge amino acid substitutions that promote the homodimerization of Fc domain monomers to form a homodimeric Fc domain. By reversing the charge of both members of two or more complementary pairs of residues in the two Fc domain monomers, mutated Fc domain monomers remain complementary to Fc domain monomers of the same mutated sequence, but have a lower complementarity to Fc domain monomers without those mutations. In one embodiment, an Fc domain includes Fc domain monomers including the double mutants K409D/D399K, K392D/D399K, E357K/K370E, D356K/K439D, K409E/D399K, K392E/D399K, E357K/K370D, or D356K/K439E. In another embodiment, an Fc domain includes Fc domain monomers including quadruple mutants combining any pair of the double mutants, e.g., K409D/D399K/E357K/K370E. Tables 4A and 4B lists various selectivity that promote homodimerization.

In further embodiments, an Fc domain monomer containing (i) at least one reverse charge mutation and (ii) at least one engineered cavity or at least one engineered protuberance may selectively combine with another Fc domain monomer containing (i) at least one reverse charge mutation and (ii) at least one engineered protuberance or at least one engineered cavity to form an Fc domain. For example, an Fc domain monomer containing reversed charge mutation K370D and engineered cavities Y349C, T366S, L368A, and Y407V and another Fc domain monomer containing reversed charge mutation E357K and engineered protuberances S354C and T366W may selectively combine to form an Fc domain.

The formation of such Fc domains is promoted by the compatible amino acid substitutions in the C_{H}3 antibody constant domains. Two dimerization selectivity modules containing incompatible amino acid substitutions, e.g., both containing engineered cavities, both containing engineered protuberances, or both containing the same charged amino acids at the C_{H}3-C_{H}3 interface, will not promote the formation of a heterodimeric Fc domain.

Furthermore, other methods used to promote the formation of Fc domains with defined Fc domain monomers include, without limitation, the LUZ-Y approach (U.S. Patent Application Publication No. WO2011034605) which includes C-terminal fusion of a monomer α-helices of a leucine zipper to each of the Fc domain monomers to allow heterodimer formation, as well as strand-exchange engineered domain (SEED) body approach (Davis et al., Protein Eng Des Sel. 23:195-202, 2010) that generates Fc domain with heterodimeric Fc domain monomers each including alternating segments of IgA and IgG C_{H}3 sequences.

### V. Engineered Cavities and Engineered Protuberances

The use of engineered cavities and engineered protuberances (or the “knob-into-hole” strategy) is described by Carter and co-workers (Ridgway et al., Protein Eng. 9:617-612, 1996; Atwell et al., J Mol Biol. 270:26-35, 1997; Merchant et al., Nat Biotechnol. 16:677-681, 1998). The knob and hole interaction favors heterodimer formation, whereas the knob-knob and the hole-hole interaction hinder homodimer formation due to steric clash and deletion of favorable interactions. The “knob-into-hole” technique is also disclosed in U.S. Pat. No. 5,731,168.

In the present disclosure, engineered cavities and engineered protuberances are used in the preparation of the Fc-antigen binding domain constructs described herein. An engineered cavity is a void that is created when an original amino acid in a protein is replaced with a different amino acid having a smaller side-chain volume. An engineered protuberance is a bump that is created when an original amino acid in a protein is replaced with a different amino acid having a larger side-chain volume. Specifically, the amino acid being replaced is in the C_{H}3 antibody constant domain of an Fc domain monomer and is involved in the dimerization of two Fc domain monomers. In some embodiments, an engineered cavity in one C_{H}3 antibody constant domain is created to accommodate an engineered protuberance in another C_{H}3 antibody constant domain, such that both C_{H}3 antibody constant domains act as dimerization selectivity modules (e.g., heterodimerizing selectivity modules) (described above) that promote or favor the dimerization of the two Fc domain monomers. In other embodiments, an engineered cavity in one C_{H}3 antibody constant domain is created to better accommodate an original amino acid in another C_{H}3 antibody constant domain. In yet other embodiments, an engineered protuberance in one C_{H}3 antibody constant domain is created to form additional interactions with original amino acids in another C_{H}3 antibody constant domain.

An engineered cavity can be constructed by replacing amino acids containing larger side chains such as tyrosine or tryptophan with amino acids containing smaller side chains such as alanine, valine, or threonine. Specifically, some dimerization selectivity modules (e.g., heterodimerizing selectivity modules) (described further above) contain engineered cavities such as Y407V mutation in the C_{H}3 antibody constant domain. Similarly, an engineered protuberance can be constructed by replacing amino acids containing smaller side chains with amino acids containing larger side chains. Specifically, some dimerization selectivity modules (e.g., heterodimerizing selectivity modules) (described further above) contain engineered protuberances such as T366W mutation in the C_{H}3 antibody constant domain. In the present disclosure, engineered cavities and engineered protuberances are also combined with inter-C_{H}3 domain disulfide bond engineering to enhance heterodimer formation. In one example, an Fc domain monomer containing engineered cavities Y349C, T366S, L368A, and Y407V may selectively combine with another Fc domain monomer containing engineered protuberances S354C and T366W to form an Fc domain. In another example, an Fc domain monomer containing an engineered cavity with the addition of Y349C and an Fc domain monomer containing an engineered protuberance with the addition of S354C may selectively combine to form an Fc domain. Other engineered cavities and engineered protuberances, in combination with either disulfide bond engineering or structural calculations (mixed HA-TF) are included, without limitation, in Table 3.

Replacing an original amino acid residue in the C_{H}3 antibody constant domain with a different amino acid residue can be achieved by altering the nucleic acid encoding the original amino acid residue. The upper limit for the number of original amino acid residues that can be replaced is the total number of residues in the interface of the C_{H}3 antibody constant domains, given that sufficient interaction at the interface is still maintained.

Combining Engineered Cavities and Engineered Protuberances with Electrostatic Steering

Electrostatic steering can be combined with knob-in-hole technology to favor heterominerization, for example, between Fc domain monomers in two different polypeptides. Electrostatic steering, described in greater detail below, is the utilization of favorable electrostatic interactions between oppositely charged amino acids in peptides, protein domains, and proteins to control the formation of higher ordered protein molecules. Electrostatic steering can be used to promote either homodimerization or heterodimerization, the latter of which can be usefully combined with knob-in-hole technology. In the case of heterodimerization, different, but compatible, mutations are introduced in each of the Fc domain monomers which are to heterodimerize. Thus, an Fc domain monomer can be modified to include one of the following positively-charged and negatively-charged amino acid substitutions: D356K, D356R, E357K, E357R, K370D, K370E, K392D, K392E, D399K, K409D, K409E, K439D, and K439E. For example, one Fc domain monomer, for example, an Fc domain monomer having a cavity (Y349C, T366S, L368A and Y407V), can also include K370D mutation and the other Fc domain monomer, for example, an Fc domain monomer having a protuberance (S354C and T366W) can include E357K.

More generally, any of the cavity mutations (or mutation combinations): Y407T, Y407A, F405A, Y407T, T394S, T394W:Y407A, T366W:T394S, T366S:L368A:Y407V:Y349C, and S3364H:F405 can be combined with an electrostatic steering mutation in Table 3 and any of the protuberance mutations (or mutation combinations): T366Y, T366W, T394W, F405W, T366Y:F405A, T366W:Y407A, T366W:S354C, and Y349T:T394F can be combined with an electrostatic steering mutation in Table 3.

### VI. Electrostatic Steering

Electrostatic steering is the utilization of favorable electrostatic interactions between oppositely charged amino acids in peptides, protein domains, and proteins to control the formation of higher ordered protein molecules. A method of using electrostatic steering effects to alter the interaction of antibody domains to reduce for formation of homodimer in favor of heterodimer formation in the generation of bi-specific antibodies is disclosed in U.S. Patent Application Publication No. 2014-0024111.

In the present disclosure, electrostatic steering is used to control the dimerization of Fc domain monomers and the formation of Fc-antigen binding domain constructs. In particular, to control the dimerization of Fc domain monomers using electrostatic steering, one or more amino acid residues that make up the C_{H}3-C_{H}3 interface are replaced with positively- or negatively-charged amino acid residues such that the interaction becomes electrostatically favorable or unfavorable depending on the specific charged amino acids introduced. In some embodiments, a positively-charged amino acid in the interface, such as lysine, arginine, or histidine, is replaced with a negatively-charged amino acid such as aspartic acid or glutamic acid. In other embodiments, a negatively-charged amino acid in the interface is replaced with a positively-charged amino acid. The charged amino acids may be introduced to one of the interacting C_{H}3 antibody constant domains, or both. By introducing charged amino acids to the interacting C_{H}3 antibody constant domains, dimerization selectivity modules (described further above) are created that can selectively form dimers of Fc domain monomers as controlled by the electrostatic steering effects resulting from the interaction between charged amino acids.

In some embodiments, to create a dimerization selectivity module including reversed charges that can selectively form dimers of Fc domain monomers as controlled by the electrostatic steering effects, the two Fc domain monomers may be selectively formed through heterodimerization or homodimerization.

Heterodimerization of Fc Domain Monomers

Heterodimerization of Fc domain monomers can be promoted by introducing different, but compatible, mutations in the two Fc domain monomers, such as the charge residue pairs included, without limitation, in Table 3. In some embodiments, an Fc domain monomer may include one of the following positively-charged and negatively-charged amino acid substitutions: D356K, D356R, E357K, E357R, K370D, K370E, K392D, K392E, D399K, K409D, K409E, K439D, and K439E. In one example, an Fc domain monomer containing a positively-charged amino acid substitution, e.g., D356K or E357K, and an Fc domain monomer containing a negatively-charged amino acid substitution, e.g., K370D or K370E, may selectively combine to form an Fc domain through favorable electrostatic steering of the charged amino acids. In another example, an Fc domain monomer containing E357K and an Fc domain monomer containing K370D may selectively combine to form an Fc domain through favorable electrostatic steering of the charged amino acids.

For example, in an Fc-antigen binding domain construct having three Fc domains, two of the three Fc domains may be formed by the heterodimerization of two Fc domain monomers, as promoted by the electrostatic steering effects. A “heterodimeric Fc domain” refers to an Fc domain that is formed by the heterodimerization of two Fc domain monomers, wherein the two Fc domain monomers contain different reverse charge mutations (heterodimerizing selectivity modules) (see, e.g., mutations in Tables 4A and 4B) that promote the favorable formation of these two Fc domain monomers. In an Fc-antigen binding domain construct having three Fc domains—one carboxyl terminal “stem” Fc domain and two amino terminal “branch” Fc domains—each of the amino terminal “branch” Fc domains may be a heterodimeric Fc domain (also called a “branch heterodimeric Fc domain”) (e.g., a heterodimeric Fc domain formed by Fc domain monomers 106 and 114 or Fc domain monomers 112 and 116 in FIG. 1; a heterodimeric Fc domain formed by Fc domain monomers 206 and 214 or Fc domain monomers 212 and 216 in FIG. 2). A branch heterodimeric Fc domain may be formed by an Fc domain monomer containing E357K and another Fc domain monomer containing K370D.

TABLE 3
Fc heterodimerization methods

| | Mutations | Mutations | |
|---|---|---|---|
| Method | (Chain A) | (Chain B) | Reference |
| Knobs-into- | Y407T | T336Y | U.S. Pat. No. |
| Holes | | | 8,216,805 |
| (Y-T) | | | |
| Knobs-into- | Y407A | T336W | U.S. Pat. No. |
| Holes | | | 8,216,805 |
| Knobs-into- | F405A | T394W | U.S. Pat. No. |
| Holes | | | 8,216,805 |
| Knobs-into- | Y407T | T366Y | U.S. Pat. No. |
| Holes | | | 8,216,805 |
| Knobs-into- | T394S | F405W | U.S. Pat. No. |
| Holes | | | 8,216,805 |
| Knobs-into- | T394W, Y407T | T366Y, F406A | U.S. Pat. No. |
| Holes | | | 8,216,805 |
| Knobs-into- | T394S, Y407A | T366W, F405W | U.S. Pat. No. |
| Holes | | | 8,216,805 |
| Knobs-into- | T366W, T394S | F405W, T407A | U.S. Pat. No. |
| Holes | | | 8,216,805 |
| Knobs-into- | S354C, T366W | Y349C, T366S, L368A, | |
| Holes | | Y407V | |
| Knobs-into- | Y349C, T366S, L368A, | S354C, T366W | Zeidler et al, |
| Holes | Y407V | | J Immunol. |
| (CW-CSAV) | | | 163: 1246-52, 1999 |
| HA-TF | S364H, F405A | Y349T, T394F | WO2011028952 |
| Electrostatic | K409D | D399K | US 2014/0024111 |
| Steering | | | |
| Electrostatic | K409D | D399R | US 2014/0024111 |
| Steering | | | |
| Electrostatic | K409E | D399K | US 2014/0024111 |
| Steering | | | |
| Electrostatic | K409E | D399R | US 2014/0024111 |
| Steering | | | |
| Electrostatic | K392D | D399K | US 2014/0024111 |
| Steering | | | |
| Electrostatic | K392D | D399R | US 2014/0024111 |
| Steering | | | |
| Electrostatic | K392E | D399K | US 2014/0024111 |
| Steering | | | |
| Electrostatic | K392E | D399R | US 2014/0024111 |
| Steering | | | |
| Electrostatic | K392D, K409D | E356K, D399K | Gunasekaran et al., |
| Steering | | | J Biol Chem. |
| (DD-KK) | | | 285: 19637-46, 2010 |
| Electrostatic | K370E, K409D, K439E | E356K, E357K, D399K | WO 2006/106905 |
| Steering | | | |
| Knobs-into- | S354C, E357K, T366W | Y349C, T366S, L368A, | WO 2015/168643 |
| Holes plus | | K370D, Y407V | |
| Electrostatic | | | |
| Steering | | | |
| VYAV-VLLW | T350V, L351Y, F405A, | T350V, T366L, K392L, | Von Kreudenstein et al, |
| | Y407V | T394W | MAbs, |
| | | | 5: 646-54,2013 |
| EEE-RRR | D221E, P228E, L368E | D221R, P228R, K409R | Strop et al, |
| | | | J Mol Biol, |
| | | | 420: 204-19,2012 |
| EW-RVT | K360E, K409W | Q347R, D399V, F405T | Choi et al, |
| | | | Mol Cancer Ther, |
| | | | 12: 2748-59, 2013 |
| EW-RVTₛ₋ₛ | K360E, K409W, Y349C | Q347R, D399V, F405T, | Choi et al, |
| | | S354C | Mol Immunol, |
| | | | 65: 377-83, 2015 |
| Charge | L351D | T366K | De Nardis, |
| Introduction | | | J Biol Chem, |
| (DK) | | | 292: 14706-17, 2017 |
| Charge | L351D, L368E | L351K, T366K | De Nardis, J Biol Chem, |
| Introduction | | | 292: 14706-17, 2017 |
| (DEKK) | | | |
| L-R | F405L | K409R | Labrijn et al, |
| | | | Proc Natl Acad Sci USA, |
| | | | 110: 5145-50, 2013 |
| | IgG/A chimera | IgG/A chimera | Davis et al, |
| | | | Protein Eng Des Sei, |
| | | | 23: 195-202, 2010 |
| | S364K, T366V, K370T, | Q347E, Y349A, L351F, | Skegro et al, |
| | K392Y, F405S, Y407V, | S364T, T366V, K370T, | J Biol Chem, |
| | K409W, T411N | T394D, V397L, D399E, | 292: 9745-59, 2017 |
| | | F405A, Y407S, K409R, | |
| | | T411R | |
| | S364K, T366V, K370T, | F405A, Y407S | Skegro et al, |
| | K392Y, K409W, T411N | | J Biol Chem, |
| | | | 292: 9745-59, 2017 |
| | Q347A, S364K, T366V, | Q347E, Y349A, L351F, | Skegro et al, |
| | K370T, K392Y, F405S, | S364T, T366V, K370T, | J Biol Chem, |
| | Y407V, K409W, T411N | T394D, V397L, D399E, | 292: 9745-59, 2017 |
| | | F405A, Y407S, K409R, | |
| | | T411R | |
| BEAT | S364K, T366V, K370T, | Q347E, Y349A, L351F, | Skegro et al, |
| (A/B-T) | K392Y, F405S, Y407V, | S364T, T366V, K370T, | J Biol Chem, |
| | K409W, T411N | T394D, V397L, D399E, | 292: 9745-59, 2017 |
| | | F405A, Y407S, K409R | |
| DMA-RRVV | K360D, D399M, Y407A | E345R, Q347R, T366V, | Leaver-Fay et al, |
| | | K409V | Structure, |
| | | | 24: 641-51, 2016 |
| SYMV-GDQA | Y349S, K370Y, T366M, | E356G, E357D, S364Q, | Leaver-Fay et al, |
| | K409V | Y407A | Structure, |
| | | | 24: 641-51, 2016 |
| Electrostatic | K370D | E357K | |
| Steering | | | |
| Electrostatic | K370D | E357R | |
| Steering | | | |
| Electrostatic | K370E | E357K | |
| Steering | | | |
| Electrostatic | K370E | E357R | |
| Steering | | | |
| Electrostatic | K370D | D356K | |
| Steering | | | |
| Electrostatic | K370D | D356R | |
| Steering | | | |
| Electrostatic | K370E | D356K | |
| Steering | | | |
| Electrostatic | K370E | D356R | |
| Steering | | | |

Note:
All residues numbered per the EU numbering scheme (Edelman et al, Proc Natl Acad Sci USA, 63: 78-85, 1969)

Homodimerization of Fc Domain Monomers

Homodimerization of Fc domain monomers can be promoted by introducing the same electrostatic steering mutations (homodimerizing selectivity modules) in both Fc domain monomers in a symmetric fashion. In some embodiments, two Fc domain monomers include homodimerizing selectivity modules containing identical reverse charge mutations in at least two positions within the ring of charged residues at the interface between C_{H}3 domains. By reversing the charge of both members of two or more complementary pairs of residues in the two Fc domain monomers, mutated Fc domain monomers remain complementary to Fc domain monomers of the same mutated sequence, but have a lower complementarity to Fc domain monomers without those mutations. Electrostatic steering mutations that may be introduced into an Fc domain monomer to promote its homodimerization are shown, without limitation, in Tables 4A and 4B In one embodiment, an Fc domain includes two Fc domain monomers each including the double reverse charge mutants (Tables 4A and 4B), e.g., K409D/D399K. In another embodiment, an Fc domain includes two Fc domain monomers each including quadruple reverse mutants (Tables 4A and 4B), e.g., K409D/D399K/K370D/E357K.

For example, in an Fc-antigen binding domain construct having three Fc domains, one of the three Fc domains may be formed by the homodimerization of two Fc domain monomers, as promoted by the electrostatic steering effects. A “homodimeric Fc domain” refers to an Fc domain that is formed by the homodimerization of two Fc domain monomers, wherein the two Fc domain monomers contain the same reverse charge mutations (see, e.g., mutations in Tables 5 and 6). In an Fc-antigen binding domain construct having three Fc domains—one carboxyl terminal “stem” Fc domain and two amino terminal “branch” Fc domains—the carboxy terminal “stem” Fc domain may be a homodimeric Fc domain (also called a “stem homodimeric Fc domain”). A stem homodimeric Fc domain may be formed by two Fc domain monomers each containing the double mutants K409D/D399K.

TABLE 4A
Fc homodimerization methods - two mutations in each chain

| | Mutations | |
|---|---|---|
| Method | (Chains A and B) | Reference |
| Wild Type | None | U.S. Pat. No. 8,216,805 |
| Electrostatic | D399K/K409D | Gunasekaran et al., |
| Steering (KD) | | J Biol Chem. |
| | | 285: 19637-46, 2010, |
| | | WO 2015/168643 |
| Electrostatic | D399K/K409E | Gunasekaran et al., |
| Steering | | J Biol Chem. |
| | | 285: 19637-46, 2010, |
| | | WO 2015/168643 |
| Electrostatic | E357K/K370D | Gunasekaran et al., |
| Steering | | J Biol Chem. |
| | | 285: 19637-46, 2010, |
| | | WO 2015/168643 |
| Electrostatic | E357K/K370E | Gunasekaran et al., |
| Steering | | J Biol Chem. |
| | | 285: 19637-46, 2010, |
| | | WO 2015/168643 |
| Electrostatic | D356K/K439D | Gunasekaran et al., |
| Steering | | J Biol Chem. |
| | | 285: 19637-46, 2010, |
| | | WO 2015/168643 |
| Electrostatic | D356K/K439E | Gunasekaran et al., |
| Steering | | J Biol Chem. |
| | | 285: 19637-46, 2010, |
| | | WO 2015/168643 |
| Electrostatic | K392D/D399K | Gunasekaran et al., |
| Steering | | J Biol Chem. |
| | | 285: 19637-46, 2010, |
| | | WO 2015/168643 |
| Electrostatic | K392E/D399K | Gunasekaran et al., |
| Steering | | J Biol Chem. |
| | | 285: 19637-46, 2010, |
| | | WO 2015/168643 |
| Electrostatic | K409D/D399R | |
| Steering | | |
| Electrostatic | K409E/D399R | |
| Steering | | |
| Electrostatic | K392D/D399R | |
| Steering | | |

TABLE 4B
Fc homodimerization methods - four mutations in each chain

Reverse charge mutation(s) in C_{H}3 antibody
constant domain of each of the two Fc domain
monomers in a homodimeric Fc domain
K409D/D399K/K370D/E357K
K409D/D399K/K370D/E357R
K409D/D399K/K370E/E357K
K409D/D399K/K370E/E357R
K409D/D399K/K370D/D356K
K409D/D399K/K370D/D356R
K409D/D399K/K370E/D356K
K409D/D399K/K370E/D356R
K409D/D399R/K370D/E357K
K409D/D399R/K370D/E357R
K409D/D399R/K370E/E357K
K409D/D399R/K370E/E357R
K409D/D399R/K370D/D356K
K409D/D399R/K370D/D356R
K409D/D399R/K370E/D356K
K409D/D399R/K370E/D356R
K409E/D399K/K370D/E357K
K409E/D399K/K370D/E357R
K409E/D399K/K370E/E357K
K409E/D399K/K370E/E357R
K409E/D399K/K370D/D356K
K409E/D399K/K370D/D356R
K409E/D399K/K370E/D356K
K409E/D399K/K370E/D356R
K409E/D399R/K370D/E357K
K409E/D399R/K370D/E357R
K409E/D399R/K370E/E357K
K409E/D399R/K370E/E357R
K409E/D399R/K370D/D356K
K409E/D399R/K370D/D356R
K409E/D399R/K370E/D356K
K409E/D399R/K370E/D356R
K392D/D399K/K370D/E357K
K392D/D399K/K370D/E357R
K392D/D399K/K370E/E357K
K392D/D399K/K370E/E357R
K392D/D399K/K370D/D356K
K392D/D399K/K370D/D356R
K392D/D399K/K370E/D356K
K392D/D399K/K370E/D356R
K392D/D399R/K370D/E357K
K392D/D399R/K370D/E357R
K392D/D399R/K370E/E357K
K392D/D399R/K370E/E357R
K392D/D399R/K370D/D356K
K392D/D399R/K370D/D356R
K392D/D399R/K370E/D356K
K392D/D399R/K370E/D356R
K392E/D399K/K370D/E357K
K392E/D399K/K370D/E357R
K392E/D399K/K370E/E357K
K392E/D399K/K370E/E357R
K392E/D399K/K370D/D356K
K392E/D399K/K370D/D356R
K392E/D399K/K370E/D356K
K392E/D399K/K370E/D356R
K392E/D399R/K370D/E357K
K392E/D399R/K370D/E357R
K392E/D399R/K370E/E357K
K392E/D399R/K370E/E357R
K392E/D399R/K370D/D356K
K392E/D399R/K370D/D356R
K392E/D399R/K370E/D356K
K392E/D399R/K370E/D356R

### VII. Linkers

In the present disclosure, a linker is used to describe a linkage or connection between polypeptides or protein domains and/or associated non-protein moieties. In some embodiments, a linker is a linkage or connection between at least two Fc domain monomers, for which the linker connects the C-terminus of the C_{H}3 antibody constant domain of a first Fc domain monomer to the N-terminus of the hinge domain of a second Fc domain monomer, such that the two Fc domain monomers are joined to each other in tandem series. In other embodiments, a linker is a linkage between an Fc domain monomer and any other protein domains that are attached to it. For example, a linker can attach the C-terminus of the C_{H}3 antibody constant domain of an Fc domain monomer to the N-terminus of an albumin-binding peptide.

A linker can be a simple covalent bond, e.g., a peptide bond, a synthetic polymer, e.g., a polyethylene glycol (PEG) polymer, or any kind of bond created from a chemical reaction, e.g., chemical conjugation. In the case that a linker is a peptide bond, the carboxylic acid group at the C-terminus of one protein domain can react with the amino group at the N-terminus of another protein domain in a condensation reaction to form a peptide bond. Specifically, the peptide bond can be formed from synthetic means through a conventional organic chemistry reaction well-known in the art, or by natural production from a host cell, wherein a polynucleotide sequence encoding the DNA sequences of both proteins, e.g., two Fc domain monomer, in tandem series can be directly transcribed and translated into a contiguous polypeptide encoding both proteins by the necessary molecular machineries, e.g., DNA polymerase and ribosome, in the host cell.

In the case that a linker is a synthetic polymer, e.g., a PEG polymer, the polymer can be functionalized with reactive chemical functional groups at each end to react with the terminal amino acids at the connecting ends of two proteins.

In the case that a linker (except peptide bond mentioned above) is made from a chemical reaction, chemical functional groups, e.g., amine, carboxylic acid, ester, azide, or other functional groups commonly used in the art, can be attached synthetically to the C-terminus of one protein and the N-terminus of another protein, respectively. The two functional groups can then react to through synthetic chemistry means to form a chemical bond, thus connecting the two proteins together. Such chemical conjugation procedures are routine for those skilled in the art.

Spacer

In the present disclosure, a linker between two Fc domain monomers can be an amino acid spacer including 3-200 amino acids (e.g., 3-200, 3-180, 3-160, 3-140, 3-120, 3-100, 3-90, 3-80, 3-70, 3-60, 3-50, 3-45, 3-40, 3-35, 3-30, 3-25, 3-20, 3-15, 3-10, 3-9, 3-8, 3-7, 3-6, 3-5, 3-4, 4-200, 5- 200, 6-200, 7-200, 8-200, 9-200, 10-200, 15-200, 20-200, 25-200, 30-200, 35-200, 40-200, 45-200, 50-200, 60-200, 70-200, 80-200, 90-200, 100-200, 120-200, 140-200, 160-200, or 180-200 amino acids). In some embodiments, a linker between two Fc domain monomers is an amino acid spacer containing at least 12 amino acids, such as 12-200 amino acids (e.g., 12-200, 12-180, 12-160, 12-140, 12-120, 12-100, 12-90, 12-80, 12-70, 12-60, 12-50, 12-40, 12-30, 12-20, 12-19, 12-18, 12-17, 12-16, 12-15, 12-14, or 12-13 amino acids) (e.g., 14-200, 16-200, 18-200, 20-200, 30-200, 40-200, 50-200, 60-200, 70-200, 80-200, 90-200, 100-200, 120-200, 140-200, 160-200, 180-200, or 190-200 amino acids). In some embodiments, a linker between two Fc domain monomers is an amino acid spacer containing 12-30 amino acids (e.g., 12, 13, 14, 15, 16, 17, 18, 19, 20, 21, 22, 23, 24, 25, 26, 27, 28, 29, or 30 amino acids). Suitable peptide spacers are known in the art, and include, for example, peptide linkers containing flexible amino acid residues such as glycine and serine. In certain embodiments, a spacer can contain motifs, e.g., multiple or repeating motifs, of GS, GGS, GGGGS (SEQ ID NO: 1), GGSG (SEQ ID NO: 2), or SGGG (SEQ ID NO: 3). In certain embodiments, a spacer can contain 2 to 12 amino acids including motifs of GS, e.g., GS, GSGS (SEQ ID NO: 4), GSGSGS (SEQ ID NO: 5), GSGSGSGS (SEQ ID NO: 6), GSGSGSGSGS (SEQ ID NO: 7), or GSGSGSGSGSGS (SEQ ID NO: 8). In certain other embodiments, a spacer can contain 3 to 12 amino acids including motifs of GGS, e.g., GGS, GGSGGS (SEQ ID NO: 9), GGSGGSGGS (SEQ ID NO: 10), and GGSGGSGGSGGS (SEQ ID NO: 11). In yet other embodiments, a spacer can contain 4 to 20 amino acids including motifs of GGSG (SEQ ID NO: 2), e.g., GGSGGGSG (SEQ ID NO: 12), GGSGGGSGGGSG (SEQ ID NO: 13), GGSGGGSGGGSGGGSG (SEQ ID NO: 14), or GGSGGGSGGGSGGGSGGGSG (SEQ ID NO: 15). In other embodiments, a spacer can contain motifs of GGGGS (SEQ ID NO: 1), e.g., GGGGSGGGGS (SEQ ID NO: 16) or GGGGSGGGGSGGGGS (SEQ ID NO: 17). In certain embodiments, a spacer is SGGGSGGGSGGGSGGGSGGG (SEQ ID NO: 18).

In some embodiments, a spacer between two Fc domain monomers contains only glycine residues, e.g., at least 4 glycine residues (e.g., 4-200 (SEQ ID NO: 235), 4-180 (SEQ ID NO: 236), 4-160 (SEQ ID NO: 237), 4-140 (SEQ ID NO: 238), 4-40 (SEQ ID NO: 239), 4-100 (SEQ ID NO: 240), 4-90 (SEQ ID NO: 241), 4-80 (SEQ ID NO: 242), 4-70 (SEQ ID NO: 243), 4-60 (SEQ ID NO: 244), 4-50 (SEQ ID NO: 245), 4-40 (SEQ ID NO: 239), 4-30 (SEQ ID NO: 214), 4-20 (SEQ ID NO: 217), 4-19 (SEQ ID NO: 246), 4-18 (SEQ ID NO: 247), 4-17 (SEQ ID NO: 248), 4-16 (SEQ ID NO: 249), 4-15 (SEQ ID NO: 250), 4-14 (SEQ ID NO: 251), 4-13 (SEQ ID NO: 252), 4-12 (SEQ ID NO: 253), 4-11 (SEQ ID NO: 254), 4-10 (SEQ ID NO: 255), 4-9 (SEQ ID NO: 256), 4-8 (SEQ ID NO: 257), 4-7 (SEQ ID NO: 258), 4-6 (SEQ ID NO: 259) or 4-5 (SEQ ID NO: 260) glycine residues) (e.g., 4-200 (SEQ ID NO: 235), 6-200 (SEQ ID NO: 261), 8-200 (SEQ ID NO: 262), 10-200 (SEQ ID NO: 263), 12-200 (SEQ ID NO: 264), 14-200 (SEQ ID NO: 265), 16-200 (SEQ ID NO: 266), 18-200 (SEQ ID NO: 267), 20-200 (SEQ ID NO: 268), 30-200 (SEQ ID NO: 269), 40-200 (SEQ ID NO: 270), 50-200 (SEQ ID NO: 271), 60-200 (SEQ ID NO: 272), 70-200 (SEQ ID NO: 273), 80-200 (SEQ ID NO: 274), 90-200 (SEQ ID NO: 275), 100-200 (SEQ ID NO: 276), 120-200 (SEQ ID NO: 277), 140-200 (SEQ ID NO: 278), 160-200 (SEQ ID NO: 279), 180-200 (SEQ ID NO: 280), or 190-200 (SEQ ID NO: 281) glycine residues). In certain embodiments, a spacer has 4-30 glycine residues (SEQ ID NO: 214) (e.g., 4, 5, 6, 7, 8, 9, 10, 11, 12, 13, 14, 15, 16, 17, 18, 19, 20, 21, 22, 23, 24, 25, 26, 27, 28, 29, or 30 glycine residues (SEQ ID NO: 214)). In some embodiments, a spacer containing only glycine residues may not be glycosylated (e.g., O-linked glycosylation, also referred to as O-glycosylation) or may have a decreased level of glycosylation (e.g., a decreased level of O-glycosylation) (e.g., a decreased level of 0-glycosylation with glycans such as xylose, mannose, sialic acids, fucose (Fuc), and/or galactose (Gal) (e.g., xylose)) as compared to, e.g., a spacer containing one or more serine residues (e.g., SGGGSGGGSGGGSGGGSGGG (SEQ ID NO: 18)).

In some embodiments, a spacer containing only glycine residues may not be 0-glycosylated (e.g., O-xylosylation) or may have a decreased level of O-glycosylation (e.g., a decreased level of O-xylosylation) as compared to, e.g., a spacer containing one or more serine residues (e.g., SGGGSGGGSGGGSGGGSGGG (SEQ ID NO: 18)).

In some embodiments, a spacer containing only glycine residues may not undergo proteolysis or may have a decreased rate of proteolysis as compared to, e.g., a spacer containing one or more serine residues (e.g., SGGGSGGGSGGGSGGGSGGG (SEQ ID NO: 18)).

In certain embodiments, a spacer can contain motifs of GGGG (SEQ ID NO: 19), e.g., GGGGGGGG (SEQ ID NO: 20), GGGGGGGGGGGG (SEQ ID NO: 21), GGGGGGGGGGGGGGGG (SEQ ID NO: 22), or GGGGGGGGGGGGGGGGGGGG (SEQ ID NO: 23). In certain embodiments, a spacer can contain motifs of GGGGG (SEQ ID NO: 24), e.g., GGGGGGGGGG (SEQ ID NO: 25), or GGGGGGGGGGGGGGG (SEQ ID NO: 26). In certain embodiments, a spacer is GGGGGGGGGGGGGGGGGGGG (SEQ ID NO: 27).

In other embodiments, a spacer can also contain amino acids other than glycine and serine, e.g., GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), or GGSGGGSEGGGSEGGGSEGGGSEGGGSEGGGSGGGS (SEQ ID NO: 34).

In certain embodiments in the present disclosure, a 12- or 20-amino acid peptide spacer is used to connect two Fc domain monomers in tandem series, the 12- and 20-amino acid peptide spacers consisting of sequences GGGSGGGSGGGS (SEQ ID NO: 35) and SGGGSGGGSGGGSGGGSGGG (SEQ ID NO: 18), respectively. In other embodiments, an 18-amino acid peptide spacer consisting of sequence GGSGGGSGGGSGGGSGGS (SEQ ID NO: 36) may be used.

In some embodiments, a spacer between two Fc domain monomers may have a sequence that is at least 75% identical (e.g., at least 77%, 79%, 81%, 83%, 85%, 87%, 89%, 91%, 93%, 95%, 97%, 99%, or 99.5% identical) to the sequence of any one of SEQ ID NOs: 1-36 described above. In certain embodiments, a spacer between two Fc domain monomers may have a sequence that is at least 80% identical (e.g., at least 82%, 85%, 87%, 90%, 92%, 95%, 97%, 99%, or 99.5% identical) to the sequence of any one of SEQ ID NOs: 17, 18, 26, and 27. In certain embodiments, a spacer between two Fc domain monomers may have a sequence that is at least 80% identical (e.g., at least 82%, 85%, 87%, 90%, 92%, 95%, 97%, 99%, or 99.5%) to the sequence of SEQ ID NO: 18 or 27.

In certain embodiments, the linker between the amino terminus of the hinge of an Fc domain monomer and the carboxy terminus of a Fc monomer that is in the same polypeptide (i.e., the linker connects the C-terminus of the C_{H}3 antibody constant domain of a first Fc domain monomer to the N-terminus of the hinge domain of a second Fc domain monomer, such that the two Fc domain monomers are joined to each other in tandem series) is a spacer having 3 or more amino acids rather than a covalent bond (e.g., 3-200 amino acids (e.g., 3-200, 3-180, 3-160, 3-140, 3-120, 3-100, 3-90, 3-80, 3-70, 3-60, 3-50, 3-45, 3-40, 3-35, 3-30, 3-25, 3-20, 3-15, 3-10, 3-9, 3-8, 3-7, 3-6, 3-5, 3-4, 4-200, 5-200, 6-200, 7-200, 8-200, 9-200, 10-200, 15-200, 20-200, 25-200, 30-200, 35-200, 40-200, 45-200, 50-200, 60-200, 70-200, 80-200, 90-200, 100-200, 120-200, 140-200, 160-200, or 180-200 amino acids) or an amino acid spacer containing at least 12 amino acids, such as 12-200 amino acids (e.g., 12-200, 12-180, 12-160, 12-140, 12-120, 12-100, 12-90, 12-80, 12-70, 12-60, 12-50, 12-40, 12-30, 12-20, 12-19, 12-18, 12-17, 12-16, 12-15, 12-14, or 12-13 amino acids) (e.g., 14-200, 16-200, 18-200, 20-200, 30-200, 40-200, 50-200, 60-200, 70-200, 80-200, 90-200, 100-200, 120-200, 140-200, 160-200, 180-200, or 190-200 amino acids)).

A spacer can also be present between the N-terminus of the hinge domain of a Fc domain monomer and the carboxy terminus of a CD38 binding domain (e.g., a CH1 domain of a CD38 heavy chain binding domain or the CL domain of a CD38 light chain binding domain) such that the domains are joined by a spacer of 3 or more amino acids (e.g., 3-200 amino acids (e.g., 3-200, 3-180, 3-160, 3-140, 3-120, 3-100, 3-90, 3-80, 3-70, 3-60, 3-50, 3-45, 3-40, 3-35, 3-30, 3-25, 3-20, 3-15, 3-10, 3-9, 3-8, 3-7, 3-6, 3-5, 3-4, 4-200, 5-200, 6-200, 7-200, 8-200, 9-200, 10-200, 15-200, 20-200, 25-200, 30-200, 35-200, 40-200, 45-200, 50-200, 60-200, 70-200, 80-200, 90-200, 100-200, 120-200, 140-200, 160-200, or 180-200 amino acids) or an amino acid spacer containing at least 12 amino acids, such as 12-200 amino acids (e.g., 12-200, 12-180, 12-160, 12-140, 12-120, 12-100, 12-90, 12-80, 12-70, 12-60, 12-50, 12-40, 12-30, 12-20, 12-19, 12-18, 12-17, 12-16, 12-15, 12-14, or 12-13 amino acids) (e.g., 14-200, 16-200, 18-200, 20-200, 30-200, 40-200, 50-200, 60-200, 70-200, 80-200, 90-200, 100-200, 120-200, 140-200, 160-200, 180-200, or 190-200 amino acids)).

### VIII. Serum Protein-Binding Peptides

Binding to serum protein peptides can improve the pharmacokinetics of protein pharmaceuticals, and in particular the Fc-antigen binding domain constructs described here may be fused with serum protein-binding peptides

As one example, albumin-binding peptides that can be used in the methods and compositions described here are generally known in the art. In one embodiment, the albumin binding peptide includes the sequence DICLPRWGCLW (SEQ ID NO: 37). In some embodiments, the albumin binding peptide has a sequence that is at least 80% identical (e.g., 80%, 90%, or 100% identical) to the sequence of SEQ ID NO: 37.

In the present disclosure, albumin-binding peptides may be attached to the N- or C-terminus of certain polypeptides in the Fc-antigen binding domain construct. In one embodiment, an albumin-binding peptide may be attached to the C-terminus of one or more polypeptides in Fc constructs containing a CD38 binding domain. In another embodiment, an albumin-binding peptide can be fused to the C-terminus of the polypeptide encoding two Fc domain monomers linked in tandem series in Fc constructs containing a CD38 binding domain. In yet another embodiment, an albumin-binding peptide can be attached to the C-terminus of Fc domain monomer (e.g., Fc domain monomers 114 and 116 in FIG. 1; Fc domain monomers 214 and 216 in FIG. 2) which is joined to the second Fc domain monomer in the polypeptide encoding the two Fc domain monomers linked in tandem series. Albumin-binding peptides can be fused genetically to Fc-antigen binding domain constructs or attached to Fc-antigen binding domain constructs through chemical means, e.g., chemical conjugation. If desired, a spacer can be inserted between the Fc-antigen binding domain construct and the albumin-binding peptide. Without being bound to a theory, it is expected that inclusion of an albumin-binding peptide in an Fc-antigen binding domain construct of the disclosure may lead to prolonged retention of the therapeutic protein through its binding to serum albumin.

### I. Fc-Antigen Binding Domain Constructs

In general, the disclosure features Fc-antigen binding domain constructs having 2-10 Fc domains and one or more CD38 binding domains attached. These may have greater binding affinity and/or avidity than a single wild-type Fc domain for an Fc receptor, e.g., FcγRIIIa. The disclosure discloses methods of engineering amino acids at the interface of two interacting C_{H}3 antibody constant domains such that the two Fc domain monomers of an Fc domain selectively form a dimer with each other, thus preventing the formation of unwanted multimers or aggregates. An Fc-antigen binding domain construct includes an even number of Fc domain monomers, with each pair of Fc domain monomers forming an Fc domain. An Fc-antigen binding domain construct includes, at a minimum, two functional Fc domains formed from dimer of four Fc domain monomers and oneCD38 binding domain. The CD38 binding domain may be joined to an Fc domain e.g., with a linker, a spacer, a peptide bond, a chemical bond or chemical moiety.

The Fc-antigen binding domain constructs can be assembled in many ways. The Fc-antigen binding domain constructs can be assembled from asymmetrical tandem Fc domains (FIG. 1-FIG. 6). The Fc-antigen binding domain constructs can be assembled from singly branched Fc domains, where the branch point is at the N-terminal Fc domain (FIG. 7-FIG. 12). The Fc-antigen binding domain constructs can be assembled from singly branched Fc domains, where the branch point is at the C-terminal Fc domain (FIG. 13-FIG. 18). The Fc-antigen binding domain constructs can be assembled from singly branched Fc domains, where the branch point is neither at the N- or C-terminal Fc domain (FIG. 19-FIG. 21).

The CD38 binding domain can be joined to the Fc-antigen binding domain construct in many ways. The CD38 binding domain can be expressed as a fusion protein of an Fc chain. The heavy chain component of a CD38 binding Fab can be expressed as a fusion protein of an Fc chain and the light chain component can be expressed as a separate polypeptide (FIG. 50, panel A). In some embodiments, a scFv is used as a CD38 binding domain. The scFv can be expressed as a fusion protein of the long Fc chain (FIG. 50, panel B). In some embodiments, the heavy chain and light chain components are expressed separately and exogenously added to the Fc-antigen binding domain construct. In some embodiments, the CD38 binding domain is expressed separately and later joined to the Fc-antigen binding domain construct with a chemical bond (FIG. 50, panel C).

In some embodiments, one or more Fc polypeptides in an Fc-antigen binding domain construct lack a C-terminal lysine residue. In some embodiments, all of the Fc polypeptides in an Fc-antigen binding domain construct lack a C-terminal lysine residue. In some embodiments, the absence of a C-terminal lysine in one or more Fc polypeptides in an Fc-antigen binding domain construct may improve the homogeneity of a population of an Fc-antigen binding domain construct (e.g., an Fc-antigen binding domain construct having three Fc domains), e.g., a population of an Fc-antigen binding domain construct having three Fc domains that is at least 85%, 90%, 95%, 98%, or 99% homogeneous.

In some embodiments, the N-terminal Asp in one or more of the first, second, third, fourth, fifth, or sixth polypeptides in an Fc-antigen binding domain construct described herein (e.g., polypeptides 102, 112, and 114 in FIGS. 1, 202, 214, 216 and 218 in FIGS. 2, 302, 320, and 322 in FIGS. 3, 402, 428, 430, and 432 in FIGS. 4, 502, 524, and 526 in FIGS. 5, 602, 632, 634, and 636 in FIGS. 6, 702, 708, 722, and 724 in FIGS. 7, 802, 804, 826, and 828 in FIGS. 8, 902, 904, 934, and 936 in FIGS. 9, 1002, 1010, 1012, 1024, 1026, and 1032 in FIGS. 10, 1102, 1104, 1106, 1144, 1146, and 1148 in FIGS. 11, 1202, 1204, 1206, 1252, 1254, and 1256 in FIGS. 12, 1302, 1306 1320, and 1324 in FIGS. 13, 1402, 1404, 1426, and 1428 in FIG. 14, 1502, 1504, 1534, and 1536 in FIGS. 15, 1602, 1606, 1608, 1626, 1628, and 1632 in FIGS. 16, 1702, 1704, 1706, 1744, 1746, and 1748 in FIGS. 17, 1802, 1804, 1806, 1852, 1854, and 1856 in FIGS. 18, 1902, 1906, 1910, 1924, 1928, and 1932 in FIGS. 19, 2002, 2004, 2006, 2044, 2046, and 2048 in FIGS. 20, 2102, 2104, 2106, 2152, 2154, and 2156 in FIG. 21 may be mutated to Gln.

For the exemplary Fc-antigen binding domain constructs described in the Examples herein, Fc-antigen binding domain constructs 1-21may contain the E357K and K370D charge pairs in the Knobs and Holes subunits, respectively.

Any one of the exemplary Fc-antigen binding domain constructs described herein (e.g. Fc-antigen binding domain constructs 1-21) can have enhanced effector function in an antibody-dependent cytotoxicity (ADCC) assay, an antibody-dependent cellular phagocytosis (ADCP) and/or complement-dependent cytotoxicity (CDC) assay relative to a construct having a single Fc domain and the CD38 binding domain, or can include a biological activity that is not exhibited by a construct having a single Fc domain and the CD38 binding domain.

### X. Host Cells and Protein Production

In the present disclosure, a host cell refers to a vehicle that includes the necessary cellular components, e.g., organelles, needed to express the polypeptides and constructs described herein from their corresponding nucleic acids. The nucleic acids may be included in nucleic acid vectors that can be introduced into the host cell by conventional techniques known in the art (transformation, transfection, electroporation, calcium phosphate precipitation, direct microinjection, etc.). Host cells can be of mammalian, bacterial, fungal or insect origin. Mammalian host cells include, but are not limited to, CHO (or CHO-derived cell strains, e.g., CHO-K1, CHO-DXB11 CHO-DG44), murine host cells (e.g., NS0, Sp2/0), VERY, HEK (e.g., HEK293), BHK, HeLa, COS, MDCK, 293, 3T3, W138, BT483, Hs578T, HTB2, BT20 and T47D, CRL7O3O and HsS78Bst cells. Host cells can also be chosen that modulate the expression of the protein constructs, or modify and process the protein product in the specific fashion desired. Different host cells have characteristic and specific mechanisms for the post-translational processing and modification of protein products. Appropriate cell lines or host systems can be chosen to ensure the correct modification and processing of the protein expressed.

For expression and secretion of protein products from their corresponding DNA plasmid constructs, host cells may be transfected or transformed with DNA controlled by appropriate expression control elements known in the art, including promoter, enhancer, sequences, transcription terminators, polyadenylation sites, and selectable markers. Methods for expression of therapeutic proteins are known in the art. See, for example, Paulina Balbas, Argelia Lorence (eds.) Recombinant Gene Expression: Reviews and Protocols (Methods in Molecular Biology), Humana Press; 2nd ed. 2004 edition (Jul. 20, 2004); Vladimir Voynov and Justin A. Caravella (eds.) Therapeutic Proteins: Methods and Protocols (Methods in Molecular Biology) Humana Press; 2nd ed. 2012 edition (Jun. 28, 2012).

### XI. Afucosylation

Each Fc monomer includes an N-glycosylation site at Asn 297. The glycan can be present in a number of different forms on a given Fc monomer. In a composition containing antibodies or the antigen-binding Fc constructs described herein, the glycans can be quite heterogeneous and the nature of the glycan present can depend on, among other things, the type of cells used to produce the antibodies or antigen-binding Fc constructs, the growth conditions for the cells (including the growth media) and post-production purification. In various instances, compositions containing a construct or polypeptide complex or polypeptide described herein are afucosylated to at least some extent. For example, at least 5%, 10%, 15%, 20%, 25%, 30%, 35%, 40%, 45%, 50%, 60%, 70%, 80%, 90% or 95% of the glycans (e.g., the Fc glycans) present in the composition lack a fucose residue. Thus, 5%-60%, 5%-50%, 5%-40%, 10%-50%, 10%-50%, 10%-40%, 20%-50%, or 20%-40% of the glycans lack a fucose residue. Compositions that are afucosylated to at least some extent can be produced by culturing cells producing the antibody in the presence of 1,3,4-Tri-O-acetyl-2-deoxy-2-fluoro-L-fucose inhibitor. Relatively afucosylated forms of the constructs and polypeptides described herein can be produced using a variety of other methods, including: expressing in cells with reduced or no expression of FUT8 (e.g, by knocking out FUT8 or reducing expression with RNAi (siRNA, miRNA or shRNA) and expressing in cells that overexpress beta-1,4-mannosyl-glycoprotein 4-beta-N-acetylglucosaminyltransferase (GnT-III).

### XII. Purification

An Fc-antigen binding domain construct can be purified by any method known in the art of protein purification, for example, by chromatography (e.g., ion exchange, affinity (e.g., Protein A affinity), and size-exclusion column chromatography), centrifugation, differential solubility, or by any other standard technique for the purification of proteins. For example, an Fc-antigen binding domain construct can be isolated and purified by appropriately selecting and combining affinity columns such as Protein A column with chromatography columns, filtration, ultrafiltration, salting-out and dialysis procedures (see, e.g., Process Scale Purification of Antibodies, Uwe Gottschalk (ed.) John Wiley & Sons, Inc., 2009; and Subramanian (ed.) Antibodies-Volume I-Production and Purification, Kluwer Academic/Plenum Publishers, New York (2004)).

In some instances, an Fc-antigen binding domain construct can be conjugated to one or more purification peptides to facilitate purification and isolation of the Fc-antigen binding domain construct from, e.g., a whole cell lysate mixture. In some embodiments, the purification peptide binds to another moiety that has a specific affinity for the purification peptide. In some embodiments, such moieties which specifically bind to the purification peptide are attached to a solid support, such as a matrix, a resin, or agarose beads. Examples of purification peptides that may be joined to an Fc-antigen binding domain construct include, but are not limited to, a hexa-histidine peptide (SEQ ID NO: 38), a FLAG peptide, a myc peptide, and a hemagglutinin (HA) peptide. A hexa-histidine peptide (SEQ ID NO: 38) (HHHHHH (SEQ ID NO: 38)) binds to nickel-functionalized agarose affinity column with micromolar affinity. In some embodiments, a FLAG peptide includes the sequence DYKDDDDK (SEQ ID NO: 39). In some embodiments, a FLAG peptide includes integer multiples of the sequence DYKDDDDK (SEQ ID NO: 39) in tandem series, e.g., 3xDYKDDDDK (SEQ ID NO: 282). In some embodiments, a myc peptide includes the sequence EQKLISEEDL (SEQ ID NO: 40). In some embodiments, a myc peptide includes integer multiples of the sequence EQKLISEEDL (SEQ ID NO: 40) in tandem series, e.g., 3xEQKLISEEDL (SEQ ID NO: 283). In some embodiments, an HA peptide includes the sequence YPYDVPDYA (SEQ ID NO: 41). In some embodiments, an HA peptide includes integer multiples of the sequence YPYDVPDYA (SEQ ID NO: 41) in tandem series, e.g., 3xYPYDVPDYA (SEQ ID NO: 284). Antibodies that specifically recognize and bind to the FLAG, myc, or HA purification peptide are well-known in the art and often commercially available. A solid support (e.g., a matrix, a resin, or agarose beads) functionalized with these antibodies may be used to purify an Fc-antigen binding domain construct that includes a FLAG, myc, or HA peptide.

For the Fc-antigen binding domain constructs, Protein A column chromatography may be employed as a purification process. Protein A ligands interact with Fc-antigen binding domain constructs through the Fc region, making Protein A chromatography a highly selective capture process that is able to remove most of the host cell proteins. In the present disclosure, Fc-antigen binding domain constructs may be purified using Protein A column chromatography as described in Example 2.

### XIII. Pharmaceutical Compositions/Preparations

The disclosure features pharmaceutical compositions that include one or more Fc-antigen binding domain constructs described herein. In one embodiment, a pharmaceutical composition includes a substantially homogenous population of Fc-antigen binding domain constructs that are identical or substantially identical in structure. In various examples, the pharmaceutical composition includes a substantially homogenous population of any one of Fc-antigen binding domain constructs 1-42.

A therapeutic protein construct, e.g., an Fc-antigen binding domain construct described herein (e.g., an Fc-antigen binding domain construct having three Fc domains), of the present disclosure can be incorporated into a pharmaceutical composition. Pharmaceutical compositions including therapeutic proteins can be formulated by methods know to those skilled in the art. The pharmaceutical composition can be administered parenterally in the form of an injectable formulation including a sterile solution or suspension in water or another pharmaceutically acceptable liquid. For example, the pharmaceutical composition can be formulated by suitably combining the Fc-antigen binding domain construct with pharmaceutically acceptable vehicles or media, such as sterile water for injection (WFI), physiological saline, emulsifier, suspension agent, surfactant, stabilizer, diluent, binder, excipient, followed by mixing in a unit dose form required for generally accepted pharmaceutical practices. The amount of active ingredient included in the pharmaceutical preparations is such that a suitable dose within the designated range is provided.

The sterile composition for injection can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™ HCO-50, and the like commonly known in the art. Formulation methods for therapeutic protein products are known in the art, see e.g., Banga (ed.) Therapeutic Peptides and Proteins: Formulation, Processing and Delivery Systems (2d ed.) Taylor & Francis Group, CRC Press (2006).

### XIV. Methods of Treatment and Dosage

The Fc antigen binding domain constructs described here in can be used to treat a variety of cancers (e.g., hematologic malignancies and solid tumors) and autoimmune diseases.

The cancer can be one that is resistant to daratumumab or any other therapeutic anti-CD38 monoclonal antibody treatment. The cancer can be selected from: gastric cancer, breast cancer, colon cancer, lung cancer, mantle cell lymphoma, acute lymphoblastic leukemia, acute myeloid leukemia, NK cell leukemia, NK/T-cell lymphoma, chronic lymphocytic leukemia, plasma cell leukemia, and multiple myeloma. The constructs can also be used to treat: Amyloid light chain Amyloidosis, Castleman's disease, Monoclonal gammopathy of undetermined significance (MGUS), Biclonal gammopathy of undetermined significance, Heavy chain diseases, Solitary plasmacytome, Extramedullary plasmacytoma. In some cases, the constructs can be used to augment immunoregulatory functions against cancer cells by immune complex mediated induction of preventative and/or therapeutic vaccinal effects.

The constructs can also be used to treat: plasma cell dyscrasias or monoclonal gammopathies such as: Light chain deposition disease, Membranoproliferative Glomerulonephritis (MGRS), Autoimmune hemolytic anemia, Tempi Syndrome (Telangiectasia-Erythrocytosis-Monoclonal Gammopathy Perinephric-Fluid Collections-Intrapulmonary Shunting), Rheumatoid Arthritis, Lupus Erythematosus POEMS Syndrome (Polyneuropathy-Organomegaly-Endocrinopathy-Monoclonal plasmaproliferative disorder-Skin) and Waldenström Macroglobulinemia

The constructs can be used to treat autoantibody-mediated diseases such as: Myasthenia Gravis (MG), MuSK-MG, Myocarditis, Lambert Eaton, Myasthenic Syndrome, Neuromyotonia, Neuromyelitis optica, Narcolepsy, Acute motor axonal neuropathy, Guillain-Barré syndrome, Fisher Syndrome, Acute Sensory Ataxic Neuropathy, Paraneoplastic Stiff Person Syndrome, Chronic Neuropathy, Peripheral Neuropathy, Acute disseminated encephalomyelitis, Multiple sclerosis, Goodpasture Syndrome, Membranous Nephropathy, Glomerulonephritis, Pulmonary Alveolar Proteinosis, CIPD, Autoimmune hemolytic anemia, Autoimmune Thrombocytopenic purpura, Pemphigus vulgaris, Pemphigus foliaceus, Bullous pemphigoid, pemphigoid gestationis, Epidermolysis bullosa aquisita, Neonatal lupus erythematosus, Dermatitis herpetiformis, Graves Disease, Addison's Disease, Ovarian insufficiency, Autoimmune Orchitis, Sjogren's Disease, Autoimmune gastritis, Rheumatoid Arthritis, SLE, Dry eye disease, Vasulitis (Acute), Carditis, and Antibody-mediated rejection.

The pharmaceutical compositions are administered in a manner compatible with the dosage formulation and in such amount as is therapeutically effective to result in an improvement or remediation of the symptoms. The pharmaceutical compositions are administered in a variety of dosage forms, e.g., intravenous dosage forms, subcutaneous dosage forms, oral dosage forms such as ingestible solutions, drug release capsules, and the like. The appropriate dosage for the individual subject depends on the therapeutic objectives, the route of administration, and the condition of the patient. Generally, recombinant proteins are dosed at 1-200 mg/kg, e.g., 1-100 mg/kg, e.g., 20-100 mg/kg. Accordingly, it will be necessary for a healthcare provider to tailor and titer the dosage and modify the route of administration as required to obtain the optimal therapeutic effect.

In addition to treating humans, the constructs can be used to treat companion animals such as dogs and cats as well as other veterinary subjects.

### XV. Complement-Dependent Cytotoxicity (CDC)

Fc-antigen binding domain constructs described in this disclosure are able to activate various Fc receptor mediated effector functions. One component of the immune system is the complement-dependent cytotoxicity (CDC) system, a part of the innate immune system that enhances the ability of antibodies and phagocytic cells to clear foreign pathogens. Three biochemical pathways activate the complement system: the classical complement pathway, the alternative complement pathway, and the lectin pathway, all of which entail a set of complex activation and signaling cascades.

In the classical complement pathway, IgG or IgM trigger complement activation. The C1q protein binds to these antibodies after they have bound an antigen, forming the C1 complex. This complex generates C1s esterase, which cleaves and activates the C4 and C2 proteins into C4a and C4b, and C2a and C2b. The C2a and C4b fragments then form a protein complex called C3 convertase, which cleaves C3 into C3a and C3b, leading to a signal amplification and formation of the membrane attack complex.

The Fc-antigen binding domain constructs of this disclosure are able to enhance CDC activity by the immune system.

CDC may be evaluated by using a colorimetric assay in which Raji cells (ATCC) are coated with a serially diluted antibody, Fc-antigen binding domain construct, or IVIg. Human serum complement (Quidel) can be added to all wells at 25% v/v and incubated for 2 h at 37° C. Cells can be incubated for 12 h at 37° C. after addition of WST-1 cell proliferation reagent (Roche Applied Science). Plates can then be placed on a shaker for 2 min and absorbance at 450 nm can be measured.

### XVI. Antibody-Dependent Cell-Mediated Cytotoxicity (ADCC)

The Fc-antigen binding domain constructs of this disclosure are also able to enhance antibody-dependent cell-mediated cytotoxicity (ADCC) activity by the immune system. ADCC is a part of the adaptive immune system where antibodies bind surface antigens of foreign pathogens and target them for death. ADCC involves activation of natural killer (NK) cells by antibodies. NK cells express Fc receptors, which bind to Fc portions of antibodies such as IgG and IgM. When the antibodies are bound to the surface of a pathogen-infected target cell, they then subsequently bind the NK cells and activate them. The NK cells release cytokines such as IFN-y, and proteins such as perform and granzymes. Perform is a pore forming cytolysin that oligomerizes in the presence of calcium. Granzymes are serine proteases that induce programmed cell death in target cells. In addition to NK cells, macrophages, neutrophils and eosinophils can also mediate ADCC.

ADCC may be evaluated using a luminescence assay. Human primary NK effector cells (Hemacare) are thawed and rested overnight at 37° C. in lymphocyte growth medium-3 (Lonza) at 5×10⁵/mL. The next day, the human lymphoblastoid cell line Raji target cells (ATCC CCL-86) are harvested, resuspended in assay media (phenol red free RPMI, 10% FBSΔ, GlutaMAX™), and plated in the presence of various concentrations of each probe of interest for 30 minutes at 37° C. The rested NK cells are then harvested, resuspended in assay media, and added to the plates containing the anti-CD20 coated Raji cells. The plates are incubated at 37° C. for 6 hours with the final ratio of effector-to-target cells at 5:1 (5×10⁴ NK cells: 1×10⁴ Raji).

The CytoTox-Glo™ Cytotoxicity Assay kit (Promega) is used to determined ADCC activity. The CytoTox-Glo™ assay uses a luminogenic peptide substrate to measure dead cell protease activity which is released by cells that have lost membrane integrity e.g. lysed Raji cells. After the 6 hour incubation period, the prepared reagent (substrate) is added to each well of the plate and placed on an orbital plate shaker for 15 minutes at room temperature. Luminescence is measured using the PHERAstar F5 μlate reader (BMG Labtech). The data is analyzed after the readings from the control conditions (NK cells+Raji only) are subtracted from the test conditions to eliminate background.

### XVII. Antibody-Dependent Cellular Phagocytosis (ADCP)

The Fc-antigen binding domain constructs of this disclosure are also able to enhance antibody-dependent cellular phagocytosis (ADCP) activity by the immune system. ADCP, also known as antibody opsonization, is the process by which a pathogen is marked for ingestion and elimination by a phagocyte. Phagocytes are cells that protect the body by ingesting harmful foreign pathogens and dead or dying cells. The process is activated by pathogen-associated molecular patterns (PAMPS), which leads to NF-κB activation. Opsonins such as C3b and antibodies can then attach to target pathogens. When a target is coated in opsonin, the Fc domains attract phagocytes via their Fc receptors. The phagocytes then engulf the cells, and the phagosome of ingested material is fused with the lysosome. The subsequent phagolysosome then proteolytically digests the cellular material.

ADCP may be evaluated using a bioluminescence assay. Antibody-dependent cell-mediated phagocytosis (ADCP) is an important mechanism of action of therapeutic antibodies. ADCP can be mediated by monocytes, macrophages, neutrophils and dendritic cells via FcγRIIa (CD32a), FcγRI (CD64), and FcγRIIIa (CD16a). All three receptors can participate in antibody recognition, immune receptor clustering, and signaling events that result in ADCP; however, blocking studies suggest that FcγRIIa is the predominant Fcγ receptor involved in this process.

The FcγRIIa-H ADCP Reporter Bioassay is a bioluminescent cell-based assay that can be used to measure the potency and stability of antibodies and other biologics with Fc domains that specifically bind and activate FcγRIIa. The assay consists of a genetically engineered Jurkat T cell line that expresses the high-affinity human FcγRIIa-H variant that contains a Histidine (H) at amino acid 131 and a luciferase reporter driven by an NFAT-response element (NFAT-RE).

When co-cultured with a target cell and relevant antibody, the FcγRIIa-H effector cells bind the Fc domain of the antibody, resulting in FcγRIIa signaling and NFAT-RE-mediated luciferase activity. The bioluminescent signal is detected and quantified with a Luciferase assay and a standard luminometer.

### EXAMPLES

The following examples are put forth so as to provide those of ordinary skill in the art with a complete disclosure and description of how the methods and compounds claimed herein are performed, made, and evaluated, and are intended to be purely exemplary of the disclosure and are not intended to limit the scope of what the inventors regard as their disclosure.

### Example 1

### Design and Purification of Fc-Antigen Binding Domain Construct 7 with a CD38 Binding Domain

### Protein Expression

Fc-antigen binding domain constructs are designed to increase folding efficiencies, to minimize uncontrolled association of subunits, which may create unwanted high molecular weight oligomers and multimers, and to generate compositions for pharmaceutical use that are substantially homogenous (e.g., at least 85%, 90%, 95%, 98%, or 99% homogeneous). With these goals in mind, a construct formed from a singly branched Fc domain where the branch point is at the N-terminal Fc domain is made as described below. Fc-antigen binding domain construct 7 (CD38) each include two distinct Fc domain monomer containing polypeptides (two copies of an anti-CD38 long Fc chain (SEQ ID NO:ZZ1), and two copies of a short Fc chain (SEQ ID NO: ZZ2)), and two copies of an anti-CD38 light chain polypeptide (SEQ ID NO: ZZ3). The long Fc chain contains an Fc domain monomer with an E357K charge mutation and S354C and T366W protuberance-forming mutations (to promote heterodimerization) in a tandem series with a charge-mutated (K409D/D399K mutations) Fc domain monomer (to promote homodimerization), and anti-CD38 VH and CH1 domains (EU positions 1-220) at the N-terminus (construct 7 (CD38)). The short Fc chain contains an Fc domain monomer with a K370D charge mutation and Y349C, T366S, L368A, and Y407V cavity-forming mutations (to promote heterodimerization). The anti-CD38 light chain can also be expressed fused to the N-terminus of the long Fc chain as part of an scFv. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences in Table 7 are encoded by three separate plasmids (one plasmid encoding the light chain (anti-CD38), one plasmid encoding the long Fc chain (anti-CD38) and one plasmid encoding the short Fc chain).

TABLE 5
Construct 7 (CD38) sequences

| | | Long Fc chain | |
|---|---|---|---|
| | | (with anti-CD38 | |
| Construct | Light chain | VH and CH1) | Short Fc chain |
| Construct 7 | SEQ ID NO: 285 | SEQ ID NO: 286 | SEQ ID NO: 48 |
| (CD38) | EIVLTQSPATLSLSPGERATLS | QLLESGGGLVQPGGSLRL | DKTHTCPPCPAPELLGGPSVF |
| | CRASQSVSSYLAWYQQKPG | SCAASGFTFDDYGMSWV | LFPPKPKDTLMISRTPEVTCV |
| | QAPRLLIYDASNRATGIPARF | RQAPGKGLEWVSDISWN | VVDVSHEDPEVKFNWYVDG |
| | SGSGSGTDFTLTISSLEPEDFA | GGKTHYVDSVKGQFTISR | VEVHNAKTKPREEQYNSTYR |
| | VYYCQQRSNWPPTFGQGTK | DNSKNTLYLQMNSLRAED | VVSVLTVLHQDWLNGKEYK |
| | VEIKRTVAAPSVFIFPPSDEQL | TAVYYCARGSLFHDSSGFY | CKVSNKALPAPIEKTISKAKG |
| | KSGTASVVCLLNNFYPREAK | FGHWGQGTLVTVSSASTK | QPREPQVCTLPPSRDELTKN |
| | VQWKVDNALQSGNSQESVT | GPSVFPLAPSSKSTSGGTA | QVSLSCAVDGFYPSDIAVEW |
| | EQDSKDSTYSLSSTLTLSKAD | ALGCLVKDYFPEPVTVSW | ESNGQPENNYKTTPPVLDSD |
| | YEKHKVYACEVTHQGLSSPV | NSGALTSGVHTFPAVLQS | GSFFLVSKLTVDKSRWQQG |
| | TKSFNRGEC | SGLYSLSSVVTVPSSSLGT | NVFSCSVMHEALHNHYTQK |
| | | QTYICNVNHKPSNTKVDK | SLSLSPG |
| | | RVEPKSCDKTHTCPPCPA | |
| | | PELLGGPSVFLFPPKPKDT | |
| | | LMISRTPEVTCVVVDVSH | |
| | | EDPEVKFNWYVDGVEVH | |
| | | NAKTKPREEQYNSTYRVV | |
| | | SVLTVLHQDWLNGKEYKC | |
| | | KVSNKALPAPIEKTISKAKG | |
| | | QPREPQVYTLPPSRDELTK | |
| | | NQVSLTCLVKGFYPSDIAV | |
| | | EWESNGQPENNYKTTPP | |
| | | VLKSDGSFFLYSDLTVDKS | |
| | | RWQQGNVFSCSVMHEA | |
| | | LHNHYTQKSLSLSPGKGG | |
| | | GGGGGGGGGGGGGGG | |
| | | GGGDKTHTCPPCPAPELL | |
| | | GGPSVFLFPPKPKDTLMIS | |
| | | RTPEVTCVVVDVSHEDPE | |
| | | VKFNWYVDGVEVHNAKT | |
| | | KPREEQYNSTYRVVSVLTV | |
| | | LHQDWLNGKEYKCKVSN | |
| | | KALPAPIEKTISKAKGQPRE | |
| | | PQVYTLPPCRDKLTKNQV | |
| | | SLWCLVKGFYPSDIAVEW | |
| | | ESNGQPENNYKTTPPVLD | |
| | | SDGSFFLYSKLTVDKSRW | |
| | | QQGNVFSCSVMHEALHN | |
| | | HYTQKSLSLSPG | |

The expressed proteins are purified from the cell culture supernatant by Protein A-based affinity column chromatography, using a Poros MabCapture A (LifeTechnologies) column and then further fractionated by ion exchange chromatography. Purified sample are concentrated to approximately 30 mg/mL and sterile filtered through a 0.2 μm filter.

### Example 2

### Design and Purification of Fc-Antigen Binding Domain Construct 13 with a CD38 Binding Domain

### Protein Expression

A construct formed from a singly branched Fc domain where the branch point is at the C-terminal Fc domain is made as described below. Fc-antigen binding domain construct 13 (CD38) each include two distinct Fc domain monomer containing polypeptides (two copies of an anti-CD38 long Fc chain (any one of SEQ ID NOs: ZZ, and two copies of a short Fc chain (SEQ ID NO: ZZ)) and two copies of an anti-CD38 light chain polypeptide (SEQ ID NO: ZZ). The long Fc chain contains a charge-mutated (K409D/D399K mutations) Fc domain monomer (to promote homodimerization) in a tandem series with an Fc domain monomer with an E357K charge mutation and S354C and T366W protuberance-forming mutations (to promote heterodimerization), and anti-CD38 VH and CH1 domains (EU positions 1-220) at the N-terminus (construct 13 (CD38)). The short Fc chain contains an Fc domain monomer with a K370D charge mutation and Y349C, T366S, L368A, and Y407V cavity-forming mutations (to promote heterodimerization). The anti-CD38 light chain and the anti-CD38 VH and CH1 are taken from an ant-CD38 monoclonal antibody. Constructs with this light chain and anti-CD38 VH and CH1 are indicated by the abbreviation CD38. A related construct can be produced using the anti-CD38 light chain and the anti-CD38 VH and CH1 taken from a fully human monoclonal antibody that cross-reacts with CD38 expressed by cynomolgus monkeys. These constructs are indicated by the abbreviation Cyno. The CD38 light chain can also be expressed fused to the N-terminus of the long Fc chain as part of an scFv. Other versions of construct 13 can be made with the anti-CD38 heavy chain, wherein each version carries a different sized glycine spacer (G4 (SEQ ID NO: 19), G10 (SEQ ID NO: 25), G15 (SEQ ID NO: 26) or G20 (SEQ ID NO: 23) linkers) between the Fc domain monomers in the long Fc chain polypeptide. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for each of the following constructs are encoded by three separate plasmids (one plasmid encoding the light chain (anti-CD38), one plasmid encoding the long Fc chain (anti-CD38) and one plasmid encoding the short Fc chain):

TABLE 6
Construct 13 (CD38) sequences

| | | Long Fc chain | |
|---|---|---|---|
| Construct | Light chain | (anti-CD38 VH and CH1) | Short Fc chain |
| Construct 13 | SEQ ID NO: 285 | SEQ ID NO: 288 | SEQ ID NO: 48 |
| (CD38), G₂₀ | EIVLTQSPATLSLSPGERATLS | EVQLLESGGGLVQPGGSLRL | DKTHTCPPCPAPELLGGPSVF |
| (SEQ ID NO: 23) | CRASQSVSSYLAWYQQKPG | SCAVSGFTFNSFAMSWVRQ | LFPPKPKDTLMISRTPEVTCV |
| linker | QAPRLLIYDASNRATGIPARF | APGKGLEWVSAISGSGGGTY | VVDVSHEDPEVKFNWYVDG |
| S3Y-CD38 | SGSGSGTDFTLTISSLEPEDFA | YADSVKGRFTISRDNSKNTLY | VEVHNAKTKPREEQYNSTYR |
| | VYYCQQRSNWPPTFGQGTK | LQMNSLRAEDTAVYFCAKDK | VVSVLTVLHQDWLNGKEYK |
| | VEIKRTVAAPSVFIFPPSDEQL | ILWFGEPVFDYWGQGTLVT | CKVSNKALPAPIEKTISKAKG |
| | KSGTASVVCLLNNFYPREAK | VSSASTKGPSVFPLAPSSKSTS | QPREPQVCTLPPSRDELTKN |
| | VQWKVDNALQSGNSQESVT | GGTAALGCLVKDYFPEPVTV | QVSLSCAVDGFYPSDIAVEW |
| | EQDSKDSTYSLSSTLTLSKAD | SWNSGALTSGVHTFPAVLQS | ESNGQPENNYKTTPPVLDSD |
| | YEKHKVYACEVTHQGLSSPV | SGLYSLSSVVTVPSSSLGTQTY | GSFFLVSKLTVDKSRWQQG |
| | TKSFNRGEC | ICNVNHKPSNTKVDKRVEPK | NVFSCSVMHEALHNHYTQK |
| | | SCDKTHTCPPCPAPELLGGPS | SLSLSPG |
| | | VFLFPPKPKDTLMISRTPEVT | |
| | | CVVVDVSHEDPEVKFNWYV | |
| | | DGVEVHNAKTKPREEQYNST | |
| | | YRVVSVLTVLHQDWLNGKE | |
| | | YKCKVSNKALPAPIEKTISKAK | |
| | | GQPREPQVYTLPPCRDKLTK | |
| | | NQVSLWCLVKGFYPSDIAVE | |
| | | WESNGQPENNYKTTPPVLD | |
| | | SDGSFFLYSKLTVDKSRWQQ | |
| | | GNVFSCSVMHEALHNHYTQ | |
| | | KSLSLSPGKGGGGGGGGGG | |
| | | GGGGGGGGGGDKTHTCPP | |
| | | CPAPELLGGPSVFLFPPKPKD | |
| | | TLMISRTPEVTCVVVDVSHE | |
| | | DPEVKFNWYVDGVEVHNAK | |
| | | TKPREEQYNSTYRVVSVLTVL | |
| | | HQDWLNGKEYKCKVSNKAL | |
| | | PAPIEKTISKAKGQPREPQVY | |
| | | TLPPSRDELTKNQVSLICLVK | |
| | | GFYPSDIAVEWESNGQPEN | |
| | | NYKTTPPVLKSDGSFFLYSDL | |
| | | TVDKSRWQQGNVFSCSVM | |
| | | HEALHNHYTQKSLSLSPG | |
| Construct 13 | SEQ ID NO: 287 | SEQ ID NO: 289 | SEQ ID NO: 48 |
| (CD38), G₂₀ | SVLTQPPSASGTPGQRV | QLLESGGGLVQPGGSLRL | DKTHTCPPCPAPELLGGPS |
| (SEQ ID NO: 23) | TISCSGSSSNIGDNYVSWY | SCAASGFTFDDYGMSWV | VFLFPPKPKDTLMISRTPE |
| linker | QQLPGTAPKLLIYRDSQRP | RQAPGKGLEWVSDISWN | VTCVVVDVSHEDPEVKFN |
| S3Y-Cyno-001 | SGVPDRFSGSKSGTSASLA | GGKTHYVDSVKGQFTISR | WYVDGVEVHNAKTKPRE |
| | ISGLRSEDEADYYCQSYDS | DNSKNTLYLQMNSLRAED | EQYNSTYRVVSVLTVLHQ |
| | SLSGSVFGGGTKLTVLGQ | TAVYYCARGSLFHDSSGFY | DWLNGKEYKCKVSNKALP |
| | PKANPTVTLFPPSSEELQA | FGHWGQGTLVTVSSASTK | APIEKTISKAKGQPREPQV |
| | NKATLVCLISDFYPGAVTV | GPSVFPLAPSSKSTSGGTA | CTLPPSRDELTKNQVSLSC |
| | AWKADGSPVKAGVETTK | ALGCLVKDYFPEPVTVSW | AVDGFYPSDIAVEWESNG |
| | PSKQSNNKYAASSYLSLTP | NSGALTSGVHTFPAVLQS | QPENNYKTTPPVLDSDGS |
| | EQWKSHRSYSCQVTHEG | SGLYSLSSVVTVPSSSLGT | FFLVSKLTVDKSRWQQGN |
| | STVEKTVAPTECS | QTYICNVNHKPSNTKVDK | VFSCSVMHEALHNHYTQ |
| | | RVEPKSCDKTHTCPPCPA | KSLSLSPG |
| | | PELLGGPSVFLFPPKPKDT | |
| | | LMISRTPEVTCVVVDVSH | |
| | | EDPEVKFNWYVDGVEVH | |
| | | NAKTKPREEQYNSTYRVV | |
| | | SVLTVLHQDWLNGKEYKC | |
| | | KVSNKALPAPIEKTISKAKG | |
| | | QPREPQVYTLPPCRDKLTK | |
| | | NQVSLWCLVKGFYPSDIA | |
| | | VEWESNGQPENNYKTTP | |
| | | PVLDSDGSFFLYSKLTVDK | |
| | | SRWQQGNVFSCSVMHE | |
| | | ALHNHYTQKSLSLSPGKG | |
| | | GGGGGGGGGGGGGGG | |
| | | GGGGDKTHTCPPCPAPEL | |
| | | LGGPSVFLFPPKPKDTLMI | |
| | | SRTPEVTCVVVDVSHEDP | |
| | | EVKFNWYVDGVEVHNAK | |
| | | TKPREEQYNSTYRVVSVLT | |
| | | VLHQDWLNGKEYKCKVS | |
| | | NKALPAPIEKTISKAKGQP | |
| | | REPQVYTLPPSRDELTKN | |
| | | QVSLTCLVKGFYPSDIAVE | |
| | | WESNGQPENNYKTTPPV | |
| | | LKSDGSFFLYSDLTVDKSR | |
| | | WQQGNVFSCSVMHEAL | |
| | | HNHYTQKSLSLSPG | |

The expressed proteins were purified from the cell culture supernatant by Protein A-based affinity column chromatography, using a Poros MabCapture A (LifeTechnologies) column and then Purified sample were concentrated to approximately 30 mg/mL and sterile filtered through a 0.2 μm filter.

### Example 3

### Design and Purification of Fc-Antigen Binding Domain Construct 1

An unbranched construct formed from asymmetrical tandem Fc domains is made as described below. Fc-antigen binding domain construct 1 (FIG. 1) includes two distinct Fc domain monomer containing polypeptides (a long Fc chain and two copies of a short Fc chain) and a light chain polypeptide. The long Fc chain contains two Fc domain monomers in a tandem series, wherein each Fc domain monomer has an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K) (to promote heterodimerization), and a CD38 binding domain at the N-terminus. The CD38 binding domain may be expressed as part of the same amino acid sequence as the long Fc chain (e.g., to form a scFv). The short Fc chain contains an Fc domain monomer with an engineered cavity that is made by introducing at least one cavity-forming mutation selected from Table 3 (e.g., the Y349C, T366S, L368A, and Y407V mutations), and, optionally, a reverse charge mutation selected from Table 4A or 4B (e.g., K370D) (to promote heterodimerization). DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and the long Fc chains are encoded by two separate plasmids. In this Example, and in each of the following Examples for Fc-antigen binding domain constructs 2-42, the cell may contain a third plasmid expressing an antibody variable light chain.

The expressed proteins are purified from the cell culture supernatant by Protein A-based affinity column chromatography, using a Poros MabCapture A (LifeTechnologies) column. Captured Fc-antigen binding domain constructs are washed with phosphate buffered saline (low-salt wash) and eluted with 100mM glycine, pH 3. The eluate is quickly neutralized by the addition of 1 M TRIS pH 7.4 and sterile filtered through a 0.2 μm filter. The proteins are further fractionated by ion exchange chromatography using Poros XS resin (Applied Biosciences). The column is pre-equilibrated with 50 mM MES, pH 6 (buffer A), and the sample is eluted with a step gradient using 50 mM MES, 400 mM sodium chloride, pH 6 (buffer B) as the elution buffer. After ion exchange, the target fraction is buffer exchanged into PBS buffer using a 10 kDa cut-off polyether sulfone (PES) membrane cartridge on a tangential flow filtration system. The samples are concentrated to approximately 30 mg/mL and sterile filtered through a 0.2 μm filter.

Samples are denatured in Laemmli sample buffer (4% SDS, Bio-Rad) at 95° C. for 10 min. Samples are run on a Criterion TGX stain-free gel (4-15% polyacrylamide, Bio-Rad). Protein bands are visualized by UV illumination or Coommassie blue staining. Gels are imaged by ChemiDoc MP Imaging System (Bio-Rad). Quantification of bands is performed using Imagelab 4.0.1 software (Bio-Rad).

### Example 4

### Design and Purification of Fc-Antigen Binding Domain Construct 2

An unbranched construct formed from asymmetrical tandem Fc domains is made as described below. Fc-antigen binding domain construct 2 (FIG. 2) includes two distinct Fc monomer containing polypeptides (a long Fc chain and three copies of a short Fc chain) and a light chain polypeptide. The long Fc chain contains three Fc domain monomers in a tandem series with a CD38 binding domain at N-terminus, wherein each Fc domain monomer has an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K). The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D). DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 5

### Design and Purification of Fc-Antigen Binding Domain Construct 3

A construct formed from asymmetrical tandem Fc domains is made as described below. Fc-antigen binding domain construct 3 (FIG. 3) includes two distinct Fc monomer containing polypeptides (a long Fc chain and two copies of a short Fc chain) and a light chain polypeptide. The long Fc chain contains two Fc domain monomers in a tandem series, wherein each Fc domain monomer has an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K). The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D), and a CD38 binding domain at N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 6

### Design and Purification of Fc-Antigen Binding Domain Construct 4

A construct formed from asymmetrical tandem Fc domains is made as described below. Fc-antigen binding domain construct 4 (FIG. 4) includes two distinct Fc monomer containing polypeptides (a long Fc chain and three copies of a short Fc chain) and a light chain polypeptide. The long Fc chain contains three Fc domain monomers in a tandem series, wherein each Fc domain monomer has an engineered protuberance that is made by introducing at least one protuberance-forming mutation selected from Table 3 (e.g., the S354C and T366W mutations) and, optionally, one or more reverse charge mutations selected from Table 4A or 4B (e.g., E357K). The short Fc chain contains an Fc domain monomer with an engineered cavity that is made by introducing at least one cavity-forming mutation selected from Table 3 (e.g., the Y349C, T366S, L368A, and Y407V mutations), and, optionally, a reverse charge mutation selected from Table 4A or 4B (e.g., K370D), and a CD38 binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 7

### Design and Purification of Fc-Antigen Binding Domain Construct 5

A construct formed from asymmetrical tandem Fc domains is made as described below. Fc-antigen binding domain construct 5 (FIG. 5) includes two distinct Fc monomer containing polypeptides (a long Fc chain and two copies of a short Fc chain) and a light chain polypeptide. The long Fc chain contains two Fc domain monomers in a tandem series with a CD38 binding domain at the N-terminus, wherein each Fc domain monomer has an engineered protuberance that is made by introducing at least one protuberance-forming mutation selected from Table 3 (e.g., the S354C and T366W mutations) and, optionally, one or more reverse charge mutations selected from Table 4A or 4B (e.g., E357K). The short Fc chain contains an Fc domain monomer with an engineered cavity that is made by introducing at least one cavity-forming mutation selected from Table 3 (e.g., the Y349C, T366S, L368A, and Y407V mutations), and, optionally, a reverse charge mutation selected from Table 4A or 4B (e.g., K370D), and a CD38 binding domain at N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 8

### Design and Purification of Fc-Antigen Binding Domain Construct 6

A construct formed from asymmetrical tandem Fc domains is made as described below. Fc-antigen binding domain construct 6 (FIG. 6) includes two distinct Fc monomer containing polypeptides (a long Fc chain and three copies of a short Fc chain) and a light chain polypeptide. The long Fc chain contains three Fc domain monomers in a tandem series with a CD38 binding domain at the N-terminus, wherein each Fc domain monomer has an engineered protuberance that is made by introducing at least one protuberance-forming mutation selected from Table 3 (e.g., the S354C and T366W mutations) and, optionally, one or more reverse charge mutations selected from Table 4A or 4B (e.g., E357K). The short Fc chain contains an Fc domain monomer with an engineered cavity that is made by introducing at least one cavity-forming mutation selected from Table 3 (e.g., the Y349C, T366S, L368A, and Y407V mutations), and, optionally, a reverse charge mutation selected from Table 4A or 4B (e.g., K370D), and a CD38 binding domain at N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 9

### Design and Purification of Fc-Antigen Binding Domain Construct 7

A construct formed from a singly branched Fc domain where the branch point is at the N-terminal Fc domain is made as described below. Fc-antigen binding domain construct 7 (FIG. 7) includes two distinct Fc monomer containing polypeptides (two copies of a long Fc chain and two copies of a short Fc chain) and a light chain polypeptide. The long Fc chain contains an Fc domain monomer with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), in a tandem series with an Fc domain monomer with reverse charge mutations selected from Table 4A or 4B (e.g., the K409D/D399K mutations), and a CD38 binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D). DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 10

### Design and Purification of Fc-Antigen Binding Domain Construct 8

A construct formed from a singly branched Fc domain where the branch point is at the N-terminal Fc domain is made as described below. Fc-antigen binding domain construct 8 (FIG. 8) includes two distinct Fc monomer containing polypeptides (two copies of a long Fc chain and two copies of a short Fc chain) and a light chain polypeptide. The long Fc chain contains an Fc domain monomer with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), in a tandem series with an Fc domain monomer with reverse charge mutations selected from Table 4A or 4B (e.g., the K409D/D399K mutations). The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D), and a CD38 binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 11

### Design and Purification of Fc-Antigen Binding Domain Construct 9

A construct formed from a singly branched Fc domain where the branch point is at the N-terminal Fc domain is made as described below. Fc-antigen binding domain construct 9 (FIG. 9) includes two distinct Fc monomer containing polypeptides (two copies of a long Fc chain and two copies of a short Fc chain) and a light chain polypeptide. The long Fc chain contains an Fc domain monomer with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), in a tandem series with an Fc domain monomer with reverse charge mutations selected from Table 4A or 4B (e.g., the K409D/D399K mutations), and a CD38 binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D), and a CD38 binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 12

### Design and Purification of Fc-Antigen Binding Domain Construct 10

A construct formed from a singly branched Fc domain where the branch point is at the N-terminal Fc domain is made as described below. Fc-antigen binding domain construct 10 (FIG. 10) includes two distinct Fc monomer containing polypeptides (two copies of a long Fc chain and four copies of a short Fc chain) and a light chain polypeptide. The long Fc chain contains two Fc domain monomers in a tandem series, wherein each Fc domain monomer has an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), in a tandem series with an Fc domain monomer with reverse charge mutations selected from Table 4A or 4B (e.g., the K409D/D399K mutations), and a CD38 binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D). DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 13

### Design and Purification of Fc-Antigen Binding Domain Construct 11

A construct formed from a singly branched Fc domain where the branch point is at the N-terminal Fc domain is made as described below. Fc-antigen binding domain construct 11 (FIG. 11) includes two distinct Fc monomer containing polypeptides (two copies of a long Fc chain and four copies of a short Fc chain) and a light chain polypeptide. The long Fc chain contains two Fc domain monomers in a tandem series, wherein each Fc domain monomer has an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), in a tandem series with an Fc domain monomer with reverse charge mutations selected from Table 4A or 4B (e.g., the K409D/D399K mutations) at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D), and an antigen-binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 14

### Design and Purification of Fc-Antigen Binding Domain Construct 12

A construct formed from a singly branched Fc domain where the branch point is at the N-terminal Fc domain is made as described below. Fc-antigen binding domain construct 12 (FIG. 12) includes two distinct Fc monomer containing polypeptides (two copies of a long Fc chain and four copies of a short Fc chain) and a light chain polypeptide. The long Fc chain contains two Fc domain monomers in a tandem series, wherein each Fc domain monomer has an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), in a tandem series with an Fc domain monomer with reverse charge mutations selected from Table 4A or 4B (e.g., the K409D/D399K mutations), and a CD38 binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D), and an antigen-binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 15

### Design and Purification of Fc-Antigen Binding Domain Construct 13

A construct formed from a singly branched Fc domain where the branch point is at the C-terminal Fc domain is made as described below. Fc-antigen binding domain construct 13 (FIG. 13) includes two distinct Fc monomer containing polypeptides (two copies of a long Fc chain and two copies of a short Fc chain) and a light chain polypeptide. The long Fc chain contains an Fc domain monomer with reverse charge mutations selected from Table 4A or 4B (e.g., the K409D/D399K mutations), in a tandem series with an Fc domain monomer with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), and a CD38 binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D). DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 16

### Design and Purification of Fc-Antigen Binding Domain Construct 14

A construct formed from a singly branched Fc domain where the branch point is at the C-terminal Fc domain is made as described below. Fc-antigen binding domain construct 14 (FIG. 14) includes two distinct Fc monomer containing polypeptides (two copies of a long Fc chain and two copies of a short Fc chain) and a light chain polypeptide. The long Fc chain contains an Fc domain monomer with reverse charge mutations selected from Table 4A or 4B (e.g., the K409D/D399K mutations), in a tandem series with an Fc domain monomer with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K) at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D), and a CD38 binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 17

### Design and Purification of Fc-Antigen Binding Domain Construct 15

A construct formed from a singly branched Fc domain where the branch point is at the C-terminal Fc domain is made as described below. Fc-antigen binding domain construct 15 (FIG. 15) includes two distinct Fc monomer containing polypeptides (two copies of a long Fc chain and two copies of a short Fc chain) and a light chain polypeptide. The long Fc chain contains an Fc domain monomer with reverse charge mutations selected from Table 4A or 4B (e.g., the K409D/D399K mutations), in a tandem series with an Fc domain monomer with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), and a CD38 binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D), and a CD38 binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 18

### Design and Purification of Fc-Antigen Binding Domain Construct 16

A construct formed from a singly branched Fc domain where the branch point is at the C-terminal Fc domain is made as described below. Fc-antigen binding domain construct 16 (FIG. 16) includes two distinct Fc monomer containing polypeptides (two copies of a long Fc chain and four copies of a short Fc chain) and a light chain polypeptide. The long Fc chain contains an Fc domain monomer with reverse charge mutations selected from Table 4A or 4B (e.g., the K409D/D399K mutations), in a tandem series with two Fc domain monomers, each with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), and a CD38 binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D). DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 19

### Design and Purification of Fc-Antigen Binding Domain Construct 17

A construct formed from a singly branched Fc domain where the branch point is at the C-terminal Fc domain is made as described below. Fc-antigen binding domain construct 17 (FIG. 17) includes two distinct Fc monomer containing polypeptides (two copies of a long Fc chain and four copies of a short Fc chain) and a light chain polypeptide. The long Fc chain contains an Fc domain monomer with reverse charge mutations selected from Table 4A or 4B (e.g., the K409D/D399K mutations), in a tandem series with two Fc domain monomers, each with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D), andCD38 binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 20

### Design and Purification of Fc-Antigen Binding Domain Construct 18

A construct formed from a singly branched Fc domain where the branch point is at the C-terminal Fc domain is made as described below. Fc-antigen binding domain construct 18 (FIG. 18) includes two distinct Fc monomer containing polypeptides (two copies of a long Fc chain and four copies of a short Fc chain) and a light chain polypeptide. The long Fc chain contains an Fc domain monomer with reverse charge mutations selected from Table 4A or 4B (e.g., the K409D/D399K mutations), in a tandem series with two Fc domain monomers, each with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), and a CD38 binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D), and a CD38 binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 21

### Design and Purification of Fc-Antigen Binding Domain Construct 19

A construct formed from a singly branched Fc domain where the branch point is neither at the N- or C-terminal Fc domain is made as described below. Fc-antigen binding domain construct 19 (FIG. 19) includes two distinct Fc monomer containing polypeptides (two copies of a long Fc chain and four copies of a short Fc chain) and a light chain polypeptide. The long Fc chain contains an Fc domain monomer with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), in a tandem series with an Fc domain monomer with reverse charge mutations selected from Table 4A or 4B (e.g., the K409D/D399K mutations), and another Fc domain monomer with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), and a CD38 binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D). DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 22

### Design and Purification of Fc-Antigen Binding Domain Construct 20

A construct formed from a singly branched Fc domain where the branch point is at the C-terminal Fc domain is made as described below. Fc-antigen binding domain construct 20 (FIG. 20) includes two distinct Fc monomer containing polypeptides (two copies of a long Fc chain and four copies of a short Fc chain) and a light chain polypeptide. The long Fc chain contains an Fc domain monomer with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), in a tandem series with an Fc domain monomer with reverse charge mutations selected from Table 4A or 4B (e.g., the K409D/D399K mutations), and another Fc domain monomer with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D), and a CD38 binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 23

### Design and Purification of Fc-Antigen Binding Domain Construct 21

A construct formed from a singly branched Fc domain where the branch point is at the C-terminal Fc domain is made as described below. Fc-antigen binding domain construct 21 (FIG. 21) includes two distinct Fc monomer containing polypeptides (two copies of a long Fc chain and four copies of a short Fc chain) and a light chain polypeptide. The long Fc chain contains an Fc domain monomer with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), in a tandem series with an Fc domain monomer with reverse charge mutations selected from Table 4A or 4B (e.g., the K409D/D399K mutations), another Fc domain monomer with an engineered protuberance that is made by introducing at least one protuberance-forming mutation selected from Table 3 (e.g., the S354C and T366W mutations) and, optionally, one or more reverse charge mutation selected from Table 4A or 4B (e.g., E357K), and a CD38 binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4A or 4B (e.g., K370D), and a CD38 binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 24

### CDC, ADCP, and ADCC Activation by Fc-Antigen Binding Domain Constructs

Three assays are used to test the activation of CDC, ADCP, and ADCC pathways by parent mAbs and various Fc-antigen binding domain constructs. Four constructs are created containing the CDRs from Gazyva (obinutuzumab), an anti-CD20 mAb. Both fucosylated and afucosylated anti-CD20 mAbs were made as well as S3Y-AA-CD20 (structure of Construct 13, FIG. 13, as described in Example 2) and SAI-AA-CD20 (structure of Construct 7, FIG. 7, as described in Example 1) Fc-antigen binding domain constructs.

A CDC assay is performed as follows:

1. The target cells used in the anti-CD20 CDC assay are the Raji cells (ATCC CCL-86). Raji cells (CD20 expressing tumor cells) were resuspended in X-VIVO 15 media at 6×10⁵ cells/ml. Cells were then transferred to a 96 well flat-bottom assay plate in a volume of 100 μl per well (6×10⁴ cells/well).

2. Anti-CD20 mAbs and Fc-antigen binding domain constructs were diluted to 3.33 μM in X-VIVO 15 media. Serial 1:3 dilutions were then performed with each molecule in 1.5 ml polypropylene tubes resulting in an 11 point dilution series.

3. Each dilution of the molecules were transferred at 50 μl/well to the appropriate wells in the assay plate. Immediately following the transfer to assay plate, 50 μl of normal human serum complement were added to each well.

4. The assay plate was incubated at 37° C. and 5% CO₂ for 2 h. Following the 2 h incubation, 20 μl of WST-1 proliferation reagent was added to each well of the assay plate. The plate was returned to the 37° C., 5% CO₂ incubator for 14 h.

5. Following the 14 h incubation, the plate was shaken for 1 min on a plate shaker and the absorbance of the wells was immediately determined at 450 nm with 600 nm correction using a spectrophotometer.

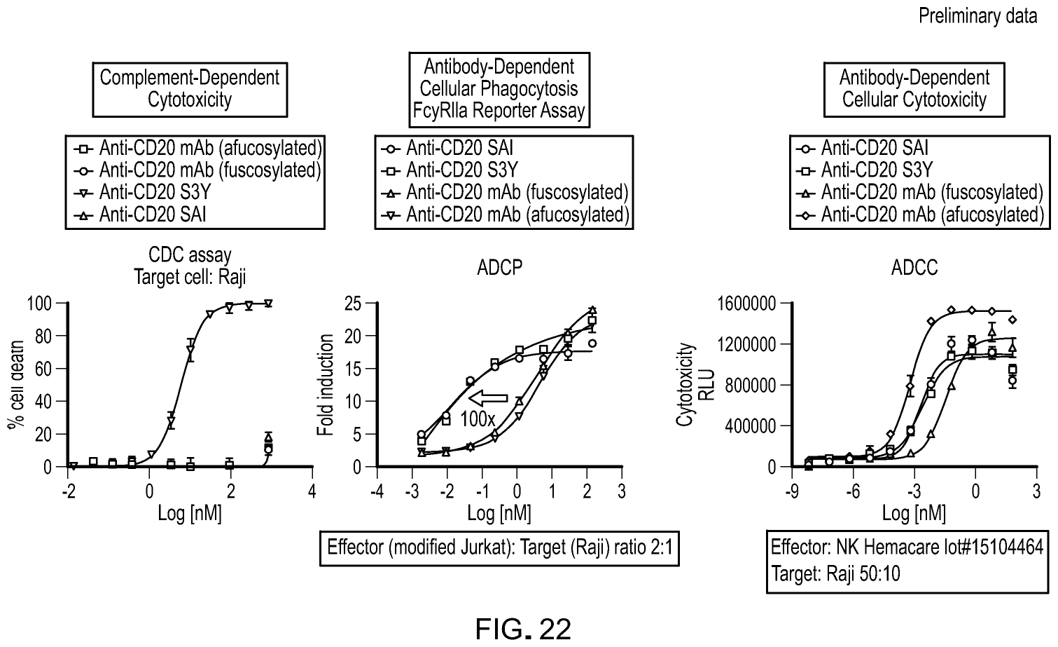

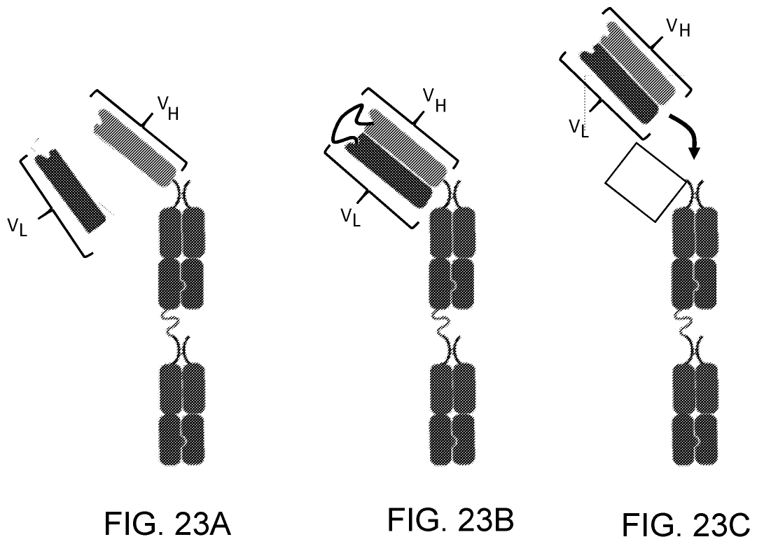

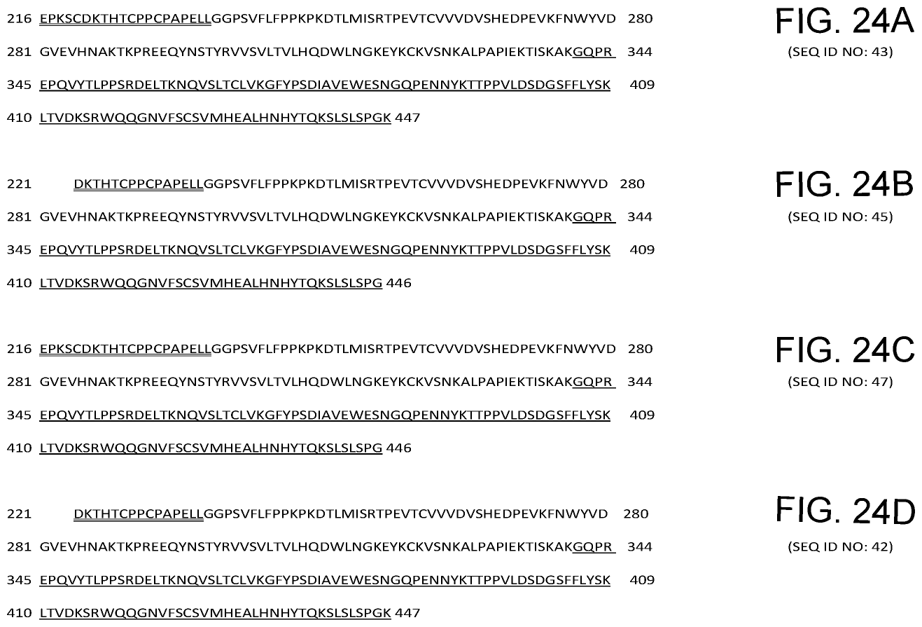

In a CDC assay in which the target cells were Raji (FIG. 22, left panel), the S3Y-AA-CD20 (construct 13 with anti-CD20 Fab) was able to mediate cytotoxicity, while the other constructs were not.

An ADCP assay was performed as follows:

The FcγRIIa-H ADCP Reporter Bioassay, Complete Kit (Promega Cat # G9901), is a bioluminescent cell-based assay that can be used to measure the potency and stability of antibodies and other biologics with Fc domains that specifically bind and activate FcγRIIa. The assay consisted of a genetically engineered Jurkat T cell line that expresses the high-affinity human FcγRIIa-H variant that contains a Histidine (H) at amino acid 131 and a luciferase reporter driven by an NFAT-response element (NFAT-RE). When co-cultured with a target cell and relevant antibody, the FcγRIIa-H effector cells upon binding to Fc domain of an antibody results in FcγRIIa signaling and NFAT-RE-mediated luciferase activity. The bioluminescent signal was detected and quantified using Bio-Glo™ Luciferase Assay System and a luminometer. Increasing concentrations of anti-CD20 mAbs and construct 7 (with an anti-CD20 Fab) or construct 13 (with an anti-CD20 Fab) were incubated with Raji target cells and FcγRIIa-H effector cells (in 2:1 ratio). After 6 hours of incubation at 37° C. Bio-Glo™ reagent was added, and luminescence was measured in a PHERAstar FS instrument. Data was fitted to a 4PL curve using GraphPad Prism software (FIG. 22, middle panel). Both the S31-AA-CD20 (construct 7 with anti-CD20 Fab) and S3Y-AA-CD20 (construct 13 with anti-CD20 Fab) constructs showed enhanced potency (EC50) >100-fold relative to the anti-CD20 mAbs.

An ADCC assay was performed as follows:

Human primary NK effector cells were thawed and rested overnight at 37° C. in lymphocyte growth medium-3 (Lonza) at 5×10⁵/mL. The next day, the Raji cells were harvested, resuspended in assay media (phenol red free RPMI, 10% FBS, GlutaMAX™), and plated in the presence of various concentrations of each molecule of interest for 30 minutes at 37° C. The rested NK cells were then harvested, resuspended in assay media, and added to the plates containing the anti-CD20 coated Raji cells. The plates were incubated at 37° C. for 6 hours with the final ratio of effector-to-target cells at 5:1 (5×10⁴ NK: 1×10⁴ Raji cells).

The CytoTox-Glo™ Cytotoxicity Assay kit (Promega) was used to determined ADCC activity. The CytoTox-Glo™ assay uses a luminogenic peptide substrate to measure dead cell protease activity which is released by cells that have lost membrane integrity e.g. lysed Raji cells. After the 6 hour incubation period, the prepared reagent (substrate) was added to each well of the plate and placed on an orbital plate shaker for 15 minutes at room temperature. Luminescence was measured using the PHERAstar F5 plate reader (BMG Labtech). The data was analyzed after the readings from the control conditions (NK cells+Raji only) were subtracted from the test conditions to eliminate background. (FIG. 47, right panel). Both the S31 (construct 7 with anti-CD20 Fab) and S3Y (construct 13 with anti-CD20 Fab) constructs showed enhanced cytotoxicity relative to the fucosylated mAb and similar cytotoxicity relative to the afucosylated mAb.

### Example 25

### Experimental Assays Used to Characterize Fc-Antigen Binding Domain Constructs

### Peptide and Glycopeptide Liquid Chromatography-MS/MS

The proteins were diluted to 1 μg/μL in 6M guanidine (Sigma). Dithiothreitol (DTT) was added to a concentration of 10 mM, to reduce the disulfide bonds under denaturing conditions at 65° C. for 30 min. After cooling on ice, the samples were incubated with 30 mM iodoacetamide (IAM) for 1 h in the dark to alkylate (carbamidomethylate) the free thiols. The protein was then dialyzed across a 10-kDa membrane into 25 mM ammonium bicarbonate buffer (pH 7.8) to remove IAM, DTT and guanidine. The protein was digested with trypsin in a Barocycler (NEP 2320; Pressure Biosciences, Inc.). The pressure was cycled between 20,000 psi and ambient pressure at 37° C. for a total of 30 cycles in 1 h. LC-MS/MS analysis of the peptides was performed on an Ultimate 3000 (Dionex) Chromatography System and an Q-Exactive (Thermo Fisher Scientific) Mass Spectrometer. Peptides were separated on a BEH PepMap (Waters) Column using 0.1% FA in water and 0.1% FA in acetonitrile as the mobile phases. The singly xylosylated linker peptide was targeted based on the doubly charged ion (z=2) m/z 842.5 with a quadrupole isolation width of ±1.5 Da.

### Intact Mass Spectrometry

The protein was diluted to a concentration of 2 μg/μL in the running buffer consisting of 78.98% water, 20% acetonitrile, 1% formic acid (FA), and 0.02% trifluoroacetic acid. Size exclusion chromatography separation was performed on two Zenix-C SEC-300 (Sepax Technologies, Newark, DE) 2.1×350 mm in tandem for a total length column length of 700 mm. The proteins were eluted from the SEC column using the running buffer described above at a flow rate of 80 μL/min. Mass spectra were acquired on an QSTAR Elite (Applied Biosystems) Q-ToF mass spectrometer operated in positive mode. The neutral masses under the individual size fractions were deconvoluted using Bayesian peak deconvolution by summing the spectra across the entire width of the chromatographic peak.

### Capillary Electrophoresis-Sodium Dodecyl Sulfate (CE-SDS) Assay

Samples were diluted to 1 mg/mL and mixed with the HT Protein Express denaturing buffer (PerkinElmer). The mixture was incubated at 40° C. for 20 min. Samples were diluted with 70 μL of water and transferred to a 96-well plate. Samples were analyzed by a Caliper GXII instrument (PerkinElmer) equipped with the HT Protein Express LabChip (PerkinElmer). Fluorescence intensity was used to calculate the relative abundance of each size variant.

### Non-Reducing SDS-PAGE

Samples were denatured in Laemmli sample buffer (4% SDS, Bio-Rad) at 95° C. for 10 min. Samples were run on a Criterion TGX stain-free gel (4-15% polyacrylamide, Bio-Rad). Protein bands were visualized by UV illumination or Coommassie blue staining. Gels were imaged by ChemiDoc MP Imaging System (Bio-Rad). Quantification of bands was performed using Imagelab 4.0.1 software (Bio-Rad).

### Complement Dependent Cytotoxicity (CDC)

CDC was evaluated as described before in Example 24.

### Example 26

### Design and Purification of Fc-Antigen Binding Domain Construct 4 with CD38 Binding Domain

### Protein Expression

A construct formed from asymmetrical tandem Fc domains was made as described below. Fc-antigen binding domain construct 4 (CD38) each includes two distinct Fc domain monomer containing polypeptides (a long Fc chain (SEQ ID NO: 66), and three copies an anti-CD38 Fc chain (SEQ ID NO: 68)) and three copies of an anti-CD38 light chain polypeptide (SEQ ID NO: 49). The long Fc chain contains three Fc domain monomers in a tandem series, wherein each Fc domain monomer has an E357K charge mutation and S354C and T366W protuberance-forming mutations (to promote heterodimerization). The short Fc chain contains an Fc domain monomer with a K370D charge mutation and Y349C, T366S, L368A, and Y407V cavity-forming mutations (to promote heterodimerization), and anti-CD38 VH and CH1 domains (EU positions 1-220) at the N-terminus (construct 4 (CD38)). The CD38 light chain can also be expressed fused to the N-terminus of the short Fc chain as part of an scFv. DNA sequences are optimized for expression in mammalian cells and cloned into the pcDNA3.4 mammalian expression vector. The DNA plasmid constructs are transfected via liposomes into human embryonic kidney (HEK) 293 cells. The following amino acid sequences for each construct in Table 7 are encoded by three separate plasmids (one plasmid encoding the light chain (anti-CD38), one plasmid encoding the long Fc chain and one plasmid encoding the short Fc chain (anti-CD38)):

TABLE 7
Construct 4 (CD38) sequences

| | | Long Fc chain | |
|---|---|---|---|
| | | (with anti-CD38 VH | |
| Construct | Light chain | and CH1) | Short Fc chain |
| Construct 4 | SEQ ID NO: 285 | SEQ ID NO: 290 | SEQ ID NO: 291 |
| S3L | EIVLTQSPATLSLSPGERATLS | DKTHTCPPCPAPELLGGPSVF | EVQLLESGGGLVQPGGS |
| (CD38) | CRASQSVSSYLAWYQQKPG | LFPPKPKDTLMISRTPEVTCV | LRLSCAVSGFTFNSFAMS |
| | QAPRLLIYDASNRATGIPARF | VVDVSHEDPEVKFNWYVDG | WVRQAPGKGLEVVVSAIS |
| | SGSGSGTDFTLTISSLEPEDFA | VEVHNAKTKPREEQYNSTYR | GSGGGTYYADSVKGRFTI |
| | VYYCQQRSNWPPTFGQGTK | VVSVLTVLHCIDWLNGKEYK | SRDNSKNTLYLQMNSLRA |
| | VEIKRTVAAPSVFIFPPSDEQL | CKVSNKALPAPEKTISKAKG | EDTAVYFCAKDKILWFGE |
| | KSGTASVVCLLNNFYPREAK | QPREPQVYTLPPCRDKLTKN | PVFDYWGQGTLVTVSSA |
| | VQWKVDNALQSGNSQESVT | QVSLWCLVKGFYPSDIAVEW | STKGPSVFPLAPSSKSTS |
| | EQDSKDSTYSLSSTLTLSKAD | ESNGQPENNYKTTPPVLDSD | GGTAALGCLVKDYFPEPV |
| | YEKHKVYACEVTHQGLSSPV | GSFFLYSKLTVDKSRWQQGN | TVSWNSGALTSGVHTFPA |
| | TKSFNRGEC | VFSCSVMHEALHNHYTQKSL | VLQSSGLYSLSSVVTVPS |
| | | SLSPGKGGGGGGGGGGGG | SSLGTQTYICNVNHKPSN |
| | | GGGGGGGGDKTHTCPPCPA | TKVDKRVEPKSCDKTHTC |
| | | PELLGGPSVFLFPPKPKDTLM | PPCPAPELLGGPSVFLFPPKP |
| | | ISRTPEVTCVVVDVSHEDPEV | KDTLMISRTPEVTCVVVDVS |
| | | KFNWYVDGVEVHNAKTKPR | HEDPEVKFNWYVDGVEVHN |
| | | EEQYNSTYRVVSVLTVLHQD | AKTKPREEQYNSTYRVVSVLT |
| | | WLNGKEYKCKVSNKALPAPI | VLHQDWLNGKEYKCKVSNK |
| | | EKTISKAKGQPREPQVYTLPP | ALPAPIEKTISKAKGQPREPQ |
| | | CRDKLTKNQVSLWCLVKGFY | VCTLPPSRDELTKNQVSLSCA |
| | | PSDIAVEWESNGQPENNYKT | VDGFYPSDIAVEWESNGQPE |
| | | TPPVLDSDGSFFLYSKLTVDK | NNYKTTPPVLDSDGSFFLVSK |
| | | SRWQQGNVFSCSVMHEAL | LTVDKSRWQQGNVFSCSVM |
| | | HNHYTQKSLSLSPGKGGGG | HEALHNHYTQKSLSLSPG |
| | | GGGGGGGGGGGGGGGGD | |
| | | KTHTCPPCPAPELLGGPSVFL | |
| | | FPPKPKDTLMISRTPEVTCVV | |
| | | VDVSHEDPEVKFNWYVDGV | |
| | | EVHNAKTKPREEQYNSTYRV | |
| | | VSVLTVLHQDWLNGKEYKCK | |
| | | VSNKALPAPIEKTISKAKGQP | |
| | | REPQVYTLPPCRDKLTKNQV | |
| | | SLWCLVKGFYPSDIAVEWES | |
| | | NGQPENNYKTTPPVLDSDGS | |
| | | FFLYSKLTVDKSRWQQGNVF | |
| | | SCSVMHEALHNHYTQKSLSL | |
| | | SPG | |

The expressed proteins are purified from the cell culture supernatant by Protein A-based affinity column chromatography, using a Poros MabCapture A (Life Technologies) column. Captured Fc-antigen binding domain constructs are washed with phosphate buffered saline (low-salt wash) and eluted with 100 mM glycine, pH 3. The eluate was quickly neutralized by the addition of 1 M TRIS pH 7.4 and sterile filtered through a 0.2 μm filter. The proteins are further fractionated by ion exchange chromatography using Poros XS resin (Applied Biosciences). The column was pre-equilibrated with 50 mM MES, pH 6 (buffer A), and the sample was eluted with a step gradient using 50 mM MES, 400 mM sodium chloride, pH 6 (buffer B) as the elution buffer. After ion exchange, the target fraction was buffer exchanged into PBS buffer using a 10 kDa cut-off polyether sulfone (PES) membrane cartridge on a tangential flow filtration system. The samples are concentrated to approximately 30 mg/mL and sterile filtered through a 0.2 μm filter.

### Example 27

### Design and Purification of Fc-Antigen Binding Domain Construct 8 with a CD38 Binding Domain

### Protein Expression

A construct formed from a singly branched Fc domain where the branch point is at the N-terminal Fc domain was made as described below. Fc-antigen binding domain construct 8 (CD38) each include two distinct Fc domain monomer containing polypeptides (two copies of a long Fc chain (SEQ ID NO: 69), and two copies of an anti-CD38 short Fc chain (SEQ ID NO: 68)) and copies of an anti-CD38 light chain polypeptide (SEQ ID NO: 49). The long Fc chain contains an Fc domain monomer with an E357K charge mutation and S354C and T366W protuberance-forming mutations (to promote heterodimerization) in a tandem series with an Fc domain monomer with reverse charge mutations K409D and D399K (to promote homodimerization). The short Fc chain contains an Fc domain monomer with a K370D charge mutation and Y349C, T366S, L368A, and Y407V cavity-forming mutations (to promote heterodimerization), and anti-CD38 VH and CH1 domains (EU positions 1-220) at the N-terminus (construct 8 (CD38)). The CD38 light chain can also be expressed fused to the N-terminus of the short Fc chain as part of an scFv. DNA sequences are optimized for expression in mammalian cells and cloned into the pcDNA3.4 mammalian expression vector. The DNA plasmid constructs are transfected via liposomes into human embryonic kidney (HEK) 293 cells. The following amino acid sequences for each construct in Table 8 are encoded by three separate plasmids (one plasmid encoding the light chain (anti-CD38), one plasmid encoding the long Fc chain and one plasmid encoding the short Fc chain (anti-CD38)):

TABLE 8
Construct 8 (CD38) sequences

| | | Long Fc chain | |
|---|---|---|---|
| | | (with anti-CD38 VH | |
| Construct | Light chain | and CH1) | Short Fc chain |
| Construct 8 | SEQ ID NO: 285 | SEQ ID NO: 292 | SEQ ID NO: 291 |
| (CD38) | EIVLTQSPATLSLSPGERATLS | DKTHTCPPCPAPELLGGPSVF | EVQLLESGGGLVQPGGS |
| | CRASQSVSSYLAWYQQKPG | LFPPKPKDTLMISRTPEVTCV | LRLSCAVSGFTFNSFAMS |
| | QAPRLLIYDASNRATGIPARF | VVDVSHEDPEVKFNWYVDG | WVRQAPGKGLEWVSAIS |
| | SGSGSGTDFTLTISSLEPEDFA | VEVHNAKTKPREEQYNSTYR | GSGGGTYYADSVKGRFTI |
| | VYYCQQRSNWPPTFGQGTK | VVSVLTVLHQDWLNGKEYK | SRDNSKNTLYLQMNSLRA |
| | VEIKRTVAAPSVFIFPPSDEQL | CKVSNKALPAPIEKTISKAKG | EDTAVYFCAKDKILWFGE |
| | KSGTASVVCLLNNFYPREAK | QPREPQVYTLPPSRDELTKN | PVFDYWGQGTLVTVSSA |
| | VQWKVDNALQSGNSQESVT | QVSLTCLVKGFYPSDIAVEW | STKGPSVFPLAPSSKSTS |
| | EQDSKDSTYSLSSTLTLSKAD | ESNGQPENNYKTTPPVLKSD | GGTAALGCLVKDYFPEPV |
| | YEKHKVYACEVTHQGLSSPV | GSFFLYSDLTVDKSRWQQG | TVSVVNSGALTSGVHTFPA |
| | TKSFNRGEC | NVFSCSVMHEALHNHYTQK | VLQSSGLYSLSSVVTVPS |
| | | SLSLSPGKGGGGGGGGGGG | SSLGTQTYICNVNHKPSN |
| | | GGGGGGGGGDKTHTCPPCP | TKVDKRVEPKSCDKTHTC |
| | | APELLGGPSVFLFPPKPKDTL | PPCPAPELLGGPSVFLFPPKP |
| | | MISRTPEVTCVVVDVSHEDP | KDTLMISRTPEVTCVVVDVS |
| | | EVKFNWYVDGVEVHNAKTK | HEDPEVKFNWYVDGVEVHN |
| | | PREEQYNSTYRVVSVLTVLH | AKTKPREEQYNSTYRVVSVLT |
| | | QDWLNGKEYKCKVSNKALP | VLHQDWLNGKEYKCKVSNK |
| | | APIEKTISKAKGQPREPQVYT | ALPAPIEKTISKAKGQPREPQ |
| | | LPPCRDKLTKNQVSLWCLVK | VCTLPPSRDELTKNQVSLSCA |
| | | GFYPSDIAVEWESNGQPEN | VDGFYPSDIAVEWESNGQPE |
| | | NYKTTPPVLDSDGSFFLYSKL | NNYKTTPPVLDSDGSFFLVSK |
| | | TVDKSRWQQGNVFSCSVM | LTVDKSRWQQGNVFSCSVM |
| | | HEALHNHYTQKSLSLSPG | HEALHNHYTQKSLSLSPG |

The expressed proteins are purified from the cell culture supernatant by Protein A-based affinity column chromatography, using a Poros MabCapture A (LifeTechnologies) column. Captured Fc-antigen binding domain constructs are washed with phosphate buffered saline (low-salt wash) and eluted with 100 mM glycine, pH 3. The eluate is quickly neutralized by the addition of 1 M TRIS pH 7.4 and sterile filtered through a 0.2 μm filter. The proteins are further fractionated by ion exchange chromatography using Poros XS resin (Applied Biosciences). The column is pre-equilibrated with 50 mM MES, pH 6 (buffer A), and the sample is eluted with a step gradient using 50 mM MES, 400 mM sodium chloride, pH 6 (buffer B) as the elution buffer. After ion exchange, the target fraction is buffer exchanged into PBS buffer using a 10 kDa cut-off polyether sulfone (PES) membrane cartridge on a tangential flow filtration system. The samples are concentrated to approximately 30 mg/mL and sterile filtered through a 0.2 μm filter.

### Example 28

### Design and Purification of Fc-Antigen Binding Domain Construct 9 with CD38 Binding Domain Protein Expression

A construct formed from a singly branched Fc domain where the branch point is at the N-terminal Fc domain was made as described below. Fc-antigen binding domain construct 9 (CD38) include two distinct Fc domain monomer containing polypeptides (two copies an anti-CD38 long Fc chain (SEQ ID NO: 54), and two copies of an anti-CD38 short Fc chain (SEQ ID NO: 68)) and copies of an anti-CD38 light chain polypeptide (SEQ ID NO: 49). The long Fc chain contains an Fc domain monomer with an E357K charge mutation and S354C and T366W protuberance-forming mutations (to promote heterodimerization) in a tandem series with an Fc domain monomer with reverse charge mutations K409D and D399K (to promote homodimerization), and anti-CD38 VH and CH1 domains (EU positions 1-220) at the N-terminus (construct 9 (CD38)). The short Fc chain contains an Fc domain monomer with a K370D charge mutation and Y349C, T366S, L368A, and Y407V cavity-forming mutations (to promote heterodimerization), and an anti-CD38 heavy chain at the N-terminus (construct 9 (CD38)). The CD38 light chain can also be expressed fused to the N-terminus of the long Fc chain and/or short Fc chain as part of an scFv. DNA sequences were optimized for expression in mammalian cells and cloned into the pcDNA3.4 mammalian expression vector. The DNA plasmid constructs were transfected via liposomes into human embryonic kidney (HEK) 293 cells. The following amino acid sequences for each construct in Table 9 were encoded by three separate plasmids (one plasmid encoding the light chain (anti-CD38), one plasmid encoding the long Fc chain (anti-CD38) and one plasmid encoding the short Fc chain (anti-CD38)):

TABLE 9
Construct 9 (CD38) sequences

| | | Long Fc chain | Short Fc chain |
|---|---|---|---|
| | | (with anti-CD38 VH | (with anti-CD38 VH and |
| Construct | Light chain | and CH1) | CH1) |
| Construct 9 | SEQ ID NO: 285 | SEQ ID NO: 293 | SEQ ID NO: 291 |
| S3A | EIVLTQSPATLSLSPGERATLS | EVQLLESGGGLVQPGGSL | EVQLLESGGGLVQPGGSLRL |
| (CD38) | CRASQSVSSYLAWYQQKPG | RLSCAVSGFTFNSFAMSW | SCAVSGFTFNSFAMSWVRQ |
| | QAPRLLIYDASNRATGIPARF | VRQAPGKGLEWVSAISGS | APGKGLEWVSAISGSGGGTY |
| | SGSGSGTDFTLTISSLEPEDFA | GGGTYYADSVKGRFTISR | YADSVKGRFTISRDNSKNTLY |
| | VYYCQQRSNWPPTFGQGTK | DNSKNTLYLQMNSLRAED | LQMNSLRAEDTAVYFCAKDK |
| | VEIKRTVAAPSVFIFPPSDEQL | TAVYFCAKDKILWFGEPVF | ILWFGEPVFDYWGQGTLVT |
| | KSGTASVVCLLNNFYPREAK | DYWGQGTLVTVSSASTKG | VSSASTKGPSVFPLAPSSKSTS |
| | VQWKVDNALQSGNSQESVT | PSVFPLAPSSKSTSGGTAA | GGTAALGCLVKDYFPEPVTV |
| | EQDSKDSTYSLSSTLTLSKAD | LGCLVKDYFPEPVTVSWN | SWNSGALTSGVHTFPAVLQS |
| | YEKHKVYACEVTHQGLSSPV | SGALTSGVHTFPAVLQSS | SGLYSLSSVVTVPSSSLGTQTY |
| | TKSFNRGEC | GLYSLSSVVTVPSSSLGTQ | ICNVNHKPSNTKVDKRVEPK |
| | | TYICNVNHKPSNTKVDKR | SCDKTHTCPPCPAPELLGGPS |
| | | VEPKSCDKTHTCPPCPAPE | VFLEPPKPKDILMISRTPEVT |
| | | LLGGPSVFLFPPKPKDTLM | CVVVDVSHEDPEVKFNWYV |
| | | ISRTPEVTCVVVDVSHEDP | DGVEVHNAKTKPREEQYNST |
| | | EVKFNWYVDGVEVHNAK | YRVVSVLTVLHQDWLNGKE |
| | | TKPREEQYNSTYRVVSVLT | YKCKVSNKALPAPIEKTISKAK |
| | | VLHQDWLNGKEYKCKVS | GQPREPQVCTLPPSRDELTK |
| | | NKALPAPIEKTISKAKGQP | NQVSLSCAVDGFYPSDIAVE |
| | | REPQVYTLPPSRDELTKN | WESNGQPENNYKTTPPVLD |
| | | QVSLTCLVKGFYPSDIAVE | SDGSFFLVSKLTVDKSRWQQ |
| | | WESNGQPENNYKTTPPV | GNVFSCSVMHEALHNHYTQ |
| | | LKSDGSFFLYSDLTVDKSR | KSLSLSPG |
| | | WQQGNVFSCSVMHIAL | |
| | | HNHYTQKSLSLSPGKGGG | |
| | | GGGGGGGGGGGGGGG | |
| | | GGDKTHTCPPCPAPELLG | |
| | | GPSVFLFPPKPKDTLMISR | |
| | | TPEVTCVVVDVSHEDPEV | |
| | | KFNWYVDGVEVHNAKTK | |
| | | PREEQYNSTYRVVSVLTVL | |
| | | HQDWLNGKEYKCKVSNK | |
| | | ALPAPIEKTISKAKGQPREP | |
| | | QVYTLPPCRDKLTKNQVS | |
| | | LWCLVKGFYPSDIAVEWE | |
| | | SNGQPENNYKTTPPVLDS | |
| | | DGSFFLYSKLTVDKSRWQ | |
| | | QGNVFSCSVMHEALHNH | |
| | | YTQKSLSLSPG | |

### Example 29

### Design and Purification of Fc-Antigen Binding Domain Construct 10 with CD38 Binding Domain

### Protein Expression

A construct formed from a singly branched Fc domain where the branch point is at the N-terminal Fc domain is made as described below. Fc-antigen binding domain construct 10 (CD38) each include two distinct Fc domain monomer containing polypeptides (two copies of an anti-CD38 long fc chain (SEQ ID NO: 71), and four copies of a short Fc chain (SEQ ID NO: 63)) and copies of an anti-CD38 light chain polypeptide (SEQ ID NO: 49), respectively. The long Fc chain contains two Fc domain monomers in a tandem series, wherein each Fc domain monomer has an E357K charge mutation and S354C and T366W protuberance-forming mutations (to promote heterodimerization), in tandem series with an Fc domain monomer with reverse charge mutations K409D and D399K (to promote homodimerization), and anti-CD38 VH and CH1 domains (EU positions 1-220) at the N-terminus (construct 10 (CD38)). The short Fc chain contains an Fc domain monomer with a K370D charge mutation and Y349C, T366S, L368A, and Y407V cavity-forming mutations (to promote heterodimerization). The anti-CD38 light chain can also be expressed fused to the N-terminus of the long Fc chain as part of an scFv. DNA sequences were optimized for expression in mammalian cells and cloned into the pcDNA3.4 mammalian expression vector. The DNA plasmid constructs were transfected via liposomes into human embryonic kidney (HEK) 293 cells. The following amino acid sequences for each construct in Table 10 were encoded by three separate plasmids (one plasmid encoding the light chain (anti-CD38), one plasmid encoding the long Fc chain (anti-CD38) and one plasmid encoding the short Fc chain:

TABLE 10
Construct 10 (CD38) sequences

| | | Long Fc chain | |
|---|---|---|---|
| Construct | Light chain | (anti-CD38 VH and CH1) | Short Fc chain |
| Construct 10 | SEQ ID NO: 285 | SEQ ID NO: 71 | SEQ ID NO: 63 |
| (CD38) | EIVLTQSPATLSLSPGERATLS | EVQLLESGGGLVQPGGSLRL | DKTHTCPPCPAPELLGGPS |
| | CRASQSVSSYLAWYQQKPG | SCAVSGFTFNSFAMSWVRQ | VFLFPPKPKDTLMISRTPE |
| | QAPRLLIYDASNRATGIPARF | APGKGLEWVSAISGSGGGTY | VTCVVVDVSHEDPEVKFN |
| | SGSGSGTDFTLTISSLEPEDFA | YADSVKGRFTISRDNSKNTLY | WYVDGVEVHNAKTKPRE |
| | VYYCQQRSNWPPTFGQGTK | LQMNSLRAEDTAVYFCAKDK | EQYNSTYRVVSVLTVLHQ |
| | VEIKRTVAAPSVFIFPPSDEQL | ILWFGEPVFDYWGQGTLVT | DWLNGKEYKCKVSNKALP |
| | KSGTASVVCLLNNFYPREAK | VSSASTKGPSVFPLAPSSKSTS | APIEKTISKAKGQPREPQV |
| | VQWKVDNALQSGNSQESVT | GGTAALGCLVKDYFPEPVTV | CTLPPSRDELTKNQVSLSC |
| | EQDSKDSTYSLSSTLTLSKAD | SWNSGALTSGVHTFPAVLQS | AVDGFYPSDIAVEWESNG |
| | YEKHKVYACEVTHQGLSSPV | SGLYSLSSVVTVPSSSLGTQTY | QPENNYKTTPPVLDSDGS |
| | TKSFNRGEC | ICNVNHKPSNTKVDKRVEPK | FFLVSKLTVDKSRWQQGN |
| | | SCDKTHTCPPCPAPELLGG | VFSCSVMHEALHNHYTQ |
| | | PSVFLFPPKPKDTLMISRT | KSLSLSPG |
| | | PEVTCVVVDVSHEDPEVK | |
| | | FNWYVDGVEVHNAKTKP | |
| | | REEQYNSTYRVVSVLTVLH | |
| | | QDWLNGKEYKCKVSNKA | |
| | | LPAPIEKTISKAKGQPREP | |
| | | QVYTLPPSRDELTKNQVSL | |
| | | TCLVKGFYPSDIAVEWES | |
| | | NGQPENNYKTTPPVLKSD | |
| | | GSFFLYSDLTVDKSRWQQ | |
| | | GNVFSCSVMHEALHNHY | |
| | | TQKSLSLSPGKGGGGGGG | |
| | | GGGGGGGGGGGGGDKT | |
| | | HTCPPCPAPELLGGPSVFL | |
| | | FPPKPKDTLMISRTPEVTC | |
| | | VVVDVSHEDPEVKFNWY | |
| | | VDGVEVHNAKTKPREEQY | |
| | | NSTYRVVSVLTVLHQDWL | |
| | | NGKEYKCKVSNKALPAPIE | |
| | | KTISKAKGQPREPQVYTLP | |
| | | PCRDKLTKNQVSLWCLVK | |
| | | GFYPSDIAVEWESNGQPE | |
| | | NNYKTTPPVLDSDGSFFLY | |
| | | SKLTVDKSRWQQGNVFS | |
| | | CSVMHEALHNHYTQKSLS | |
| | | LSPGKGGGGGGGGGGG | |
| | | GGGGGGGGGDKTHTCPP | |
| | | CPAPELLGGPSVFLFPPKP | |
| | | KDTLMISRTPEVTCVVVD | |
| | | VSHEDPEVKFNWYVDGV | |
| | | EVHNAKTKPREEQYNSTY | |
| | | RVVSVLTVLHQDWLNGK | |
| | | EYKCKVSNKALPAPIEKTIS | |
| | | KAKGQPREPQVYTLPPCR | |
| | | DKLTKNQVSLWCLVKGFY | |
| | | PSDIAVEWESNGQPENNY | |
| | | KTTPPVLDSDGSFFLYSKLT | |
| | | VDKSRWQQGNVFSCSV | |
| | | MHEALHNHYTQKSLSLSP | |
| | | G | |

The expressed proteins were purified from the cell culture supernatant by Protein A-based affinity column chromatography, using a Poros MabCapture A (LifeTechnologies) column. Captured Fc-antigen binding domain constructs were washed with phosphate buffered saline (low-salt wash) and eluted with 100 mM glycine, pH 3. The eluate is quickly neutralized by the addition of 1 M TRIS pH 7.4 and sterile filtered through a 0.2 μm filter. The proteins are further fractionated by ion exchange chromatography using Poros XS resin (Applied Biosciences). The column is pre-equilibrated with 50 mM MES, pH 6 (buffer A), and the sample is eluted with a step gradient using 50 mM MES, 400 mM sodium chloride, pH 6 (buffer B) as the elution buffer. After ion exchange, the target fraction is buffer exchanged into PBS buffer using a 10 kDa cut-off polyether sulfone (PES) membrane cartridge on a tangential flow filtration system. The samples are concentrated to approximately 30 mg/mL and sterile filtered through a 0.2 μm filter.

### Example 30

### Design and Purification of Fc-Antigen Binding Domain Construct 16 with a CD38 Binding Domain Protein Expression

A construct formed from a singly branched Fc domain where the branch point is at the C-terminal Fc domain is made as described below. Fc-antigen binding domain construct 16 (CD38) each includes two distinct Fc domain monomer containing polypeptides (two copies of an anti-CD38 long Fc chain (SEQ ID NO: 73), and four copies of a short Fc chain (SEQ ID NO: 63)) and three copies of an anti-CD38 light chain polypeptide (SEQ ID NO: 49), respectively. The long Fc chain contains an Fc domain monomer with reverse charge mutations K409D and D399K (to promote homodimerization) in a tandem series with two Fc domain monomers, in tandem, that each have an E357K charge mutation and S354C and T366W protuberance-forming mutations (to promote heterodimerization), and anti-CD38 VH and CH1 domains (EU positions 1-220) at the N-terminus (construct 10 (CD38)). The short Fc chain contains an Fc domain monomer with a K370D charge mutation and Y349C, T366S, L368A, and Y407V cavity-forming mutations (to promote heterodimerization). The anti-CD38 light chain can also be expressed fused to the N-terminus of the long Fc chain as part of an scFv. DNA sequences are optimized for expression in mammalian cells and cloned into the pcDNA3.4 mammalian expression vector. The DNA plasmid constructs are transfected via liposomes into human embryonic kidney (HEK) 293 cells. The following amino acid sequences for each construct in Table 11 are encoded by three separate plasmids (one plasmid encoding the light chain (anti-CD38), one plasmid encoding the long Fc chain (anti-CD38) and one plasmid encoding the short Fc chain:

TABLE 11
Construct 16 (CD38) sequences

| | | Long Fc chain | |
|---|---|---|---|
| | | (with anti-CD38 VH | |
| Construct | Light chain | and CH1) | Short Fc chain |
| Construct 16 | SEQ ID NO: 285 | SEQ ID NO: 73 | SEQ ID NO: 63 |
| S5Y | EIVLTQSPATLSLSPGERATLS | EVQLLESGGGLVQPGGSLRL | DKTHTCPPCPAPELLGGPS |
| (CD38) | CRASQSVSSYLAWYQQKPG | SCAVSGFTFNSFAMSWVRQ | VFLFPPKPKDTLMISRTPE |
| | QAPRLLIYDASNRATGIPARF | APGKGLEWVSAISGSGGGTY | VTCVVVDVSHEDPEVKFN |
| | SGSGSGTDFTLTISSLEPEDFA | YADSVKGRFTISRDNSKNTLY | WYVDGVEVHNAKTKPRE |
| | VYYCQQRSNWPPTFGQGTK | LQMNSLRAEDTAVYFCAKDK | EQYNSTYRVVSVLTVLHQ |
| | VEIKRTVAAPSVFIFPPSDEQL | ILWFGEPVFDYWGQGTLVT | DWLNGKEYKCKVSNKALP |
| | KSGTASVVCLLNNFYPREAK | VSSASTKGPSVFPLAPSSKSTS | APIEKTISKAKGQPREPQV |
| | VQWKVDNALQSGNSQESVT | GGTAALGCLVKDYFPEPVTV | CTLPPSRDELTKNQVSLSC |
| | EQDSKDSTYSLSSTLTLSKAD | SWNSGALTSGVHTFPAVLQS | AVDGFYPSDIAVEWESNG |
| | YEKHKVYACEVTHQGLSSPV | SGLYSLSSVVTVPSSSLGTQTY | QPENNYKTTPPVLDSDGS |
| | TKSFNRGEC | ICNVNHKPSNTKVDKRVEPK | FFLVSKLTVDKSRWQQGN |
| | | SCDKTHTCPPCPAPELLGG | VFSCSVMHEALHNHYTQ |
| | | PSVFLFPPKPKDTLMISRT | KSLSLSPG |
| | | PEVTCVVVDVSHEDPEVK | |
| | | FNWYVDGVEVHNAKTKP | |
| | | REEQYNSTYRVVSVLTVLH | |
| | | QDWLNGKEYKCKVSNKA | |
| | | LPAPIEKTISKAKGQPREP | |
| | | QVYTLPPCRDKLTKNQVS | |
| | | LWCLVKGFYPSDIAVEWE | |
| | | SNGQPENNYKTTPPVLDS | |
| | | DGSFFLYSKLTVDKSRWQ | |
| | | QGNVFSCSVMHEALHNH | |
| | | YTQKSLSLSPGKGGGGGG | |
| | | GGGGGGGGGGGGGGDK | |
| | | THTCPPCPAPELLGGPSVF | |
| | | LFPPKPKDTLMISRTPEVT | |
| | | CVVVDVSHEDPEVKFNW | |
| | | YVDGVEVHNAKTKPREEQ | |
| | | YNSTYRVVSVLTVLHQDW | |
| | | LNGKEYKCKVSNKALPAPI | |
| | | EKTISKAKGQPREPQVYTL | |
| | | PPCRDKLTKNQVSLWCLV | |
| | | KGFYPSDIAVEWESNGQP | |
| | | ENNYKTTPPVLDSDGSFFL | |
| | | YSKLTVDKSRWQQGNVF | |
| | | SCSVMHEALHNHYTQKSL | |
| | | SLSPGKGGGGGGGGGGG | |
| | | GGGGGGGGGDKTHTCPP | |
| | | CPAPELLGGPSVFLFPPKP | |
| | | KDTLMISRTPEVTCVVVD | |
| | | VSHEDPEVKFNWYVDGV | |
| | | EVHNAKTKPREEQYNSTY | |
| | | RVVSVLTVLHQDWLNGK | |
| | | EYKCKVSNKALPAPIEKTIS | |
| | | KAKGQPREPQVYTLPPSR | |
| | | DELTKNQVSLTCLVKGFYP | |
| | | SDIAVEWESNGQPENNYK | |
| | | TTPPVLKSDGSFFLYSDLTV | |
| | | DKSRWQQGNVFSCSVM | |
| | | HEALHNHYTQKSLSLSPG | |

The expressed proteins are purified from the cell culture supernatant by Protein A-based affinity column chromatography, using a Poros MabCapture A (LifeTechnologies) column. Captured Fc-antigen binding domain constructs are washed with phosphate buffered saline (low-salt wash) and eluted with 100 mM glycine, pH 3. The eluate is quickly neutralized by the addition of 1 M TRIS pH 7.4 and sterile filtered through a 0.2 μm filter. The proteins are further fractionated by ion exchange chromatography using Poros XS resin (Applied Biosciences). The column is pre-equilibrated with 50 mM MES, pH 6 (buffer A), and the sample is eluted with a step gradient using 50 mM MES, 400 mM sodium chloride, pH 6 (buffer B) as the elution buffer. After ion exchange, the target fraction is buffer exchanged into PBS buffer using a 10 kDa cut-off polyether sulfone (PES) membrane cartridge on a tangential flow filtration system. The samples are concentrated to approximately 30 mg/mL and sterile filtered through a 0.2 μm filter.

### Example 31

### Design and Purification of Fc-Antigen Binding Domain Construct 19 with a CD38 Binding Domain

### Protein Expression

A construct formed from a singly branched Fc domain where the branch point is at neither the N-terminal or C-terminal Fc domain was made as described below. Fc-antigen binding domain construct 19 (CD38) includes two distinct Fc domain monomer containing polypeptides (two copies of an anti-CD38 long Fc chain (SEQ ID NO: 75), and four copies of a short Fc chain (SEQ ID NO: 63)) and copies of an anti-CD38 light chain polypeptide (SEQ ID NO: 49), respectively. The long Fc chain contains an Fc domain monomer with an E357K charge mutation and S354C and T366W protuberance-forming mutations (to promote heterodimerization), in a tandem series with an Fc domain monomer with reverse charge mutations K409D and D399K (to promote homodimerization), in a tandem series with an Fc domain monomer with an E357K charge mutation and S354C and T366W protuberance-forming mutations (to promote heterodimerization), and anti-CD38 VH and CH1 domains (EU positions 1-220) at the N-terminus (construct 19 (CD38)) . The short Fc chain contains an Fc domain monomer with a K370D charge mutation and Y349C, T366S, L368A, and Y407V cavity-forming mutations (to promote heterodimerization). The anti-CD38 light chain can also be expressed fused to the N-terminus of the long Fc chain as part of an scFv. DNA sequences were optimized for expression in mammalian cells and cloned into the pcDNA3.4 mammalian expression vector. The DNA plasmid constructs were transfected via liposomes into human embryonic kidney (HEK) 293 cells. The following amino acid sequences for each construct in Table 12 were encoded by three separate plasmids (one plasmid encoding the light chain (anti-CD38), one plasmid encoding the long Fc chain (anti-CD38) and one plasmid encoding the short Fc chain:

TABLE 12
Construct 19 (CD38) sequences

| | | Long Fc chain | Short Fc chain |
|---|---|---|---|
| | | (with anti-CD38 VH | |
| Construct | Light chain | and CH1) | |
| Construct 19 | SEQ ID NO: 285 | SEQ ID NO: 294 | SEQ ID NO: 63 |
| S5X | EIVLTQSPATLSLSPGERATLS | EVQLLESGGGLVQPGGSLRL | DKTHTCPPCPAPELLGGPSVF |
| (CD38) | CRASQSVSSYLAWYQQKPG | SCAVSGFTFNSFAMSWVRQ | LFPPKPKDTLMISRTPEVTCV |
| | QAPRLLIYDASNRATGIPARF | APGKGLEWVSAISGSGGGTY | VVDVSHEDPEVKFNWYVDG |
| | SGSGSGTDFTLTISSLEPEDFA | YADSVKGRFTISRDNSKNTLY | VEVHNAKTKPREEQYNSTYR |
| | VYYCQQRSNWPPTFGQGTK | LQMNSLRAEDTAVYFCAKDK | VVSVLTVLHQDWLNGKEYK |
| | VEIKRTVAAPSVFIFPPSDEQL | ILWFGEPVFDYWGQGTLVT | CKVSNKALPAPIEKTISKAKG |
| | KSGTASVVCLLNNFYPREAK | VSSASTKGPSVFPLAPSSKSTS | QPREPQVCTLPPSRDELTKN |
| | VQWKVDNALQSGNSQESVT | GGTAALGCLVKDYFPEPVTV | QVSLSCAVDGFYPSDIAVEW |
| | EQDSKDSTYSLSSTLTLSKAD | SWNSGALTSGVHTFPAVLQS | ESNGQPENNYKTTPPVLDSD |
| | YEKHKVYACEVTHQGLSSPV | SGLYSLSSVVTVPSSSLGTQTY | GSFFLVSKLTVDKSRWQQG |
| | TKSFNRGEC | ICNVNHKPSNTKVDKRVEPK | NVFSCSVMHEALHNHYTQK |
| | | SCDKTHTCPPCPAPELLGGPS | SLSLSPG |
| | | VFLFPPKPKDTLMISRTPEVT | |
| | | CVVVDVSHEDPEVKFNWYV | |
| | | DGVEVHNAKTKPREEQYNST | |
| | | YRVVSVLTVLHQDWLNGKE | |
| | | YKCKVSNKALPAPIEKTISKAK | |
| | | GQPREPQVYTLPPCRDKLTK | |
| | | NQVSLWCLVKGFYPSDIAVE | |
| | | WESNGQPENNYKTTPPVLD | |
| | | SDGSFFLYSKLTVDKSRWQQ | |
| | | GNVFSCSVMHEALHNHYTQ | |
| | | KSLSLSPGKGGGGGGGGGG | |
| | | GGGGGGGGGGDKTHTCPP | |
| | | CPAPELLGGPSVFLFPPKPKD | |
| | | TLMISRTPEVTCVVVDVSHE | |
| | | DPEVKFNWYVDGVEVHNAK | |
| | | TKPREEQYNSTYRVVSVLTVL | |
| | | HQDWLNGKEYKCKVSNKAL | |
| | | PAPIEKTISKAKGQPREPQVY | |
| | | TLPPSRDELTKNQVSLTCLVK | |
| | | GFYPSDIAVEWESNGQPEN | |
| | | NYKTTPPVLKSDGSFFLYSDL | |
| | | TVDKSRWQQGNVFSCSVM | |
| | | HEALHNHYTQKSLSLSPGKG | |
| | | GGGGGGGGGGGGGGGGG | |
| | | GGDKTHTCPPCPAPELLGGP | |
| | | SVFLFPPKPKDTLMISRTPEV | |
| | | TCVVVDVSHEDPEVKFNWY | |
| | | VDGVEVHNAKTKPREEQYN | |
| | | STYRVVSVLTVLHQDWLNGK | |
| | | EYKCKVSNKALPAPIEKTISKA | |
| | | KGQPREPQVYTLPPCRDKLT | |
| | | KNQVSLWCLVKGFYPSDIAV | |
| | | EWESNGQPENNYKTTPPVL | |
| | | DSDGSFFLYSKLTVDKSRWQ | |
| | | QGNVFSCSVMHEALHNHYT | |
| | | QKSLSLSPG | |

### Example 32

### Binding of Anti-CD38 Constructs to Human Tumor Cell Lines and Stable Cell Lines Expressing Human and Cynomolgus Monkey CD38

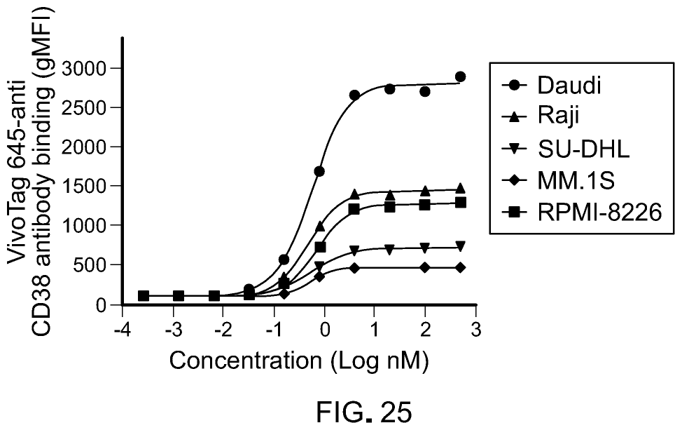

Tumor cell suspension in media containing 10% FBS was incubated with increasing concentrations of VivoTag645-labeled anti-CD38 antibody at 4° C. for 1 hour. Cells were then washed in cold buffer and suspended in FACS buffer. Labeled cell suspensions were then read on APC channel on BD FACS Verse flow cytometer. Live cell population were gated using unlabeled cells. Geometric mean fluorescence intensity (gMFI) values were calculated from the gated population using FlowJo software. The results of this analysis are presented in FIG. 25.

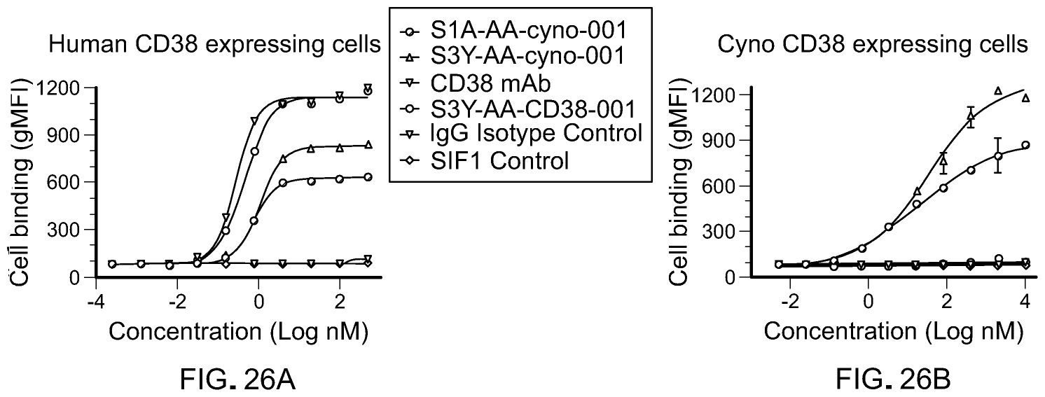

Raji cells were used to evaluate dose-dependent relative binding of parental IgG1 anti-CD38 antibody and the corresponding anti-CD38 constructs. Since the anti-CD38 mAb (that was the source of the Fabs for the various anti-CD38 Fc constructs) does not cross react with monkey CD38, we generated a surrogate anti-CD38 human monoclonal IgG1 antibody that reacts with the cynomolgus monkey CD38 (S1A-AA-Cyno CD38) and a surrogate anti-CD38 construct 13 using the same Fab sequences, that reacts with cynomolgus monkey CD38 (S3Y-AA-Cyno CD38); this was used for evaluating CDC activity in the presence of cynomolgus monkey serum complement and pharmacodynamic response of targeting endogenous cynomolgus monkey CD38 in non-human primate whole blood. The results of these binding studies are presented in FIG. 26.

### Example 33

### CDC Activity of Anti-CD38 Constructs

The ability of anti-CD38 antibodies and anti-CD38 Fc constructs to promote cell killing of a CD38-expressing tumor cell lines (Daudi and Raji), was assessed by an in vitro CDC assay. Human serum complement was used as the complement source. RPMI-1640 media containing 0.1% BSA was used as a buffer for preparing cell suspensions, antibody, and serum dilutions. CD38 positive tumor cells were first washed in buffer and resuspended at a density of 10⁶ cells/ml. In a typical assay, 50 μl of antibody or anti-CD38 Fc construct, 50 μl of diluted complement (5× dilution), and 50 μl of a cell suspension (50,000 cells/well) were added to a flat-bottom tissue culture 96-well plate. The mixture was then incubated for 2 hours at 37° C. in a 5% CO2 incubator to facilitate complement-mediated cell lysis. Then, 50 μl of Alamar Blue was added to each well and incubated for 18 hours at 37° C. Fluorescence was read using a 96-well fluorometer with excitation at 530 nm and emission at 590 nm.

The assay was performed with Daudi cells and Raji cells in the presence of human or cyno serum complement to evaluate relative CDC mediated tumor cell lysis induced by either anti-CD38 mAb or anti-CD38 constructs. The result, presented in Table 13, are expressed in relative fluorescence units (RFU) that are proportional to the number of viable cells. The activity of the various mutants was examined by plotting the percent CDC activity against the log of Ab concentration (final concentration before the addition of Alamar Blue). The percent CDC activity was calculated as follows: % CDC activity=(RFU test—RFU background)×100 (RFU at total cell lysis—RFU background). Values represent mean ±SD from a representative experiment (from n=3 separate experiments). This study demonstrates that the anti-CD38 constructs exhibit greater efficacy (maximum tumor cell killing) and potency than anti-CD38 mAb in in anti-CD38 mAb-CDC sensitive cells (Daudi) as well as in anti-CD38 mAb-CDC resistant cells (Raji). Anti-CD38 mAb-sensitive or -resistant term refers to sensitivity or resistance towards anti-CD38 mAb mediated target cell lysis in cell based CDC assays.

TABLE 13
CDC activity of CD38 constructs in Daudi cell and Raji Cells

| | Daudi cells | | | Raji cells | |
|---|---|---|---|---|---|
| | | | EC50, fold- | | Max % lysis, |
| | Max % | EC50 | change vs. anti- | Max % | fold-change vs. |
| Treatment | lysis | nM | CD38 mAb | lysis | anti-CD38 mAb |
| anti-CD38 mAb | 100 | 0.7749 | 1.00 | 14.7 | 1.00 |
| Construct 13 | 100 | 0.0725 | 10.685 | 92.1 | 6.28 |
| S3Y-AA-CD38 | | | | | |
| Construct 7 | 100 | 0.1960 | 3.954 | 15.5 | 1.06 |
| S3I-AA-CD38 | | | | | |
| Construct 9 | 100 | 0.0372 | 20.831 | 39.9 | 2.72 |
| S3A-AA-CD38 | | | | | |
| Construct 19 | 100 | 0.0993 | 7.801 | 83.3 | 5.67 |
| S5X-AA-CD38 | | | | | |

¹All constructs included G20 (SEQ ID NO: 23) linkers unless otherwise noted.

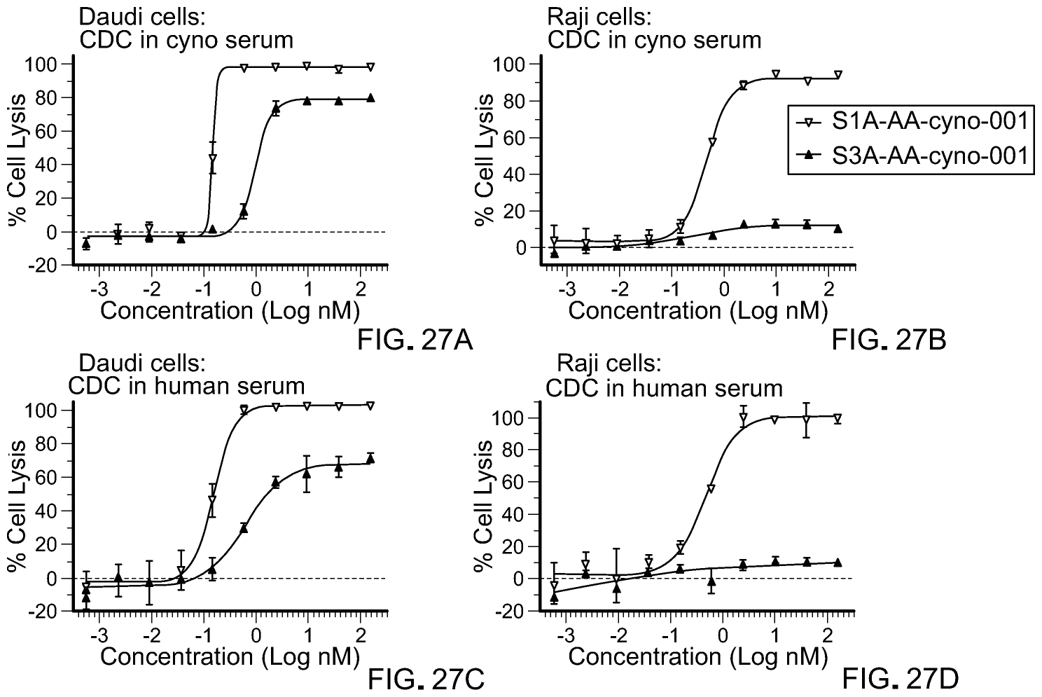

A Cynomolgus monkey CD38 cross-reactive anti-CD38 construct 13 (S3Y-AA-Cyno CD38) showed significantly high potency and efficacy in inducing CDC in both sensitive and resistant tumor cells than the corresponding mAb (S1A-AA-Cyno (anti-Cyno CD38 mAb). This assay was performed in a similar fashion as described above, but using Daudi tumor cells and monkey serum complement (FIG. 27, panel A), Raji tumor cells and monkey serum complement (FIG. 27, panel B), Daudi tumor cells and human serum complement (FIG. 27, panel C), Raji tumor cells and human serum complement (FIG. 27, panel D). The CDC activities of these constructs are presented in Table 14 show significant enhancement in efficacy and potency of S3Y-AA-Cyno CD38 over S1A-AA-Cyno (anti-Cyno CD38 mAb) in inducing CDC against Daudi and Raji cells.

TABLE 14
CDC activity of cynomolgus monkey CD38 reactive
constructs in Daudi cell and Raji Cells

| Treatment | Daudi cells | | | Raji cells | |
|---|---|---|---|---|---|
| (in the presence | | | EC50, fold- | | Max % lysis, |
| of cyno serum | Max % | EC50 | change vs. S1A- | Max % | fold-change vs. |
| complement) | lysis | nM | AA-Cyno | lysis | S1A-AA-Cyno |
| S1A-AA-Cyno | 78.62 | 0.9593 | | 12.76 | |
| Construct 13 | 97.84 | 0.1486 | 6.46 | 92.78 | 7.27 |
| S3Y-AA-Cyno | | | | | |
| Treatment | Max % | EC50 | EC50, fold- | Max % | Max % lysis, fold- |
| (in the presence of | lysis | nM | change vs. S1A- | lysis | change vs. S1A- |
| human serum | | | AA-Cyno | | AA-Cyno |
| complement) | | | | | |
| S1A-AA-Cyno | 67.96 | 0.6301 | | 12.7 | |
| Construct 13 | 102.4 | 0.1547 | 4.07 | 100.6 | 7.92 |
| S3Y-AA-Cyno | | | | | |

### Example 34

### Antibody-Dependent Cellular Phagocytosis (ADCP) activation by anti-CD38 Fc Constructs

Monocytes were isolated from human whole blood and allowed to differentiate into macrophages by treating them with human M-CSF and IL-10 in a 6-well plate. These adherent macrophages were then detached using chilled PBS +2 mM EDTA for subsequent seeding into assay wells. 2×10⁵ macrophages were seeded in a 96 well flat bottom plate in RPMI-1640 media containing 2% ultra-low FBS. Plates were briefly centrifuged and incubated for 1 hour at 37° C. to adhere macrophages to the bottom of the 96-well plate. Raji tumor cells were stained with Calcein-AM followed by addition on macrophage containing plate at an effector (macrophages): target (tumor cells) ratio of 3:1 in the presence of serial dilutions of anti-CD38 mAb or various anti-CD38 constructs. Plates were then incubated for 2 hours at 37° C. in a CO₂ incubator. Supernatants were collected in a V-bottom 96 well plate. Adherent cells were collected by detachment with chilled PBS containing 2 mM EDTA. Cells from supernatants and detached adherent cells were pooled together. These cells were then stained with anti-CD11 b APC and -CD19 BV421 antibodies by incubating with these antibodies for 1 hour at 4° C. The labeled cell suspensions were read on FACS Verse flow cytometer. Double positive macrophages (CD11b+/Calcein-AM+) that are negative for surface CD19 staining were considered as phagocytic events. The results are in Table 15 show superior potencies of anti-CD38 constructs in inducing phagocytosis of opsonized Raji cells by primary human macrophages.

TABLE 15
Potency of anti-CD38 Fc constructs in an ADCP assay

| Construct | | EC50 (nM) | | |
|---|---|---|---|---|
| Number¹ | n | Range | Mean | SD |
| anti-CD38 mAb | 2 | 0.14-0.08 | 0.11 | 0.045 |
| S3Y-AA-CD38 | 2 | 0.1-0.03 | 0.069 | 0.05 |
| Construct 13 | | | | |
| S3A-AA-CD38 | 2 | 0.034-0.028 | 0.031 | 0.004 |
| Construct 9 | | | | |
| S5X-AA-CD38 | 2 | 0.02-0.04 | 0.03 | 0.011 |
| Construct 19 | | | | |

¹All constructs included G20 (SEQ ID NO: 23) linkers unless otherwise noted.

### Example 35

### Antibody-Dependent Cell-Mediated Cytotoxicity (ADCC) Activation by Anti-CD38 Fc Constructs

Raji cells were suspended in RPMI media containing 10% ultralow IgG FBS at concentration of 5000 cells/50 μL media/well in a 96 well plate. Samples were then incubated for 15 minutes at 25° C. in with increasing concentrations of antibodies and constructs (10 uL/well). Primary human NK cells (effector cells) were added in effector to target ratio of 5:1. Effector and target cells mix were then incubated for 5 hours at 37° C. in a 5% CO₂ incubator. CytoTox Glo reagent (50 μL) was added and plates were incubated for 15 minutes at 25° C. to label dead cells. Samples were then read on Pherastar Luminometer to measure luminescence signal from the dead cells. Our results demonstrate 5-7× higher potency of S3Y (construct 13) molecule over the anti-CD38 mAb in inducing ADCC. As shown in Table 16, below, anti-CD38 Construct 13 demonstrated superior potency than anti-CD38 mAb in inducing primary human NK cell-mediated ADCC against Raji tumor cells. Target and effector cells were treated with drug molecules for 5 hours at 37C followed by detection of dead cells by CytoTox Glo reagent. Assay controls, Spontaneous Release Control (Target Cells Only); No Antibody Control; NK cells only+Antibody; IgGk Isotype Control

TABLE 16
Potency of anti-CD38 Fc constructs in an ADCC assay

| Construct | | EC50 (nM) | | |
|---|---|---|---|---|
| Number¹ | n | Range | Mean | SD |
| anti-CD38 mAb | 2 | 0.013-0.003 | 0.01567 | 0.007541 |
| S3Y-AA-CD38 | 2 | 0.0019-0.0006 | 0.0012 | 0.0009 |
| Construct 13 | | | | |
| G₂₀ (SEQ ID NO: | | | | |
| 23) linker | | | | |

### Example 36

### Tumor Cell Killing by Anti-CD38 Constructs in Human Whole Blood

Daudi cells were suspended in 50 μl of media (RPMI-1640+10% ULow IgG FBS) and seeded into each well of 96 well plate. 50 μL of whole human blood or ACK-lysed human whole blood cells (without serum and RBCs) were added to the tumor cell suspension. This was followed by addition of 50 μL of antibody and anti-CD38 construct dilutions (in RPMI-1640 media+10% FBS). Samples were mixed and then incubated for 4 hours at 37° C. in a CO₂ incubator. After the incubation, remaining live Daudi cells were assessed by adding 50 μL of freshly prepared luciferin solution (stock concentration, 50 mg/mL). Plate was then placed on a plate shaker for 5 minutes. Luminescence emitted from live Daudi-luciferase cells was read using Pherastar Luminometer.

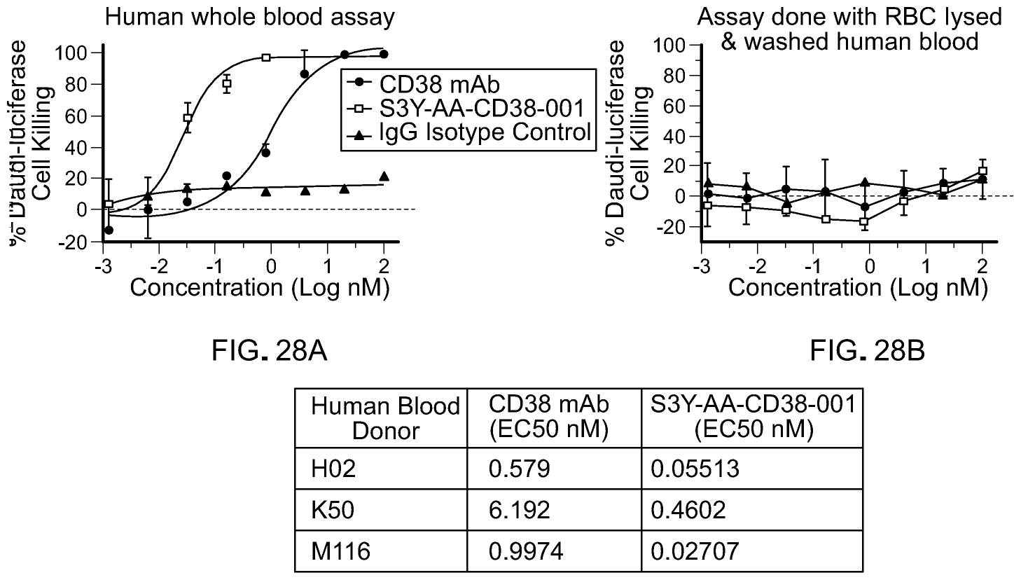

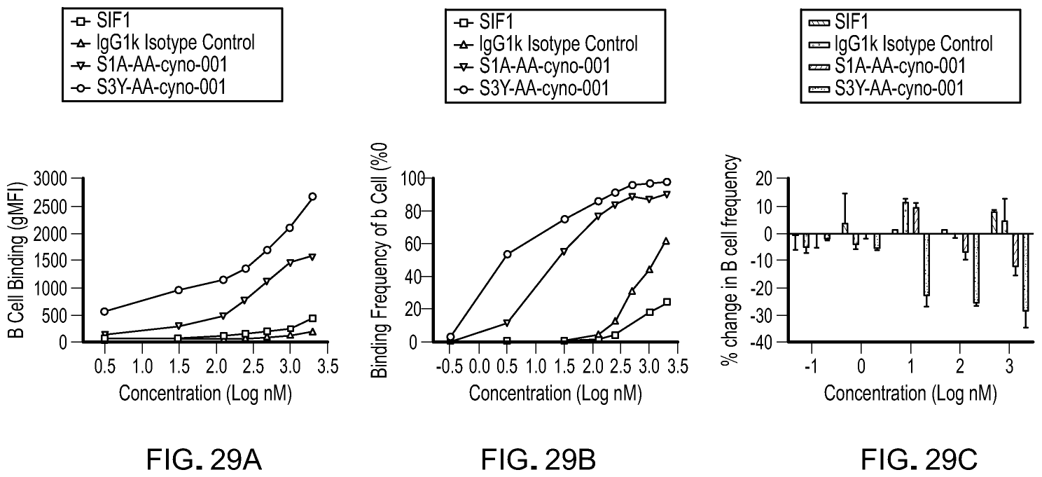

Results presented in FIG. 28 suggest that anti-CD38 construct 13 (S3Y-AA-CD38) is 10×-36× more potent than anti-CD38 mAb in target cell killing in whole human blood collected from 3 separate donors. However, with RBC lysed & washed whole blood no tumor cell depletion was observed with anti-CD38 mAb or anti-CD38 constructs. Replenishing RBC lysed & washed whole human blood cells with autologous serum prepared from the same donor restored tumor cell depletion, suggesting a role for serum proteins in facilitating anti-CD38 mAb and anti-CD38 construct-induced tumor cell killing in the whole blood.

### Example 37

### Depletion of Endogenous CD38 Expressing B Cells from Monkey Whole Blood

Cyno whole blood was mixed with serial dilutions of each VivoTag645-labeled molecules (SIF1, IgG isotype control, S1A-AA-Cyno-001 (anti-cyno CD38 mAb), anti-cyno CD38 construct 13 S3Y-AA-Cyno-001) separately along with cell surface marker antibody cocktail. Blood samples were then either incubated at 4° C. for 30 min to determine cell surface binding or separately incubated at 37° C. for 3 hours in a CO₂ incubator for determining effect of treatment on cell depletion. After these treatments, RBCs were lysed by mixing samples with cold ammonium chloride solution. Samples were then washed and re-suspended in buffer containing 1% paraformaldehyde and FACS analysis was performed the following day. CD38+ B cell population was assessed based on CD38-binding & binding-frequency data. Frequency of CD38+B cell type was measured to determine depletion due to treatment with construct molecule for 3 hours. B cell depletion was observed for anti-CD38 construct 13 (S3Y-AA-Cyno-001) at doses 10 nM (1 Log nM) and above, in a dose-dependent manner. Depletion with begins to appear at 100-1000 nM (2-3 Log nM). Greater depletion was observed with anti-cynoCD38 construct 13 (S3Y-AA-Cyno-001) compared to anti-cyno CD38 mAb (S1A-AA-Cyno-001).

### Example 38

### In Vivo Lymphoma Model

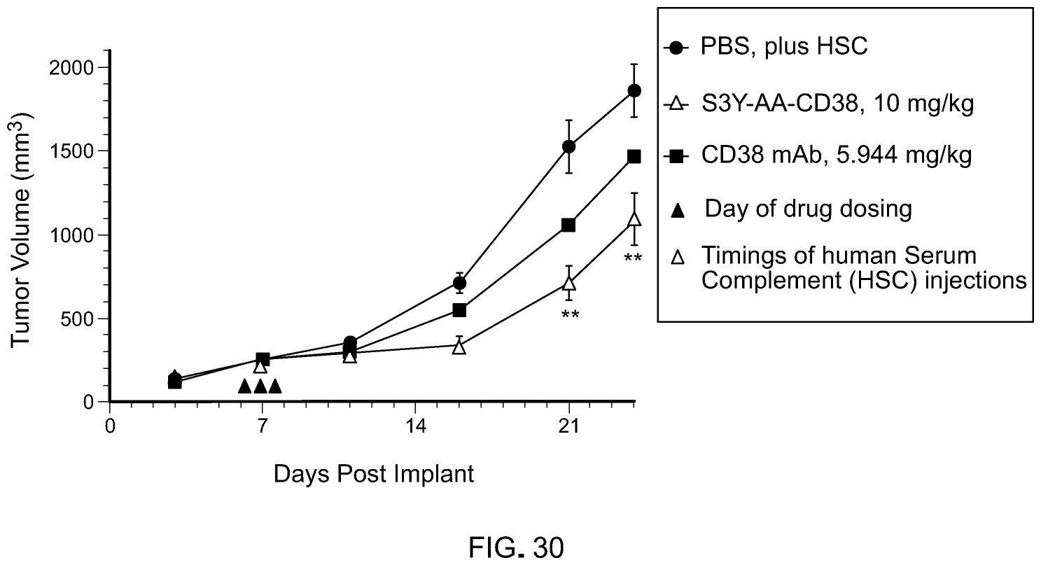

Effects of agents on disease progression and therapeutic response was evaluated in a subcutaneous tumor model for human lymphoma by tumor volume measurements. CB17-severe combined immunodeficiency (SCID) mice (female, 6-7 weeks old, average weight of 20 grams, strain 236 from Charles River Laboratories) were housed in Momenta animal care facility for 48 hours prior to use according to IACUC protocol. Water and food were provided ad libitum. All experiments were approved by the institutional animal ethics committee. Mice were checked daily for signs of discomfort and for general appearance. For subcutaneous tumor xenograft model, 5×10⁶ human Burkitt's lymphoma Raji cells suspended in high concentration Matrigel were injected subcutaneously into the right flank of mice. Tumor volume was measured twice weekly until tumors reach approximately 250 mm³ (approximately by day 6-7) at which time mice were assigned into treatment groups (8 mice/group). Mice in all 3 groups were injected intraperitoneally with 0.5 mL normal human serum complement a day before treatment, immediately prior to intravenous treatment injections (with PBS, anti-CD38 mAb, or S3Y-AA-CD38), and a day after treatment. Body weight and tumor volume was recorded twice weekly. Tumors were measured daily when volume approached 2000 mm³. All animals were observed daily; morbid animals were euthanized according to the IACUC protocol. The results shown in FIG. 30 suggest that the anti-CD38 construct 13 (S3Y-AA-CD38) is more efficacious than anti-CD38 mAb in this human lymphoma mouse model when the treatment was given in the presence of human serum complement.

### Example 39

### Fc Domains in Constructs Retain Similar Binding to Fc Gamma Receptors to that of Fc Domains in Antibodies

Anti-CD20 and anti-CD38 constructs were utilized to evaluate whether the various combinations of homodimerization mutations, heterodimerization mutations, polypeptide linkers, and Fab domains affected the binding to Fc gamma receptors. Surface Plasmon Resonance (SPR) was utilized to assess 1:1 binding with CD64 (Fc gamma receptor I). The constructs were captured on the chip surface, and binding to the soluble receptor was measured to ensure 1:1 binding. In this format, binding valency is the most sensitive readout to alterations in Fc function; kinetic and equilibrium constants are insensitive to alterations in a subset of Fc domains.

### Cell Culture

DNA sequences were optimized for expression in mammalian cells and cloned into the pcDNA3.4 mammalian expression vector. The DNA plasmid constructs were transfected via liposomes into human embryonic kidney (HEK) 293 cells. Antibodies were expressed from two different plasmids: one encoding the heavy chain and a second one encoding the light chain. SIF-bodies were expressed from three separate plasmids: in most cases one plasmid encoded the antibody light chain, one plasmid encoded the long Fc chain containing the CH1-VH FAB portion attached to the amino-terminal Fc and a third plasmid encoded the short Fc chain. The exceptions were the S3A and S3W Sif-Bodies. For S3W, one plasmid encoded the antibody light chain, the second plasmid encoded the long chain containing two Fc domains and a third plasmid encoded a single Fc chain containing a CH1-VH FAB portion. For S3A, one plasmid encoded the antibody light chain, a second plasmid encoded the long Fc chain containing the CH1-VH FAB portion attached to the amino-terminal Fc and one plasmid encoded the short Fc chain also containing a CH1-VH FAB portion.

### Protein Purification

The expressed proteins were purified from the cell culture supernatant by Protein A-based affinity column chromatography, using a Poros MabCapture A column. Captured SIF-Body constructs were washed with phosphate buffered saline (PBS, pH 7.0) after loading and further washed with intermediate wash buffer 50 mM citrate buffer (pH 5.5) to remove additional process related impurities. The bound SIF-Body material is eluted with 100 mM glycine, pH 3 and the eluate was quickly neutralized by the addition of 1 M TRIS pH 7.4 then centrifuged and sterile filtered through a 0.2 μm filter. The proteins were further fractionated by ion exchange chromatography using Poros XS resin. The column was pre-equilibrated with 50 mM MES, pH 6 (buffer A), and the sample was diluted (1:3) in the equilibration buffer for loading. The sample was eluted using a 12-15CV's linear gradient from 50 mM MES (100% A) to 400 mM sodium chloride, pH 6 (100%B) as the elution buffer. All fractions collected during elution were analyzed by analytical size exclusion chromatography (SEC) and target fractions were pooled to produce the purified SIF-Body material.

After ion-exchange, the pooled material was buffer exchanged into 1×-PBS buffer using a 30 kDa cutoff polyether sulfone (PES) membrane cartridge on a tangential flow filtration system. The samples were concentrated to approximately 10-15 mg/mL and sterile filtered through a 0.2 μm filter.

### Physicochemical Analyses

Analytical size exclusion chromatography (SEC) was used for the purity assessment on post Protein A, pooled ion-exchange fractions, and the final purified material.

The purified material was diluted to 1 mg/ml using 1×-PBS and analyzed on Agilent 1200 system with UV & FLD detector using Zenix SEC-300 (4.6×300 mm, 3μm, 300Å, Sepax, Cat. #213300-4630) as the analytical column.

The column was equilibrated with 100 mM sodium phosphate, 200 mM arginine, 300 mM sodium chloride pH=6.7 with 0.05% w/v sodium azide buffer at 0.3m1/min for an hour before the analysis. Injection amount approx. 10-15 ul, column temperature: 300C with UV detection at 280 nm and FLD with Excitation at 280 mm and Emission at 330 nm with total run time of 15 min.

The size purity results are shown in Table 17. All materials showed only low levels of high order species (HOS).

TABLE 17
Size purity of constructs used in Fc binding assays

| | | Size Purity by SEC | Size Purity by SEC |
|---|---|---|---|
| Construct | Antigen | (Target Species %) | (HOS %) |
| mAb | CD20 | 97.0% | 1.7% |
| Construct 13 (S3Y) | CD20 | 89.6% | 0.0% |
| Construct 7 (S3I) | CD20 | 89.0% | 1.7% |
| Construct 8 (S3W) | CD20 | 83.4% | 0.0% |
| Construct 9 (S3A) | CD20 | 92.4% | 1.5% |
| Construct 10 (S5I) | CD20 | 98.4% | 1.6% |
| (Construct 19 (S5X) | CD20 | 90.0% | 0.4% |
| Construct 16 (S5Y) | CD20 | 73.8% | 1.6% |
| mAb | CD20 | 97.0% | 1.7% |

### Binding Analyses

Binding experiments were performed on a Biacore T200 instrument (GE Healthcare) using a CM3 Series S sensor chip. For valency analyses of FcgR binding, native Protein A was immobilized via direct amine coupling. Ligands were diluted in running buffer and captured. A 6-point dilution series of human recombinant CD32a or CD64 (R&D Systems) was flowed over the captured ligands. The valency of each ligand was calculated as:

Ligand Valency=Rmax/[(MW analyte/MW ligand)*Ligand Capture Level].

The results from analyses of CD64 binding to anti-CD20 constructs are shown in Table 18. In all cases, the CD64 binding valency was equal to the number of Fc domains, indicating that all Fc domains were functional to bind CD64. A control compound identical in sequence to S3Y-AA-OBI and S3Y-AA-AVE, but lacking the Fab domains, bound CD64 comparably to those constructs, demonstrating that the inclusion of Fab domains did not alter the binding to Fc receptors.

TABLE 18
Valency of certain anti-CD20 constructs

| | | Number of Fc | CD64 Valency by |
|---|---|---|---|
| Construct | Antigen | Domains | SPR |
| mAb | CD20 | 1 | 1.5 |
| Construct 13 | CD20 | 3 | 3.4 |
| (S3Y) | | | |
| Construct 7 | CD20 | 3 | 3.0 |
| (S3I) | | | |
| Construct 8 | CD20 | 3 | 2.9 |
| (S3W) | | | |
| Construct 9 | CD20 | 3 | 3.1 |
| (S3A) | | | |
| Construct 10 | CD20 | 5 | 5.5 |
| (S5I) | | | |
| (Construct 19 | CD20 | 5 | 4.9 |
| (S5X) | | | |
| Construct 16 | CD20 | 5 | 5.5 |
| (S5Y) | | | |
| Control (S3Y) | No antigen binding | 3 | 3. |
| | domains | | |

### Example 40

### Constructs Bind More Avidly to Cell Surface Fc Gamma Receptors

Relative binding of constructs to cell surface CD32a was evaluated in a time-resolved fluorescence resonance energy transfer (TR-FRET) assay (CisBio) using anti-CD20 constructs. Assay reagents were prepared according to the manufacturer's instructions. A Freedom EVOware 150 automated liquid handler (Tecan) was used to generate a 10-point, 3-fold serial dilution series for each sample which were added to the cells bearing the labeled receptor. The labeled competitor antibody was then added and the plates incubated at room temperature. A PHERAstar fluorescent reader (BMG Labtech GmbH) was used to read assay plates at 665 and 620 nm. Log-transformed sample concentrations were plotted against corresponding HTRF signal ratios (665 nm/620 nm). A four-parameter non-linear regression analysis (least squares fit) was performed on the XY-plot to calculate EC50 of the unlabeled sample, with EC50 being inversely proportional to the sample's affinity for Fc gamma receptor. Measurements of competitive binding to CD32a determined by TR-FRET are summarized in Table 19. Increasing the number of Fc domains greatly increased the ability of constructs to compete with immunoglobulin for CD32a, as reflected by the decreased IC50 values. A control compound identical in sequence to S3Y-AA-OBI and S3Y-AA-AVE, but lacking the Fab domains, competed for cell surface CD32a comparably to those constructs, demonstrating that the inclusion of Fab domains did not alter the binding to Fc receptors.

TABLE 19
Fc receptor binding of certain anti-CD20 constructs

| | | FcγRIIIaV158 | FcγRIIaH131 | FcγRIIb |
|---|---|---|---|---|
| Construct | Antigen | IC50 (nM) | IC50 (nM) | IC50 (nM) |
| mAb | CD20 | 428 | 1273 | 3291 |
| Construct 13 | CD20 | 0.076 | 0.009 | 2.146 |
| (S3Y) | | | | |
| Construct 7 | CD20 | 0.230 | 0.014 | 29.220 |
| (S3I) | | | | |
| Construct 8 | CD20 | 0.476 | 0.026 | 34.925 |
| (S3W) | | | | |
| Construct 9 | CD20 | 0.539 | 0.018 | 17.361 |
| (S3A) | | | | |
| Construct 10 | CD20 | 0.045 | 0.002 | 4.427 |
| (S5I) | | | | |
| (Construct 15 | CD20 | 0.055 | 0.012 | 0.086 |
| (S5X) | | | | |
| Construct 1 | CD20 | 0.017 | 0.014 | 1.231 |
| (S5Y) | | | | |
| Control | No antigen | 0.097 | 0.025 | 3.297 |
| (S3Y) | binding | | | |
| | domains | | | |

### Example 41

### Antigen Binding is Preserved in Anti-CD38 Constructs

Antigen binding was evaluated using SPR. Recombinant, Histidine tagged, CD38 (9049-B7 R&D Systems) protein was captured on the sensor using a previously immobilized anti-6X His (SEQ ID NO: 38) antibody. Dilution series of the cognate antibodies and SIF-bodies were passed over the sensors, which were regenerated with a low pH glycine solution between analyte injections. Binding was calculated using a 1:1 Langmuir interaction model.

The binding of anti-CD38 constructs is shown in Table 20. All of the tested compounds were no less than 93% pure by SEC. Constructs had comparable antigen binding to that of the corresponding monoclonal antibody in an assay that favored 1:1 binding.

TABLE 20
CD38 binding to anti-CD38 constructs by SPR

| Construct | KD (nM) |
|---|---|
| mAb | 670 |
| S3Y | 703 |
| S3A | 757 |

Table 21 provides data on binding of anti-CD38 constructs in a separate study.

TABLE 21
Human CD38 binding by certain anti-CD38 constructs

| Construct | KD (nM) at 25 C. | KD (nM) at 37 C. |
|---|---|---|
| anti-CD38 mAb | 129 | 410 |
| S3Y-AA-CD38 | 142 | 661 |
| S3I-AA-CD38 | 132 | 442 |
| S5X-AA-CD38 | 166 | 553 |
| S3A-AA-CD38 | 126 | 410 |

### Example 42

### Anti-CD38 Fc Construct Exhibits Increased Cytolytic Activity Against Human Lymphoma Cells

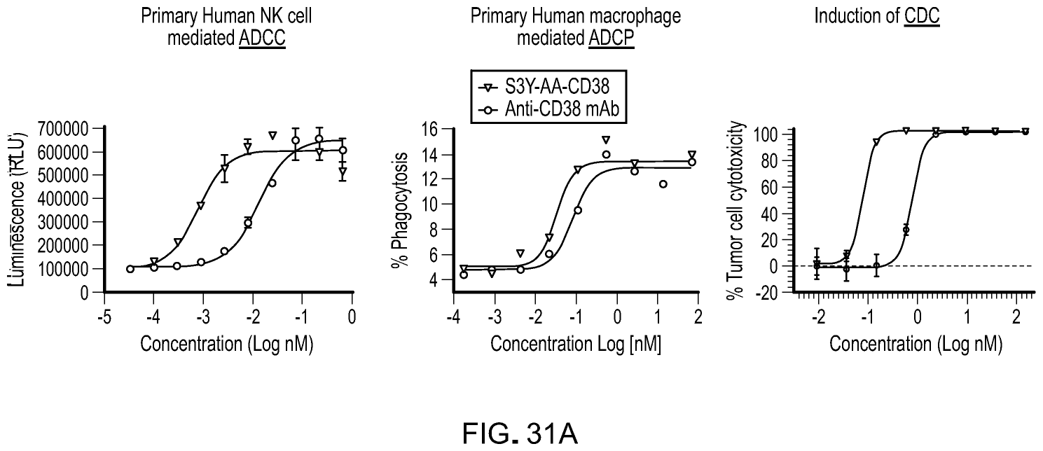

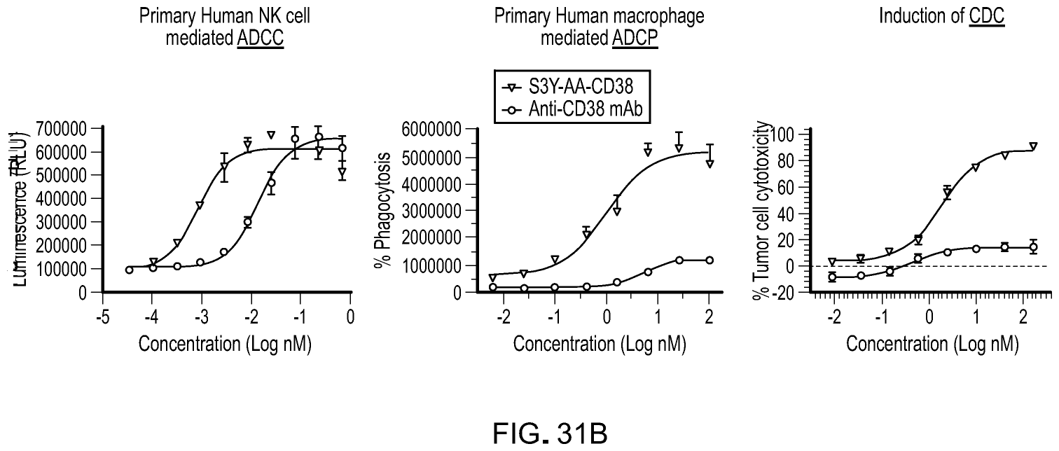

As shown in FIG. 31A and FIG. 31B, S3Y-AA-CD38 anti-CD38 Fc construct was more potent than an anti-CD38 mAb having the same Fabs in ADCC (primary human NK cell mediated), ADCP (primary human macrophage mediated) and CDC.

### Example 43

### Anti-CD38 Fc Construct Enhances Tumor Cell Depletion from Whole Blood with Better Potency and Efficacy that an Anti-CD38 Antibody

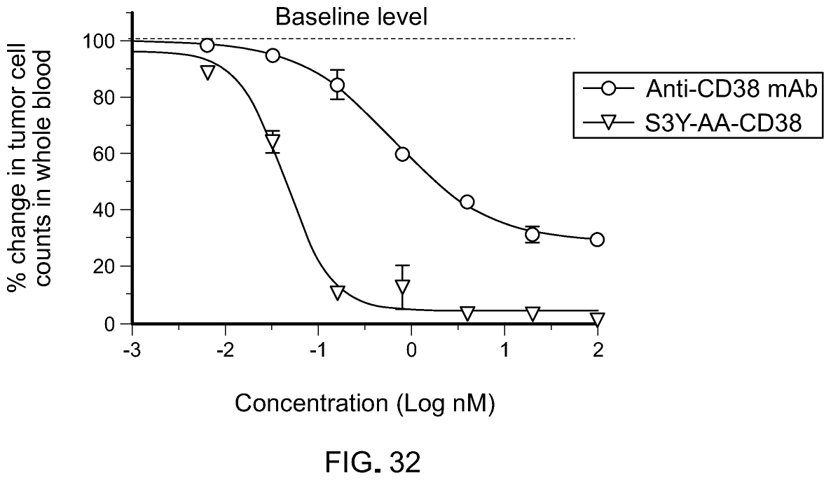

In this assay, the results of which are shown in FIG. 32, human whole blood was spiked with CFSE-labeled Daudi cells and then treated with S3Y-AA-CD38 or an anti-CD38 mAb having the same Fabs. The change in tumor cell population (CFSE+CD19+) in whole blood from baseline was measured by flow cytometry. The anti-CD38 Fc construct demonstrated 40-100 × higher potency than the anti-anti-CD38 mAb (n=5 donors).

### Example 44

### Anti-CD38 Fc Construct Mediates Cytotoxicity in Both High and Low CD38 Complement Inhibitory Protein Expressing Tumor Cell Lines

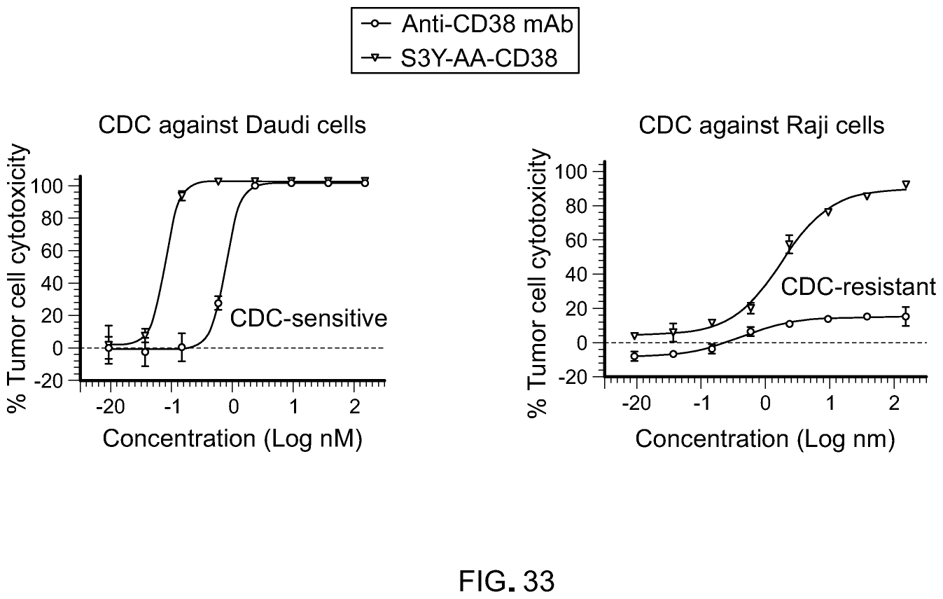

Response to the CD38-targeting antibody anti-CD38 mAb is correlated with CD38 expression levels on tumor cells. In addition, increased expression of complement inhibitory proteins (CD55, CD59) significantly decreases anti-CD38 mAb induced tumor cell depletion resulting in disease progression (Nijhof et al. (2016) Blood 128:959). As shown in FIG. 33, S3Y-AA-CD38 anti-CD38 Fc construct (inverted triangles) was more potent CDC activity than an anti-CD38 mAb having the same Fabs (circles) in both Daudi cells (relatively high CD38 expression and relatively low CD55 and CD59 expression) and, importantly, in Raji cells (relatively low CD38 expression and relatively high CD55 and CD59 expression).

### Example 45

### Anti-CD38 Fc Construct Mediates Cytotoxicity in Both High and Low CD38 Complement Inhibitory Protein Expressing Tumor Cell Lines

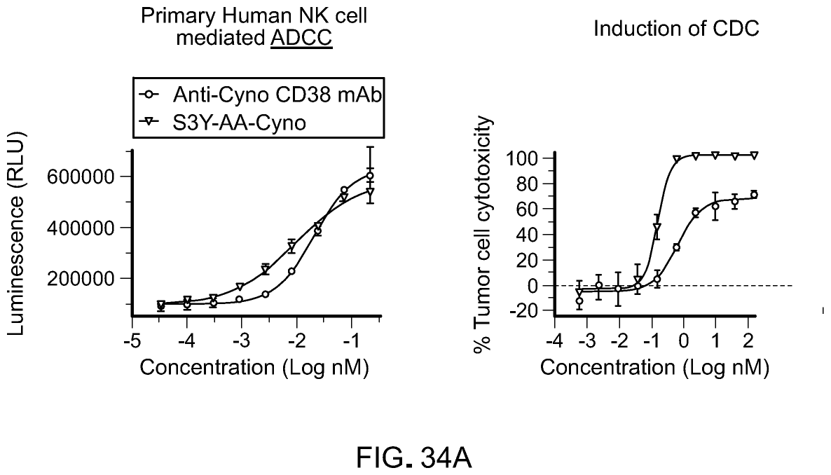

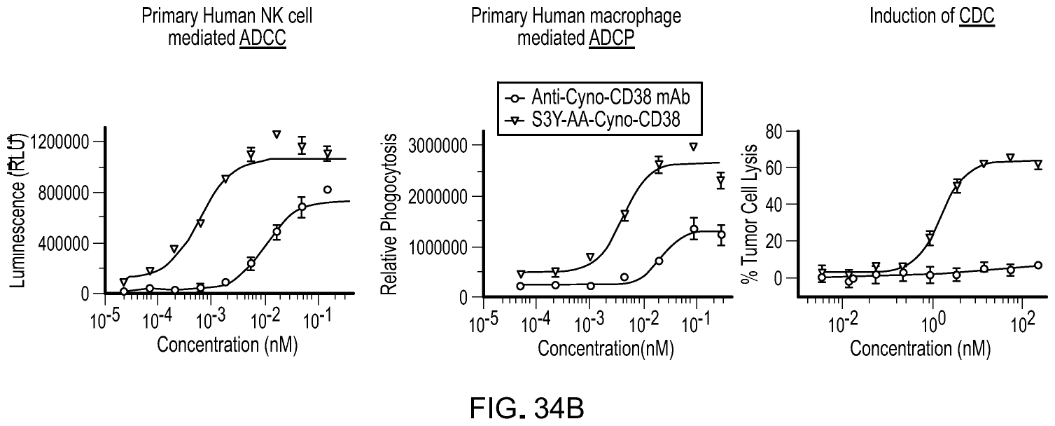

S3Y-AA-Cyno, the anti-cyno CD38 Fc construct described above in Table 6, which binds to both human and cynomolgus monkey CD38, and demonstrated improved ADCC, ADCP, and CDC activities against human lymphoma cells compared to a mAb with the same Fabs (anti-cyno CD38 mAb), as shown in FIG. 34A and 34B.

### Example 46

### Anti-Cyno CD38 Fc Construct Enhances Tumor Cell Depletion from Cynomolgus Monkey Whole Blood with Better Potency and Efficacy Than an Anti-Cyno CD38 Antibody

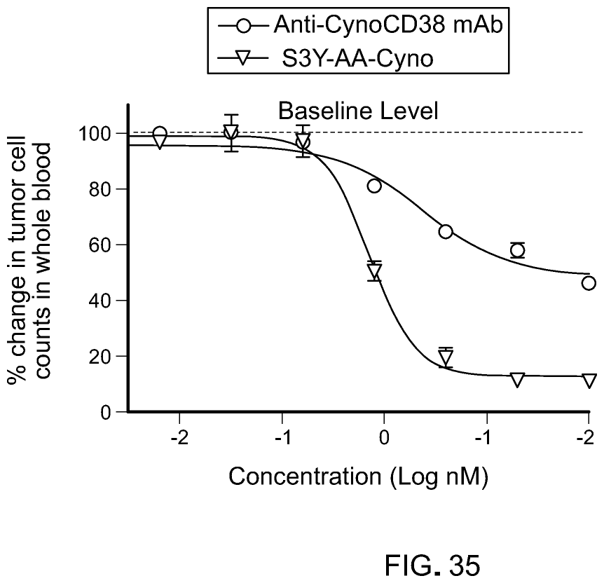

In this assay, the results of which are shown in FIG. 35, Cynomolgus monkey whole blood was spiked with CFSE-labeled Daudi cells and then treated with S3Y-AA-Cyno CD38 or an anti-CD38 mAb having the same Fabs. The change in tumor cell population (CFSE+CD19+) in whole blood from baseline was measured by flow cytometry. The anti-Cyno CD38 Fc construct demonstrated significantly higher potency than anti-cyno CD38 mAb (n=3).

### Example 47

### Anti-Cyno CD38 Fc Construct Demonstrates Superior CD38^{high} B Cell Depletion Than Anti-Cyno CD38 mAb in Cynomolgus Monkeys

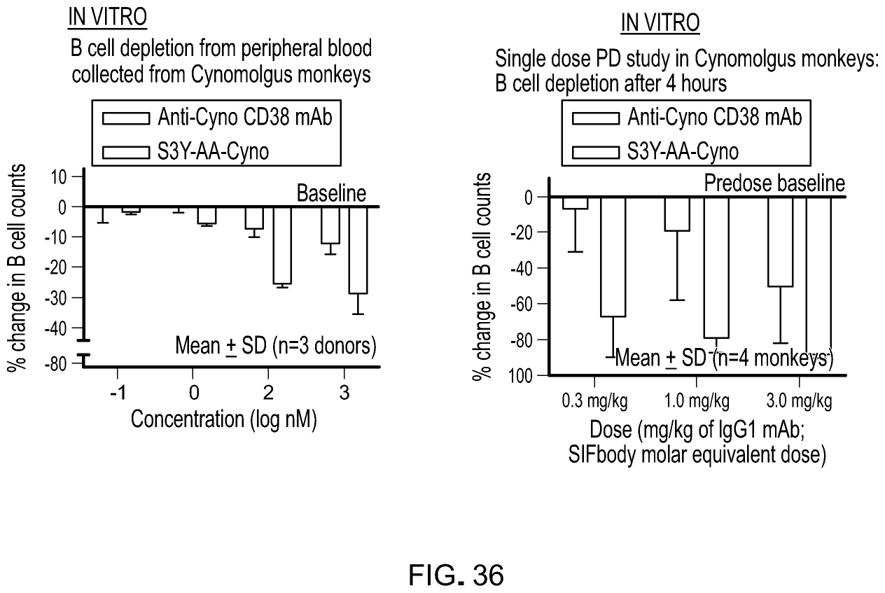

In this assay, the results of which are shown in FIG. 36, S3A-AA-Cyno was superior to the anti-Cyno CD38 mAb both in vitro, as measured by B cell depletion from peripheral blood collected from Cynomolgus monkeys (left panel), and in vivo, as measured in a single dose PD study in Cynomolgus monkeys that examined B cell depletion after 4 hours (right panel)

### Example 48

### Anti-CD38 Fc Construct Demonstrates Superior Depletion of Plasma Cells from a Multiple Myeloma Patient with a High Bone Marrow Plasma Cell Load

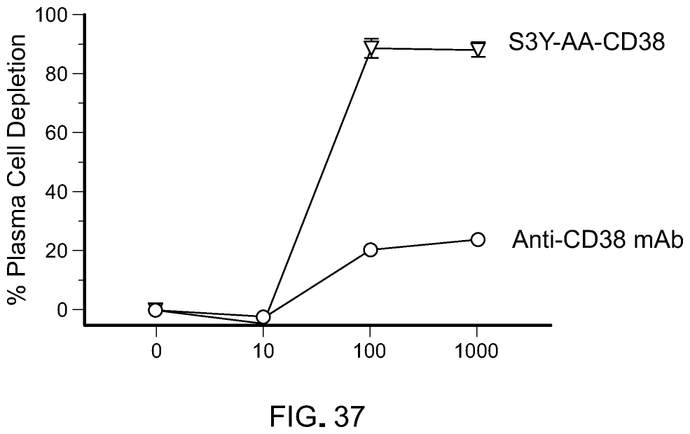

In this assay, the results of which are shown in FIG. 37, S3Y-AA-CD38 was superior to an anti-CD38 mAb having the same Fab sequences. Frozen bone marrow mononuclear cells (BM-MNCs) from multiple myeloma patient MM536, a relapsed patient with a BM plasma cell load of 82%, were obtained from the vendor. BM-MNCs were thawed and incubated in RPMI 1640+20% human serum complement (to allow for CDC mediated cell killing), in the presence or absence of varying concentrations of either anti-CD38 mAb or S3Y-AA-CD38 for 18 hours. The following day, samples were stained and analyzed by FACS to assess depletion of CD138+cells, used as a surrogate marker for CD38 expressing plasma/myeloma cells based on co-expression of the two markers determined by phenotyping of untreated cells. Cell depletion was determined using the viable CD138+ cell frequency out of total single cells, with all relative cell frequencies normalized to a baseline frequency observed in untreated controls (set to 0% change).

Depletion of CD138+ cells from total BM-MNCs of patient MM536 was observed following either S3Y-AA-CD38 or anti-CD38 mAb treatment at 100 or 1000 nM, while no depletion was observed for either treatment at a concentration of 10 nM. Saturating depletion of >90% of viable CD138+ cells at S3Y-AA-CD38 concentrations of 100 or 1000 nM was observed. Anti-CD38 mAb-mediated depletion was considerably lower than that observed by S3Y-AA-CD38, with maximum depletion levels 24% at concentrations of 100 and 1000 nM, which appear to be at or near saturating. Considering the high BM plasma cell frequency in patient MM536 (about 82%), these results may indicate potential for greater response using an anti-CD38 Fc construct in MM patients with high bone marrow plasma cell loads, which have been shown to have lower objective response rate to anti-CD38 mAb treatment in clinical applications.

### Example 48

### Anti-CD38 Fc Construct Demonstrates Enhanced Binding to Cell Surface FcγRs and Human Serum Complement

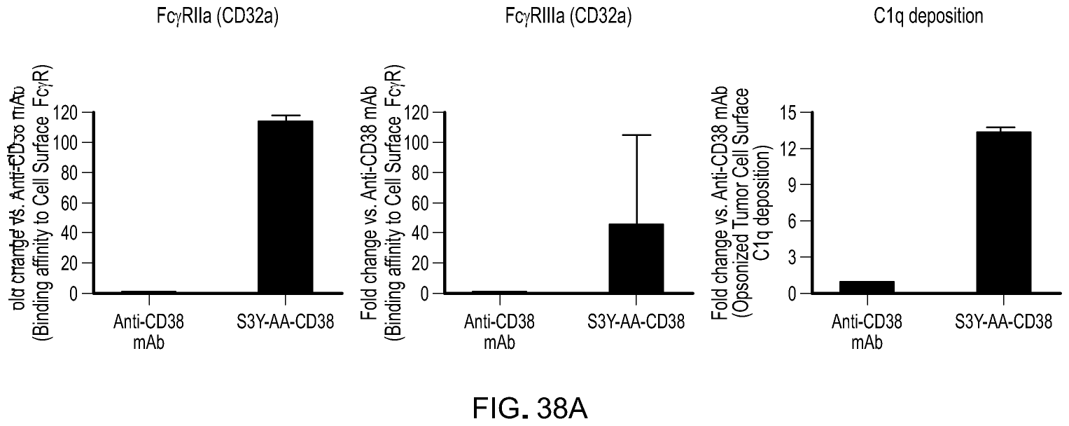

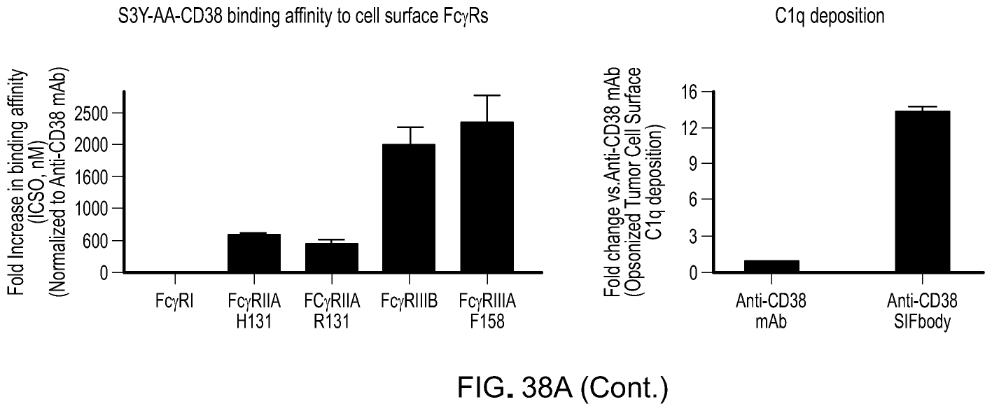

FIG. 38A depicts the results of a study showing that S3Y-AA-CD38 binding to FcgRIIa, FcgRIIIa and complement is at least 100-fold greater than an anti-CD38 mAb.

FIG. 38B depicts the results of a study showing >500× enhanced binding of S3Y-AA-CD38 to FcγRIIa, FcγRIIIa on immune cell surface and 12× enhanced C1q complement protein binding than an anti-CD38 mAb.

All publications, patents, and patent applications mentioned in this specification are incorporated herein by reference to the same extent as if each independent publication or patent application was specifically and individually indicated to be incorporated by reference.

While the disclosure has been described in connection with specific embodiments thereof, it will be understood that it is capable of further modifications and this application is intended to cover any variations, uses, or adaptations of the disclosure following, in general, the principles of the disclosure and including such departures from the disclosure that come within known or customary practice within the art to which the disclosure pertains and may be applied to the essential features hereinbefore set forth, and follows in the scope of the claims.

Other embodiments are within the claims.

1. An Fc-antigen binding domain construct comprising a CD38 binding domain and a first Fc domain joined to a second Fc domain by a linker, wherein each of the first and second Fc domains comprise either a heterodimerizing selectivity module or a homodimerizing selectivity module.

2. A polypeptide comprising an CD38 binding domain; a linker; a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain, wherein at least two Fc domain monomers comprise either a heterodimerizing selectivity module or a homodimerizing selectivity module.

3.-38. (canceled)

39. The polypeptide of claim 2, wherein each of the Fc domain monomers independently comprises the amino acid sequence of any of SEQ ID NOs:42, 43, 45, and 47 having up to 10 single amino acid substitutions.

40.-56. (canceled)

57. A polypeptide complex comprising two copies of the polypeptide of claim 2 joined by disulfide bonds between cysteine residues within the hinge of first or second IgG1 Fc domain monomers.

58. A polypeptide complex comprising a polypeptide of claim 2 joined to a second polypeptide comprising an IgG1 Fc domain monomer comprising a hinge domain, a CH2 domain and a CH3 domain, wherein the polypeptide and the second polypeptide are joined by disulfide bonds between cysteine residues within the hinge domain of the first, second or third IgG1 Fc domain monomer of the polypeptide and the hinge domain of the second polypeptide.

59.-61. (canceled)

62. A polypeptide comprising: an CD38 binding domain; a linker; a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain,
wherein at least one Fc domain monomer comprises one, two or three reverse charge amino acid mutations.

63.-98. (canceled)

99. The polypeptide of claim 62, wherein each of the Fc domain monomers independently comprises the amino acid sequence of any of SEQ ID NOs:42, 43, 45, and 47 having up to 10 single amino acid substitutions.

100.-116. (canceled)

117. A polypeptide complex comprising two copies of the polypeptide of claim 62 joined by disulfide bonds between cysteine residues within the hinge of first or second IgG1 Fc domain monomers.

118. A polypeptide complex comprising a polypeptide of claim 62 joined to a second polypeptide comprising and IgG1 Fc domain monomer comprising a hinge domain, a CH2 domain and a CH3 domain, wherein the polypeptide and the second polypeptide are joined by disulfide bonds between cysteine residues within the hinge domain of the first, second or third IgG1 Fc domain monomer of the polypeptide and the hinge domain of the second polypeptide.

119.-121. (canceled)

122. A polypeptide comprising: a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain,
wherein at least one Fc domain monomer comprises mutations forming an engineered protuberance.

123.-171. (canceled)

172. The polypeptide of claim 122, wherein each of the Fc domain monomers independently comprises the amino acid sequence of any of SEQ ID NOs:42, 43, 45, and 47 having up to 10 single amino acid substitutions.

173.-186. (canceled)

187. A nucleic acid molecule encoding the polypeptide of claim 2.

188. An expression vector comprising the nucleic acid molecule of claim 187.

189. A host cell comprising the nucleic acid molecule of claim 187.

190. A host cell comprising the expression vector of claim 188.

191. A method of producing the polypeptide of claim 2 comprising culturing the host cell of claim 189 under conditions to express the polypeptide.

192.-199. (canceled)

200. A pharmaceutical composition comprising the polypeptide of claim 2.

201.-318. (canceled)

319. An Fc-antigen binding domain construct comprising:
a) a first polypeptide comprising:
i) a first Fc domain monomer,
ii) a second Fc domain monomer
iii) a first CD38 heavy chain binding domain, and
iv) a linker joining the first and second Fc domain monomers;
b) a second polypeptide comprising:
i) a third Fc domain monomer,
ii) a fourth Fc domain monomer
iii) a second CD38 heavy chain binding domain and
iv) a linker joining the third and fourth Fc domain monomers;
c) a third polypeptide comprising a fifth Fc domain monomer;
d) a fourth polypeptide comprising a sixth Fc domain monomer;
e) a fifth polypeptide comprising a first CD38 light chain binding domain; and
f) a sixth polypeptide comprising a second CD38 light chain binding domain;
wherein the first and third Fc domain monomers together form a first Fc domain, the second and fifth Fc domain monomers together form a second Fc domain, the fourth and sixth Fc monomers together form a third Fc domain, the first CD38 heavy chain binding domain and first CD38 light chain binding domain together form a first Fab; and the second CD38 heavy chain binding domain and second CD38 light chain binding domain together form a second Fab.

320.-325. (canceled)

326. The Fc antigen domain construct of any of claims 319 claim 319, wherein each of the Fc domain monomers independently comprises the amino acid sequence of any of SEQ ID NOs:42, 43, 45, and 47 having up to 10, 8, 7, 6, 5, 4, 3, 2 or 1 single amino acid substitutions.

327.-331. (canceled)

332. An Fc-antigen binding domain construct comprising:
a) a first polypeptide comprising:
i) a first Fc domain monomer,
ii) a second Fc domain monomer
iii) a first CD38 heavy chain binding domain, and
iv) a linker joining the first and second Fc domain monomers;
b) a second polypeptide comprising:
i) a third Fc domain monomer,
ii) a fourth Fc domain monomer
iii) a second CD38 heavy chain binding domain and
iv) a linker joining the third and fourth Fc domain monomers;
c) a third polypeptide comprising a fifth Fc domain monomer and a first CD38 light chain binding domain; and
d) a fourth polypeptide comprising a sixth Fc domain monomer and a second CD38 light chain binding domain;
wherein the first and third Fc domain monomers together form a first Fc domain, the second and fifth Fc domain monomers together form a second Fc domain, the fourth and sixth Fc monomers together form a third Fc domain, the first CD38 heavy chain binding domain and first CD38 light chain binding domain together form a first Fab; and the second CD38 heavy chain binding domain and second CD38 light chain binding domain together form a second Fab.

333. An Fc-antigen binding domain construct, comprising:
a) a first polypeptide comprising:
i) a first Fc domain monomer,
ii) a second Fc domain monomer
iii) a first CD38 heavy chain binding domain ,and
iv) a linker joining the first and second Fc domain monomers;
b) a second polypeptide comprising:
i) a third Fc domain monomer,
ii) a fourth Fc domain monomer
iii) a second CD38 heavy chain binding domain and
iv) a linker joining the third and fourth Fc domain monomers;
c) a third polypeptide comprising a fifth Fc domain monomer;
d) a fourth polypeptide comprising a sixth Fc domain monomer;
e) a fifth polypeptide comprising a first CD38 light chain binding domain; and
f) a sixth polypeptide comprising a second CD38 light chain binding domain;
wherein the first and fifth Fc domain monomers together form a first Fc domain, the third and sixth Fc domain monomers together form an second Fc domain, the second and fourth Fc monomers together form a third Fc domain, the first CD38 heavy chain binding domain and first CD38 light chain binding domain together form a first Fab; and the second CD38 heavy chain binding domain and second CD38 light chain binding domain together form a second Fab.

334.-339. (canceled)

340. The Fc antigen domain construct of claim 332, wherein each of the Fc domain monomers independently comprises the amino acid sequence of any of SEQ ID NOs:42, 43, 45, and 47 having up to 10, 8, 7, 6, 5, 4, 3, 2 or 1 single amino acid substitutions.

341.-375. (canceled)

376. An Fc-antigen binding domain construct, comprising:
a) a first polypeptide comprising:
i) a first Fc domain monomer,
ii) a second Fc domain monomer,
iii) a linker joining the first and second Fc domain monomers, and
b) a second polypeptide comprising:
i) a third Fc domain monomer,
ii) a fourth Fc domain monomer
iii) a linker joining the third and fourth Fc domain monomers;
c) a third polypeptide comprising a fifth Fc domain monomer and a first CD38 heavy chain binding domain and;
d) a fourth polypeptide comprising a sixth Fc domain monomer a second CD38 heavy chain binding domain;
e) a fifth polypeptide comprising a first CD38 light chain binding domain; and
f) a sixth polypeptide comprising a second CD38 light chain binding domain;
wherein the first and fifth Fc domain monomers together form a first Fc domain, the third and sixth Fc domain monomers together form an second Fc domain, the second and fourth Fc domain monomers together form a third Fc domain, the first CD38 heavy chain binding domain and first CD38 light chain binding domain together form a first Fab; and the second CD38 heavy chain binding domain and second CD38 light chain binding domain together form a second Fab.

377.-382. (canceled)

383. The Fc antigen domain construct of claim 375, wherein each of the Fc domain monomers independently comprises the amino acid sequence of any of SEQ ID NOs:42, 43, 45, and 47 having up to 10, 8, 7, 6, 5, 4, 3, 2 or 1 single amino acid substitutions.

384.-388. (canceled)

389. An Fc-antigen binding domain construct, comprising:
a) a first polypeptide comprising:
i) a first Fc domain monomer,
ii) a second Fc domain monomer,
iii) a first CD38 heavy chain binding domain, and
iv) a linker joining the first and second Fc domain monomers,
b) a second polypeptide comprising:
i) a third Fc domain monomer,
ii) a fourth Fc domain monomer,
iii) a second CD38 heavy chain binding domain, and
iv) a linker joining the third and fourth Fc domain monomers,
c) a third polypeptide comprising a fifth Fc domain monomer and a third CD38 heavy chain binding domain;
d) a fourth polypeptide comprising a sixth Fc domain monomer and a fourth CD38 light chain binding domain;
e) a fifth polypeptide comprising a first CD38 light chain binding domain;
f) a sixth polypeptide comprising a second CD38 light chain binding domain;
g) a seventh polypeptide comprising a third CD38 light chain binding domain; and
h) an eighth polypeptide comprising a fourth CD38 light chain binding domain;
wherein the first and fifth Fc domain monomers together form a first Fc domain, the third and sixth Fc domain monomers together form an second Fc domain, the second and fourth Fc monomers together form a third Fc domain, the first CD38 light chain binding domain and third CD38 heavy chain binding domain together form a first Fab, the second CD38 light chain binding domain and fourth CD38 heavy chain binding domain together form a second Fab, the third CD38 light chain binding domain and first CD38 heavy chain binding domain together form a third Fab; and the fourth CD38 light chain binding domain and second CD38 heavy chain binding domain together form a second Fab.

390.-395. (canceled)

396. The Fc antigen domain construct of claim 389, wherein each of the Fc domain monomers independently comprises the amino acid sequence of any of SEQ ID NOs:42, 43, 45, and 47 having up to 10, 8, 7, 6, 5, 4, 3, 2 or 1 single amino acid substitutions.

397.-409. (canceled)

410. A method of treating cancer or autoimmune diseases comprising administering a composition comprising the construct of claim 1.

411.-415. (canceled)
